SEQUENCE LISTING (1434 sequences)

SEQ ID NO: 1 - DNA, 10 bp, Artificial Sequence
  Synthetic Polynucleotide
tatgtagatc

SEQ ID NO: 2 - DNA, 11 bp, Artificial Sequence
  Synthetic Polynucleotide
ttatctacata

SEQ ID NO: 3 - DNA, 21 bp, Artificial Sequence
  Synthetic Polynucleotide
gaatcggtcacagtacaaccg

SEQ ID NO: 4 - DNA, 23 bp, Artificial Sequence
  Synthetic Polynucleotide
ttcggttgtactgtgaccgattc

SEQ ID NO: 5 - DNA, 46 bp, Artificial Sequence
  Synthetic Polynucleotide
tgaggccaacgctcatggacgtactatggtttttacagcctccgga

SEQ ID NO: 6 - DNA, 38 bp, Artificial Sequence
  Synthetic Polynucleotide
acgtattacgccaccaaacatcccttagccagcgaaag

SEQ ID NO: 7 - DNA, 35 bp, Artificial Sequence
  Synthetic Polynucleotide
tcgattgcaacaggaaaaccgagtgtttttttggt

SEQ ID NO: 8 - DNA, 42 bp, Artificial Sequence
  Synthetic Polynucleotide
cactcggccttgctggtagcaatataattacatttatgtatt

SEQ ID NO: 9 - DNA, 35 bp, Artificial Sequence
  Synthetic Polynucleotide
aacataaatcaaaagaagcagcaagtttttctcca

SEQ ID NO: 10 - DNA, 42 bp, Artificial Sequence
  Synthetic Polynucleotide
attgtgccggcactgcggcacgcggtcatagctgtttccata

SEQ ID NO: 11 - DNA, 31 bp, Artificial Sequence
  Synthetic Polynucleotide
agtgacggattcgcctgtcgctggtaatcag

SEQ ID NO: 12 - DNA, 35 bp, Artificial Sequence
  Synthetic Polynucleotide
atgtgaatacacctttttgatcaatataatctttc

SEQ ID NO: 13 - DNA, 35 bp, Artificial Sequence
  Synthetic Polynucleotide
gaccatcgccattaaaaatgaaaatggtcagtaca

SEQ ID NO: 14 - DNA, 42 bp, Artificial Sequence
  Synthetic Polynucleotide
tgggcgcagaagatgaatttggattcctgattatcagaatta

SEQ ID NO: 15 - DNA, 42 bp, Artificial Sequence
  Synthetic Polynucleotide
accttcaatttagatttatggaagggagcggaattatcttat

SEQ ID NO: 16 - DNA, 35 bp, Artificial Sequence
  Synthetic Polynucleotide
cttgtggactcgtaacctttcctcgttagaaaggg

SEQ ID NO: 17 - DNA, 25 bp, Artificial Sequence
  Synthetic Polynucleotide
ccgaagagtcgcttaattgacgagc

SEQ ID NO: 18 - DNA, 42 bp, Artificial Sequence
  Synthetic Polynucleotide
cgagtaagaatttacatagaacaatattaccatcacgcccgt

SEQ ID NO: 19 - DNA, 46 bp, Artificial Sequence
  Synthetic Polynucleotide
cccttcagttaatggtctttgcgaatacctacattttgacgcttga

SEQ ID NO: 20 - DNA, 42 bp, Artificial Sequence
  Synthetic Polynucleotide
tatgccagctatacgagccggaagctgtgtggggggtttaat

SEQ ID NO: 21 - DNA, 42 bp, Artificial Sequence
  Synthetic Polynucleotide
gcacgttgcgtgagtgagctaactgggtaccagcctcccaaa

SEQ ID NO: 22 - DNA, 42 bp, Artificial Sequence
  Synthetic Polynucleotide
ctggagaaacaataacggtccgtggagctcgaattcgttgcc

SEQ ID NO: 23 - DNA, 32 bp, Artificial Sequence
  Synthetic Polynucleotide
atcaaacattagactttaccattaattgacag

SEQ ID NO: 24 - DNA, 35 bp, Artificial Sequence
  Synthetic Polynucleotide
atcatctaaagcatcaccctaaaaaatattttcaa

SEQ ID NO: 25 - DNA, 28 bp, Artificial Sequence
  Synthetic Polynucleotide
gtctgtaaagcctggggaatcatgtgcc

SEQ ID NO: 26 - DNA, 35 bp, Artificial Sequence
  Synthetic Polynucleotide
tttcctttgcccgaacgatcatattatacttaaat

SEQ ID NO: 27 - DNA, 28 bp, Artificial Sequence
  Synthetic Polynucleotide
tgtcagggtggcggtccacgctggatcc

SEQ ID NO: 28 - DNA, 28 bp, Artificial Sequence
  Synthetic Polynucleotide
agccagtgaggccctgagagagtttagc

SEQ ID NO: 29 - DNA, 28 bp, Artificial Sequence
  Synthetic Polynucleotide
tgtccaacgcataacggaacgtgccggc

SEQ ID NO: 30 - DNA, 35 bp, Artificial Sequence
  Synthetic Polynucleotide
atatcaggttatcaacaagagccagcagcaaatac

SEQ ID NO: 31 - DNA, 38 bp, Artificial Sequence
  Synthetic Polynucleotide
cttgctattacgcgaactgatagccttgctgaaccttg

SEQ ID NO: 32 - DNA, 35 bp, Artificial Sequence
  Synthetic Polynucleotide
cattgaaagcacgaaccaccagcacacgctggttg

SEQ ID NO: 33 - DNA, 42 bp, Artificial Sequence
  Synthetic Polynucleotide
ggtttagacaggaacggaacgtgcaccacacccgccgccact

SEQ ID NO: 34 - DNA, 42 bp, Artificial Sequence
  Synthetic Polynucleotide
catgaatcctgagaagtgttgcttgcgccgctacagggttcc

SEQ ID NO: 35 - DNA, 42 bp, Artificial Sequence
  Synthetic Polynucleotide
cagtgcatcattggaacagatagggttgagtccgcctgacgg

SEQ ID NO: 36 - DNA, 42 bp, Artificial Sequence
  Synthetic Polynucleotide
tccaaaagagtctgtccgccagcctctgaaatggattatacg

SEQ ID NO: 37 - DNA, 42 bp, Artificial Sequence
  Synthetic Polynucleotide
tccgggtaaacgctattaattaatctgattgtatacagcaat

SEQ ID NO: 38 - DNA, 42 bp, Artificial Sequence
  Synthetic Polynucleotide
ttgaaattaaccgttgtaatatcctggcagattcaccatctg

SEQ ID NO: 39 - DNA, 28 bp, Artificial Sequence
  Synthetic Polynucleotide
cttttaccagtataaagtcttcgcatcc

SEQ ID NO: 40 - DNA, 38 bp, Artificial Sequence
  Synthetic Polynucleotide
gcttcatatgcgttatatcacagtacatcggatcaaat

SEQ ID NO: 41 - DNA, 49 bp, Artificial Sequence
  Synthetic Polynucleotide
tgaaggtttctttgctcgtcattctcaacagtagggcttctgccacgcc

SEQ ID NO: 42 - DNA, 28 bp, Artificial Sequence
  Synthetic Polynucleotide
ttcgtagaacgtcagcgcgtctcgattg

SEQ ID NO: 43 - DNA, 35 bp, Artificial Sequence
  Synthetic Polynucleotide
cctgctttagtgatgaaggcaaaccaaaatccaca

SEQ ID NO: 44 - DNA, 42 bp, Artificial Sequence
  Synthetic Polynucleotide
cgtgttaaacgaacaatttcatttaaccttgcttctgtctga

SEQ ID NO: 45 - DNA, 42 bp, Artificial Sequence
  Synthetic Polynucleotide
aaggggaaacctgtcgttgggcgcgcactctacctgcacact

SEQ ID NO: 46 - DNA, 42 bp, Artificial Sequence
  Synthetic Polynucleotide
taactcactgcccgcttttttcacgcagtgttgcccccagca

SEQ ID NO: 47 - DNA, 42 bp, Artificial Sequence
  Synthetic Polynucleotide
acaattcgacaactcgttgatggcaattcaggatcccccaaa

SEQ ID NO: 48 - DNA, 42 bp, Artificial Sequence
  Synthetic Polynucleotide
aatgaggatttagaagtcctcaattaacagtcaagttagcgg

SEQ ID NO: 49 - DNA, 42 bp, Artificial Sequence
  Synthetic Polynucleotide
taaccgtcaatagataattggcaataacgtcggcgaatctga

SEQ ID NO: 50 - DNA, 42 bp, Artificial Sequence
  Synthetic Polynucleotide
gtctggtcagcagcaaccgcaaaaaaaagccgcacaggcggc

SEQ ID NO: 51 - DNA, 42 bp, Artificial Sequence
  Synthetic Polynucleotide
atcgacataaaaaaatcccgtagaatgccaacggcagcaccg

SEQ ID NO: 52 - DNA, 42 bp, Artificial Sequence
  Synthetic Polynucleotide
agcagttgggcggttgtgtactcggtggtgccatcccacgca

SEQ ID NO: 53 - DNA, 40 bp, Artificial Sequence
  Synthetic Polynucleotide
atttctgctcatttgccgccaccagcttacggctggaggt

SEQ ID NO: 54 - DNA, 28 bp, Artificial Sequence
  Synthetic Polynucleotide
gaactgaccaactttgaatcaagataat

SEQ ID NO: 55 - DNA, 39 bp, Artificial Sequence
  Synthetic Polynucleotide
catttcgagctaaatcggtgagcttaatttgaccaagag

SEQ ID NO: 56 - DNA, 42 bp, Artificial Sequence
  Synthetic Polynucleotide
ataagcagcgccgctttagaaacagcggatcggaagattatt

SEQ ID NO: 57 - DNA, 28 bp, Artificial Sequence
  Synthetic Polynucleotide
catctccttttgataagcgcgtttgtaa

SEQ ID NO: 58 - DNA, 21 bp, Artificial Sequence
  Synthetic Polynucleotide
gaattttgcggatggctagcc

SEQ ID NO: 59 - DNA, 35 bp, Artificial Sequence
  Synthetic Polynucleotide
ttggttttaaatatgcatataacacagatgaacgg

SEQ ID NO: 60 - DNA, 28 bp, Artificial Sequence
  Synthetic Polynucleotide
gtagcctcagagcataacaaatggaacg

SEQ ID NO: 61 - DNA, 42 bp, Artificial Sequence
  Synthetic Polynucleotide
aaatcatacaggcaagggcgagctcggcgaaacgtagtcagt

SEQ ID NO: 62 - DNA, 35 bp, Artificial Sequence
  Synthetic Polynucleotide
tcgtcagaagcaaagcgccccctcgtaataggcaa

SEQ ID NO: 63 - DNA, 35 bp, Artificial Sequence
  Synthetic Polynucleotide
ctttcaaaaagattaagcgtcatatggataggaat

SEQ ID NO: 64 - DNA, 31 bp, Artificial Sequence
  Synthetic Polynucleotide
cgataattaagttgggtcggctacttagata

SEQ ID NO: 65 - DNA, 35 bp, Artificial Sequence
  Synthetic Polynucleotide
atcgggttttgcgaaagttgtatcggcctcaaaac

SEQ ID NO: 66 - DNA, 35 bp, Artificial Sequence
  Synthetic Polynucleotide
ccgtaatgccggagagggcatgtcgtataagaaaa

SEQ ID NO: 67 - DNA, 42 bp, Artificial Sequence
  Synthetic Polynucleotide
agatgtaaaatcttcgccgcactctctgccagtttgagtgag

SEQ ID NO: 68 - DNA, 42 bp, Artificial Sequence
  Synthetic Polynucleotide
aggaagctttgaagggcgcaccgctgggcgcatcgtaagatt

SEQ ID NO: 69 - DNA, 35 bp, Artificial Sequence
  Synthetic Polynucleotide
gcacaaatataggtcattataatgctgtagcctgc

SEQ ID NO: 70 - DNA, 35 bp, Artificial Sequence
  Synthetic Polynucleotide
ctatcaaaaggaagcctttagcaaaattaagagct

SEQ ID NO: 71 - DNA, 42 bp, Artificial Sequence
  Synthetic Polynucleotide
cggttgataatcctgcggaatagatattcaaccgttctagct

SEQ ID NO: 72 - DNA, 42 bp, Artificial Sequence
  Synthetic Polynucleotide
aagtttaccaagaaagattcatcattaataaattgggcgttg

SEQ ID NO: 73 - DNA, 42 bp, Artificial Sequence
  Synthetic Polynucleotide
atgcaaatcatgacaagctaaagacgagtagatttagttgct

SEQ ID NO: 74 - DNA, 34 bp, Artificial Sequence
  Synthetic Polynucleotide
cactttaggaataccaccgttgggtttcaacgca

SEQ ID NO: 75 - DNA, 35 bp, Artificial Sequence
  Synthetic Polynucleotide
tactaatgcagatacatggctcatattacctgggg

SEQ ID NO: 76 - DNA, 39 bp, Artificial Sequence
  Synthetic Polynucleotide
gccagcgccaaaagcgtccaatgctgcaaggcgttattg

SEQ ID NO: 77 - DNA, 35 bp, Artificial Sequence
  Synthetic Polynucleotide
taagtaacaacccgtcgccgtgcacagccaggaga

SEQ ID NO: 78 - DNA, 42 bp, Artificial Sequence
  Synthetic Polynucleotide
ctgagaggggaaatgctttaaacaattatagagcttcattaa

SEQ ID NO: 79 - DNA, 28 bp, Artificial Sequence
  Synthetic Polynucleotide
acctttagacaatattcattgaatgatt

SEQ ID NO: 80 - DNA, 24 bp, Artificial Sequence
  Synthetic Polynucleotide
atgtaagaaaagccccatcctgta

SEQ ID NO: 81 - DNA, 42 bp, Artificial Sequence
  Synthetic Polynucleotide
acggaagattaatcatatgtacccgataaatgagacagccct

SEQ ID NO: 82 - DNA, 42 bp, Artificial Sequence
  Synthetic Polynucleotide
tgatataccagtcaggaattcaacgaggcatagtaagataaa

SEQ ID NO: 83 - DNA, 36 bp, Artificial Sequence
  Synthetic Polynucleotide
tccggatcggtttaaatttaatcgtaaaactagtag

SEQ ID NO: 84 - DNA, 28 bp, Artificial Sequence
  Synthetic Polynucleotide
ttcaagaggagttgattcccaatttcaa

SEQ ID NO: 85 - DNA, 35 bp, Artificial Sequence
  Synthetic Polynucleotide
tctacgtaacggtttaaaagaaaaatctacggttg

SEQ ID NO: 86 - DNA, 42 bp, Artificial Sequence
  Synthetic Polynucleotide
ccaaccatcaatatggatatgtaccaaaaacattatgatcaa

SEQ ID NO: 87 - DNA, 49 bp, Artificial Sequence
  Synthetic Polynucleotide
gtcgcatcggtcaataacctgtttcaataaaatacttttgcgggaggtg

SEQ ID NO: 88 - DNA, 35 bp, Artificial Sequence
  Synthetic Polynucleotide
gcctaaagattttttgagagatcttgaacgggtaa

SEQ ID NO: 89 - DNA, 35 bp, Artificial Sequence
  Synthetic Polynucleotide
gcttccattattgcaggcgctttctttaatccatt

SEQ ID NO: 90 - DNA, 35 bp, Artificial Sequence
  Synthetic Polynucleotide
agggtaatgcagtccagcatcagctatgcgagggg

SEQ ID NO: 91 - DNA, 42 bp, Artificial Sequence
  Synthetic Polynucleotide
ctcttttcatttggggccaaagaattatttcaacgcaagtgt

SEQ ID NO: 92 - DNA, 49 bp, Artificial Sequence
  Synthetic Polynucleotide
cggatcataagggaaccgaactttatccgccgggcgcgttgagataaag

SEQ ID NO: 93 - DNA, 36 bp, Artificial Sequence
  Synthetic Polynucleotide
ctcattcatgaggaagttttgaggaaaccggaaaga

SEQ ID NO: 94 - DNA, 28 bp, Artificial Sequence
  Synthetic Polynucleotide
tcaaacgggtaaaatacgtagcaaaacg

SEQ ID NO: 95 - DNA, 35 bp, Artificial Sequence
  Synthetic Polynucleotide
ttacagggagttaaaggaaagacaacgacgtaagg

SEQ ID NO: 96 - DNA, 41 bp, Artificial Sequence
  Synthetic Polynucleotide
cgctgcgggatccagcgccatgttctctcacggaaaaactt

SEQ ID NO: 97 - DNA, 35 bp, Artificial Sequence
  Synthetic Polynucleotide
agatatcataaccctcgttttgccctcattcgacc

SEQ ID NO: 98 - DNA, 42 bp, Artificial Sequence
  Synthetic Polynucleotide
atcaacattaaatggggacgacgacattaagaactaactttc

SEQ ID NO: 99 - DNA, 35 bp, Artificial Sequence
  Synthetic Polynucleotide
cgattcgcgtctggcctaaaacagccagctgccca

SEQ ID NO: 100 - DNA, 42 bp, Artificial Sequence
  Synthetic Polynucleotide
ctctaggaacgccatcacaaatatgcgggcccgacggccacc

SEQ ID NO: 101 - DNA, 42 bp, Artificial Sequence
  Synthetic Polynucleotide
actacgaaggcaccaacctaatattcggtcgctgaggcttgc

SEQ ID NO: 102 - DNA, 42 bp, Artificial Sequence
  Synthetic Polynucleotide
atcgcccacgcataaccgataaacgaaagaggcaaaagaata

SEQ ID NO: 103 - DNA, 42 bp, Artificial Sequence
  Synthetic Polynucleotide
gcgccgacaatgacaacaacccactaaaacactcatctttga

SEQ ID NO: 104 - DNA, 40 bp, Artificial Sequence
  Synthetic Polynucleotide
acagcttgataccgatagttcccccagcgattataccaag

SEQ ID NO: 105 - DNA, 28 bp, Artificial Sequence
  Synthetic Polynucleotide
tataataagagaatataatgttcaagca

SEQ ID NO: 106 - DNA, 22 bp, Artificial Sequence
  Synthetic Polynucleotide
ggtttaccaaggccggaaactg

SEQ ID NO: 107 - DNA, 42 bp, Artificial Sequence
  Synthetic Polynucleotide
ttctaactataacctccgctttcgaggtgaacgccaccaact

SEQ ID NO: 108 - DNA, 35 bp, Artificial Sequence
  Synthetic Polynucleotide
ttaccgaggaaacgcaaatgaaatgctaatgtcct

SEQ ID NO: 109 - DNA, 21 bp, Artificial Sequence
  Synthetic Polynucleotide
gacggaatacccaaaagcaat

SEQ ID NO: 110 - DNA, 34 bp, Artificial Sequence
  Synthetic Polynucleotide
gcatgatagaaaaagaacgcttcatctagatttg

SEQ ID NO: 111 - DNA, 35 bp, Artificial Sequence
  Synthetic Polynucleotide
aaagcaaacgtagaaaaacgcaaagacaaaaaggc

SEQ ID NO: 112 - DNA, 45 bp, Artificial Sequence
  Synthetic Polynucleotide
gcaaccattaccattagcagcgccgcaaatcaatggttacgcgaa

SEQ ID NO: 113 - DNA, 42 bp, Artificial Sequence
  Synthetic Polynucleotide
gcgttgagccatttggggggaaggacaactaaaggatgtctg

SEQ ID NO: 114 - DNA, 35 bp, Artificial Sequence
  Synthetic Polynucleotide
atataatatcagagagaaataacacccaatcaatt

SEQ ID NO: 115 - DNA, 35 bp, Artificial Sequence
  Synthetic Polynucleotide
gcacaagaattgagttaaatagcattttttgtgct

SEQ ID NO: 116 - DNA, 31 bp, Artificial Sequence
  Synthetic Polynucleotide
aatttttagcgtaacgaaagacaattcatat

SEQ ID NO: 117 - DNA, 32 bp, Artificial Sequence
  Synthetic Polynucleotide
ggaacccaacgtcaccaatgaaaccatcccag

SEQ ID NO: 118 - DNA, 35 bp, Artificial Sequence
  Synthetic Polynucleotide
agcttttgtctagcattacgaggtttagtactttc

SEQ ID NO: 119 - DNA, 35 bp, Artificial Sequence
  Synthetic Polynucleotide
atcgaaccgccaccctctattcacaccgttccagt

SEQ ID NO: 120 - DNA, 42 bp, Artificial Sequence
  Synthetic Polynucleotide
aattagtaaacagtacactcagaacggaataggtgtatatta

SEQ ID NO: 121 - DNA, 42 bp, Artificial Sequence
  Synthetic Polynucleotide
taggggatttcgtaacaaccgccaagggttgatataagaaga

SEQ ID NO: 122 - DNA, 35 bp, Artificial Sequence
  Synthetic Polynucleotide
ccaagaaacataataactccttattacgcagagtt

SEQ ID NO: 123 - DNA, 35 bp, Artificial Sequence
  Synthetic Polynucleotide
ccacatctttagcgacagccagcaaaatcacgaca

SEQ ID NO: 124 - DNA, 42 bp, Artificial Sequence
  Synthetic Polynucleotide
tcattaaagccaaaaaatgaaagcgcctccctcagagccgcc

SEQ ID NO: 125 - DNA, 42 bp, Artificial Sequence
  Synthetic Polynucleotide
acaaacgctagaacgcgaggcgttaagcaaagtctttctccg

SEQ ID NO: 126 - DNA, 42 bp, Artificial Sequence
  Synthetic Polynucleotide
taaagataagcagaacgctttttctttgtcacaatcaattaa

SEQ ID NO: 127 - DNA, 21 bp, Artificial Sequence
  Synthetic Polynucleotide
ataacgattggccttgaagag

SEQ ID NO: 128 - DNA, 35 bp, Artificial Sequence
  Synthetic Polynucleotide
ttaacctcccgacttgcatcattaaacgggtgcct

SEQ ID NO: 129 - DNA, 35 bp, Artificial Sequence
  Synthetic Polynucleotide
atttttgaagccttaaagtttttacgcactcacaa

SEQ ID NO: 130 - DNA, 39 bp, Artificial Sequence
  Synthetic Polynucleotide
cctataagattagttttaacgcagccctcatagatcaag

SEQ ID NO: 131 - DNA, 45 bp, Artificial Sequence
  Synthetic Polynucleotide
taaggctgagactcctctatagccccgccactcagcttggcttag

SEQ ID NO: 132 - DNA, 28 bp, Artificial Sequence
  Synthetic Polynucleotide
gaattccaagccgcgcccaatagcttag

SEQ ID NO: 133 - DNA, 42 bp, Artificial Sequence
  Synthetic Polynucleotide
acatgaatttaaacaaataaatccaccctcaaccggaagata

SEQ ID NO: 134 - DNA, 49 bp, Artificial Sequence
  Synthetic Polynucleotide
tcacaagaaatatttattaaaaacagggaagtgagcgcgctatctaagg

SEQ ID NO: 135 - DNA, 42 bp, Artificial Sequence
  Synthetic Polynucleotide
tacttttcatcgtaggagggaggtttgcacccagctaccaaa

SEQ ID NO: 136 - DNA, 28 bp, Artificial Sequence
  Synthetic Polynucleotide
aacaagtaccgacaccacggaatatatg

SEQ ID NO: 137 - DNA, 49 bp, Artificial Sequence
  Synthetic Polynucleotide
ttctgctgataaagacaaaagggccagtagcgcaccgtaatcagttcat

SEQ ID NO: 138 - DNA, 42 bp, Artificial Sequence
  Synthetic Polynucleotide
tatcgtttgcccaccctcagagccaggtcagcatggctgagt

SEQ ID NO: 139 - DNA, 42 bp, Artificial Sequence
  Synthetic Polynucleotide
ataaaccgattgagggaaattagagaatcaagtttgccttat

SEQ ID NO: 140 - DNA, 46 bp, Artificial Sequence
  Synthetic Polynucleotide
gtattgcgaataatattgtatcggtttacctcagactgagttcgtc

SEQ ID NO: 141 - DNA, 42 bp, Artificial Sequence
  Synthetic Polynucleotide
cgaggcattttcgagccagtaaataaattgtgtcgaaactta

SEQ ID NO: 142 - DNA, 35 bp, Artificial Sequence
  Synthetic Polynucleotide
gatatattttagttaatgagaaaacgcctgtaaga

SEQ ID NO: 143 - DNA, 46 bp, Artificial Sequence
  Synthetic Polynucleotide
tatcatcattaaaccaacaatgaaacgagcctttacagagagtaac

SEQ ID NO: 144 - DNA, 38 bp, Artificial Sequence
  Synthetic Polynucleotide
cggtctgacctaaatttcaatcgctctaaagcaccacc

SEQ ID NO: 145 - DNA, 42 bp, Artificial Sequence
  Synthetic Polynucleotide
acaaagtatcgagaccacagatcgaatggaaagcgttcggaa

SEQ ID NO: 146 - DNA, 35 bp, Artificial Sequence
  Synthetic Polynucleotide
ttatagactacctttttatgtaaacagacgtcaaa

SEQ ID NO: 147 - DNA, 42 bp, Artificial Sequence
  Synthetic Polynucleotide
caccgtactcagaagcaagcctctattctgaaacatgaaagt

SEQ ID NO: 148 - DNA, 35 bp, Artificial Sequence
  Synthetic Polynucleotide
cgatcctgaatcttaccgccatataataataaaac

SEQ ID NO: 149 - DNA, 35 bp, Artificial Sequence
  Synthetic Polynucleotide
agatgccccctgcctatcagtctcacgcctggtct

SEQ ID NO: 150 - DNA, 42 bp, Artificial Sequence
  Synthetic Polynucleotide
gaaagtgcccgtataaacagtaagtcgtcactgaatttggtt

SEQ ID NO: 151 - DNA, 42 bp, Artificial Sequence
  Synthetic Polynucleotide
gaaataccgaccgtgtgataatatcaaaatcataggtctgag

SEQ ID NO: 152 - DNA, 42 bp, Artificial Sequence
  Synthetic Polynucleotide
gagaagagtcaatagtgaattataaggcgttaaataagaata

SEQ ID NO: 153 - DNA, 42 bp, Artificial Sequence
  Synthetic Polynucleotide
gatagcttagattaagacgctaacaccggaatcataattact

SEQ ID NO: 154 - DNA, 40 bp, Artificial Sequence
  Synthetic Polynucleotide
agaatccttgaaaacatagcagaaaaagcctgtttagtat

SEQ ID NO: 155 - DNA, 42 bp, Artificial Sequence
  Synthetic Polynucleotide
aaaattagagttttaaaagtttgaaccagaaggttagaagtg

SEQ ID NO: 156 - DNA, 35 bp, Artificial Sequence
  Synthetic Polynucleotide
agggcctgcaacagtgcgaagatagaaccctgtca

SEQ ID NO: 157 - DNA, 32 bp, Artificial Sequence
  Synthetic Polynucleotide
ctaatagggaattgaattgcgacctgagacaa

SEQ ID NO: 158 - DNA, 28 bp, Artificial Sequence
  Synthetic Polynucleotide
aatgaattaccttttttcaagaaacaaa

SEQ ID NO: 159 - DNA, 42 bp, Artificial Sequence
  Synthetic Polynucleotide
acgtaaccaacgtgggaacaaacggtgtagattctggtggga

SEQ ID NO: 160 - DNA, 35 bp, Artificial Sequence
  Synthetic Polynucleotide
ttaaacaagagaatcgaacaaagggagtaatggat

SEQ ID NO: 161 - DNA, 32 bp, Artificial Sequence
  Synthetic Polynucleotide
catttttttaatatctgttggcagaggtaaac

SEQ ID NO: 162 - DNA, 28 bp, Artificial Sequence
  Synthetic Polynucleotide
tagtaccagtcccggaatcaccggggag

SEQ ID NO: 163 - DNA, 35 bp, Artificial Sequence
  Synthetic Polynucleotide
aggcaggaggttgaggcgccaccaagcccccttta

SEQ ID NO: 164 - DNA, 35 bp, Artificial Sequence
  Synthetic Polynucleotide
aacggattaggattagccgtcgagccctcaggcct

SEQ ID NO: 165 - DNA, 32 bp, Artificial Sequence
  Synthetic Polynucleotide
gtgcctttttgatgcatgtactgctaaagaaa

SEQ ID NO: 166 - DNA, 28 bp, Artificial Sequence
  Synthetic Polynucleotide
ttaaattttttcacgttgagaatacaac

SEQ ID NO: 167 - DNA, 30 bp, Artificial Sequence
  Synthetic Polynucleotide
gagtagaagaactaataacatcacttgcgc

SEQ ID NO: 168 - DNA, 21 bp, Artificial Sequence
  Synthetic Polynucleotide
tctggccaacagatgatgagc

SEQ ID NO: 169 - DNA, 24 bp, Artificial Sequence
  Synthetic Polynucleotide
tattaacaccttatctaaaataat

SEQ ID NO: 170 - DNA, 21 bp, Artificial Sequence
  Synthetic Polynucleotide
tttaggagcatatcattttct

SEQ ID NO: 171 - DNA, 30 bp, Artificial Sequence
  Synthetic Polynucleotide
acgtaaaacagaaatatcaaaattatttaa

SEQ ID NO: 172 - DNA, 31 bp, Artificial Sequence
  Synthetic Polynucleotide
agaagagataaaacagaggtgaggcggtcag

SEQ ID NO: 173 - DNA, 45 bp, Artificial Sequence
  Synthetic Polynucleotide
aatcttctttgattagtcaaactagaccagtaataaaagggactc

SEQ ID NO: 174 - DNA, 21 bp, Artificial Sequence
  Synthetic Polynucleotide
caaacataatggaaacagtac

SEQ ID NO: 175 - DNA, 35 bp, Artificial Sequence
  Synthetic Polynucleotide
ataaatcaatatatgtgacctaccataaagaagga

SEQ ID NO: 176 - DNA, 28 bp, Artificial Sequence
  Synthetic Polynucleotide
ggaacaaagaaaccgtaacatctaacaa

SEQ ID NO: 177 - DNA, 30 bp, Artificial Sequence
  Synthetic Polynucleotide
tagcattaacatcaattctactaatagtgg

SEQ ID NO: 178 - DNA, 21 bp, Artificial Sequence
  Synthetic Polynucleotide
ttttaaatgcccacgggaaat

SEQ ID NO: 179 - DNA, 24 bp, Artificial Sequence
  Synthetic Polynucleotide
gtctggagcaaaattcgcattata

SEQ ID NO: 180 - DNA, 21 bp, Artificial Sequence
  Synthetic Polynucleotide
tttttgttaagaccgtaatag

SEQ ID NO: 181 - DNA, 30 bp, Artificial Sequence
  Synthetic Polynucleotide
tcgccattcaggcaccaggcaaagcgcccg

SEQ ID NO: 182 - DNA, 31 bp, Artificial Sequence
  Synthetic Polynucleotide
ccgaatgcctctatcaggtcattgcctgaga

SEQ ID NO: 183 - DNA, 45 bp, Artificial Sequence
  Synthetic Polynucleotide
aatgaaaaggtggcatccaataaaaatttttagaaccctcataaa

SEQ ID NO: 184 - DNA, 21 bp, Artificial Sequence
  Synthetic Polynucleotide
gataacctttgtgagagatag

SEQ ID NO: 185 - DNA, 35 bp, Artificial Sequence
  Synthetic Polynucleotide
actttctccgtggtgaagccggaatgcgcaatttg

SEQ ID NO: 186 - DNA, 28 bp, Artificial Sequence
  Synthetic Polynucleotide
gataggtcacgttggcggattatcagct

SEQ ID NO: 187 - DNA, 30 bp, Artificial Sequence
  Synthetic Polynucleotide
gaattatcaccgtaattattcattaaagcc

SEQ ID NO: 188 - DNA, 21 bp, Artificial Sequence
  Synthetic Polynucleotide
tcggcattttcaacagtttga

SEQ ID NO: 189 - DNA, 24 bp, Artificial Sequence
  Synthetic Polynucleotide
ccagcattgaagtgtactggtaca

SEQ ID NO: 190 - DNA, 21 bp, Artificial Sequence
  Synthetic Polynucleotide
aagttttaactgctcagtagt

SEQ ID NO: 191 - DNA, 30 bp, Artificial Sequence
  Synthetic Polynucleotide
tagcaagcccaataccctcattttcaggca

SEQ ID NO: 192 - DNA, 31 bp, Artificial Sequence
  Synthetic Polynucleotide
tttcggtcatgaaccaccaccagagccgccg

SEQ ID NO: 193 - DNA, 45 bp, Artificial Sequence
  Synthetic Polynucleotide
ggataaatattgacggacaccgactcagactgtagcgcgttttat

SEQ ID NO: 194 - DNA, 21 bp, Artificial Sequence
  Synthetic Polynucleotide
gcggagtgaaaatctccaaaa

SEQ ID NO: 195 - DNA, 35 bp, Artificial Sequence
  Synthetic Polynucleotide
aaaaggctccaaaaggaagccaccaggaaccatac

SEQ ID NO: 196 - DNA, 28 bp, Artificial Sequence
  Synthetic Polynucleotide
aggcggataagtgcggggtttggggtca

SEQ ID NO: 197 - DNA, 41 bp, Artificial Sequence
  Synthetic Polynucleotide
acaggaggccgattaatcagagcgcggtcacgctgcgccaa

SEQ ID NO: 198 - DNA, 38 bp, Artificial Sequence
  Synthetic Polynucleotide
attgtgttcatgggtaagaatcgccatatttaacaacg

SEQ ID NO: 199 - DNA, 21 bp, Artificial Sequence
  Synthetic Polynucleotide
tatcaaagtgtagggagctaa

SEQ ID NO: 200 - DNA, 43 bp, Artificial Sequence
  Synthetic Polynucleotide
cgtccgggttgtggtgctcataccaaattgttatccgctcaca

SEQ ID NO: 201 - DNA, 24 bp, Artificial Sequence
  Synthetic Polynucleotide
ttgatggtggttcgaaaaaccgtc

SEQ ID NO: 202 - DNA, 21 bp, Artificial Sequence
  Synthetic Polynucleotide
cgcgcggggagaagaatgcgg

SEQ ID NO: 203 - DNA, 49 bp, Artificial Sequence
  Synthetic Polynucleotide
cgggccgttttcacggtgcggccggcggttcagcaggcgaaaatcctgt

SEQ ID NO: 204 - DNA, 44 bp, Artificial Sequence
  Synthetic Polynucleotide
cggcatcagatgcaaagggccgaaatcggcaaatttgccctgcg

SEQ ID NO: 205 - DNA, 26 bp, Artificial Sequence
  Synthetic Polynucleotide
cctgcggctggtaagcaaatcgttaa

SEQ ID NO: 206 - DNA, 31 bp, Artificial Sequence
  Synthetic Polynucleotide
attccacacaacgcattaatgaatcggccaa

SEQ ID NO: 207 - DNA, 40 bp, Artificial Sequence
  Synthetic Polynucleotide
tggaagtttcattccaactaaagattagagagtacctaag

SEQ ID NO: 208 - DNA, 21 bp, Artificial Sequence
  Synthetic Polynucleotide
caacaggtcaggtacggtgtc

SEQ ID NO: 209 - DNA, 44 bp, Artificial Sequence
  Synthetic Polynucleotide
cgaagctggctagtgaatgtagtaaaacgaactaacggaacaac

SEQ ID NO: 210 - DNA, 24 bp, Artificial Sequence
  Synthetic Polynucleotide
tcaaaaatcaggggaagcaaactc

SEQ ID NO: 211 - DNA, 21 bp, Artificial Sequence
  Synthetic Polynucleotide
atagcgagaggcgccctgacg

SEQ ID NO: 212 - DNA, 49 bp, Artificial Sequence
  Synthetic Polynucleotide
agaaacaccagaacgaaaggcttttttgcaaaacgagaatgaccataaa

SEQ ID NO: 213 - DNA, 44 bp, Artificial Sequence
  Synthetic Polynucleotide
ccaggcgcatagccagacctctttaccctgactgttcagaaaag

SEQ ID NO: 214 - DNA, 26 bp, Artificial Sequence
  Synthetic Polynucleotide
ggaacgaggcgcagacggtgtacaga

SEQ ID NO: 215 - DNA, 24 bp, Artificial Sequence
  Synthetic Polynucleotide
tcatatgagccgggtcactgttgc

SEQ ID NO: 216 - DNA, 31 bp, Artificial Sequence
  Synthetic Polynucleotide
attattacaggtgacgacgataaaaaccaaa

SEQ ID NO: 217 - DNA, 41 bp, Artificial Sequence
  Synthetic Polynucleotide
gcaacatataaaagaatacatacaacaaagttaccagtacc

SEQ ID NO: 218 - DNA, 21 bp, Artificial Sequence
  Synthetic Polynucleotide
agcagatagccgataaaggtg

SEQ ID NO: 219 - DNA, 43 bp, Artificial Sequence
  Synthetic Polynucleotide
gaacgacaattcccatcatcggcttcagatatagaaggcttat

SEQ ID NO: 220 - DNA, 24 bp, Artificial Sequence
  Synthetic Polynucleotide
caccctgaacaattaagaaaagta

SEQ ID NO: 221 - DNA, 21 bp, Artificial Sequence
  Synthetic Polynucleotide
ctaatttgccagacgagcatg

SEQ ID NO: 222 - DNA, 49 bp, Artificial Sequence
  Synthetic Polynucleotide
tagaaaccaatcaatactaatttttacaaagacgggagaattaactgaa

SEQ ID NO: 223 - DNA, 44 bp, Artificial Sequence
  Synthetic Polynucleotide
ctgtccagacgagccctttagtcagagggtaatcgcattaataa

SEQ ID NO: 224 - DNA, 26 bp, Artificial Sequence
  Synthetic Polynucleotide
ccaacatgtaatttggtaaagtaatt

SEQ ID NO: 225 - DNA, 24 bp, Artificial Sequence
  Synthetic Polynucleotide
agacctgctccatgttacttagcc

SEQ ID NO: 226 - DNA, 31 bp, Artificial Sequence
  Synthetic Polynucleotide
ccggtattctaaacgagcgtctttccagagc

SEQ ID NO: 227 - DNA, 28 bp, Artificial Sequence
  Synthetic Polynucleotide
cgccaaccgcaagaaaagttacctgtcc

SEQ ID NO: 228 - DNA, 39 bp, Artificial Sequence
  Synthetic Polynucleotide
agtgaggaaaacgctcatgcgcgtactagtgtttttggt

SEQ ID NO: 229 - DNA, 28 bp, Artificial Sequence
  Synthetic Polynucleotide
cgtccaccacacccgccaacaagagcag

SEQ ID NO: 230 - DNA, 45 bp, Artificial Sequence
  Synthetic Polynucleotide
aatccattgcaacaggaccaccgacggacttgcggtcccttagaa

SEQ ID NO: 231 - DNA, 42 bp, Artificial Sequence
  Synthetic Polynucleotide
cactatcggccttgctggtagcaaattaattacattgcatta

SEQ ID NO: 232 - DNA, 35 bp, Artificial Sequence
  Synthetic Polynucleotide
actaaaatcccttataatgagagacgccaggctgc

SEQ ID NO: 233 - DNA, 28 bp, Artificial Sequence
  Synthetic Polynucleotide
tccgaatagcccgagatttgccctcacc

SEQ ID NO: 234 - DNA, 31 bp, Artificial Sequence
  Synthetic Polynucleotide
gtgccaacggattcgccgtcagcgtataatc

SEQ ID NO: 235 - DNA, 35 bp, Artificial Sequence
  Synthetic Polynucleotide
gaatttgaatgtacctttctcatcaatataaattt

SEQ ID NO: 236 - DNA, 35 bp, Artificial Sequence
  Synthetic Polynucleotide
cagaacatcgccattaaaaatgaatctggtcaata

SEQ ID NO: 237 - DNA, 42 bp, Artificial Sequence
  Synthetic Polynucleotide
cgttcgcgcatcagatgtgtttggattcctgattatcagtat

SEQ ID NO: 238 - DNA, 42 bp, Artificial Sequence
  Synthetic Polynucleotide
tgaatttcaacgtagattaatggaaaggagcggaattacgtt

SEQ ID NO: 239 - DNA, 35 bp, Artificial Sequence
  Synthetic Polynucleotide
aaaagtttgggcgcttatttgacgagcacgtggta

SEQ ID NO: 240 - DNA, 42 bp, Artificial Sequence
  Synthetic Polynucleotide
accgcgtaagtatttacccagaacaatattaccatcaccatc

SEQ ID NO: 241 - DNA, 35 bp, Artificial Sequence
  Synthetic Polynucleotide
caagcggaatcggcattaaagcgcgtaagctttcc

SEQ ID NO: 242 - DNA, 42 bp, Artificial Sequence
  Synthetic Polynucleotide
accttgctgaacaacagctgaagtttaatgcgcgaactgata

SEQ ID NO: 243 - DNA, 35 bp, Artificial Sequence
  Synthetic Polynucleotide
cgccagttgaagattagaattttaaaagtttccac

SEQ ID NO: 244 - DNA, 42 bp, Artificial Sequence
  Synthetic Polynucleotide
gcgaacctgttccacacaacatactagctgtcggtcattgag

SEQ ID NO: 245 - DNA, 21 bp, Artificial Sequence
  Synthetic Polynucleotide
tttacgatccgcggtgctcag

SEQ ID NO: 246 - DNA, 42 bp, Artificial Sequence
  Synthetic Polynucleotide
agtacattaagggtgcctaatgaggaggatccgcgtccaaac

SEQ ID NO: 247 - DNA, 35 bp, Artificial Sequence
  Synthetic Polynucleotide
ataaaatctaaagcatcgccctaaacaatatgctc

SEQ ID NO: 248 - DNA, 28 bp, Artificial Sequence
  Synthetic Polynucleotide
ccgaagcataaagtgtatcgaattccag

SEQ ID NO: 249 - DNA, 49 bp, Artificial Sequence
  Synthetic Polynucleotide
actttagctaactcgagacgggggagaaacaatcttgttcttcccgggt

SEQ ID NO: 250 - DNA, 35 bp, Artificial Sequence
  Synthetic Polynucleotide
catatcctttgcccgaatcatcatattatacgtaa

SEQ ID NO: 251 - DNA, 28 bp, Artificial Sequence
  Synthetic Polynucleotide
cagttctttttcaccgcctggcccatca

SEQ ID NO: 252 - DNA, 42 bp, Artificial Sequence
  Synthetic Polynucleotide
caccgctcaacaccgtcggtgatgggtctggcggtgccttgt

SEQ ID NO: 253 - DNA, 35 bp, Artificial Sequence
  Synthetic Polynucleotide
gaatttcaggaaatcaatgagagccagcagcaaat

SEQ ID NO: 254 - DNA, 28 bp, Artificial Sequence
  Synthetic Polynucleotide
cggacatcccttttagacaggaacataa

SEQ ID NO: 255 - DNA, 35 bp, Artificial Sequence
  Synthetic Polynucleotide
ccaagcgcaggtttctgcgtaatcatggtcagagc

SEQ ID NO: 256 - DNA, 42 bp, Artificial Sequence
  Synthetic Polynucleotide
tgctggctattagtcgggggaaatacctacattttgactttt

SEQ ID NO: 257 - DNA, 28 bp, Artificial Sequence
  Synthetic Polynucleotide
ttccctgaaagaacgaaccaccaggcca

SEQ ID NO: 258 - DNA, 42 bp, Artificial Sequence
  Synthetic Polynucleotide
cagcagaatcctgagaatggttgcatgcgccgctacagttga

SEQ ID NO: 259 - DNA, 35 bp, Artificial Sequence
  Synthetic Polynucleotide
gctctgattgccgttccggcaaacgtagaactgat

SEQ ID NO: 260 - DNA, 42 bp, Artificial Sequence
  Synthetic Polynucleotide
tgcgtaaaagagtctgtccgccagcgtctgaaatggataata

SEQ ID NO: 261 - DNA, 42 bp, Artificial Sequence
  Synthetic Polynucleotide
ctctcgctgggtcgctattaattatcctgataatatacatca

SEQ ID NO: 262 - DNA, 42 bp, Artificial Sequence
  Synthetic Polynucleotide
gcagcaaattaaccgttgtaatatattggcagattcaccttc

SEQ ID NO: 263 - DNA, 31 bp, Artificial Sequence
  Synthetic Polynucleotide
aatgctcgtcattgccaacggcagcagtagg

SEQ ID NO: 264 - DNA, 35 bp, Artificial Sequence
  Synthetic Polynucleotide
gcttaataccggggtgtcacttattggggttgcag

SEQ ID NO: 265 - DNA, 28 bp, Artificial Sequence
  Synthetic Polynucleotide
atagcgatagcttacaagcgtgccgcat

SEQ ID NO: 266 - DNA, 42 bp, Artificial Sequence
  Synthetic Polynucleotide
tccttgagtgagccttacatcgcctcaaatatcaagtattag

SEQ ID NO: 267 - DNA, 35 bp, Artificial Sequence
  Synthetic Polynucleotide
tccgttttttcgtctcgataacggtacaaaaggca

SEQ ID NO: 268 - DNA, 42 bp, Artificial Sequence
  Synthetic Polynucleotide
atccagcctccgtaacaatttcatataaccttgcttctttct

SEQ ID NO: 269 - DNA, 32 bp, Artificial Sequence
  Synthetic Polynucleotide
accgagcaagcctgttgcgttgcgctcagtgg

SEQ ID NO: 270 - DNA, 35 bp, Artificial Sequence
  Synthetic Polynucleotide
cggctttccagtcgggagtttgcggcgcgccatgc

SEQ ID NO: 271 - DNA, 42 bp, Artificial Sequence
  Synthetic Polynucleotide
acaactcgatgatggcaatctcacagtttgacaaacaattcg

SEQ ID NO: 272 - DNA, 42 bp, Artificial Sequence
  Synthetic Polynucleotide
taattgaggatttagaaaccctcaagtaacaaccaagtaacg

SEQ ID NO: 273 - DNA, 42 bp, Artificial Sequence
  Synthetic Polynucleotide
attagccgtcaatagatagttggctttaacggaggcgacaga

SEQ ID NO: 274 - DNA, 34 bp, Artificial Sequence
  Synthetic Polynucleotide
gtgccatcccacgcaacaagggtaaagttaaacg

SEQ ID NO: 275 - DNA, 42 bp, Artificial Sequence
  Synthetic Polynucleotide
cacaggcggcctttagtgatgcagcttacggctggaggtgtc

SEQ ID NO: 276 - DNA, 42 bp, Artificial Sequence
  Synthetic Polynucleotide
aaaatcccgtaaaaaaagccgcagcatcagcggggtcattgc

SEQ ID NO: 277 - DNA, 34 bp, Artificial Sequence
  Synthetic Polynucleotide
gtgtacatcgacataaaaggcgctttcgcactca

SEQ ID NO: 278 - DNA, 28 bp, Artificial Sequence
  Synthetic Polynucleotide
gagcaccaacctaaagaagagtaatcga

SEQ ID NO: 279 - DNA, 39 bp, Artificial Sequence
  Synthetic Polynucleotide
tcgcaaaaaatcggttgtattaattgctccattagtacg

SEQ ID NO: 280 - DNA, 28 bp, Artificial Sequence
  Synthetic Polynucleotide
tttttttgataagaggtttttaattctt

SEQ ID NO: 281 - DNA, 45 bp, Artificial Sequence
  Synthetic Polynucleotide
taccagagcataaagcttggtcaagtttccaacagcattctgctc

SEQ ID NO: 282 - DNA, 42 bp, Artificial Sequence
  Synthetic Polynucleotide
attacaggcaaggcaaagctgaaagaaacgtacagcttgcca

SEQ ID NO: 283 - DNA, 35 bp, Artificial Sequence
  Synthetic Polynucleotide
gctaagcaaagcggattctcaaattagtaaacact

SEQ ID NO: 284 - DNA, 28 bp, Artificial Sequence
  Synthetic Polynucleotide
aaaaaagattaagaggaataaatatagc

SEQ ID NO: 285 - DNA, 31 bp, Artificial Sequence
  Synthetic Polynucleotide
agacaagttgggtaacgggtaaaaatacatt

SEQ ID NO: 286 - DNA, 35 bp, Artificial Sequence
  Synthetic Polynucleotide
ccatttcccaaagggggaacggcctcaggaattaa

SEQ ID NO: 287 - DNA, 35 bp, Artificial Sequence
  Synthetic Polynucleotide
agagccggagagggtaggtcaatcaagcaaataat

SEQ ID NO: 288 - DNA, 42 bp, Artificial Sequence
  Synthetic Polynucleotide
aggaaacgaccgctattctccagcccagtttgaggggacgag

SEQ ID NO: 289 - DNA, 42 bp, Artificial Sequence
  Synthetic Polynucleotide
aaatttcagaggcgatccgcttctcgcatcgtaaccgtctcc

SEQ ID NO: 290 - DNA, 35 bp, Artificial Sequence
  Synthetic Polynucleotide
caatatcgcgcatttttatgctgtagctcaagaac

SEQ ID NO: 291 - DNA, 42 bp, Artificial Sequence
  Synthetic Polynucleotide
tttaagggtgcctttatcaaaattaagcaatatatttttaaa

SEQ ID NO: 292 - DNA, 35 bp, Artificial Sequence
  Synthetic Polynucleotide
acagttctagtcagtcaaagcttgctcctaaatat

SEQ ID NO: 293 - DNA, 42 bp, Artificial Sequence
  Synthetic Polynucleotide
tgataatcagaaggaatcgtcagtcaaccgttctagctgata

SEQ ID NO: 294 - DNA, 35 bp, Artificial Sequence
  Synthetic Polynucleotide
aatacgttaacaataggggaacaaacggcggagat

SEQ ID NO: 295 - DNA, 42 bp, Artificial Sequence
  Synthetic Polynucleotide
tttccagacgagattcatcagttgtaaaacgggcttgagagc

SEQ ID NO: 296 - DNA, 21 bp, Artificial Sequence
  Synthetic Polynucleotide
ttatcaacgtaagaaccacga

SEQ ID NO: 297 - DNA, 42 bp, Artificial Sequence
  Synthetic Polynucleotide
gtctacgagggcagatacataacgcattataccttatggcca

SEQ ID NO: 298 - DNA, 28 bp, Artificial Sequence
  Synthetic Polynucleotide
atcggaataccacattcgggaagaaact

SEQ ID NO: 299 - DNA, 49 bp, Artificial Sequence
  Synthetic Polynucleotide
gctttaaaaggaatcaatactgcaaggcgattatttgaattaccagtca

SEQ ID NO: 300 - DNA, 35 bp, Artificial Sequence
  Synthetic Polynucleotide
tcgcaacccgtcggattgcatctgcagctttcgca

SEQ ID NO: 301 - DNA, 28 bp, Artificial Sequence
  Synthetic Polynucleotide
aaagactggattcattgaatccccgcat

SEQ ID NO: 302 - DNA, 42 bp, Artificial Sequence
  Synthetic Polynucleotide
cagattgtatatatgtaccccggtaattaatcagtcaagtaa

SEQ ID NO: 303 - DNA, 42 bp, Artificial Sequence
  Synthetic Polynucleotide
ttacgccgggaaagaatacacgattgccactggatattcttc

SEQ ID NO: 304 - DNA, 36 bp, Artificial Sequence
  Synthetic Polynucleotide
gcacggtgcggattgtaacgtaaaactagcatctat

SEQ ID NO: 305 - DNA, 28 bp, Artificial Sequence
  Synthetic Polynucleotide
tcaggacagaattcccaattctgccatg

SEQ ID NO: 306 - DNA, 35 bp, Artificial Sequence
  Synthetic Polynucleotide
gacaacaaagtaatttcaaaatctacgttaaagat

SEQ ID NO: 307 - DNA, 42 bp, Artificial Sequence
  Synthetic Polynucleotide
ggttcaatatgatatccgcccaaaaacattatgaccctatca

SEQ ID NO: 308 - DNA, 28 bp, Artificial Sequence
  Synthetic Polynucleotide
agcgattcaatgagagatctacaacggt

SEQ ID NO: 309 - DNA, 42 bp, Artificial Sequence
  Synthetic Polynucleotide
aggtagatttagtttgagaatatagcggatggcttagacgaa

SEQ ID NO: 310 - DNA, 35 bp, Artificial Sequence
  Synthetic Polynucleotide
taacgtcaccctcagcagcgaaagttaaacgccag

SEQ ID NO: 311 - DNA, 42 bp, Artificial Sequence
  Synthetic Polynucleotide
gaataacctgtttagctaaagcctttttgcgggagaagagaa

SEQ ID NO: 312 - DNA, 35 bp, Artificial Sequence
  Synthetic Polynucleotide
gaccaacggcacagcggatcaaacgatcgcaacgc

SEQ ID NO: 313 - DNA, 42 bp, Artificial Sequence
  Synthetic Polynucleotide
gaccatttggggcgcgagaattagttcaacgcaaggataggt

SEQ ID NO: 314 - DNA, 31 bp, Artificial Sequence
  Synthetic Polynucleotide
cggactttgaaaacgaaagaggcacgcggtt

SEQ ID NO: 315 - DNA, 35 bp, Artificial Sequence
  Synthetic Polynucleotide
gcggtatgatggttctgctcaggggtaagctttaa

SEQ ID NO: 316 - DNA, 28 bp, Artificial Sequence
  Synthetic Polynucleotide
gcagttgggcggttatcatcattgaccc

SEQ ID NO: 317 - DNA, 42 bp, Artificial Sequence
  Synthetic Polynucleotide
atttgcccgattttatgtgctgcaagccccaaaaagtagcca

SEQ ID NO: 318 - DNA, 35 bp, Artificial Sequence
  Synthetic Polynucleotide
attcggaacgagggtagtttttcacgttgtaccgg

SEQ ID NO: 319 - DNA, 41 bp, Artificial Sequence
  Synthetic Polynucleotide
gaatacagaggcgccatgtttacccacggaaaaagagaccg

SEQ ID NO: 320 - DNA, 32 bp, Artificial Sequence
  Synthetic Polynucleotide
ggacgttaactaatcatagtaagagcaaatgt

SEQ ID NO: 321 - DNA, 35 bp, Artificial Sequence
  Synthetic Polynucleotide
ttaataaccctcgtttagccagagttcagtgttca

SEQ ID NO: 322 - DNA, 42 bp, Artificial Sequence
  Synthetic Polynucleotide
atgtgagcgacgacagtatgaactggctcccatcaacattaa

SEQ ID NO: 323 - DNA, 42 bp, Artificial Sequence
  Synthetic Polynucleotide
taacgtctggccttcctcaggaagctggcgagtcacgatgag

SEQ ID NO: 324 - DNA, 42 bp, Artificial Sequence
  Synthetic Polynucleotide
gtgaacgccatcaaaaatatttaagcctcttggccagttgag

SEQ ID NO: 325 - DNA, 30 bp, Artificial Sequence
  Synthetic Polynucleotide
taaaacactcatcttaggccgcttttgcgg

SEQ ID NO: 326 - DNA, 30 bp, Artificial Sequence
  Synthetic Polynucleotide
tagttgcgccgacaataaattgtgtcgaaa

SEQ ID NO: 327 - DNA, 28 bp, Artificial Sequence
  Synthetic Polynucleotide
caccgaccgtgtgatcagacgacacaag

SEQ ID NO: 328 - DNA, 39 bp, Artificial Sequence
  Synthetic Polynucleotide
aatagaagcaccattaccaggaatacccattttgtaaat

SEQ ID NO: 329 - DNA, 28 bp, Artificial Sequence
  Synthetic Polynucleotide
cttagttaccagaaggaataagagataa

SEQ ID NO: 330 - DNA, 21 bp, Artificial Sequence
  Synthetic Polynucleotide
gaagaaacgcaataataagaa

SEQ ID NO: 331 - DNA, 45 bp, Artificial Sequence
  Synthetic Polynucleotide
aatcaaaatcaccagtaaattcatgttaatttgtaaatcgaggtg

SEQ ID NO: 332 - DNA, 42 bp, Artificial Sequence
  Synthetic Polynucleotide
atctatcaccgtcaccgtcaaccggtgagaatagaaacgtta

SEQ ID NO: 333 - DNA, 35 bp, Artificial Sequence
  Synthetic Polynucleotide
aaagagggtaattgagccagccttcagccattttt

SEQ ID NO: 334 - DNA, 28 bp, Artificial Sequence
  Synthetic Polynucleotide
aagtcagagagataacctaacgtctcca

SEQ ID NO: 335 - DNA, 31 bp, Artificial Sequence
  Synthetic Polynucleotide
ttgtgcagacagccctcctgacctcacaatc

SEQ ID NO: 336 - DNA, 35 bp, Artificial Sequence
  Synthetic Polynucleotide
aaagcgtaaccaaactaacgtatcaccgtacttgc

SEQ ID NO: 337 - DNA, 35 bp, Artificial Sequence
  Synthetic Polynucleotide
tctagagccgccaccctagacgatcgcagtcacag

SEQ ID NO: 338 - DNA, 42 bp, Artificial Sequence
  Synthetic Polynucleotide
ttttcgtcttcactgaggtttagttgatataagtatagtctg

SEQ ID NO: 339 - DNA, 42 bp, Artificial Sequence
  Synthetic Polynucleotide
gtcaatgaatataggaaaaccgccgataagtgccgtcggagg

SEQ ID NO: 340 - DNA, 35 bp, Artificial Sequence
  Synthetic Polynucleotide
atacccaataaaccgagctggcatgattaagaaga

SEQ ID NO: 341 - DNA, 42 bp, Artificial Sequence
  Synthetic Polynucleotide
accccttattcagcaccccatttgggaattaccaaagaaact

SEQ ID NO: 342 - DNA, 35 bp, Artificial Sequence
  Synthetic Polynucleotide
agaataaaaagtcacaatgaacgaacaaattacgc

SEQ ID NO: 343 - DNA, 42 bp, Artificial Sequence
  Synthetic Polynucleotide
acaaacaaataattttttgttcagagccaccaccggaaccgc

SEQ ID NO: 344 - DNA, 35 bp, Artificial Sequence
  Synthetic Polynucleotide
ggatccagtaacggggtagactcctcaagagccag

SEQ ID NO: 345 - DNA, 42 bp, Artificial Sequence
  Synthetic Polynucleotide
gcctatcctgttatccggtattcttaccgcgcaatcaaagcc

SEQ ID NO: 346 - DNA, 21 bp, Artificial Sequence
  Synthetic Polynucleotide
tttcctgtttacatgttgaaa

SEQ ID NO: 347 - DNA, 42 bp, Artificial Sequence
  Synthetic Polynucleotide
aatttaaatcccgacttgcgggagcgagaacgtattaataaa

SEQ ID NO: 348 - DNA, 28 bp, Artificial Sequence
  Synthetic Polynucleotide
gcacgaggcgttttagctattttctcct

SEQ ID NO: 349 - DNA, 49 bp, Artificial Sequence
  Synthetic Polynucleotide
cctgctttgaagccaagaaactgtagcattccacaagaacggaagcaag

SEQ ID NO: 350 - DNA, 35 bp, Artificial Sequence
  Synthetic Polynucleotide
tgccatgaaagtattaaagagggtaccgccataat

SEQ ID NO: 351 - DNA, 28 bp, Artificial Sequence
  Synthetic Polynucleotide
gcgatcccaaaaaaatgaaaataggcta

SEQ ID NO: 352 - DNA, 42 bp, Artificial Sequence
  Synthetic Polynucleotide
gtctggaaagtggccttgatattcctccctctttcatacacc

SEQ ID NO: 353 - DNA, 42 bp, Artificial Sequence
  Synthetic Polynucleotide
tatgcgacctaaataagaatacttatggtttcagctaaagtt

SEQ ID NO: 354 - DNA, 36 bp, Artificial Sequence
  Synthetic Polynucleotide
tcagcccatgtttaccgtggttgaggcaggtccaga

SEQ ID NO: 355 - DNA, 28 bp, Artificial Sequence
  Synthetic Polynucleotide
gacgtaataaataaaagaaacgcaactc

SEQ ID NO: 356 - DNA, 35 bp, Artificial Sequence
  Synthetic Polynucleotide
acaatcaacactgtcttatcgtaggaatcataaga

SEQ ID NO: 357 - DNA, 42 bp, Artificial Sequence
  Synthetic Polynucleotide
ttatcaccggaaccacaacttagcaaggccggaaacgtatca

SEQ ID NO: 358 - DNA, 28 bp, Artificial Sequence
  Synthetic Polynucleotide
gtaatagcccgccaccctcagagcgaca

SEQ ID NO: 359 - DNA, 21 bp, Artificial Sequence
  Synthetic Polynucleotide
taccacggaataagtttaaaa

SEQ ID NO: 360 - DNA, 35 bp, Artificial Sequence
  Synthetic Polynucleotide
ttaaggttgggttatataactatatcatcttatag

SEQ ID NO: 361 - DNA, 42 bp, Artificial Sequence
  Synthetic Polynucleotide
ttaatggtttaccagcggagccaggaaaccatcgatagagcg

SEQ ID NO: 362 - DNA, 35 bp, Artificial Sequence
  Synthetic Polynucleotide
tttaatcgcaatcggtttatcagctcaggagtttc

SEQ ID NO: 363 - DNA, 42 bp, Artificial Sequence
  Synthetic Polynucleotide
gaacaaaagggcgacatacttgaggtaatcagtagcgattcg

SEQ ID NO: 364 - DNA, 31 bp, Artificial Sequence
  Synthetic Polynucleotide
ggattttcgagcaaataaggcgttgctccat

SEQ ID NO: 365 - DNA, 35 bp, Artificial Sequence
  Synthetic Polynucleotide
gttactttaatcggatagataaaataaatacagag

SEQ ID NO: 366 - DNA, 28 bp, Artificial Sequence
  Synthetic Polynucleotide
cagcttgataccgatcccattccagaac

SEQ ID NO: 367 - DNA, 42 bp, Artificial Sequence
  Synthetic Polynucleotide
aatttctaccaagtcaacgccgaatcctcattaaaaatgccc

SEQ ID NO: 368 - DNA, 35 bp, Artificial Sequence
  Synthetic Polynucleotide
tttgctgatgcaaatcctcaaataagttttggcca

SEQ ID NO: 369 - DNA, 41 bp, Artificial Sequence
  Synthetic Polynucleotide
tgtagacaaagaaggaacaactaaccaaaaggagccttccc

SEQ ID NO: 370 - DNA, 32 bp, Artificial Sequence
  Synthetic Polynucleotide
ccgttttgaacctcaagattagttgctaatta

SEQ ID NO: 371 - DNA, 35 bp, Artificial Sequence
  Synthetic Polynucleotide
acgcccagctacaatttagttacaagtcctgtcca

SEQ ID NO: 372 - DNA, 42 bp, Artificial Sequence
  Synthetic Polynucleotide
ctattatcccggaataggtcgcactcatgtctatttcggaac

SEQ ID NO: 373 - DNA, 42 bp, Artificial Sequence
  Synthetic Polynucleotide
aaaccgtataaacagttgccagaaaccagtagatctaatatt

SEQ ID NO: 374 - DNA, 42 bp, Artificial Sequence
  Synthetic Polynucleotide
ctgcagtgccttgagtatctgaataccgtaatccagacgcga

SEQ ID NO: 375 - DNA, 34 bp, Artificial Sequence
  Synthetic Polynucleotide
aacaccggaatcataatacctttttaacctccgg

SEQ ID NO: 376 - DNA, 42 bp, Artificial Sequence
  Synthetic Polynucleotide
aaatcataggtctgagagacttactagaaaaagcctgtttag

SEQ ID NO: 377 - DNA, 42 bp, Artificial Sequence
  Synthetic Polynucleotide
gagtcaatagtgaatttatcatatcatatgcgttatacaaat

SEQ ID NO: 378 - DNA, 34 bp, Artificial Sequence
  Synthetic Polynucleotide
gattaagacgctgagaatcttaccagtataaagc

SEQ ID NO: 379 - DNA, 48 bp, Artificial Sequence
  Synthetic Polynucleotide
tgacaacaaccagcagggagttaatgacccccagcgatcatcgcctga

SEQ ID NO: 380 - DNA, 21 bp, Artificial Sequence
  Synthetic Polynucleotide
gtggttccgatccacgcagag

SEQ ID NO: 381 - DNA, 21 bp, Artificial Sequence
  Synthetic Polynucleotide
ctgactattaagaaaacaagt

SEQ ID NO: 382 - DNA, 21 bp, Artificial Sequence
  Synthetic Polynucleotide
caccctgaaccataaaaattt

SEQ ID NO: 383 - DNA, 30 bp, Artificial Sequence
  Synthetic Polynucleotide
ctgagtagaagaactcaaacacgaccagta

SEQ ID NO: 384 - DNA, 27 bp, Artificial Sequence
  Synthetic Polynucleotide
attctggccaacagagataaaacagag

SEQ ID NO: 385 - DNA, 42 bp, Artificial Sequence
  Synthetic Polynucleotide
agtattaacaccgcctgcaacagtcagaagatagaacccagt

SEQ ID NO: 386 - DNA, 35 bp, Artificial Sequence
  Synthetic Polynucleotide
tctttaggagcactaacaactaataaggaatgaaa

SEQ ID NO: 387 - DNA, 37 bp, Artificial Sequence
  Synthetic Polynucleotide
ttgttacctgaaacaaatacttctttgattagtaata

SEQ ID NO: 388 - DNA, 30 bp, Artificial Sequence
  Synthetic Polynucleotide
gcacgtaaaacagaaataaatgaggaaggt

SEQ ID NO: 389 - DNA, 21 bp, Artificial Sequence
  Synthetic Polynucleotide
aacaaacatcaagaagcaaaa

SEQ ID NO: 390 - DNA, 30 bp, Artificial Sequence
  Synthetic Polynucleotide
acataaatcaatatatggaacctaccatat

SEQ ID NO: 391 - DNA, 34 bp, Artificial Sequence
  Synthetic Polynucleotide
cagagggttatgagtgattgaattacctttttta

SEQ ID NO: 392 - DNA, 21 bp, Artificial Sequence
  Synthetic Polynucleotide
gcggaacaaagaaagagtaac

SEQ ID NO: 393 - DNA, 30 bp, Artificial Sequence
  Synthetic Polynucleotide
attaacatccaataaatcattttagaaccc

SEQ ID NO: 394 - DNA, 27 bp, Artificial Sequence
  Synthetic Polynucleotide
aaatgcaatgcctgagtcaggtcattg

SEQ ID NO: 395 - DNA, 42 bp, Artificial Sequence
  Synthetic Polynucleotide
ggagcaaacaagagaatcgatgaaaggctataatgtgtaaaa

SEQ ID NO: 396 - DNA, 35 bp, Artificial Sequence
  Synthetic Polynucleotide
tgttaaatcagctcattttttaactattttgtggg

SEQ ID NO: 397 - DNA, 37 bp, Artificial Sequence
  Synthetic Polynucleotide
aagggtggagaatcggcaggtggcatcaattctacta

SEQ ID NO: 398 - DNA, 30 bp, Artificial Sequence
  Synthetic Polynucleotide
cattcaggctgcgcaactgtttaaaattcg

SEQ ID NO: 399 - DNA, 21 bp, Artificial Sequence
  Synthetic Polynucleotide
acctcaccggaaacccgccac

SEQ ID NO: 400 - DNA, 30 bp, Artificial Sequence
  Synthetic Polynucleotide
tctccgtggtgaagggagaaaccaggcaaa

SEQ ID NO: 401 - DNA, 34 bp, Artificial Sequence
  Synthetic Polynucleotide
gggggtgccgtagctctagtcccggaatttgtga

SEQ ID NO: 402 - DNA, 21 bp, Artificial Sequence
  Synthetic Polynucleotide
ggtcacgttggtgtattgacc

SEQ ID NO: 403 - DNA, 30 bp, Artificial Sequence
  Synthetic Polynucleotide
attattcattaaaggtgaataagtttgcct

SEQ ID NO: 404 - DNA, 27 bp, Artificial Sequence
  Synthetic Polynucleotide
ctgtagcgcgttttcatctcagagccg

SEQ ID NO: 405 - DNA, 42 bp, Artificial Sequence
  Synthetic Polynucleotide
accaccagagccgccgccagcattcaccacccggcattcaga

SEQ ID NO: 406 - DNA, 35 bp, Artificial Sequence
  Synthetic Polynucleotide
ggagtgtactggtaataagttttaagcgtcaaagc

SEQ ID NO: 407 - DNA, 37 bp, Artificial Sequence
  Synthetic Polynucleotide
ccatttctgtcagcggaattgagggagggaaggtaaa

SEQ ID NO: 408 - DNA, 30 bp, Artificial Sequence
  Synthetic Polynucleotide
ccctcattttcagggatagctacatggctt

SEQ ID NO: 409 - DNA, 21 bp, Artificial Sequence
  Synthetic Polynucleotide
actttcaacagtttatgggat

SEQ ID NO: 410 - DNA, 30 bp, Artificial Sequence
  Synthetic Polynucleotide
ttgaaaatctccaaaaagaaccgccaccct

SEQ ID NO: 411 - DNA, 34 bp, Artificial Sequence
  Synthetic Polynucleotide
gcgaccctcaaaaggctaggaattgcgaataata

SEQ ID NO: 412 - DNA, 21 bp, Artificial Sequence
  Synthetic Polynucleotide
ggttttgctcagtaaaggatt

SEQ ID NO: 413 - DNA, 11 bp, Artificial Sequence
  Synthetic Polynucleotide
caaaattatga

SEQ ID NO: 414 - DNA, 11 bp, Artificial Sequence
  Synthetic Polynucleotide
gcgccattcca

SEQ ID NO: 415 - DNA, 11 bp, Artificial Sequence
  Synthetic Polynucleotide
cagagccacta

SEQ ID NO: 416 - DNA, 11 bp, Artificial Sequence
  Synthetic Polynucleotide
gaagatgattt

SEQ ID NO: 417 - DNA, 11 bp, Artificial Sequence
  Synthetic Polynucleotide
gggaacggaca

SEQ ID NO: 418 - DNA, 11 bp, Artificial Sequence
  Synthetic Polynucleotide
tttgctaaagc

SEQ ID NO: 419 - DNA, 11 bp, Artificial Sequence
  Synthetic Polynucleotide
tatctaaaaac

SEQ ID NO: 420 - DNA, 11 bp, Artificial Sequence
  Synthetic Polynucleotide
cattaaattga

SEQ ID NO: 421 - DNA, 11 bp, Artificial Sequence
  Synthetic Polynucleotide
ttgatgatatt

SEQ ID NO: 422 - DNA, 11 bp, Artificial Sequence
  Synthetic Polynucleotide
acatcactttt

SEQ ID NO: 423 - DNA, 11 bp, Artificial Sequence
  Synthetic Polynucleotide
atagtagtagg

SEQ ID NO: 424 - DNA, 11 bp, Artificial Sequence
  Synthetic Polynucleotide
tattgacggta

SEQ ID NO: 425 - DNA, 11 bp, Artificial Sequence
  Synthetic Polynucleotide
ataaaagggta

SEQ ID NO: 426 - DNA, 11 bp, Artificial Sequence
  Synthetic Polynucleotide
gtgaggcggtc

SEQ ID NO: 427 - DNA, 11 bp, Artificial Sequence
  Synthetic Polynucleotide
atggaaacagt

SEQ ID NO: 428 - DNA, 11 bp, Artificial Sequence
  Synthetic Polynucleotide
attatcattgc

SEQ ID NO: 429 - DNA, 11 bp, Artificial Sequence
  Synthetic Polynucleotide
tcatatattca

SEQ ID NO: 430 - DNA, 11 bp, Artificial Sequence
  Synthetic Polynucleotide
cctgagagtcc

SEQ ID NO: 431 - DNA, 11 bp, Artificial Sequence
  Synthetic Polynucleotide
gagatagaccg

SEQ ID NO: 432 - DNA, 11 bp, Artificial Sequence
  Synthetic Polynucleotide
gtaatgggaaa

SEQ ID NO: 433 - DNA, 11 bp, Artificial Sequence
  Synthetic Polynucleotide
ttagcgtcatt

SEQ ID NO: 434 - DNA, 11 bp, Artificial Sequence
  Synthetic Polynucleotide
ccaccagaact

SEQ ID NO: 435 - DNA, 11 bp, Artificial Sequence
  Synthetic Polynucleotide
attttttcatt

SEQ ID NO: 436 - DNA, 11 bp, Artificial Sequence
  Synthetic Polynucleotide
aggattagcgc

SEQ ID NO: 437 - DNA, 46 bp, Artificial Sequence
  Synthetic Polynucleotide
tttttaaacaggaggccgattaatcagatcacggtcacgctgaacg

SEQ ID NO: 438 - DNA, 54 bp, Artificial Sequence
  Synthetic Polynucleotide
tcgttagaaagggattacacttttctttcgccatatttaacaacgccaattttt

SEQ ID NO: 439 - DNA, 31 bp, Artificial Sequence
  Synthetic Polynucleotide
tttttaaaaaccgtctagcgggagctttttt

SEQ ID NO: 440 - DNA, 48 bp, Artificial Sequence
  Synthetic Polynucleotide
tgggcatcagtgtgcacgttttcattcctgtgtgaaattgttattttt

SEQ ID NO: 441 - DNA, 31 bp, Artificial Sequence
  Synthetic Polynucleotide
tttttcagaatgcggcgggcctctgtggcgc

SEQ ID NO: 442 - DNA, 36 bp, Artificial Sequence
  Synthetic Polynucleotide
tttttgtaatgggtaaaggggtgtgttcagcttttt

SEQ ID NO: 443 - DNA, 41 bp, Artificial Sequence
  Synthetic Polynucleotide
ttttttccgctcacaatcgtgccagctgcattaatgttttt

SEQ ID NO: 444 - DNA, 45 bp, Artificial Sequence
  Synthetic Polynucleotide
tttttagtttcattccatataaagtacggagagtacctttaagaa

SEQ ID NO: 445 - DNA, 54 bp, Artificial Sequence
  Synthetic Polynucleotide
gcaactaacagttgtgaacggctgaccagtcactgttgccctgcggctgttttt

SEQ ID NO: 446 - DNA, 31 bp, Artificial Sequence
  Synthetic Polynucleotide
tttttaggtcaggattagtgtctggattttt

SEQ ID NO: 447 - DNA, 49 bp, Artificial Sequence
  Synthetic Polynucleotide
ccaggctgaccaataaggtaaattgaactaacggaacaacattattttt

SEQ ID NO: 448 - DNA, 31 bp, Artificial Sequence
  Synthetic Polynucleotide
tttttacaccagaacgagtagcttgcccgca

SEQ ID NO: 449 - DNA, 36 bp, Artificial Sequence
  Synthetic Polynucleotide
tttttataagggaaccgaatgtacagaccagttttt

SEQ ID NO: 450 - DNA, 41 bp, Artificial Sequence
  Synthetic Polynucleotide
tttttttacaggtagaaacgataaaaaccaaaatagttttt

SEQ ID NO: 451 - DNA, 46 bp, Artificial Sequence
  Synthetic Polynucleotide
ttttttacatacataaaggtgtagcaaaagtaagcagatagcatag

SEQ ID NO: 452 - DNA, 54 bp, Artificial Sequence
  Synthetic Polynucleotide
agtatgtgcaacatgagaataagaggcaacgaggcgcagacggtcaatcttttt

SEQ ID NO: 453 - DNA, 31 bp, Artificial Sequence
  Synthetic Polynucleotide
tttttctttttaagaaacgtagaaaattttt

SEQ ID NO: 454 - DNA, 48 bp, Artificial Sequence
  Synthetic Polynucleotide
caaaattctgaacaagatagaaaccccaatagcaagcaaatcattttt

SEQ ID NO: 455 - DNA, 31 bp, Artificial Sequence
  Synthetic Polynucleotide
tttttctaatttacgagcatgaaaataagag

SEQ ID NO: 456 - DNA, 36 bp, Artificial Sequence
  Synthetic Polynucleotide
tttttcatgtaatttaggctaaagtaccgacttttt

SEQ ID NO: 457 - DNA, 41 bp, Artificial Sequence
  Synthetic Polynucleotide
tttttgatatagaaggcaatcttaccaacgctaacgttttt

SEQ ID NO: 458 - DNA, 34 bp, Artificial Sequence
  Synthetic Polynucleotide
tttttaaaatcctgtttcgtcaaagggcgttttt

SEQ ID NO: 459 - DNA, 28 bp, Artificial Sequence
  Synthetic Polynucleotide
ggggtggtttgccccagcaggcgttttt

SEQ ID NO: 460 - DNA, 34 bp, Artificial Sequence
  Synthetic Polynucleotide
tttttaaatcaggtcttgcaaactccaacttttt

SEQ ID NO: 461 - DNA, 28 bp, Artificial Sequence
  Synthetic Polynucleotide
aaaggagaatgaccataaatcaattttt

SEQ ID NO: 462 - DNA, 34 bp, Artificial Sequence
  Synthetic Polynucleotide
tttttgggagaattaaccttaccgaagccttttt

SEQ ID NO: 463 - DNA, 28 bp, Artificial Sequence
  Synthetic Polynucleotide
cctaacagggaagcgcattagacttttt

SEQ ID NO: 464 - DNA, 64 bp, Artificial Sequence
  Synthetic Polynucleotide
tttttaatcggccaacgtgctgcggcttcactaatctgatgaaaaggtaaagttagctattgaa

SEQ ID NO: 465 - DNA, 64 bp, Artificial Sequence
  Synthetic Polynucleotide
tttttcgagaggctttttgacgagaagcaaaattctcattgaaatcgttaacgactccaagatg

SEQ ID NO: 466 - DNA, 45 bp, Artificial Sequence
  Synthetic Polynucleotide
tttttagcgtctttccatatcccatcttcactaatcttatgtact

SEQ ID NO: 467 - DNA, 23 bp, Artificial Sequence
  Synthetic Polynucleotide
gcgcataggctgaccggaatacc

SEQ ID NO: 468 - DNA, 15 bp, Artificial Sequence
  Synthetic Polynucleotide
catcagattagtgaa

SEQ ID NO: 469 - DNA, 15 bp, Artificial Sequence
  Synthetic Polynucleotide
caatgagaattttgc

SEQ ID NO: 470 - DNA, 19 bp, Artificial Sequence
  Synthetic Polynucleotide
agtacataagattagtgaa

SEQ ID NO: 471 - DNA, 46 bp, Artificial Sequence
  Synthetic Polynucleotide
aacggtatatccagaacaaaccaccacaggattttaacggaatggt

SEQ ID NO: 472 - DNA, 38 bp, Artificial Sequence
  Synthetic Polynucleotide
gcgccgtaaacagagtgctcgtcataagttacctgtcc

SEQ ID NO: 473 - DNA, 35 bp, Artificial Sequence
  Synthetic Polynucleotide
ggaggccttgctggtaacgccagaccggccaagtt

SEQ ID NO: 474 - DNA, 42 bp, Artificial Sequence
  Synthetic Polynucleotide
gtcagtaataacatcaccgagtaagcaaaagaagattctgct

SEQ ID NO: 475 - DNA, 35 bp, Artificial Sequence; identical to SEQ ID NO: 232
  Synthetic Polynucleotide

SEQ ID NO: 476 - DNA, 42 bp, Artificial Sequence
  Synthetic Polynucleotide
agagcagccaagcgcaggtttctgcgtaatcatggtcagagc

SEQ ID NO: 477 - DNA, 31 bp, Artificial Sequence
  Synthetic Polynucleotide
gtgcctatacagtaacatcctcatagacagg

SEQ ID NO: 478 - DNA, 35 bp, Artificial Sequence
  Synthetic Polynucleotide
ctgttacatcgattttctcaattatcatcattgaa

SEQ ID NO: 479 - DNA, 35 bp, Artificial Sequence
  Synthetic Polynucleotide
agatggctattagtcttacaccgcaccttgcgagc

SEQ ID NO: 480 - DNA, 42 bp, Artificial Sequence
  Synthetic Polynucleotide
cagcggattccagaaatattatcaaacaaagaaaccacttta

SEQ ID NO: 481 - DNA, 42 bp, Artificial Sequence
  Synthetic Polynucleotide
taaaataccacaaaattatcaataagtaacattatcataaac

SEQ ID NO: 482 - DNA, 21 bp, Artificial Sequence
  Synthetic Polynucleotide
gtggttccgatccacgcagag

SEQ ID NO: 483 - DNA, 25 bp, Artificial Sequence
  Synthetic Polynucleotide
aaaagtttgggtgtagccgcttaat

SEQ ID NO: 484 - DNA, 42 bp, Artificial Sequence
  Synthetic Polynucleotide
gcgattctggaatacctagtagaagaactcattttatatcgt

SEQ ID NO: 485 - DNA, 35 bp, Artificial Sequence
  Synthetic Polynucleotide
caagcggaatcggcattaaagcgggcgcgcgcgta

SEQ ID NO: 486 - DNA, 46 bp, Artificial Sequence
  Synthetic Polynucleotide
cagctgaagtacgtaagaaggtatattaccgccagccattgctgac

SEQ ID NO: 487 - DNA, 42 bp, Artificial Sequence
  Synthetic Polynucleotide
gcgaacctgttccacacaacatactagctgtcggtcatagta

SEQ ID NO: 488 - DNA, 42 bp, Artificial Sequence
  Synthetic Polynucleotide
agtacattaagggtgcctaatgaggaggatccgcgtccatcg

SEQ ID NO: 489 - DNA, 42 bp, Artificial Sequence
  Synthetic Polynucleotide
cggacgtcagatgaacttgttcttcccgggtaccgagcaagc

SEQ ID NO: 490 - DNA, 32 bp, Artificial Sequence
  Synthetic Polynucleotide
aaatgaatagagccgtcaaagctaactcgaga

SEQ ID NO: 491 - DNA, 35 bp, Artificial Sequence
  Synthetic Polynucleotide
atcctgcaacagtgccattttgaaacccttcaaca

SEQ ID NO: 492 - DNA, 28 bp, Artificial Sequence
  Synthetic Polynucleotide
ccgaagcataaagtgtatcgaattccag

SEQ ID NO: 493 - DNA, 35 bp, Artificial Sequence
  Synthetic Polynucleotide
actgtattagactttactttgcgggatgatgacat

SEQ ID NO: 494 - DNA, 28 bp, Artificial Sequence
  Synthetic Polynucleotide
cagttctttttcaccgcctggcccatca

SEQ ID NO: 495 - DNA, 28 bp, Artificial Sequence
  Synthetic Polynucleotide
cactgcgttacgtcagcgtggtgccgtg

SEQ ID NO: 496 - DNA, 35 bp, Artificial Sequence
  Synthetic Polynucleotide
ttcatttgcacaaatatggcggtcagtattataat

SEQ ID NO: 497 - DNA, 38 bp, Artificial Sequence
  Synthetic Polynucleotide
cttaaagcgtggcacagacaatatcgctgagagccaaa

SEQ ID NO: 498 - DNA, 35 bp, Artificial Sequence
  Synthetic Polynucleotide
ttgaagggaccgaactgatagcccgaggtgacaaa

SEQ ID NO: 499 - DNA, 42 bp, Artificial Sequence
  Synthetic Polynucleotide
cccatcagagcgggagcctacaggtagggcgctggcaaaaca

SEQ ID NO: 500 - DNA, 42 bp, Artificial Sequence
  Synthetic Polynucleotide
tgtgaggccgattaaagcccgccgggtcacgctgcgcgttga

SEQ ID NO: 501 - DNA, 42 bp, Artificial Sequence
  Synthetic Polynucleotide
ccgcggtgccttgttccgaatagcccgagatttgccctcacc

SEQ ID NO: 502 - DNA, 42 bp, Artificial Sequence
  Synthetic Polynucleotide
cctatcctgagaagtgtaactatcaaaacgctcatggaccaa

SEQ ID NO: 503 - DNA, 42 bp, Artificial Sequence
  Synthetic Polynucleotide
ctcgttccggtcaatatatgtgagattcctgaaagaaaaagc

SEQ ID NO: 504 - DNA, 42 bp, Artificial Sequence
  Synthetic Polynucleotide
tttatcagtgaggccacttgcctgacattttgacgctcgtaa

SEQ ID NO: 505 - DNA, 28 bp, Artificial Sequence
  Synthetic Polynucleotide
ctggtgatgaagggtaagagcacagtac

SEQ ID NO: 506 - DNA, 38 bp, Artificial Sequence
  Synthetic Polynucleotide
aaaccttgcttctgtaagtgagccaggtttagcgcagc

SEQ ID NO: 507 - DNA, 31 bp, Artificial Sequence
  Synthetic Polynucleotide
taataatgggtaaaggtttcttaatacaaat

SEQ ID NO: 508 - DNA, 35 bp, Artificial Sequence
  Synthetic Polynucleotide
tcttaccaccggggtgtcacttattggggttgcag

SEQ ID NO: 509 - DNA, 28 bp, Artificial Sequence
  Synthetic Polynucleotide
tcgcttttagtatcatagcgtgccgcat

SEQ ID NO: 510 - DNA, 35 bp, Artificial Sequence
  Synthetic Polynucleotide
taacgatgctgattgccgtcgctgacaataaagat

SEQ ID NO: 511 - DNA, 42 bp, Artificial Sequence
  Synthetic Polynucleotide
aaacaaacgcgggatgaaacaaacttaatggaaacagtgcaa

SEQ ID NO: 512 - DNA, 42 bp, Artificial Sequence
  Synthetic Polynucleotide
cggctttccagtcgggagtttgcggcgcgccatgccggacat

SEQ ID NO: 513 - DNA, 42 bp, Artificial Sequence
  Synthetic Polynucleotide
ctgttgcgttgcgctcagtggtttacgatccgcggtgcgact

SEQ ID NO: 514 - DNA, 42 bp, Artificial Sequence
  Synthetic Polynucleotide
gataatacatttgaggacagaaggagcggctcacagtttgta

SEQ ID NO: 515 - DNA, 42 bp, Artificial Sequence
  Synthetic Polynucleotide
gaaaacaactaatagataaatctattgcgtagggagaagcag

SEQ ID NO: 516 - DNA, 42 bp, Artificial Sequence
  Synthetic Polynucleotide
aattaaaatatctttagtgaacctcgtaaaagcctgatcgtt

SEQ ID NO: 517 - DNA, 26 bp, Artificial Sequence
  Synthetic Polynucleotide
cagcagcaaccgcggcggcctttagt

SEQ ID NO: 518 - DNA, 42 bp, Artificial Sequence
  Synthetic Polynucleotide
tcccgtaaaaaaagccgcacaaagaatgccaacggcagcacc

SEQ ID NO: 519 - DNA, 42 bp, Artificial Sequence
  Synthetic Polynucleotide
gtgtacatcgacataaaaaaagtcggtggtgccatcccacgc

SEQ ID NO: 520 - DNA, 42 bp, Artificial Sequence
  Synthetic Polynucleotide
gccgccagcagttgggcggttaaccagcttacggctggaggt

SEQ ID NO: 521 - DNA, 24 bp, Artificial Sequence
  Synthetic Polynucleotide
ttctgctcatttgtccagcatcag

SEQ ID NO: 522 - DNA, 28 bp, Artificial Sequence
  Synthetic Polynucleotide
cagttaatcataagggagcataggagac

SEQ ID NO: 523 - DNA, 39 bp, Artificial Sequence
  Synthetic Polynucleotide
tttagttaataaagcctcatcatttttgtgcgaacaaga

SEQ ID NO: 524 - DNA, 42 bp, Artificial Sequence
  Synthetic Polynucleotide
ggttcggaactcacccttctcacggaaaaagcgacgacatcg

SEQ ID NO: 525 - DNA, 28 bp, Artificial Sequence
  Synthetic Polynucleotide
aatttagagagtaccttgcccgaactgg

SEQ ID NO: 526 - DNA, 21 bp, Artificial Sequence
  Synthetic Polynucleotide
tggtccttttgataagacatc

SEQ ID NO: 527 - DNA, 28 bp, Artificial Sequence
  Synthetic Polynucleotide
acctagcaaaattaagctgaccatctac

SEQ ID NO: 528 - DNA, 42 bp, Artificial Sequence
  Synthetic Polynucleotide
ctttagcattaacatccgctatatataacctcaccgaacgac

SEQ ID NO: 529 - DNA, 35 bp, Artificial Sequence
  Synthetic Polynucleotide
ttcctttaccctgactagtcataaaagaagtaatt

SEQ ID NO: 530 - DNA, 35 bp, Artificial Sequence
  Synthetic Polynucleotide
ttacagaagcaaagcggagcgtcctaatagtcaga

SEQ ID NO: 531 - DNA, 31 bp, Artificial Sequence
  Synthetic Polynucleotide
aaatagggggatgtgctaggactagagtaga

SEQ ID NO: 532 - DNA, 35 bp, Artificial Sequence
  Synthetic Polynucleotide
gaagattaagcttcgctttagtttgaggggaagac

SEQ ID NO: 533 - DNA, 35 bp, Artificial Sequence
  Synthetic Polynucleotide
attaaccgttctagctggaacggtgccccaaaacc

SEQ ID NO: 534 - DNA, 42 bp, Artificial Sequence
  Synthetic Polynucleotide
ggtggttttcaagggcgagtatcggggcgcatcgtaacgctt

SEQ ID NO: 535 - DNA, 42 bp, Artificial Sequence
  Synthetic Polynucleotide
gcagtaaaactcaggctgcactccataggtcacgttgggagc

SEQ ID NO: 536 - DNA, 21 bp, Artificial Sequence
  Synthetic Polynucleotide
taaatcaaaacccctcaaata

SEQ ID NO: 537 - DNA, 35 bp, Artificial Sequence
  Synthetic Polynucleotide
agtagaggaataattgccttagagcttaattataa

SEQ ID NO: 538 - DNA, 35 bp, Artificial Sequence
  Synthetic Polynucleotide
attagtaatgcctgtaacatacaggcaaggcaaat

SEQ ID NO: 539 - DNA, 35 bp, Artificial Sequence
  Synthetic Polynucleotide
ttgaatcatcaggtaaatatcgtcaggaataatgc

SEQ ID NO: 540 - DNA, 42 bp, Artificial Sequence
  Synthetic Polynucleotide
catgtcaatcatagactggatatgtcaaatcaccatcaatat

SEQ ID NO: 541 - DNA, 42 bp, Artificial Sequence
  Synthetic Polynucleotide
gcgcaacactggaacaacattattgttgggaaacaccagccg

SEQ ID NO: 542 - DNA, 42 bp, Artificial Sequence
  Synthetic Polynucleotide
ccaagaaccgaccttcaaggaagtttgattcccaattccgga

SEQ ID NO: 543 - DNA, 34 bp, Artificial Sequence
  Synthetic Polynucleotide
acggaaagattcatcaggctcattttgggctagg

SEQ ID NO: 544 - DNA, 35 bp, Artificial Sequence
  Synthetic Polynucleotide
tacttaggaataccacacttatgcttcaactaact

SEQ ID NO: 545 - DNA, 39 bp, Artificial Sequence
  Synthetic Polynucleotide
tcgcgcaactaatgaaaatgtcagctggcgaaaatgttt

SEQ ID NO: 546 - DNA, 35 bp, Artificial Sequence
  Synthetic Polynucleotide
aattcaacattaaatgttgtagatgcctcagggat

SEQ ID NO: 547 - DNA, 28 bp, Artificial Sequence
  Synthetic Polynucleotide
acagagggggaatactgcggaatcttat

SEQ ID NO: 548 - DNA, 24 bp, Artificial Sequence
  Synthetic Polynucleotide
cgcttatgtaccccggtaaataat

SEQ ID NO: 549 - DNA, 42 bp, Artificial Sequence
  Synthetic Polynucleotide
gtgcagaaaaaatcgtaaaactaggatattccaaaaggttgt

SEQ ID NO: 550 - DNA, 42 bp, Artificial Sequence
  Synthetic Polynucleotide
aatgattttaagaactgttgagatataacgccaaaaggtttg

SEQ ID NO: 551 - DNA, 36 bp, Artificial Sequence
  Synthetic Polynucleotide
gatcgcgcaacaagattgacaagagaatcgatataa

SEQ ID NO: 552 - DNA, 28 bp, Artificial Sequence
  Synthetic Polynucleotide
ggcaccgaacaagtttcattccatgctg

SEQ ID NO: 553 - DNA, 35 bp, Artificial Sequence
  Synthetic Polynucleotide
ctggatattctagtaaaataccagtcaggacacag

SEQ ID NO: 554 - DNA, 42 bp, Artificial Sequence
  Synthetic Polynucleotide
ggcaggccggagacatggggagcataaagctaaatcgggtga

SEQ ID NO: 555 - DNA, 49 bp, Artificial Sequence
  Synthetic Polynucleotide
gtagcaacggtagatacatttcgcaaagaataaaaacattatgactgta

SEQ ID NO: 556 - DNA, 35 bp, Artificial Sequence
  Synthetic Polynucleotide
gttatgcctgaatgccggagagggggagcaatata

SEQ ID NO: 557 - DNA, 35 bp, Artificial Sequence
  Synthetic Polynucleotide
cttatacgtaattgcagggagttaggctttggcaa

SEQ ID NO: 558 - DNA, 35 bp, Artificial Sequence
  Synthetic Polynucleotide
agaaaggccggaaacagcggatcattaatcaatta

SEQ ID NO: 559 - DNA, 42 bp, Artificial Sequence
  Synthetic Polynucleotide
gcacaataacctgtttaaataaattacttttgcgggagaaat

SEQ ID NO: 560 - DNA, 31 bp, Artificial Sequence
  Synthetic Polynucleotide
ggcgaacgaggcgcagacggtcccttcgcac

SEQ ID NO: 561 - DNA, 35 bp, Artificial Sequence
  Synthetic Polynucleotide
tcaatccgaacgagattaccctttgcaaatattca

SEQ ID NO: 562 - DNA, 36 bp, Artificial Sequence
  Synthetic Polynucleotide
cgctattaaacgggtaaatttcatgtcaagagaaga

SEQ ID NO: 563 - DNA, 28 bp, Artificial Sequence
  Synthetic Polynucleotide
taaatcggggtcattgctgagatgcttg

SEQ ID NO: 564 - DNA, 35 bp, Artificial Sequence
  Synthetic Polynucleotide
gcacttttgcgggatcggagggtaacgccagaaag

SEQ ID NO: 565 - DNA, 41 bp, Artificial Sequence
  Synthetic Polynucleotide
agccagcagcgagaaacaatcggctctccgtggtgaaggaa

SEQ ID NO: 566 - DNA, 35 bp, Artificial Sequence
  Synthetic Polynucleotide
gtaaggcatagtaagagagaggctaaatcaaacca

SEQ ID NO: 567 - DNA, 42 bp, Artificial Sequence
  Synthetic Polynucleotide
ccttcctgtagccacgtgcatctgccgtgaattactttctgg

SEQ ID NO: 568 - DNA, 35 bp, Artificial Sequence
  Synthetic Polynucleotide
tcaaggaacgccatcaatgataatcgggcctttgg

SEQ ID NO: 569 - DNA, 42 bp, Artificial Sequence
  Synthetic Polynucleotide
gagtcagctcattttttaaacaggtgttgggccagtcagaca

SEQ ID NO: 570 - DNA, 26 bp, Artificial Sequence
  Synthetic Polynucleotide
gccactacgaaggggtcgctgaggct

SEQ ID NO: 571 - DNA, 42 bp, Artificial Sequence
  Synthetic Polynucleotide
ccacgcataaccgatatattccaccaacctaaaacgaaagag

SEQ ID NO: 572 - DNA, 42 bp, Artificial Sequence
  Synthetic Polynucleotide
gacaatgacaacaaccatcgcgcaaaagaatacactaaaaca

SEQ ID NO: 573 - DNA, 42 bp, Artificial Sequence
  Synthetic Polynucleotide
cttgataccgatagttgcgccctcatctttgacccccagcga

SEQ ID NO: 574 - DNA, 24 bp, Artificial Sequence
  Synthetic Polynucleotide
tttcttaaacagttataccaagcg

SEQ ID NO: 575 - DNA, 28 bp, Artificial Sequence
  Synthetic Polynucleotide
aagttatttaggcagagaattctgccca

SEQ ID NO: 576 - DNA, 22 bp, Artificial Sequence
  Synthetic Polynucleotide
attttgtcaaaatcaccagaac

SEQ ID NO: 577 - DNA, 42 bp, Artificial Sequence
  Synthetic Polynucleotide
tttatgtaaaggcttaggagcctttaattgtgtgtatcaccg

SEQ ID NO: 578 - DNA, 35 bp, Artificial Sequence
  Synthetic Polynucleotide
catagatagccgaacaaagttaagtccagacgaac

SEQ ID NO: 579 - DNA, 21 bp, Artificial Sequence
  Synthetic Polynucleotide
cggagaaggaaaccgagagag

SEQ ID NO: 580 - DNA, 34 bp, Artificial Sequence
  Synthetic Polynucleotide
gcaatacacggaagagaaaatctgacctatcata

SEQ ID NO: 581 - DNA, 45 bp, Artificial Sequence
  Synthetic Polynucleotide
ccgggaattagagccagcacaatccaatcgcgagactatatcagc

SEQ ID NO: 582 - DNA, 42 bp, Artificial Sequence
  Synthetic Polynucleotide
tcacattaaaggtgaatcaaaaggacagtttcagcgtatcgt

SEQ ID NO: 583 - DNA, 35 bp, Artificial Sequence
  Synthetic Polynucleotide
atacctgaacaaagtcaaaaaatgagttacaaaga

SEQ ID NO: 584 - DNA, 35 bp, Artificial Sequence
  Synthetic Polynucleotide
acaattgagcgctaataaacgattattatttgagg

SEQ ID NO: 585 - DNA, 31 bp, Artificial Sequence
  Synthetic Polynucleotide
ataaccctgtagcattcagaacgctaagttt

SEQ ID NO: 586 - DNA, 32 bp, Artificial Sequence
  Synthetic Polynucleotide
atcaaaggatagcaccattaccattagcgcca

SEQ ID NO: 587 - DNA, 35 bp, Artificial Sequence
  Synthetic Polynucleotide
tctagccctctttcgtcgtagcccggaatagatcg

SEQ ID NO: 588 - DNA, 35 bp, Artificial Sequence
  Synthetic Polynucleotide
attgaaccgcctccctcggttgaggccagaacagt

SEQ ID NO: 589 - DNA, 42 bp, Artificial Sequence
  Synthetic Polynucleotide
cccgatctaacccatgtaccgtacgccgtcgagagggttcgg

SEQ ID NO: 590 - DNA, 42 bp, Artificial Sequence
  Synthetic Polynucleotide
cattccagacggatagcaccgccactcagtaccaggcgcatg

SEQ ID NO: 591 - DNA, 21 bp, Artificial Sequence
  Synthetic Polynucleotide
gagaattaactacagagcttt

SEQ ID NO: 592 - DNA, 35 bp, Artificial Sequence
  Synthetic Polynucleotide
gtaagaattgagttaccaatacccaaaagaaataa

SEQ ID NO: 593 - DNA, 35 bp, Artificial Sequence
  Synthetic Polynucleotide
ccgttcggtcgaaaccagtcaccgacttgagatgg

SEQ ID NO: 594 - DNA, 35 bp, Artificial Sequence
  Synthetic Polynucleotide
cagcctttgaacacataagagagtaagcgattaag

SEQ ID NO: 595 - DNA, 42 bp, Artificial Sequence
  Synthetic Polynucleotide
tggccttgatatcaaataagatcaatcaccggaaccagagcc

SEQ ID NO: 596 - DNA, 42 bp, Artificial Sequence
  Synthetic Polynucleotide
ccacccagctcagatatagaaggcatcgtaggagcatgcctg

SEQ ID NO: 597 - DNA, 42 bp, Artificial Sequence
  Synthetic Polynucleotide
aaataatgcagacgacaaaatataaaacgcaaagacacataa

SEQ ID NO: 598 - DNA, 21 bp, Artificial Sequence
  Synthetic Polynucleotide
gtccagcattgacaggaagag

SEQ ID NO: 599 - DNA, 35 bp, Artificial Sequence
  Synthetic Polynucleotide
ttagtattctaagaacgaagcaagtaatcggcaac

SEQ ID NO: 600 - DNA, 45 bp, Artificial Sequence
  Synthetic Polynucleotide
tttttttagcgaacctcagtaccgcattccacgaggtgaacgaaa

SEQ ID NO: 601 - DNA, 39 bp, Artificial Sequence
  Synthetic Polynucleotide
aacaggacttgcggatcccaacaaactacaacgattcct

SEQ ID NO: 602 - DNA, 45 bp, Artificial Sequence
  Synthetic Polynucleotide
gccctattattctgaaagataagttcaggagccaaaaggttgggt

SEQ ID NO: 603 - DNA, 28 bp, Artificial Sequence
  Synthetic Polynucleotide
gcgcaatcaaccgtttttattttcttat

SEQ ID NO: 604 - DNA, 42 bp, Artificial Sequence
  Synthetic Polynucleotide
taacattaaagcaggtcagacgataccaccgagcgtttaagg

SEQ ID NO: 605 - DNA, 42 bp, Artificial Sequence
  Synthetic Polynucleotide
tatcactcatcgagaaccgaggcgtgaagccttaaatcaaat

SEQ ID NO: 606 - DNA, 28 bp, Artificial Sequence
  Synthetic Polynucleotide
agtgcattttaaaggtggcaacatctgg

SEQ ID NO: 607 - DNA, 49 bp, Artificial Sequence
  Synthetic Polynucleotide
ttagcaaatcaatagaaaattcatccatttggaaacgtcaccaatatag

SEQ ID NO: 608 - DNA, 42 bp, Artificial Sequence
  Synthetic Polynucleotide
cttcggcattccaccctcagaaccccgccgctctgaatggta

SEQ ID NO: 609 - DNA, 42 bp, Artificial Sequence
  Synthetic Polynucleotide
tataccagcgccaaagatatcacctcgatagcagcacctttt

SEQ ID NO: 610 - DNA, 28 bp, Artificial Sequence
  Synthetic Polynucleotide
ggtctgaaagacaacacagactttcata

SEQ ID NO: 611 - DNA, 46 bp, Artificial Sequence
  Synthetic Polynucleotide
tagagtgagaatagccaaaaaaaaggctgtttagtaagcccacgca

SEQ ID NO: 612 - DNA, 31 bp, Artificial Sequence
  Synthetic Polynucleotide
atattaacaacgccaacatgtattgatttgt

SEQ ID NO: 613 - DNA, 35 bp, Artificial Sequence
  Synthetic Polynucleotide
atcatcgtagaaaccctgtttatttgccaaaatag

SEQ ID NO: 614 - DNA, 35 bp, Artificial Sequence
  Synthetic Polynucleotide
ggaagttaatttcatctctttttcataaacaaccc

SEQ ID NO: 615 - DNA, 46 bp, Artificial Sequence
  Synthetic Polynucleotide
caaagtactgtcttgttcagccagccatttttgtttaacgtcgagg

SEQ ID NO: 616 - DNA, 42 bp, Artificial Sequence
  Synthetic Polynucleotide
ttgctttagaacggaccagtatctcacaaacaaatccgtata

SEQ ID NO: 617 - DNA, 35 bp, Artificial Sequence
  Synthetic Polynucleotide
gttcctttttaacctcctgctgatgcgtaaccctt

SEQ ID NO: 618 - DNA, 42 bp, Artificial Sequence
  Synthetic Polynucleotide
tgatataagtatattaaaccaccttaatgccccctgcctatt

SEQ ID NO: 619 - DNA, 35 bp, Artificial Sequence
  Synthetic Polynucleotide
ccggttgctattttgcagagcctaatcaacagtaa

SEQ ID NO: 620 - DNA, 35 bp, Artificial Sequence
  Synthetic Polynucleotide
aacttgagtaacagtgcaaatcctcactgagatag

SEQ ID NO: 621 - DNA, 42 bp, Artificial Sequence
  Synthetic Polynucleotide
aaaagttttaacggggttggaaagataggaaagttttgtaac

SEQ ID NO: 622 - DNA, 26 bp, Artificial Sequence
  Synthetic Polynucleotide
aatttaatggtttgaatttatcaaaa

SEQ ID NO: 623 - DNA, 42 bp, Artificial Sequence
  Synthetic Polynucleotide
acgctgagaagagtcaatagtgaaataccgaccgtgtgataa

SEQ ID NO: 624 - DNA, 42 bp, Artificial Sequence
  Synthetic Polynucleotide
atagcgatagcttagattaagataaggcgttaaataagaata

SEQ ID NO: 625 - DNA, 42 bp, Artificial Sequence
  Synthetic Polynucleotide
tcccttagaatccttgaaaacaacaccggaatcataattact

SEQ ID NO: 626 - DNA, 24 bp, Artificial Sequence
  Synthetic Polynucleotide
attaattaatttagaaaaagcctg

SEQ ID NO: 627 - DNA, 42 bp, Artificial Sequence
  Synthetic Polynucleotide
cccggttatctcgacaactcgtataagtttgtaatcctacct

SEQ ID NO: 628 - DNA, 35 bp, Artificial Sequence
  Synthetic Polynucleotide
ctgcagaagataaaacataaaacaacgaccaaatc

SEQ ID NO: 629 - DNA, 42 bp, Artificial Sequence
  Synthetic Polynucleotide
tgaggaatcaatcaaccatatagttacatacctgaaagagtc

SEQ ID NO: 630 - DNA, 38 bp, Artificial Sequence
  Synthetic Polynucleotide
tttatcaagaaaacaaatttcaataaatcgccagtcac

SEQ ID NO: 631 - DNA, 35 bp, Artificial Sequence
  Synthetic Polynucleotide
acaatttcatttgaattgattgttagaacctatat

SEQ ID NO: 632 - DNA, 28 bp, Artificial Sequence
  Synthetic Polynucleotide
gttattaattttaataaatccaaggaat

SEQ ID NO: 633 - DNA, 42 bp, Artificial Sequence
  Synthetic Polynucleotide
agctgttaaataacaacccgtcggtaatgggagccagctaga

SEQ ID NO: 634 - DNA, 35 bp, Artificial Sequence
  Synthetic Polynucleotide
ttgttgcctgagagtcttagctatatattttaagc

SEQ ID NO: 635 - DNA, 42 bp, Artificial Sequence
  Synthetic Polynucleotide
aaattttaaatatttcgccatgacggccggaacggtttcatt

SEQ ID NO: 636 - DNA, 38 bp, Artificial Sequence
  Synthetic Polynucleotide
cttgaaacgtacagcgccgccacgagtgccaccctcat

SEQ ID NO: 637 - DNA, 35 bp, Artificial Sequence
  Synthetic Polynucleotide
ccggaatttgtgagagatttccgggcgccattaaa

SEQ ID NO: 638 - DNA, 28 bp, Artificial Sequence
  Synthetic Polynucleotide
cggcggattgaccgattctcctcgcatt

SEQ ID NO: 639 - DNA, 35 bp, Artificial Sequence
  Synthetic Polynucleotide
gtaaaccaccaccagaggccaccctagcgcggtaa

SEQ ID NO: 640 - DNA, 35 bp, Artificial Sequence
  Synthetic Polynucleotide
atagtattaagaggctgggttttgccctcagaaaa

SEQ ID NO: 641 - DNA, 42 bp, Artificial Sequence
  Synthetic Polynucleotide
gtgtactttaccgtttttcaggttagtaactttcagcgacat

SEQ ID NO: 642 - DNA, 38 bp, Artificial Sequence
  Synthetic Polynucleotide
tctaaaggaacaactaactaaacaaatgaatcagactg

SEQ ID NO: 643 - DNA, 35 bp, Artificial Sequence
  Synthetic Polynucleotide
ataattttttcacgttgaaccgccaccctcatcca

SEQ ID NO: 644 - DNA, 28 bp, Artificial Sequence
  Synthetic Polynucleotide
attaggattagcggagactcctacagga

SEQ ID NO: 645 - DNA, 21 bp, Artificial Sequence
  Synthetic Polynucleotide
ttattcaattaattacattta

SEQ ID NO: 646 - DNA, 21 bp, Artificial Sequence
  Synthetic Polynucleotide
gtggagccatgtttaccagta

SEQ ID NO: 647 - DNA, 21 bp, Artificial Sequence
  Synthetic Polynucleotide
gattttgaggaattgcgaatc

SEQ ID NO: 648 - DNA, 30 bp, Artificial Sequence
  Synthetic Polynucleotide
taatggaagggtttggattatacttctgaa

SEQ ID NO: 649 - DNA, 30 bp, Artificial Sequence
  Synthetic Polynucleotide
gaaaccaggcaaacaccgcttctggtgcgg

SEQ ID NO: 650 - DNA, 30 bp, Artificial Sequence
  Synthetic Polynucleotide
cctcagagccaccaccctcagaaccgccag

SEQ ID NO: 651 - DNA, 21 bp, Artificial Sequence
  Synthetic Polynucleotide
gcagattcacgcagaggcgaa

SEQ ID NO: 652 - DNA, 21 bp, Artificial Sequence
  Synthetic Polynucleotide
atttttagaaagctttcagac

SEQ ID NO: 653 - DNA, 21 bp, Artificial Sequence
  Synthetic Polynucleotide
cctttagcgttttctgtatcg

SEQ ID NO: 654 - DNA, 24 bp, Artificial Sequence
  Synthetic Polynucleotide
gaaccaccaggtcagttggcaatg

SEQ ID NO: 655 - DNA, 24 bp, Artificial Sequence
  Synthetic Polynucleotide
tatcaggtcataaacgttaatatg

SEQ ID NO: 656 - DNA, 24 bp, Artificial Sequence
  Synthetic Polynucleotide
ccgccaccagagcgtcatacataa

SEQ ID NO: 657 - DNA, 21 bp, Artificial Sequence
  Synthetic Polynucleotide
tcgccattaaaaataccgaac

SEQ ID NO: 658 - DNA, 21 bp, Artificial Sequence
  Synthetic Polynucleotide
ttttgagagatctacaaagag

SEQ ID NO: 659 - DNA, 21 bp, Artificial Sequence
  Synthetic Polynucleotide
tcagagccaccaccctcaggc

SEQ ID NO: 660 - DNA, 35 bp, Artificial Sequence
  Synthetic Polynucleotide
tgtccattttgatttgaaatggattatttacatat

SEQ ID NO: 661 - DNA, 35 bp, Artificial Sequence
  Synthetic Polynucleotide
tggggcgatagtagtatttcaacgcaaggataagg

SEQ ID NO: 662 - DNA, 35 bp, Artificial Sequence
  Synthetic Polynucleotide
tcaaccgaattattgtagcgacagaatcaagtttt

SEQ ID NO: 663 - DNA, 21 bp, Artificial Sequence
  Synthetic Polynucleotide
caacagttgatttgcccgatt

SEQ ID NO: 664 - DNA, 21 bp, Artificial Sequence
  Synthetic Polynucleotide
ttgttaaaatgtgggaacagt

SEQ ID NO: 665 - DNA, 21 bp, Artificial Sequence
  Synthetic Polynucleotide
cttttgatgatcaagagaagc

SEQ ID NO: 666 - DNA, 30 bp, Artificial Sequence
  Synthetic Polynucleotide
gtagcaatacttccacgcaaattaaccgac

SEQ ID NO: 667 - DNA, 30 bp, Artificial Sequence
  Synthetic Polynucleotide
atcaattctactacgagctgaaaaggtggg

SEQ ID NO: 668 - DNA, 30 bp, Artificial Sequence
  Synthetic Polynucleotide
aaatattgacggaattgagggagggaagaa

SEQ ID NO: 669 - DNA, 31 bp, Artificial Sequence; identical to SEQ ID NO: 441
  Synthetic Polynucleotide

SEQ ID NO: 670 - DNA, 41 bp, Artificial Sequence; identical to SEQ ID NO: 443
  Synthetic Polynucleotide

SEQ ID NO: 671 - DNA, 48 bp, Artificial Sequence
  Synthetic Polynucleotide
aaaacaaaagatagataaatttacgaatcattaccgcgcccaattttt

SEQ ID NO: 672 - DNA, 54 bp, Artificial Sequence
  Synthetic Polynucleotide
actccttcatacatcgagccagccatataattgtgtcgaaatccgcgacttttt

SEQ ID NO: 673 - DNA, 36 bp, Artificial Sequence
  Synthetic Polynucleotide
tttttcttaattgagaatcgtaataagagaattttt

SEQ ID NO: 674 - DNA, 31 bp, Artificial Sequence
  Synthetic Polynucleotide
tttttaataatatcccatcctagtcctgcga

SEQ ID NO: 675 - DNA, 41 bp, Artificial Sequence
  Synthetic Polynucleotide
ttttttagcaagcaaatacaattttatcctgaatctttttt

SEQ ID NO: 676 - DNA, 46 bp, Artificial Sequence
  Synthetic Polynucleotide
tttttgcaaacgtagaaaataattacgcccctttttaagaaacaag

SEQ ID NO: 677 - DNA, 31 bp, Artificial Sequence
  Synthetic Polynucleotide
tttttatcttaccgaagagtatgttattttt

SEQ ID NO: 678 - DNA, 49 bp, Artificial Sequence
  Synthetic Polynucleotide
tttttgtacagcgtaacagacgagaagaaaaatctacgttaatattttt

SEQ ID NO: 679 - DNA, 54 bp, Artificial Sequence
  Synthetic Polynucleotide
tgtagcttgtctggtgaccaattagccggcggttgcggtatgagccgggttttt

SEQ ID NO: 680 - DNA, 36 bp, Artificial Sequence
  Synthetic Polynucleotide
tttttctgctccatgttacctttgaaagaggttttt

SEQ ID NO: 681 - DNA, 31 bp, Artificial Sequence
  Synthetic Polynucleotide
tttttgaataaggcttgccctaagctgcaaa

SEQ ID NO: 682 - DNA, 41 bp, Artificial Sequence
  Synthetic Polynucleotide
tttttaaacgaactaacatcataaccctcgtttaccttttt

SEQ ID NO: 683 - DNA, 45 bp, Artificial Sequence
  Synthetic Polynucleotide
ttttttgcaactaaagtacggcaacatggcaaactccaacaggcg

SEQ ID NO: 684 - DNA, 46 bp, Artificial Sequence
  Synthetic Polynucleotide
ttttttataacgtgctttccttgctttgtcaagcgaaaggagaacg

SEQ ID NO: 685 - DNA, 31 bp, Artificial Sequence
  Synthetic Polynucleotide
tttttaccagaccggaattttaaatattttt

SEQ ID NO: 686 - DNA, 48 bp, Artificial Sequence; identical to SEQ ID NO: 440
  Synthetic Polynucleotide

SEQ ID NO: 687 - DNA, 54 bp, Artificial Sequence
  Synthetic Polynucleotide
ctatggtcgttagattacactcggctggagccaacgctcaacagtagggttttt

SEQ ID NO: 688 - DNA, 36 bp, Artificial Sequence
  Synthetic Polynucleotide
ttttttcactgttgccctgggtgtgttcagcttttt

SEQ ID NO: 689 - DNA, 31 bp, Artificial Sequence
  Synthetic Polynucleotide
tttttaaaaaccgtctaacgagcacgttttt

SEQ ID NO: 690 - DNA, 67 bp, Artificial Sequence
  Synthetic Polynucleotide
ggggtggtttgccccagcaggcgttcactaatctgatggaagcgcattagatagcaatagctttttt

SEQ ID NO: 691 - DNA, 67 bp, Artificial Sequence
  Synthetic Polynucleotide
ccaaaatgctttaaacagttcaggcaaaattctcattgaaaatcctgtttcgtcaaagggcgttttt

SEQ ID NO: 692 - DNA, 67 bp, Artificial Sequence
  Synthetic Polynucleotide
gcgtagaataacataaaaacaggaatgtcgatatctagaaaacgagaatggcttcaaagcgattttt

SEQ ID NO: 693 - DNA, 64 bp, Artificial Sequence
  Synthetic Polynucleotide
tttttaatcggccaacgtgctgcggcttcactaatctgatgtataaagtaccgcaatgaaacgg

SEQ ID NO: 694 - DNA, 64 bp, Artificial Sequence
  Synthetic Polynucleotide
tttttagacgacgataatcattcagtgcaaaattctcattgaaatcgttaacgactccaagatg

SEQ ID NO: 695 - DNA, 64 bp, Artificial Sequence
  Synthetic Polynucleotide
ttttttaccaacgctaaaacaagaaaaatgtcgatatctagacagatgaacggaattcgaacca

SEQ ID NO: 696 - DNA, 15 bp, Artificial Sequence
  Synthetic Polynucleotide
catcagattagtgaa

SEQ ID NO: 697 - DNA, 15 bp, Artificial Sequence
  Synthetic Polynucleotide
caatgagaattttgc

SEQ ID NO: 698 - DNA, 15 bp, Artificial Sequence
  Synthetic Polynucleotide
ctagatatcgacatt

SEQ ID NO: 699 - DNA, 46 bp, Artificial Sequence; identical to SEQ ID NO: 471
  Synthetic Polynucleotide

SEQ ID NO: 700 - DNA, 38 bp, Artificial Sequence; identical to SEQ ID NO: 472
  Synthetic Polynucleotide

SEQ ID NO: 701 - DNA, 35 bp, Artificial Sequence; identical to SEQ ID NO: 473
  Synthetic Polynucleotide

SEQ ID NO: 702 - DNA, 42 bp, Artificial Sequence; identical to SEQ ID NO: 474
  Synthetic Polynucleotide

SEQ ID NO: 703 - DNA, 35 bp, Artificial Sequence; identical to SEQ ID NO: 232
  Synthetic Polynucleotide

SEQ ID NO: 704 - DNA, 42 bp, Artificial Sequence; identical to SEQ ID NO: 476
  Synthetic Polynucleotide

SEQ ID NO: 705 - DNA, 31 bp, Artificial Sequence; identical to SEQ ID NO: 477
  Synthetic Polynucleotide

SEQ ID NO: 706 - DNA, 35 bp, Artificial Sequence; identical to SEQ ID NO: 478
  Synthetic Polynucleotide

SEQ ID NO: 707 - DNA, 35 bp, Artificial Sequence; identical to SEQ ID NO: 479
  Synthetic Polynucleotide

SEQ ID NO: 708 - DNA, 42 bp, Artificial Sequence; identical to SEQ ID NO: 480
  Synthetic Polynucleotide

SEQ ID NO: 709 - DNA, 42 bp, Artificial Sequence; identical to SEQ ID NO: 481
  Synthetic Polynucleotide

SEQ ID NO: 710 - DNA, 21 bp, Artificial Sequence
  Synthetic Polynucleotide
gtggttccgatccacgcagag

SEQ ID NO: 711 - DNA, 25 bp, Artificial Sequence
  Synthetic Polynucleotide
aaaagtttgggtgtagccgcttaat

SEQ ID NO: 712 - DNA, 42 bp, Artificial Sequence; identical to SEQ ID NO: 484
  Synthetic Polynucleotide

SEQ ID NO: 713 - DNA, 35 bp, Artificial Sequence; identical to SEQ ID NO: 485
  Synthetic Polynucleotide

SEQ ID NO: 714 - DNA, 46 bp, Artificial Sequence; identical to SEQ ID NO: 486
  Synthetic Polynucleotide

SEQ ID NO: 715 - DNA, 42 bp, Artificial Sequence; identical to SEQ ID NO: 487
  Synthetic Polynucleotide

SEQ ID NO: 716 - DNA, 42 bp, Artificial Sequence; identical to SEQ ID NO: 488
  Synthetic Polynucleotide

SEQ ID NO: 717 - DNA, 42 bp, Artificial Sequence; identical to SEQ ID NO: 489
  Synthetic Polynucleotide

SEQ ID NO: 718 - DNA, 32 bp, Artificial Sequence; identical to SEQ ID NO: 490
  Synthetic Polynucleotide

SEQ ID NO: 719 - DNA, 35 bp, Artificial Sequence; identical to SEQ ID NO: 491
  Synthetic Polynucleotide

SEQ ID NO: 720 - DNA, 28 bp, Artificial Sequence
  Synthetic Polynucleotide
ccgaagcataaagtgtatcgaattccag

SEQ ID NO: 721 - DNA, 35 bp, Artificial Sequence; identical to SEQ ID NO: 493
  Synthetic Polynucleotide

SEQ ID NO: 722 - DNA, 28 bp, Artificial Sequence
  Synthetic Polynucleotide
cagttctttttcaccgcctggcccatca

SEQ ID NO: 723 - DNA, 28 bp, Artificial Sequence
  Synthetic Polynucleotide
cactgcgttacgtcagcgtggtgccgtg

SEQ ID NO: 724 - DNA, 35 bp, Artificial Sequence; identical to SEQ ID NO: 496
  Synthetic Polynucleotide

SEQ ID NO: 725 - DNA, 38 bp, Artificial Sequence; identical to SEQ ID NO: 497
  Synthetic Polynucleotide

SEQ ID NO: 726 - DNA, 35 bp, Artificial Sequence; identical to SEQ ID NO: 498
  Synthetic Polynucleotide

SEQ ID NO: 727 - DNA, 42 bp, Artificial Sequence; identical to SEQ ID NO: 499
  Synthetic Polynucleotide

SEQ ID NO: 728 - DNA, 42 bp, Artificial Sequence; identical to SEQ ID NO: 500
  Synthetic Polynucleotide

SEQ ID NO: 729 - DNA, 42 bp, Artificial Sequence; identical to SEQ ID NO: 501
  Synthetic Polynucleotide

SEQ ID NO: 730 - DNA, 42 bp, Artificial Sequence; identical to SEQ ID NO: 502
  Synthetic Polynucleotide

SEQ ID NO: 731 - DNA, 42 bp, Artificial Sequence; identical to SEQ ID NO: 503
  Synthetic Polynucleotide

SEQ ID NO: 732 - DNA, 42 bp, Artificial Sequence; identical to SEQ ID NO: 504
  Synthetic Polynucleotide

SEQ ID NO: 733 - DNA, 28 bp, Artificial Sequence
  Synthetic Polynucleotide
ctggtgatgaagggtaagagcacagtac

SEQ ID NO: 734 - DNA, 38 bp, Artificial Sequence; identical to SEQ ID NO: 506
  Synthetic Polynucleotide

SEQ ID NO: 735 - DNA, 31 bp, Artificial Sequence; identical to SEQ ID NO: 507
  Synthetic Polynucleotide

SEQ ID NO: 736 - DNA, 35 bp, Artificial Sequence; identical to SEQ ID NO: 508
  Synthetic Polynucleotide

SEQ ID NO: 737 - DNA, 28 bp, Artificial Sequence
  Synthetic Polynucleotide
tcgcttttagtatcatagcgtgccgcat

SEQ ID NO: 738 - DNA, 35 bp, Artificial Sequence; identical to SEQ ID NO: 510
  Synthetic Polynucleotide

SEQ ID NO: 739 - DNA, 42 bp, Artificial Sequence; identical to SEQ ID NO: 511
  Synthetic Polynucleotide

SEQ ID NO: 740 - DNA, 42 bp, Artificial Sequence; identical to SEQ ID NO: 512
  Synthetic Polynucleotide

SEQ ID NO: 741 - DNA, 42 bp, Artificial Sequence; identical to SEQ ID NO: 513
  Synthetic Polynucleotide

SEQ ID NO: 742 - DNA, 42 bp, Artificial Sequence; identical to SEQ ID NO: 514
  Synthetic Polynucleotide

SEQ ID NO: 743 - DNA, 42 bp, Artificial Sequence; identical to SEQ ID NO: 515
  Synthetic Polynucleotide

SEQ ID NO: 744 - DNA, 42 bp, Artificial Sequence; identical to SEQ ID NO: 516
  Synthetic Polynucleotide

SEQ ID NO: 745 - DNA, 26 bp, Artificial Sequence
  Synthetic Polynucleotide
cagcagcaaccgcggcggcctttagt

SEQ ID NO: 746 - DNA, 42 bp, Artificial Sequence; identical to SEQ ID NO: 518
  Synthetic Polynucleotide

SEQ ID NO: 747 - DNA, 42 bp, Artificial Sequence; identical to SEQ ID NO: 519
  Synthetic Polynucleotide

SEQ ID NO: 748 - DNA, 42 bp, Artificial Sequence; identical to SEQ ID NO: 520
  Synthetic Polynucleotide

SEQ ID NO: 749 - DNA, 24 bp, Artificial Sequence
  Synthetic Polynucleotide
ttctgctcatttgtccagcatcag

SEQ ID NO: 750 - DNA, 28 bp, Artificial Sequence
  Synthetic Polynucleotide
cagttaatcataagggagcataggagac

SEQ ID NO: 751 - DNA, 39 bp, Artificial Sequence; identical to SEQ ID NO: 523
  Synthetic Polynucleotide

SEQ ID NO: 752 - DNA, 42 bp, Artificial Sequence; identical to SEQ ID NO: 524
  Synthetic Polynucleotide

SEQ ID NO: 753 - DNA, 28 bp, Artificial Sequence
  Synthetic Polynucleotide
aatttagagagtaccttgcccgaactgg

SEQ ID NO: 754 - DNA, 21 bp, Artificial Sequence
  Synthetic Polynucleotide
tggtccttttgataagacatc

SEQ ID NO: 755 - DNA, 28 bp, Artificial Sequence
  Synthetic Polynucleotide
acctagcaaaattaagctgaccatctac

SEQ ID NO: 756 - DNA, 42 bp, Artificial Sequence; identical to SEQ ID NO: 528
  Synthetic Polynucleotide

SEQ ID NO: 757 - DNA, 35 bp, Artificial Sequence; identical to SEQ ID NO: 529
  Synthetic Polynucleotide

SEQ ID NO: 758 - DNA, 35 bp, Artificial Sequence; identical to SEQ ID NO: 530
  Synthetic Polynucleotide

SEQ ID NO: 759 - DNA, 31 bp, Artificial Sequence; identical to SEQ ID NO: 531
  Synthetic Polynucleotide

SEQ ID NO: 760 - DNA, 35 bp, Artificial Sequence; identical to SEQ ID NO: 532
  Synthetic Polynucleotide

SEQ ID NO: 761 - DNA, 35 bp, Artificial Sequence; identical to SEQ ID NO: 533
  Synthetic Polynucleotide

SEQ ID NO: 762 - DNA, 42 bp, Artificial Sequence; identical to SEQ ID NO: 534
  Synthetic Polynucleotide

SEQ ID NO: 763 - DNA, 42 bp, Artificial Sequence; identical to SEQ ID NO: 535
  Synthetic Polynucleotide

SEQ ID NO: 764 - DNA, 21 bp, Artificial Sequence
  Synthetic Polynucleotide
taaatcaaaacccctcaaata

SEQ ID NO: 765 - DNA, 35 bp, Artificial Sequence; identical to SEQ ID NO: 537
  Synthetic Polynucleotide

SEQ ID NO: 766 - DNA, 35 bp, Artificial Sequence; identical to SEQ ID NO: 538
  Synthetic Polynucleotide

SEQ ID NO: 767 - DNA, 35 bp, Artificial Sequence; identical to SEQ ID NO: 539
  Synthetic Polynucleotide

SEQ ID NO: 768 - DNA, 42 bp, Artificial Sequence; identical to SEQ ID NO: 540
  Synthetic Polynucleotide

SEQ ID NO: 769 - DNA, 42 bp, Artificial Sequence; identical to SEQ ID NO: 541
  Synthetic Polynucleotide

SEQ ID NO: 770 - DNA, 42 bp, Artificial Sequence; identical to SEQ ID NO: 542
  Synthetic Polynucleotide

SEQ ID NO: 771 - DNA, 34 bp, Artificial Sequence; identical to SEQ ID NO: 543
  Synthetic Polynucleotide

SEQ ID NO: 772 - DNA, 35 bp, Artificial Sequence; identical to SEQ ID NO: 544
  Synthetic Polynucleotide

SEQ ID NO: 773 - DNA, 39 bp, Artificial Sequence; identical to SEQ ID NO: 545
  Synthetic Polynucleotide

SEQ ID NO: 774 - DNA, 35 bp, Artificial Sequence; identical to SEQ ID NO: 546
  Synthetic Polynucleotide

SEQ ID NO: 775 - DNA, 28 bp, Artificial Sequence
  Synthetic Polynucleotide
acagagggggaatactgcggaatcttat

SEQ ID NO: 776 - DNA, 24 bp, Artificial Sequence
  Synthetic Polynucleotide
cgcttatgtaccccggtaaataat

SEQ ID NO: 777 - DNA, 42 bp, Artificial Sequence; identical to SEQ ID NO: 549
  Synthetic Polynucleotide

SEQ ID NO: 778 - DNA, 42 bp, Artificial Sequence; identical to SEQ ID NO: 550
  Synthetic Polynucleotide

SEQ ID NO: 779 - DNA, 36 bp, Artificial Sequence; identical to SEQ ID NO: 551
  Synthetic Polynucleotide

SEQ ID NO: 780 - DNA, 28 bp, Artificial Sequence
  Synthetic Polynucleotide
ggcaccgaacaagtttcattccatgctg

SEQ ID NO: 781 - DNA, 35 bp, Artificial Sequence; identical to SEQ ID NO: 553
  Synthetic Polynucleotide

SEQ ID NO: 782 - DNA, 42 bp, Artificial Sequence; identical to SEQ ID NO: 554
  Synthetic Polynucleotide

SEQ ID NO: 783 - DNA, 49 bp, Artificial Sequence; identical to SEQ ID NO: 555
  Synthetic Polynucleotide

SEQ ID NO: 784 - DNA, 35 bp, Artificial Sequence; identical to SEQ ID NO: 556
  Synthetic Polynucleotide

SEQ ID NO: 785 - DNA, 35 bp, Artificial Sequence; identical to SEQ ID NO: 557
  Synthetic Polynucleotide

SEQ ID NO: 786 - DNA, 35 bp, Artificial Sequence; identical to SEQ ID NO: 558
  Synthetic Polynucleotide

SEQ ID NO: 787 - DNA, 42 bp, Artificial Sequence; identical to SEQ ID NO: 559
  Synthetic Polynucleotide

SEQ ID NO: 788 - DNA, 31 bp, Artificial Sequence; identical to SEQ ID NO: 560
  Synthetic Polynucleotide

SEQ ID NO: 789 - DNA, 35 bp, Artificial Sequence; identical to SEQ ID NO: 561
  Synthetic Polynucleotide

SEQ ID NO: 790 - DNA, 36 bp, Artificial Sequence; identical to SEQ ID NO: 562
  Synthetic Polynucleotide

SEQ ID NO: 791 - DNA, 28 bp, Artificial Sequence
  Synthetic Polynucleotide
taaatcggggtcattgctgagatgcttg

SEQ ID NO: 792 - DNA, 35 bp, Artificial Sequence; identical to SEQ ID NO: 564
  Synthetic Polynucleotide

SEQ ID NO: 793 - DNA, 41 bp, Artificial Sequence; identical to SEQ ID NO: 565
  Synthetic Polynucleotide

SEQ ID NO: 794 - DNA, 35 bp, Artificial Sequence; identical to SEQ ID NO: 566
  Synthetic Polynucleotide

SEQ ID NO: 795 - DNA, 42 bp, Artificial Sequence; identical to SEQ ID NO: 567
  Synthetic Polynucleotide

SEQ ID NO: 796 - DNA, 35 bp, Artificial Sequence; identical to SEQ ID NO: 568
  Synthetic Polynucleotide

SEQ ID NO: 797 - DNA, 42 bp, Artificial Sequence; identical to SEQ ID NO: 569
  Synthetic Polynucleotide

SEQ ID NO: 798 - DNA, 26 bp, Artificial Sequence
  Synthetic Polynucleotide
gccactacgaaggggtcgctgaggct

SEQ ID NO: 799 - DNA, 42 bp, Artificial Sequence; identical to SEQ ID NO: 571
  Synthetic Polynucleotide

SEQ ID NO: 800 - DNA, 42 bp, Artificial Sequence; identical to SEQ ID NO: 572
  Synthetic Polynucleotide

SEQ ID NO: 801 - DNA, 42 bp, Artificial Sequence; identical to SEQ ID NO: 573
  Synthetic Polynucleotide

SEQ ID NO: 802 - DNA, 24 bp, Artificial Sequence
  Synthetic Polynucleotide
tttcttaaacagttataccaagcg

SEQ ID NO: 803 - DNA, 28 bp, Artificial Sequence
  Synthetic Polynucleotide
aagttatttaggcagagaattctgccca

SEQ ID NO: 804 - DNA, 22 bp, Artificial Sequence
  Synthetic Polynucleotide
attttgtcaaaatcaccagaac

SEQ ID NO: 805 - DNA, 42 bp, Artificial Sequence; identical to SEQ ID NO: 577
  Synthetic Polynucleotide

SEQ ID NO: 806 - DNA, 35 bp, Artificial Sequence; identical to SEQ ID NO: 578
  Synthetic Polynucleotide

SEQ ID NO: 807 - DNA, 21 bp, Artificial Sequence
  Synthetic Polynucleotide
cggagaaggaaaccgagagag

SEQ ID NO: 808 - DNA, 34 bp, Artificial Sequence; identical to SEQ ID NO: 580
  Synthetic Polynucleotide

SEQ ID NO: 809 - DNA, 45 bp, Artificial Sequence; identical to SEQ ID NO: 581
  Synthetic Polynucleotide

SEQ ID NO: 810 - DNA, 42 bp, Artificial Sequence; identical to SEQ ID NO: 582
  Synthetic Polynucleotide

SEQ ID NO: 811 - DNA, 35 bp, Artificial Sequence; identical to SEQ ID NO: 583
  Synthetic Polynucleotide

SEQ ID NO: 812 - DNA, 35 bp, Artificial Sequence; identical to SEQ ID NO: 584
  Synthetic Polynucleotide

SEQ ID NO: 813 - DNA, 31 bp, Artificial Sequence; identical to SEQ ID NO: 585
  Synthetic Polynucleotide

SEQ ID NO: 814 - DNA, 32 bp, Artificial Sequence; identical to SEQ ID NO: 586
  Synthetic Polynucleotide

SEQ ID NO: 815 - DNA, 35 bp, Artificial Sequence; identical to SEQ ID NO: 587
  Synthetic Polynucleotide

SEQ ID NO: 816 - DNA, 35 bp, Artificial Sequence; identical to SEQ ID NO: 588
  Synthetic Polynucleotide

SEQ ID NO: 817 - DNA, 42 bp, Artificial Sequence; identical to SEQ ID NO: 589
  Synthetic Polynucleotide

SEQ ID NO: 818 - DNA, 42 bp, Artificial Sequence; identical to SEQ ID NO: 590
  Synthetic Polynucleotide

SEQ ID NO: 819 - DNA, 21 bp, Artificial Sequence
  Synthetic Polynucleotide
gagaattaactacagagcttt

SEQ ID NO: 820 - DNA, 35 bp, Artificial Sequence; identical to SEQ ID NO: 592
  Synthetic Polynucleotide

SEQ ID NO: 821 - DNA, 35 bp, Artificial Sequence; identical to SEQ ID NO: 593
  Synthetic Polynucleotide

SEQ ID NO: 822 - DNA, 35 bp, Artificial Sequence; identical to SEQ ID NO: 594
  Synthetic Polynucleotide

SEQ ID NO: 823 - DNA, 42 bp, Artificial Sequence; identical to SEQ ID NO: 595
  Synthetic Polynucleotide

SEQ ID NO: 824 - DNA, 42 bp, Artificial Sequence; identical to SEQ ID NO: 596
  Synthetic Polynucleotide

SEQ ID NO: 825 - DNA, 42 bp, Artificial Sequence; identical to SEQ ID NO: 597
  Synthetic Polynucleotide

SEQ ID NO: 826 - DNA, 21 bp, Artificial Sequence
  Synthetic Polynucleotide
gtccagcattgacaggaagag

SEQ ID NO: 827 - DNA, 35 bp, Artificial Sequence; identical to SEQ ID NO: 599
  Synthetic Polynucleotide

SEQ ID NO: 828 - DNA, 45 bp, Artificial Sequence; identical to SEQ ID NO: 600
  Synthetic Polynucleotide

SEQ ID NO: 829 - DNA, 39 bp, Artificial Sequence; identical to SEQ ID NO: 601
  Synthetic Polynucleotide

SEQ ID NO: 830 - DNA, 45 bp, Artificial Sequence; identical to SEQ ID NO: 602
  Synthetic Polynucleotide

SEQ ID NO: 831 - DNA, 28 bp, Artificial Sequence
  Synthetic Polynucleotide
gcgcaatcaaccgtttttattttcttat

SEQ ID NO: 832 - DNA, 42 bp, Artificial Sequence; identical to SEQ ID NO: 604
  Synthetic Polynucleotide

SEQ ID NO: 833 - DNA, 42 bp, Artificial Sequence; identical to SEQ ID NO: 605
  Synthetic Polynucleotide

SEQ ID NO: 834 - DNA, 28 bp, Artificial Sequence
  Synthetic Polynucleotide
agtgcattttaaaggtggcaacatctgg

SEQ ID NO: 835 - DNA, 49 bp, Artificial Sequence; identical to SEQ ID NO: 607
  Synthetic Polynucleotide

SEQ ID NO: 836 - DNA, 42 bp, Artificial Sequence; identical to SEQ ID NO: 608
  Synthetic Polynucleotide

SEQ ID NO: 837 - DNA, 42 bp, Artificial Sequence; identical to SEQ ID NO: 609
  Synthetic Polynucleotide

SEQ ID NO: 838 - DNA, 28 bp, Artificial Sequence
  Synthetic Polynucleotide
ggtctgaaagacaacacagactttcata

SEQ ID NO: 839 - DNA, 46 bp, Artificial Sequence; identical to SEQ ID NO: 611
  Synthetic Polynucleotide

SEQ ID NO: 840 - DNA, 31 bp, Artificial Sequence; identical to SEQ ID NO: 612
  Synthetic Polynucleotide

SEQ ID NO: 841 - DNA, 35 bp, Artificial Sequence; identical to SEQ ID NO: 613
  Synthetic Polynucleotide

SEQ ID NO: 842 - DNA, 35 bp, Artificial Sequence; identical to SEQ ID NO: 614
  Synthetic Polynucleotide

SEQ ID NO: 843 - DNA, 46 bp, Artificial Sequence; identical to SEQ ID NO: 615
  Synthetic Polynucleotide

SEQ ID NO: 844 - DNA, 42 bp, Artificial Sequence; identical to SEQ ID NO: 616
  Synthetic Polynucleotide

SEQ ID NO: 845 - DNA, 35 bp, Artificial Sequence; identical to SEQ ID NO: 617
  Synthetic Polynucleotide

SEQ ID NO: 846 - DNA, 42 bp, Artificial Sequence; identical to SEQ ID NO: 618
  Synthetic Polynucleotide

SEQ ID NO: 847 - DNA, 35 bp, Artificial Sequence; identical to SEQ ID NO: 619
  Synthetic Polynucleotide

SEQ ID NO: 848 - DNA, 35 bp, Artificial Sequence; identical to SEQ ID NO: 620
  Synthetic Polynucleotide

SEQ ID NO: 849 - DNA, 42 bp, Artificial Sequence; identical to SEQ ID NO: 621
  Synthetic Polynucleotide

SEQ ID NO: 850 - DNA, 26 bp, Artificial Sequence
  Synthetic Polynucleotide
aatttaatggtttgaatttatcaaaa

SEQ ID NO: 851 - DNA, 42 bp, Artificial Sequence; identical to SEQ ID NO: 623
  Synthetic Polynucleotide

SEQ ID NO: 852 - DNA, 42 bp, Artificial Sequence; identical to SEQ ID NO: 624
  Synthetic Polynucleotide

SEQ ID NO: 853 - DNA, 42 bp, Artificial Sequence; identical to SEQ ID NO: 625
  Synthetic Polynucleotide

SEQ ID NO: 854 - DNA, 24 bp, Artificial Sequence
  Synthetic Polynucleotide
attaattaatttagaaaaagcctg

SEQ ID NO: 855 - DNA, 30 bp, Artificial Sequence
  Synthetic Polynucleotide
gtagcaatacttctttgatttgaaatggat

SEQ ID NO: 856 - DNA, 27 bp, Artificial Sequence
  Synthetic Polynucleotide
gcagattcaccagtcactcgccattaa

SEQ ID NO: 857 - DNA, 42 bp, Artificial Sequence
  Synthetic Polynucleotide
gaaccaccagcagaagataaaacataaaacaacgaccaaatc

SEQ ID NO: 858 - DNA, 42 bp, Artificial Sequence; identical to SEQ ID NO: 627
  Synthetic Polynucleotide

SEQ ID NO: 859 - DNA, 49 bp, Artificial Sequence
  Synthetic Polynucleotide
caacagttgaaaggaattgaggaatcaatcaaccatatagttacatacc

SEQ ID NO: 860 - DNA, 30 bp, Artificial Sequence
  Synthetic Polynucleotide
taatggaagggttagaacctatatctggtc

SEQ ID NO: 861 - DNA, 23 bp, Artificial Sequence
  Synthetic Polynucleotide
tgaaagagtctgtccatcacgca

SEQ ID NO: 862 - DNA, 21 bp, Artificial Sequence
  Synthetic Polynucleotide
ttattcatttcaataaatcgc

SEQ ID NO: 863 - DNA, 20 bp, Artificial Sequence
  Synthetic Polynucleotide
tttatcaagaaaacaaaatt

SEQ ID NO: 864 - DNA, 30 bp, Artificial Sequence
  Synthetic Polynucleotide
acaatttcatttgaattgattgtttggatt

SEQ ID NO: 865 - DNA, 21 bp, Artificial Sequence
  Synthetic Polynucleotide
gttattaattttaataaatcc

SEQ ID NO: 866 - DNA, 30 bp, Artificial Sequence
  Synthetic Polynucleotide
atcaattctactaatagtagtatttcaacg

SEQ ID NO: 867 - DNA, 27 bp, Artificial Sequence
  Synthetic Polynucleotide
atttttagaaccctcatttttgagaga

SEQ ID NO: 868 - DNA, 42 bp, Artificial Sequence
  Synthetic Polynucleotide
tatcaggtcattgcctgagagtcttagctatatattttaagc

SEQ ID NO: 869 - DNA, 42 bp, Artificial Sequence; identical to SEQ ID NO: 633
  Synthetic Polynucleotide

SEQ ID NO: 870 - DNA, 49 bp, Artificial Sequence
  Synthetic Polynucleotide
ttgttaaaattcgcattaaattttaaatatttcgccatgacggccggaa

SEQ ID NO: 871 - DNA, 30 bp, Artificial Sequence
  Synthetic Polynucleotide
gaaaccaggcaaagcgccattaaattgtaa

SEQ ID NO: 872 - DNA, 23 bp, Artificial Sequence
  Synthetic Polynucleotide
cggtttcatttggggcgcgagct

SEQ ID NO: 873 - DNA, 21 bp, Artificial Sequence
  Synthetic Polynucleotide
gtggagccgccacgagtgcca

SEQ ID NO: 874 - DNA, 20 bp, Artificial Sequence
  Synthetic Polynucleotide
cttgaaacgtacagcgccat

SEQ ID NO: 875 - DNA, 30 bp, Artificial Sequence
  Synthetic Polynucleotide
ccggaatttgtgagagatttccggcaccgc

SEQ ID NO: 876 - DNA, 21 bp, Artificial Sequence
  Synthetic Polynucleotide
cggcggattgaccgattctcc

SEQ ID NO: 877 - DNA, 30 bp, Artificial Sequence
  Synthetic Polynucleotide
aaatattgacggaaattattgtagcgacag

SEQ ID NO: 878 - DNA, 27 bp, Artificial Sequence
  Synthetic Polynucleotide
cctttagcgtcagactgtcagagccac

SEQ ID NO: 879 - DNA, 42 bp, Artificial Sequence
  Synthetic Polynucleotide
ccgccaccagaaccaccaccagaggccaccctagcgcggtaa

SEQ ID NO: 880 - DNA, 35 bp, Artificial Sequence; identical to SEQ ID NO: 640
  Synthetic Polynucleotide

SEQ ID NO: 881 - DNA, 49 bp, Artificial Sequence
  Synthetic Polynucleotide
cttttgatgatacaggagtgtactttaccgtttttcaggttagtaactt

SEQ ID NO: 882 - DNA, 30 bp, Artificial Sequence
  Synthetic Polynucleotide
cctcagagccaccaccctcatccagtaagc

SEQ ID NO: 883 - DNA, 23 bp, Artificial Sequence
  Synthetic Polynucleotide
tcagcgacattcaaccgattgag

SEQ ID NO: 884 - DNA, 21 bp, Artificial Sequence
  Synthetic Polynucleotide
gattttgctaaacaaatgaat

SEQ ID NO: 885 - DNA, 20 bp, Artificial Sequence
  Synthetic Polynucleotide
tctaaaggaacaactaaagg

SEQ ID NO: 886 - DNA, 30 bp, Artificial Sequence
  Synthetic Polynucleotide
ataattttttcacgttgaaccgccaccctc

SEQ ID NO: 887 - DNA, 21 bp, Artificial Sequence
  Synthetic Polynucleotide
attaggattagcggagactcc

SEQ ID NO: 888 - DNA, 11 bp, Artificial Sequence
  Synthetic Polynucleotide
aattacattta

SEQ ID NO: 889 - DNA, 11 bp, Artificial Sequence
  Synthetic Polynucleotide
gtttaccagta

SEQ ID NO: 890 - DNA, 11 bp, Artificial Sequence
  Synthetic Polynucleotide
aattgcgaatc

SEQ ID NO: 891 - DNA, 11 bp, Artificial Sequence
  Synthetic Polynucleotide
atacttctgaa

SEQ ID NO: 892 - DNA, 11 bp, Artificial Sequence
  Synthetic Polynucleotide
ttctggtgcgg

SEQ ID NO: 893 - DNA, 11 bp, Artificial Sequence
  Synthetic Polynucleotide
agaaccgccag

SEQ ID NO: 894 - DNA, 11 bp, Artificial Sequence
  Synthetic Polynucleotide
gcagaggcgaa

SEQ ID NO: 895 - DNA, 11 bp, Artificial Sequence
  Synthetic Polynucleotide
agctttcagac

SEQ ID NO: 896 - DNA, 11 bp, Artificial Sequence
  Synthetic Polynucleotide
tttctgtatcg

SEQ ID NO: 897 - DNA, 11 bp, Artificial Sequence
  Synthetic Polynucleotide
agttggcaatg

SEQ ID NO: 898 - DNA, 11 bp, Artificial Sequence
  Synthetic Polynucleotide
acgttaatatg

SEQ ID NO: 899 - DNA, 11 bp, Artificial Sequence
  Synthetic Polynucleotide
gtcatacataa

SEQ ID NO: 900 - DNA, 11 bp, Artificial Sequence
  Synthetic Polynucleotide
aaataccgaac

SEQ ID NO: 901 - DNA, 11 bp, Artificial Sequence
  Synthetic Polynucleotide
tctacaaagag

SEQ ID NO: 902 - DNA, 11 bp, Artificial Sequence
  Synthetic Polynucleotide
caccctcaggc

SEQ ID NO: 903 - DNA, 11 bp, Artificial Sequence
  Synthetic Polynucleotide
tatttacatat

SEQ ID NO: 904 - DNA, 11 bp, Artificial Sequence
  Synthetic Polynucleotide
caaggataagg

SEQ ID NO: 905 - DNA, 11 bp, Artificial Sequence
  Synthetic Polynucleotide
aatcaagtttt

SEQ ID NO: 906 - DNA, 11 bp, Artificial Sequence
  Synthetic Polynucleotide
tttgcccgatt

SEQ ID NO: 907 - DNA, 11 bp, Artificial Sequence
  Synthetic Polynucleotide
gtgggaacagt

SEQ ID NO: 908 - DNA, 11 bp, Artificial Sequence
  Synthetic Polynucleotide
tcaagagaagc

SEQ ID NO: 909 - DNA, 11 bp, Artificial Sequence
  Synthetic Polynucleotide
aattaaccgac

SEQ ID NO: 910 - DNA, 11 bp, Artificial Sequence
  Synthetic Polynucleotide
gaaaaggtggg

SEQ ID NO: 911 - DNA, 11 bp, Artificial Sequence
  Synthetic Polynucleotide
ggagggaagaa

SEQ ID NO: 912 - DNA, 31 bp, Artificial Sequence; identical to SEQ ID NO: 441
  Synthetic Polynucleotide

SEQ ID NO: 913 - DNA, 41 bp, Artificial Sequence; identical to SEQ ID NO: 443
  Synthetic Polynucleotide

SEQ ID NO: 914 - DNA, 48 bp, Artificial Sequence; identical to SEQ ID NO: 671
  Synthetic Polynucleotide

SEQ ID NO: 915 - DNA, 54 bp, Artificial Sequence; identical to SEQ ID NO: 672
  Synthetic Polynucleotide

SEQ ID NO: 916 - DNA, 36 bp, Artificial Sequence; identical to SEQ ID NO: 673
  Synthetic Polynucleotide

SEQ ID NO: 917 - DNA, 31 bp, Artificial Sequence; identical to SEQ ID NO: 674
  Synthetic Polynucleotide

SEQ ID NO: 918 - DNA, 41 bp, Artificial Sequence; identical to SEQ ID NO: 675
  Synthetic Polynucleotide

SEQ ID NO: 919 - DNA, 46 bp, Artificial Sequence; identical to SEQ ID NO: 676
  Synthetic Polynucleotide

SEQ ID NO: 920 - DNA, 31 bp, Artificial Sequence; identical to SEQ ID NO: 677
  Synthetic Polynucleotide

SEQ ID NO: 921 - DNA, 49 bp, Artificial Sequence; identical to SEQ ID NO: 678
  Synthetic Polynucleotide

SEQ ID NO: 922 - DNA, 54 bp, Artificial Sequence; identical to SEQ ID NO: 679
  Synthetic Polynucleotide

SEQ ID NO: 923 - DNA, 36 bp, Artificial Sequence; identical to SEQ ID NO: 680
  Synthetic Polynucleotide

SEQ ID NO: 924 - DNA, 31 bp, Artificial Sequence; identical to SEQ ID NO: 681
  Synthetic Polynucleotide

SEQ ID NO: 925 - DNA, 41 bp, Artificial Sequence; identical to SEQ ID NO: 682
  Synthetic Polynucleotide

SEQ ID NO: 926 - DNA, 45 bp, Artificial Sequence; identical to SEQ ID NO: 683
  Synthetic Polynucleotide

SEQ ID NO: 927 - DNA, 46 bp, Artificial Sequence; identical to SEQ ID NO: 684
  Synthetic Polynucleotide

SEQ ID NO: 928 - DNA, 31 bp, Artificial Sequence; identical to SEQ ID NO: 685
  Synthetic Polynucleotide

SEQ ID NO: 929 - DNA, 48 bp, Artificial Sequence; identical to SEQ ID NO: 440
  Synthetic Polynucleotide

SEQ ID NO: 930 - DNA, 54 bp, Artificial Sequence; identical to SEQ ID NO: 687
  Synthetic Polynucleotide

SEQ ID NO: 931 - DNA, 36 bp, Artificial Sequence; identical to SEQ ID NO: 688
  Synthetic Polynucleotide

SEQ ID NO: 932 - DNA, 31 bp, Artificial Sequence; identical to SEQ ID NO: 689
  Synthetic Polynucleotide

SEQ ID NO: 933 - DNA, 67 bp, Artificial Sequence; identical to SEQ ID NO: 690
  Synthetic Polynucleotide

SEQ ID NO: 934 - DNA, 67 bp, Artificial Sequence; identical to SEQ ID NO: 691
  Synthetic Polynucleotide

SEQ ID NO: 935 - DNA, 67 bp, Artificial Sequence; identical to SEQ ID NO: 692
  Synthetic Polynucleotide

SEQ ID NO: 936 - DNA, 64 bp, Artificial Sequence; identical to SEQ ID NO: 693
  Synthetic Polynucleotide

SEQ ID NO: 937 - DNA, 64 bp, Artificial Sequence; identical to SEQ ID NO: 694
  Synthetic Polynucleotide

SEQ ID NO: 938 - DNA, 64 bp, Artificial Sequence; identical to SEQ ID NO: 695
  Synthetic Polynucleotide

SEQ ID NO: 939 - DNA, 15 bp, Artificial Sequence
  Synthetic Polynucleotide
catcagattagtgaa

SEQ ID NO: 940 - DNA, 15 bp, Artificial Sequence
  Synthetic Polynucleotide
caatgagaattttgc

SEQ ID NO: 941 - DNA, 15 bp, Artificial Sequence
  Synthetic Polynucleotide
ctagatatcgacatt

SEQ ID NO: 942 - DNA, 28 bp, Artificial Sequence
  Synthetic Polynucleotide
cgccaaccgcaagaaaagttacctgtcc

SEQ ID NO: 943 - DNA, 39 bp, Artificial Sequence; identical to SEQ ID NO: 228
  Synthetic Polynucleotide

SEQ ID NO: 944 - DNA, 28 bp, Artificial Sequence
  Synthetic Polynucleotide
cgtccaccacacccgccaacaagagcag

SEQ ID NO: 945 - DNA, 45 bp, Artificial Sequence; identical to SEQ ID NO: 230
  Synthetic Polynucleotide

SEQ ID NO: 946 - DNA, 42 bp, Artificial Sequence; identical to SEQ ID NO: 231
  Synthetic Polynucleotide

SEQ ID NO: 947 - DNA, 35 bp, Artificial Sequence; identical to SEQ ID NO: 232
  Synthetic Polynucleotide

SEQ ID NO: 948 - DNA, 28 bp, Artificial Sequence
  Synthetic Polynucleotide
tccgaatagcccgagatttgccctcacc

SEQ ID NO: 949 - DNA, 31 bp, Artificial Sequence; identical to SEQ ID NO: 234
  Synthetic Polynucleotide

SEQ ID NO: 950 - DNA, 35 bp, Artificial Sequence; identical to SEQ ID NO: 235
  Synthetic Polynucleotide

SEQ ID NO: 951 - DNA, 35 bp, Artificial Sequence; identical to SEQ ID NO: 236
  Synthetic Polynucleotide

SEQ ID NO: 952 - DNA, 42 bp, Artificial Sequence; identical to SEQ ID NO: 237
  Synthetic Polynucleotide

SEQ ID NO: 953 - DNA, 42 bp, Artificial Sequence; identical to SEQ ID NO: 238
  Synthetic Polynucleotide

SEQ ID NO: 954 - DNA, 21 bp, Artificial Sequence
  Synthetic Polynucleotide
gtggttccgatccacgcagag

SEQ ID NO: 955 - DNA, 35 bp, Artificial Sequence; identical to SEQ ID NO: 239
  Synthetic Polynucleotide

SEQ ID NO: 956 - DNA, 42 bp, Artificial Sequence; identical to SEQ ID NO: 240
  Synthetic Polynucleotide

SEQ ID NO: 957 - DNA, 35 bp, Artificial Sequence; identical to SEQ ID NO: 241
  Synthetic Polynucleotide

SEQ ID NO: 958 - DNA, 42 bp, Artificial Sequence; identical to SEQ ID NO: 242
  Synthetic Polynucleotide

SEQ ID NO: 959 - DNA, 35 bp, Artificial Sequence; identical to SEQ ID NO: 243
  Synthetic Polynucleotide

SEQ ID NO: 960 - DNA, 42 bp, Artificial Sequence; identical to SEQ ID NO: 244
  Synthetic Polynucleotide

SEQ ID NO: 961 - DNA, 21 bp, Artificial Sequence
  Synthetic Polynucleotide
tttacgatccgcggtgctcag

SEQ ID NO: 962 - DNA, 42 bp, Artificial Sequence; identical to SEQ ID NO: 246
  Synthetic Polynucleotide

SEQ ID NO: 963 - DNA, 35 bp, Artificial Sequence; identical to SEQ ID NO: 247
  Synthetic Polynucleotide

SEQ ID NO: 964 - DNA, 28 bp, Artificial Sequence
  Synthetic Polynucleotide
ccgaagcataaagtgtatcgaattccag

SEQ ID NO: 965 - DNA, 49 bp, Artificial Sequence; identical to SEQ ID NO: 249
  Synthetic Polynucleotide

SEQ ID NO: 966 - DNA, 35 bp, Artificial Sequence; identical to SEQ ID NO: 250
  Synthetic Polynucleotide

SEQ ID NO: 967 - DNA, 28 bp, Artificial Sequence
  Synthetic Polynucleotide
cagttctttttcaccgcctggcccatca

SEQ ID NO: 968 - DNA, 42 bp, Artificial Sequence; identical to SEQ ID NO: 252
  Synthetic Polynucleotide

SEQ ID NO: 969 - DNA, 35 bp, Artificial Sequence; identical to SEQ ID NO: 253
  Synthetic Polynucleotide

SEQ ID NO: 970 - DNA, 28 bp, Artificial Sequence
  Synthetic Polynucleotide
cggacatcccttttagacaggaacataa

SEQ ID NO: 971 - DNA, 35 bp, Artificial Sequence; identical to SEQ ID NO: 255
  Synthetic Polynucleotide

SEQ ID NO: 972 - DNA, 42 bp, Artificial Sequence; identical to SEQ ID NO: 256
  Synthetic Polynucleotide

SEQ ID NO: 973 - DNA, 28 bp, Artificial Sequence
  Synthetic Polynucleotide
ttccctgaaagaacgaaccaccaggcca

SEQ ID NO: 974 - DNA, 42 bp, Artificial Sequence; identical to SEQ ID NO: 258
  Synthetic Polynucleotide

SEQ ID NO: 975 - DNA, 35 bp, Artificial Sequence; identical to SEQ ID NO: 259
  Synthetic Polynucleotide

SEQ ID NO: 976 - DNA, 42 bp, Artificial Sequence; identical to SEQ ID NO: 260
  Synthetic Polynucleotide

SEQ ID NO: 977 - DNA, 42 bp, Artificial Sequence; identical to SEQ ID NO: 261
  Synthetic Polynucleotide

SEQ ID NO: 978 - DNA, 42 bp, Artificial Sequence; identical to SEQ ID NO: 262
  Synthetic Polynucleotide

SEQ ID NO: 979 - DNA, 31 bp, Artificial Sequence; identical to SEQ ID NO: 263
  Synthetic Polynucleotide

SEQ ID NO: 980 - DNA, 35 bp, Artificial Sequence; identical to SEQ ID NO: 264
  Synthetic Polynucleotide

SEQ ID NO: 981 - DNA, 28 bp, Artificial Sequence
  Synthetic Polynucleotide
atagcgatagcttacaagcgtgccgcat

SEQ ID NO: 982 - DNA, 42 bp, Artificial Sequence; identical to SEQ ID NO: 266
  Synthetic Polynucleotide

SEQ ID NO: 983 - DNA, 35 bp, Artificial Sequence; identical to SEQ ID NO: 267
  Synthetic Polynucleotide

SEQ ID NO: 984 - DNA, 42 bp, Artificial Sequence; identical to SEQ ID NO: 268
  Synthetic Polynucleotide

SEQ ID NO: 985 - DNA, 32 bp, Artificial Sequence; identical to SEQ ID NO: 269
  Synthetic Polynucleotide

SEQ ID NO: 986 - DNA, 35 bp, Artificial Sequence; identical to SEQ ID NO: 270
  Synthetic Polynucleotide

SEQ ID NO: 987 - DNA, 42 bp, Artificial Sequence; identical to SEQ ID NO: 271
  Synthetic Polynucleotide

SEQ ID NO: 988 - DNA, 42 bp, Artificial Sequence; identical to SEQ ID NO: 272
  Synthetic Polynucleotide

SEQ ID NO: 989 - DNA, 42 bp, Artificial Sequence; identical to SEQ ID NO: 273
  Synthetic Polynucleotide

SEQ ID NO: 990 - DNA, 34 bp, Artificial Sequence; identical to SEQ ID NO: 274
  Synthetic Polynucleotide

SEQ ID NO: 991 - DNA, 42 bp, Artificial Sequence; identical to SEQ ID NO: 275
  Synthetic Polynucleotide

SEQ ID NO: 992 - DNA, 42 bp, Artificial Sequence; identical to SEQ ID NO: 276
  Synthetic Polynucleotide

SEQ ID NO: 993 - DNA, 34 bp, Artificial Sequence; identical to SEQ ID NO: 277
  Synthetic Polynucleotide

SEQ ID NO: 994 - DNA, 28 bp, Artificial Sequence
  Synthetic Polynucleotide
gagcaccaacctaaagaagagtaatcga

SEQ ID NO: 995 - DNA, 39 bp, Artificial Sequence; identical to SEQ ID NO: 279
  Synthetic Polynucleotide

SEQ ID NO: 996 - DNA, 28 bp, Artificial Sequence
  Synthetic Polynucleotide
tttttttgataagaggtttttaattctt

SEQ ID NO: 997 - DNA, 45 bp, Artificial Sequence; identical to SEQ ID NO: 281
  Synthetic Polynucleotide

SEQ ID NO: 998 - DNA, 42 bp, Artificial Sequence; identical to SEQ ID NO: 282
  Synthetic Polynucleotide

SEQ ID NO: 999 - DNA, 35 bp, Artificial Sequence; identical to SEQ ID NO: 283
  Synthetic Polynucleotide

SEQ ID NO: 1000 - DNA, 28 bp, Artificial Sequence
  Synthetic Polynucleotide
aaaaaagattaagaggaataaatatagc

SEQ ID NO: 1001 - DNA, 31 bp, Artificial Sequence; identical to SEQ ID NO: 285
  Synthetic Polynucleotide

SEQ ID NO: 1002 - DNA, 35 bp, Artificial Sequence; identical to SEQ ID NO: 286
  Synthetic Polynucleotide

SEQ ID NO: 1003 - DNA, 35 bp, Artificial Sequence; identical to SEQ ID NO: 287
  Synthetic Polynucleotide

SEQ ID NO: 1004 - DNA, 42 bp, Artificial Sequence; identical to SEQ ID NO: 288
  Synthetic Polynucleotide

SEQ ID NO: 1005 - DNA, 42 bp, Artificial Sequence; identical to SEQ ID NO: 289
  Synthetic Polynucleotide

SEQ ID NO: 1006 - DNA, 21 bp, Artificial Sequence
  Synthetic Polynucleotide
ctgactattaagaaaacaagt

SEQ ID NO: 1007 - DNA, 35 bp, Artificial Sequence; identical to SEQ ID NO: 290
  Synthetic Polynucleotide

SEQ ID NO: 1008 - DNA, 42 bp, Artificial Sequence; identical to SEQ ID NO: 291
  Synthetic Polynucleotide

SEQ ID NO: 1009 - DNA, 35 bp, Artificial Sequence; identical to SEQ ID NO: 292
  Synthetic Polynucleotide

SEQ ID NO: 1010 - DNA, 42 bp, Artificial Sequence; identical to SEQ ID NO: 293
  Synthetic Polynucleotide

SEQ ID NO: 1011 - DNA, 35 bp, Artificial Sequence; identical to SEQ ID NO: 294
  Synthetic Polynucleotide

SEQ ID NO: 1012 - DNA, 42 bp, Artificial Sequence; identical to SEQ ID NO: 295
  Synthetic Polynucleotide

SEQ ID NO: 1013 - DNA, 21 bp, Artificial Sequence
  Synthetic Polynucleotide
ttatcaacgtaagaaccacga

SEQ ID NO: 1014 - DNA, 42 bp, Artificial Sequence; identical to SEQ ID NO: 297
  Synthetic Polynucleotide

SEQ ID NO: 1015 - DNA, 28 bp, Artificial Sequence
  Synthetic Polynucleotide
atcggaataccacattcgggaagaaact

SEQ ID NO: 1016 - DNA, 49 bp, Artificial Sequence; identical to SEQ ID NO: 299
  Synthetic Polynucleotide

SEQ ID NO: 1017 - DNA, 35 bp, Artificial Sequence; identical to SEQ ID NO: 300
  Synthetic Polynucleotide

SEQ ID NO: 1018 - DNA, 28 bp, Artificial Sequence
  Synthetic Polynucleotide
aaagactggattcattgaatccccgcat

SEQ ID NO: 1019 - DNA, 42 bp, Artificial Sequence; identical to SEQ ID NO: 302
  Synthetic Polynucleotide

SEQ ID NO: 1020 - DNA, 42 bp, Artificial Sequence; identical to SEQ ID NO: 303
  Synthetic Polynucleotide

SEQ ID NO: 1021 - DNA, 36 bp, Artificial Sequence; identical to SEQ ID NO: 304
  Synthetic Polynucleotide

SEQ ID NO: 1022 - DNA, 28 bp, Artificial Sequence
  Synthetic Polynucleotide
tcaggacagaattcccaattctgccatg

SEQ ID NO: 1023 - DNA, 35 bp, Artificial Sequence; identical to SEQ ID NO: 306
  Synthetic Polynucleotide

SEQ ID NO: 1024 - DNA, 42 bp, Artificial Sequence; identical to SEQ ID NO: 307
  Synthetic Polynucleotide

SEQ ID NO: 1025 - DNA, 28 bp, Artificial Sequence
  Synthetic Polynucleotide
agcgattcaatgagagatctacaacggt

SEQ ID NO: 1026 - DNA, 42 bp, Artificial Sequence; identical to SEQ ID NO: 309
  Synthetic Polynucleotide

SEQ ID NO: 1027 - DNA, 35 bp, Artificial Sequence; identical to SEQ ID NO: 310
  Synthetic Polynucleotide

SEQ ID NO: 1028 - DNA, 42 bp, Artificial Sequence; identical to SEQ ID NO: 311
  Synthetic Polynucleotide

SEQ ID NO: 1029 - DNA, 35 bp, Artificial Sequence; identical to SEQ ID NO: 312
  Synthetic Polynucleotide

SEQ ID NO: 1030 - DNA, 42 bp, Artificial Sequence; identical to SEQ ID NO: 313
  Synthetic Polynucleotide

SEQ ID NO: 1031 - DNA, 31 bp, Artificial Sequence; identical to SEQ ID NO: 314
  Synthetic Polynucleotide

SEQ ID NO: 1032 - DNA, 35 bp, Artificial Sequence; identical to SEQ ID NO: 315
  Synthetic Polynucleotide

SEQ ID NO: 1033 - DNA, 28 bp, Artificial Sequence
  Synthetic Polynucleotide
gcagttgggcggttatcatcattgaccc

SEQ ID NO: 1034 - DNA, 42 bp, Artificial Sequence; identical to SEQ ID NO: 317
  Synthetic Polynucleotide

SEQ ID NO: 1035 - DNA, 35 bp, Artificial Sequence; identical to SEQ ID NO: 318
  Synthetic Polynucleotide

SEQ ID NO: 1036 - DNA, 41 bp, Artificial Sequence; identical to SEQ ID NO: 319
  Synthetic Polynucleotide

SEQ ID NO: 1037 - DNA, 32 bp, Artificial Sequence; identical to SEQ ID NO: 320
  Synthetic Polynucleotide

SEQ ID NO: 1038 - DNA, 35 bp, Artificial Sequence; identical to SEQ ID NO: 321
  Synthetic Polynucleotide

SEQ ID NO: 1039 - DNA, 42 bp, Artificial Sequence; identical to SEQ ID NO: 322
  Synthetic Polynucleotide

SEQ ID NO: 1040 - DNA, 42 bp, Artificial Sequence; identical to SEQ ID NO: 323
  Synthetic Polynucleotide

SEQ ID NO: 1041 - DNA, 42 bp, Artificial Sequence; identical to SEQ ID NO: 324
  Synthetic Polynucleotide

SEQ ID NO: 1042 - DNA, 30 bp, Artificial Sequence; identical to SEQ ID NO: 325
  Synthetic Polynucleotide

SEQ ID NO: 1043 - DNA, 30 bp, Artificial Sequence; identical to SEQ ID NO: 326
  Synthetic Polynucleotide

SEQ ID NO: 1044 - DNA, 28 bp, Artificial Sequence
  Synthetic Polynucleotide
caccgaccgtgtgatcagacgacacaag

SEQ ID NO: 1045 - DNA, 39 bp, Artificial Sequence; identical to SEQ ID NO: 328
  Synthetic Polynucleotide

SEQ ID NO: 1046 - DNA, 28 bp, Artificial Sequence
  Synthetic Polynucleotide
cttagttaccagaaggaataagagataa

SEQ ID NO: 1047 - DNA, 21 bp, Artificial Sequence
  Synthetic Polynucleotide
gaagaaacgcaataataagaa

SEQ ID NO: 1048 - DNA, 45 bp, Artificial Sequence; identical to SEQ ID NO: 331
  Synthetic Polynucleotide

SEQ ID NO: 1049 - DNA, 42 bp, Artificial Sequence; identical to SEQ ID NO: 332
  Synthetic Polynucleotide

SEQ ID NO: 1050 - DNA, 35 bp, Artificial Sequence; identical to SEQ ID NO: 333
  Synthetic Polynucleotide

SEQ ID NO: 1051 - DNA, 28 bp, Artificial Sequence
  Synthetic Polynucleotide
aagtcagagagataacctaacgtctcca

SEQ ID NO: 1052 - DNA, 31 bp, Artificial Sequence; identical to SEQ ID NO: 335
  Synthetic Polynucleotide

SEQ ID NO: 1053 - DNA, 35 bp, Artificial Sequence; identical to SEQ ID NO: 336
  Synthetic Polynucleotide

SEQ ID NO: 1054 - DNA, 35 bp, Artificial Sequence; identical to SEQ ID NO: 337
  Synthetic Polynucleotide

SEQ ID NO: 1055 - DNA, 42 bp, Artificial Sequence; identical to SEQ ID NO: 338
  Synthetic Polynucleotide

SEQ ID NO: 1056 - DNA, 42 bp, Artificial Sequence; identical to SEQ ID NO: 339
  Synthetic Polynucleotide

SEQ ID NO: 1057 - DNA, 21 bp, Artificial Sequence
  Synthetic Polynucleotide
caccctgaaccataaaaattt

SEQ ID NO: 1058 - DNA, 35 bp, Artificial Sequence; identical to SEQ ID NO: 340
  Synthetic Polynucleotide

SEQ ID NO: 1059 - DNA, 42 bp, Artificial Sequence; identical to SEQ ID NO: 341
  Synthetic Polynucleotide

SEQ ID NO: 1060 - DNA, 35 bp, Artificial Sequence; identical to SEQ ID NO: 342
  Synthetic Polynucleotide

SEQ ID NO: 1061 - DNA, 42 bp, Artificial Sequence; identical to SEQ ID NO: 343
  Synthetic Polynucleotide

SEQ ID NO: 1062 - DNA, 35 bp, Artificial Sequence; identical to SEQ ID NO: 344
  Synthetic Polynucleotide

SEQ ID NO: 1063 - DNA, 42 bp, Artificial Sequence; identical to SEQ ID NO: 345
  Synthetic Polynucleotide

SEQ ID NO: 1064 - DNA, 21 bp, Artificial Sequence
  Synthetic Polynucleotide
tttcctgtttacatgttgaaa

SEQ ID NO: 1065 - DNA, 42 bp, Artificial Sequence; identical to SEQ ID NO: 347
  Synthetic Polynucleotide

SEQ ID NO: 1066 - DNA, 28 bp, Artificial Sequence
  Synthetic Polynucleotide
gcacgaggcgttttagctattttctcct

SEQ ID NO: 1067 - DNA, 49 bp, Artificial Sequence; identical to SEQ ID NO: 349
  Synthetic Polynucleotide

SEQ ID NO: 1068 - DNA, 35 bp, Artificial Sequence; identical to SEQ ID NO: 350
  Synthetic Polynucleotide

SEQ ID NO: 1069 - DNA, 28 bp, Artificial Sequence
  Synthetic Polynucleotide
gcgatcccaaaaaaatgaaaataggcta

SEQ ID NO: 1070 - DNA, 42 bp, Artificial Sequence; identical to SEQ ID NO: 352
  Synthetic Polynucleotide

SEQ ID NO: 1071 - DNA, 42 bp, Artificial Sequence; identical to SEQ ID NO: 353
  Synthetic Polynucleotide

SEQ ID NO: 1072 - DNA, 36 bp, Artificial Sequence; identical to SEQ ID NO: 354
  Synthetic Polynucleotide

SEQ ID NO: 1073 - DNA, 28 bp, Artificial Sequence
  Synthetic Polynucleotide
gacgtaataaataaaagaaacgcaactc

SEQ ID NO: 1074 - DNA, 35 bp, Artificial Sequence; identical to SEQ ID NO: 356
  Synthetic Polynucleotide

SEQ ID NO: 1075 - DNA, 42 bp, Artificial Sequence; identical to SEQ ID NO: 357
  Synthetic Polynucleotide

SEQ ID NO: 1076 - DNA, 28 bp, Artificial Sequence
  Synthetic Polynucleotide
gtaatagcccgccaccctcagagcgaca

SEQ ID NO: 1077 - DNA, 21 bp, Artificial Sequence
  Synthetic Polynucleotide
taccacggaataagtttaaaa

SEQ ID NO: 1078 - DNA, 35 bp, Artificial Sequence; identical to SEQ ID NO: 360
  Synthetic Polynucleotide

SEQ ID NO: 1079 - DNA, 42 bp, Artificial Sequence; identical to SEQ ID NO: 361
  Synthetic Polynucleotide

SEQ ID NO: 1080 - DNA, 35 bp, Artificial Sequence; identical to SEQ ID NO: 362
  Synthetic Polynucleotide

SEQ ID NO: 1081 - DNA, 42 bp, Artificial Sequence; identical to SEQ ID NO: 363
  Synthetic Polynucleotide

SEQ ID NO: 1082 - DNA, 31 bp, Artificial Sequence; identical to SEQ ID NO: 364
  Synthetic Polynucleotide

SEQ ID NO: 1083 - DNA, 35 bp, Artificial Sequence; identical to SEQ ID NO: 365
  Synthetic Polynucleotide

SEQ ID NO: 1084 - DNA, 28 bp, Artificial Sequence
  Synthetic Polynucleotide
cagcttgataccgatcccattccagaac

SEQ ID NO: 1085 - DNA, 42 bp, Artificial Sequence; identical to SEQ ID NO: 367
  Synthetic Polynucleotide

SEQ ID NO: 1086 - DNA, 35 bp, Artificial Sequence; identical to SEQ ID NO: 368
  Synthetic Polynucleotide

SEQ ID NO: 1087 - DNA, 41 bp, Artificial Sequence; identical to SEQ ID NO: 369
  Synthetic Polynucleotide

SEQ ID NO: 1088 - DNA, 32 bp, Artificial Sequence; identical to SEQ ID NO: 370
  Synthetic Polynucleotide

SEQ ID NO: 1089 - DNA, 35 bp, Artificial Sequence; identical to SEQ ID NO: 371
  Synthetic Polynucleotide

SEQ ID NO: 1090 - DNA, 42 bp, Artificial Sequence; identical to SEQ ID NO: 372
  Synthetic Polynucleotide

SEQ ID NO: 1091 - DNA, 42 bp, Artificial Sequence; identical to SEQ ID NO: 373
  Synthetic Polynucleotide

SEQ ID NO: 1092 - DNA, 42 bp, Artificial Sequence; identical to SEQ ID NO: 374
  Synthetic Polynucleotide

SEQ ID NO: 1093 - DNA, 34 bp, Artificial Sequence; identical to SEQ ID NO: 375
  Synthetic Polynucleotide

SEQ ID NO: 1094 - DNA, 42 bp, Artificial Sequence; identical to SEQ ID NO: 376
  Synthetic Polynucleotide

SEQ ID NO: 1095 - DNA, 42 bp, Artificial Sequence; identical to SEQ ID NO: 377
  Synthetic Polynucleotide

SEQ ID NO: 1096 - DNA, 34 bp, Artificial Sequence; identical to SEQ ID NO: 378
  Synthetic Polynucleotide

SEQ ID NO: 1097 - DNA, 42 bp, Artificial Sequence
  Synthetic Polynucleotide
ctgaggcttgcagggagttaatgacccccagcgattatacca

SEQ ID NO: 1098 - DNA, 42 bp, Artificial Sequence
  Synthetic Polynucleotide
cataaccgatatattcggtcgagcgcgaaacaaagtacaacg

SEQ ID NO: 1099 - DNA, 42 bp, Artificial Sequence
  Synthetic Polynucleotide
tgacaacaaccatcgcccacggagatttgtatcatcgcctga

SEQ ID NO: 1100 - DNA, 21 bp, Artificial Sequence
  Synthetic Polynucleotide
gtggttccgatccacgcagag

SEQ ID NO: 1101 - DNA, 21 bp, Artificial Sequence
  Synthetic Polynucleotide
ctgactattaagaaaacaagt

SEQ ID NO: 1102 - DNA, 21 bp, Artificial Sequence
  Synthetic Polynucleotide
caccctgaaccataaaaattt

SEQ ID NO: 1103 - DNA, 30 bp, Artificial Sequence; identical to SEQ ID NO: 383
  Synthetic Polynucleotide

SEQ ID NO: 1104 - DNA, 27 bp, Artificial Sequence
  Synthetic Polynucleotide
attctggccaacagagataaaacagag

SEQ ID NO: 1105 - DNA, 42 bp, Artificial Sequence; identical to SEQ ID NO: 385
  Synthetic Polynucleotide

SEQ ID NO: 1106 - DNA, 35 bp, Artificial Sequence; identical to SEQ ID NO: 386
  Synthetic Polynucleotide

SEQ ID NO: 1107 - DNA, 37 bp, Artificial Sequence; identical to SEQ ID NO: 387
  Synthetic Polynucleotide

SEQ ID NO: 1108 - DNA, 30 bp, Artificial Sequence; identical to SEQ ID NO: 388
  Synthetic Polynucleotide

SEQ ID NO: 1109 - DNA, 21 bp, Artificial Sequence
  Synthetic Polynucleotide
aacaaacatcaagaagcaaaa

SEQ ID NO: 1110 - DNA, 30 bp, Artificial Sequence; identical to SEQ ID NO: 390
  Synthetic Polynucleotide

SEQ ID NO: 1111 - DNA, 34 bp, Artificial Sequence; identical to SEQ ID NO: 391
  Synthetic Polynucleotide

SEQ ID NO: 1112 - DNA, 21 bp, Artificial Sequence
  Synthetic Polynucleotide
gcggaacaaagaaagagtaac

SEQ ID NO: 1113 - DNA, 30 bp, Artificial Sequence; identical to SEQ ID NO: 393
  Synthetic Polynucleotide

SEQ ID NO: 1114 - DNA, 27 bp, Artificial Sequence
  Synthetic Polynucleotide
aaatgcaatgcctgagtcaggtcattg

SEQ ID NO: 1115 - DNA, 42 bp, Artificial Sequence; identical to SEQ ID NO: 395
  Synthetic Polynucleotide

SEQ ID NO: 1116 - DNA, 35 bp, Artificial Sequence; identical to SEQ ID NO: 396
  Synthetic Polynucleotide

SEQ ID NO: 1117 - DNA, 37 bp, Artificial Sequence; identical to SEQ ID NO: 397
  Synthetic Polynucleotide

SEQ ID NO: 1118 - DNA, 30 bp, Artificial Sequence; identical to SEQ ID NO: 398
  Synthetic Polynucleotide

SEQ ID NO: 1119 - DNA, 21 bp, Artificial Sequence
  Synthetic Polynucleotide
acctcaccggaaacccgccac

SEQ ID NO: 1120 - DNA, 30 bp, Artificial Sequence; identical to SEQ ID NO: 400
  Synthetic Polynucleotide

SEQ ID NO: 1121 - DNA, 34 bp, Artificial Sequence; identical to SEQ ID NO: 401
  Synthetic Polynucleotide

SEQ ID NO: 1122 - DNA, 21 bp, Artificial Sequence
  Synthetic Polynucleotide
ggtcacgttggtgtattgacc

SEQ ID NO: 1123 - DNA, 30 bp, Artificial Sequence; identical to SEQ ID NO: 403
  Synthetic Polynucleotide

SEQ ID NO: 1124 - DNA, 27 bp, Artificial Sequence
  Synthetic Polynucleotide
ctgtagcgcgttttcatctcagagccg

SEQ ID NO: 1125 - DNA, 42 bp, Artificial Sequence; identical to SEQ ID NO: 405
  Synthetic Polynucleotide

SEQ ID NO: 1126 - DNA, 35 bp, Artificial Sequence; identical to SEQ ID NO: 406
  Synthetic Polynucleotide

SEQ ID NO: 1127 - DNA, 37 bp, Artificial Sequence; identical to SEQ ID NO: 407
  Synthetic Polynucleotide

SEQ ID NO: 1128 - DNA, 30 bp, Artificial Sequence; identical to SEQ ID NO: 408
  Synthetic Polynucleotide

SEQ ID NO: 1129 - DNA, 21 bp, Artificial Sequence
  Synthetic Polynucleotide
actttcaacagtttatgggat

SEQ ID NO: 1130 - DNA, 30 bp, Artificial Sequence; identical to SEQ ID NO: 410
  Synthetic Polynucleotide

SEQ ID NO: 1131 - DNA, 34 bp, Artificial Sequence; identical to SEQ ID NO: 411
  Synthetic Polynucleotide

SEQ ID NO: 1132 - DNA, 21 bp, Artificial Sequence
  Synthetic Polynucleotide
ggttttgctcagtaaaggatt

SEQ ID NO: 1133 - DNA, 11 bp, Artificial Sequence
  Synthetic Polynucleotide
caaaattatga

SEQ ID NO: 1134 - DNA, 11 bp, Artificial Sequence
  Synthetic Polynucleotide
gcgccattcca

SEQ ID NO: 1135 - DNA, 11 bp, Artificial Sequence
  Synthetic Polynucleotide
cagagccacta

SEQ ID NO: 1136 - DNA, 11 bp, Artificial Sequence
  Synthetic Polynucleotide
gaagatgattt

SEQ ID NO: 1137 - DNA, 11 bp, Artificial Sequence
  Synthetic Polynucleotide
gggaacggaca

SEQ ID NO: 1138 - DNA, 11 bp, Artificial Sequence
  Synthetic Polynucleotide
tttgctaaagc

SEQ ID NO: 1139 - DNA, 11 bp, Artificial Sequence
  Synthetic Polynucleotide
tatctaaaaac

SEQ ID NO: 1140 - DNA, 11 bp, Artificial Sequence
  Synthetic Polynucleotide
cattaaattga

SEQ ID NO: 1141 - DNA, 11 bp, Artificial Sequence
  Synthetic Polynucleotide
ttgatgatatt

SEQ ID NO: 1142 - DNA, 11 bp, Artificial Sequence
  Synthetic Polynucleotide
acatcactttt

SEQ ID NO: 1143 - DNA, 11 bp, Artificial Sequence
  Synthetic Polynucleotide
atagtagtagg

SEQ ID NO: 1144 - DNA, 11 bp, Artificial Sequence
  Synthetic Polynucleotide
tattgacggta

SEQ ID NO: 1145 - DNA, 11 bp, Artificial Sequence
  Synthetic Polynucleotide
atggaaacagt

SEQ ID NO: 1146 - DNA, 11 bp, Artificial Sequence
  Synthetic Polynucleotide
gagatagaccg

SEQ ID NO: 1147 - DNA, 11 bp, Artificial Sequence
  Synthetic Polynucleotide
attttttcatt

SEQ ID NO: 1148 - DNA, 11 bp, Artificial Sequence
  Synthetic Polynucleotide
ataaaagggta

SEQ ID NO: 1149 - DNA, 11 bp, Artificial Sequence
  Synthetic Polynucleotide
gtgaggcggtc

SEQ ID NO: 1150 - DNA, 11 bp, Artificial Sequence
  Synthetic Polynucleotide
attatcattgc

SEQ ID NO: 1151 - DNA, 11 bp, Artificial Sequence
  Synthetic Polynucleotide
tcatatattca

SEQ ID NO: 1152 - DNA, 11 bp, Artificial Sequence
  Synthetic Polynucleotide
cctgagagtcc

SEQ ID NO: 1153 - DNA, 11 bp, Artificial Sequence
  Synthetic Polynucleotide
gtaatgggaaa

SEQ ID NO: 1154 - DNA, 11 bp, Artificial Sequence
  Synthetic Polynucleotide
ttagcgtcatt

SEQ ID NO: 1155 - DNA, 11 bp, Artificial Sequence
  Synthetic Polynucleotide
ccaccagaact

SEQ ID NO: 1156 - DNA, 11 bp, Artificial Sequence
  Synthetic Polynucleotide
aggattagcgc

SEQ ID NO: 1157 - DNA, 46 bp, Artificial Sequence; identical to SEQ ID NO: 437
  Synthetic Polynucleotide

SEQ ID NO: 1158 - DNA, 54 bp, Artificial Sequence; identical to SEQ ID NO: 438
  Synthetic Polynucleotide

SEQ ID NO: 1159 - DNA, 31 bp, Artificial Sequence; identical to SEQ ID NO: 439
  Synthetic Polynucleotide

SEQ ID NO: 1160 - DNA, 48 bp, Artificial Sequence; identical to SEQ ID NO: 440
  Synthetic Polynucleotide

SEQ ID NO: 1161 - DNA, 31 bp, Artificial Sequence; identical to SEQ ID NO: 441
  Synthetic Polynucleotide

SEQ ID NO: 1162 - DNA, 36 bp, Artificial Sequence; identical to SEQ ID NO: 442
  Synthetic Polynucleotide

SEQ ID NO: 1163 - DNA, 41 bp, Artificial Sequence; identical to SEQ ID NO: 443
  Synthetic Polynucleotide

SEQ ID NO: 1164 - DNA, 45 bp, Artificial Sequence; identical to SEQ ID NO: 444
  Synthetic Polynucleotide

SEQ ID NO: 1165 - DNA, 54 bp, Artificial Sequence; identical to SEQ ID NO: 445
  Synthetic Polynucleotide

SEQ ID NO: 1166 - DNA, 31 bp, Artificial Sequence; identical to SEQ ID NO: 446
  Synthetic Polynucleotide

SEQ ID NO: 1167 - DNA, 49 bp, Artificial Sequence; identical to SEQ ID NO: 447
  Synthetic Polynucleotide

SEQ ID NO: 1168 - DNA, 31 bp, Artificial Sequence; identical to SEQ ID NO: 448
  Synthetic Polynucleotide

SEQ ID NO: 1169 - DNA, 36 bp, Artificial Sequence; identical to SEQ ID NO: 449
  Synthetic Polynucleotide

SEQ ID NO: 1170 - DNA, 41 bp, Artificial Sequence; identical to SEQ ID NO: 450
  Synthetic Polynucleotide

SEQ ID NO: 1171 - DNA, 46 bp, Artificial Sequence; identical to SEQ ID NO: 451
  Synthetic Polynucleotide

SEQ ID NO: 1172 - DNA, 54 bp, Artificial Sequence; identical to SEQ ID NO: 452
  Synthetic Polynucleotide

SEQ ID NO: 1173 - DNA, 31 bp, Artificial Sequence; identical to SEQ ID NO: 453
  Synthetic Polynucleotide

SEQ ID NO: 1174 - DNA, 48 bp, Artificial Sequence; identical to SEQ ID NO: 454
  Synthetic Polynucleotide

SEQ ID NO: 1175 - DNA, 31 bp, Artificial Sequence; identical to SEQ ID NO: 455
  Synthetic Polynucleotide

SEQ ID NO: 1176 - DNA, 36 bp, Artificial Sequence; identical to SEQ ID NO: 456
  Synthetic Polynucleotide

SEQ ID NO: 1177 - DNA, 41 bp, Artificial Sequence; identical to SEQ ID NO: 457
  Synthetic Polynucleotide

SEQ ID NO: 1178 - DNA, 34 bp, Artificial Sequence; identical to SEQ ID NO: 458
  Synthetic Polynucleotide

SEQ ID NO: 1179 - DNA, 28 bp, Artificial Sequence
  Synthetic Polynucleotide
ggggtggtttgccccagcaggcgttttt

SEQ ID NO: 1180 - DNA, 34 bp, Artificial Sequence; identical to SEQ ID NO: 460
  Synthetic Polynucleotide

SEQ ID NO: 1181 - DNA, 28 bp, Artificial Sequence
  Synthetic Polynucleotide
aaaggagaatgaccataaatcaattttt

SEQ ID NO: 1182 - DNA, 34 bp, Artificial Sequence; identical to SEQ ID NO: 462
  Synthetic Polynucleotide

SEQ ID NO: 1183 - DNA, 28 bp, Artificial Sequence
  Synthetic Polynucleotide
cctaacagggaagcgcattagacttttt

SEQ ID NO: 1184 - DNA, 64 bp, Artificial Sequence; identical to SEQ ID NO: 464
  Synthetic Polynucleotide

SEQ ID NO: 1185 - DNA, 64 bp, Artificial Sequence; identical to SEQ ID NO: 465
  Synthetic Polynucleotide

SEQ ID NO: 1186 - DNA, 61 bp, Artificial Sequence
  Synthetic Polynucleotide
tttttagcgtctttccatatcccatcagtggcgatatcgcgcataggctgaccggaatacc

SEQ ID NO: 1187 - DNA, 15 bp, Artificial Sequence
  Synthetic Polynucleotide
catcagattagtgaa

SEQ ID NO: 1188 - DNA, 15 bp, Artificial Sequence
  Synthetic Polynucleotide
caatgagaattttgc

SEQ ID NO: 1189 - DNA, 12 bp, Artificial Sequence
  Synthetic Polynucleotide
gatatcgccact

SEQ ID NO: 1190 - DNA, 28 bp, Artificial Sequence
  Synthetic Polynucleotide
ccgagcgtggtgctgaagttacctgtcc

SEQ ID NO: 1191 - DNA, 39 bp, Artificial Sequence
  Synthetic Polynucleotide
gtactattccatcacgcaagacggggaaccgctacgtgc

SEQ ID NO: 1192 - DNA, 28 bp, Artificial Sequence
  Synthetic Polynucleotide
aggaatcggaaccctaaaacaagagcag

SEQ ID NO: 1193 - DNA, 45 bp, Artificial Sequence
  Synthetic Polynucleotide
tttagtaaaagagtctgggttgctagcacatgatgctgaaacatc

SEQ ID NO: 1194 - DNA, 42 bp, Artificial Sequence
  Synthetic Polynucleotide
aacccagaatcctgagaatcagagcttttacatcggttaaat

SEQ ID NO: 1195 - DNA, 35 bp, Artificial Sequence; identical to SEQ ID NO: 232
  Synthetic Polynucleotide

SEQ ID NO: 1196 - DNA, 28 bp, Artificial Sequence
  Synthetic Polynucleotide
tccgaatagcccgagatttgccctcacc

SEQ ID NO: 1197 - DNA, 31 bp, Artificial Sequence
  Synthetic Polynucleotide
gtgccgaataatggaagacggaacagggcgc

SEQ ID NO: 1198 - DNA, 35 bp, Artificial Sequence
  Synthetic Polynucleotide
aatacctaccatcctgatcgacaactcgtatatga

SEQ ID NO: 1199 - DNA, 35 bp, Artificial Sequence
  Synthetic Polynucleotide
acatcacacgaccagtatctttaaccagcagttgc

SEQ ID NO: 1200 - DNA, 42 bp, Artificial Sequence
  Synthetic Polynucleotide
aattgcacgttgatggctttgcccgaagtattagactttcaa

SEQ ID NO: 1201 - DNA, 42 bp, Artificial Sequence
  Synthetic Polynucleotide
aacgaaattgatcatatttaaaaggataatacatttgaggaa

SEQ ID NO: 1202 - DNA, 48 bp, Artificial Sequence
  Synthetic Polynucleotide
gtggttccgatccacgcagaggcgaacctgttccacacaacatactag

SEQ ID NO: 1203 - DNA, 42 bp, Artificial Sequence
  Synthetic Polynucleotide
ggcattaaagagcactagaagaaagcgaaaggtcacgcttac

SEQ ID NO: 1204 - DNA, 35 bp, Artificial Sequence
  Synthetic Polynucleotide
aaaagtttggagggagcgaacgtggcgagaaacac

SEQ ID NO: 1205 - DNA, 42 bp, Artificial Sequence
  Synthetic Polynucleotide
aagacgctcatcacttgttataatcagtgagtaacgtgtcgc

SEQ ID NO: 1206 - DNA, 42 bp, Artificial Sequence
  Synthetic Polynucleotide
gccctaaaacataacagctgaagattatttacattggcagat

SEQ ID NO: 1207 - DNA, 35 bp, Artificial Sequence
  Synthetic Polynucleotide
tttgtgaggctgaaaaatatctaaaatatctgtca

SEQ ID NO: 1208 - DNA, 21 bp, Artificial Sequence
  Synthetic Polynucleotide
tttacgatccgcggtgcgaac

SEQ ID NO: 1209 - DNA, 42 bp, Artificial Sequence
  Synthetic Polynucleotide
agtacattaagggtgcctaatgaggaggatccgcgtcccaaa

SEQ ID NO: 1210 - DNA, 35 bp, Artificial Sequence
  Synthetic Polynucleotide
ccatgcgcgaactgatatcaccagttttgaccttc

SEQ ID NO: 1211 - DNA, 28 bp, Artificial Sequence
  Synthetic Polynucleotide
ccgaagcataaagtgtatcgaattccag

SEQ ID NO: 1212 - DNA, 49 bp, Artificial Sequence
  Synthetic Polynucleotide
atcaaagctaactcgagacgggattatacttctcttgttcttcccgggt

SEQ ID NO: 1213 - DNA, 35 bp, Artificial Sequence
  Synthetic Polynucleotide
tgattgaaaggaattgaggatttagaacgttttac

SEQ ID NO: 1214 - DNA, 28 bp, Artificial Sequence
  Synthetic Polynucleotide
cagttctttttcaccgcctggcccatca

SEQ ID NO: 1215 - DNA, 42 bp, Artificial Sequence
  Synthetic Polynucleotide
cactgataaagcaaccgcaagtagacttgtacggtgccttgt

SEQ ID NO: 1216 - DNA, 35 bp, Artificial Sequence
  Synthetic Polynucleotide
atttcctgataacagagtgaatggctattagataa

SEQ ID NO: 1217 - DNA, 28 bp, Artificial Sequence
  Synthetic Polynucleotide
cggacatccctgcgcgtaaccaccagga

SEQ ID NO: 1218 - DNA, 35 bp, Artificial Sequence; identical to SEQ ID NO: 255
  Synthetic Polynucleotide

SEQ ID NO: 1219 - DNA, 42 bp, Artificial Sequence
  Synthetic Polynucleotide
agacgtctgaaatggggttattaaccgttgtagcaatagctc

SEQ ID NO: 1220 - DNA, 28 bp, Artificial Sequence
  Synthetic Polynucleotide
aaaaggaaaaggacattctggccaatat

SEQ ID NO: 1221 - DNA, 42 bp, Artificial Sequence
  Synthetic Polynucleotide
gtcccgcgcttaatgcgagccggcccccgatttagagcttga

SEQ ID NO: 1222 - DNA, 35 bp, Artificial Sequence
  Synthetic Polynucleotide
cggtgatgaagggtaaagttaaaccctcataggtt

SEQ ID NO: 1223 - DNA, 42 bp, Artificial Sequence
  Synthetic Polynucleotide
cagttgacgagcacgtagccaccggattagtaataacatgga

SEQ ID NO: 1224 - DNA, 42 bp, Artificial Sequence
  Synthetic Polynucleotide
tggaaacgcgagcaaaagaagatgtaaatccaattcatcgaa

SEQ ID NO: 1225 - DNA, 42 bp, Artificial Sequence
  Synthetic Polynucleotide
tcgctttcctcgttagaagtgtttcctgagtagaagaattgc

SEQ ID NO: 1226 - DNA, 46 bp, Artificial Sequence
  Synthetic Polynucleotide
ttaaataaccggggtgtcacttattggggttgcagcaagcggaatc

SEQ ID NO: 1227 - DNA, 28 bp, Artificial Sequence
  Synthetic Polynucleotide
attaattacatttagtggcgtgccgcat

SEQ ID NO: 1228 - DNA, 42 bp, Artificial Sequence
  Synthetic Polynucleotide
aagaaaagtgagccttgtttggccgccattaaaaaaccctca

SEQ ID NO: 1229 - DNA, 35 bp, Artificial Sequence
  Synthetic Polynucleotide
aacattgccgttccggccagcctcaattattacct

SEQ ID NO: 1230 - DNA, 42 bp, Artificial Sequence
  Synthetic Polynucleotide
ctggtccgttttgagaaacaataaattattcatttcaaatta

SEQ ID NO: 1231 - DNA, 46 bp, Artificial Sequence
  Synthetic Polynucleotide
ctgtcggtcatagaataagctcgtcatgtctggtcagcataaggcg

SEQ ID NO: 1232 - DNA, 32 bp, Artificial Sequence; identical to SEQ ID NO: 269
  Synthetic Polynucleotide

SEQ ID NO: 1233 - DNA, 35 bp, Artificial Sequence; identical to SEQ ID NO: 270
  Synthetic Polynucleotide

SEQ ID NO: 1234 - DNA, 42 bp, Artificial Sequence
  Synthetic Polynucleotide
tggcaaatacaaacaattcctcacagtttgtatctggtcagt

SEQ ID NO: 1235 - DNA, 42 bp, Artificial Sequence
  Synthetic Polynucleotide
cagacctcaaatatcaataccgaacaatataatatcaacggc

SEQ ID NO: 1236 - DNA, 42 bp, Artificial Sequence
  Synthetic Polynucleotide
ggttctaaagcatcaccaagataatatcagaaaaacagcgtc

SEQ ID NO: 1237 - DNA, 28 bp, Artificial Sequence
  Synthetic Polynucleotide
aatgccaacggcaggcacaggcggcctt

SEQ ID NO: 1238 - DNA, 34 bp, Artificial Sequence
  Synthetic Polynucleotide
caccgtcggtgcatcccaaaaatcccgtaaagcc

SEQ ID NO: 1239 - DNA, 42 bp, Artificial Sequence
  Synthetic Polynucleotide
acgcaaccagcttacggctggcggttgtgtacatcgacataa

SEQ ID NO: 1240 - DNA, 34 bp, Artificial Sequence
  Synthetic Polynucleotide
aggtgtccagcgcggggcatttgccgccgttggg

SEQ ID NO: 1241 - DNA, 28 bp, Artificial Sequence
  Synthetic Polynucleotide
cttaaatttctgcttcattgcaggcgct

SEQ ID NO: 1242 - DNA, 28 bp, Artificial Sequence
  Synthetic Polynucleotide
gttctttgaggactaacggtgtactaag

SEQ ID NO: 1243 - DNA, 39 bp, Artificial Sequence
  Synthetic Polynucleotide
tctgcgaattagcaaaatttccttttgaagttgatgggt

SEQ ID NO: 1244 - DNA, 28 bp, Artificial Sequence
  Synthetic Polynucleotide
tagctccaacaggtcagaaaagatagac

SEQ ID NO: 1245 - DNA, 45 bp, Artificial Sequence
  Synthetic Polynucleotide
aagaggcaaggcaaagaacgagtacgaaagaatatattcggaaaa

SEQ ID NO: 1246 - DNA, 42 bp, Artificial Sequence
  Synthetic Polynucleotide
cttattctactaatagtgtcaatagccgccacgggaccaggg

SEQ ID NO: 1247 - DNA, 35 bp, Artificial Sequence
  Synthetic Polynucleotide
aggaaatcaaaaatcagccaataccgagaggacat

SEQ ID NO: 1248 - DNA, 28 bp, Artificial Sequence
  Synthetic Polynucleotide
gatccctgactattataaatgtttgttt

SEQ ID NO: 1249 - DNA, 31 bp, Artificial Sequence
  Synthetic Polynucleotide
caatgacgccagctggcggaacgatcccaat

SEQ ID NO: 1250 - DNA, 35 bp, Artificial Sequence
  Synthetic Polynucleotide
agaggatgtgcgatcggattaaccgtgcatcgctc

SEQ ID NO: 1251 - DNA, 35 bp, Artificial Sequence
  Synthetic Polynucleotide
taacatcaatatgatataaacaaggttgataaatc

SEQ ID NO: 1252 - DNA, 42 bp, Artificial Sequence
  Synthetic Polynucleotide
gccagttgggctgcgcattgagggtcacgttggtgtagggcc

SEQ ID NO: 1253 - DNA, 42 bp, Artificial Sequence
  Synthetic Polynucleotide
ctctcccagtaagcgcccggcctcgattgaccgtaatgcatc

SEQ ID NO: 1254 - DNA, 48 bp, Artificial Sequence
  Synthetic Polynucleotide
aaaacgagaaaaatattcgacgatcgaggcaaataaaacgaactatta

SEQ ID NO: 1255 - DNA, 42 bp, Artificial Sequence
  Synthetic Polynucleotide
cataagcccgaagcaaaagcttaattgctgatgcaactcata

SEQ ID NO: 1256 - DNA, 35 bp, Artificial Sequence
  Synthetic Polynucleotide
ttatgcatcagattagatcatttttgcggatggaa

SEQ ID NO: 1257 - DNA, 42 bp, Artificial Sequence
  Synthetic Polynucleotide
ccgttaaatgccaaaaattaacatccaataaattagatcggg

SEQ ID NO: 1258 - DNA, 42 bp, Artificial Sequence
  Synthetic Polynucleotide
gtaatcgtaaaataatagtaagtagaaaggccggagacagtc

SEQ ID NO: 1259 - DNA, 35 bp, Artificial Sequence
  Synthetic Polynucleotide
gccaaaaacaattcgcaattaaatgtgagcgaacg

SEQ ID NO: 1260 - DNA, 21 bp, Artificial Sequence
  Synthetic Polynucleotide
tgcaagagtagcgcataacag

SEQ ID NO: 1261 - DNA, 42 bp, Artificial Sequence
  Synthetic Polynucleotide
tgcccacattattcatcagttgagaatcattcttgagacaga

SEQ ID NO: 1262 - DNA, 28 bp, Artificial Sequence
  Synthetic Polynucleotide
aacaacattattacagggcgatttcaga

SEQ ID NO: 1263 - DNA, 49 bp, Artificial Sequence
  Synthetic Polynucleotide
cgccattaggaatacagagggctcttcgctattacaattggggtgaatt

SEQ ID NO: 1264 - DNA, 35 bp, Artificial Sequence
  Synthetic Polynucleotide
agcctgtagccagctttggatagggacgacgtttc

SEQ ID NO: 1265 - DNA, 28 bp, Artificial Sequence
  Synthetic Polynucleotide
atcaaaagaaagactggatagcgtgtct

SEQ ID NO: 1266 - DNA, 42 bp, Artificial Sequence
  Synthetic Polynucleotide
ttgtaccccgagaatcgatgaacgaaatcactgtgtagcata

SEQ ID NO: 1267 - DNA, 42 bp, Artificial Sequence
  Synthetic Polynucleotide
acggcactcatgaggaagtttacaaacggctggctggcagcg

SEQ ID NO: 1268 - DNA, 36 bp, Artificial Sequence
  Synthetic Polynucleotide
gtatattcgccaagcccctgagagtctggagctcaa

SEQ ID NO: 1269 - DNA, 28 bp, Artificial Sequence
  Synthetic Polynucleotide
aacggtcaataaagtacggtgtctggct

SEQ ID NO: 1270 - DNA, 35 bp, Artificial Sequence
  Synthetic Polynucleotide
cagatcttgagaaacactaagaactggctcaacgg

SEQ ID NO: 1271 - DNA, 42 bp, Artificial Sequence
  Synthetic Polynucleotide
gggttcaaaagggtgcagcaagcaataaagcctcagaggtaa

SEQ ID NO: 1272 - DNA, 28 bp, Artificial Sequence
  Synthetic Polynucleotide
tttatatattttctagctgataaacatt

SEQ ID NO: 1273 - DNA, 42 bp, Artificial Sequence
  Synthetic Polynucleotide
aggtcattccatataactaagagggagtacctttaattgaag

SEQ ID NO: 1274 - DNA, 35 bp, Artificial Sequence
  Synthetic Polynucleotide
agcaccatcgcccacgcataaccgcagcatcgaaa

SEQ ID NO: 1275 - DNA, 42 bp, Artificial Sequence
  Synthetic Polynucleotide
caggatttagtttgaccatcatacctaaatcggttgtacaat

SEQ ID NO: 1276 - DNA, 35 bp, Artificial Sequence
  Synthetic Polynucleotide
atctgcaggggtggtgaagggatatgccagtactg

SEQ ID NO: 1277 - DNA, 42 bp, Artificial Sequence
  Synthetic Polynucleotide
ttgacatttcgcaaatgagtagcacattatgaccctgtaacc

SEQ ID NO: 1278 - DNA, 46 bp, Artificial Sequence
  Synthetic Polynucleotide
gggcgcgctgacgacaagaacaaaatagtgcggaatcgtcattgac

SEQ ID NO: 1279 - DNA, 28 bp, Artificial Sequence
  Synthetic Polynucleotide
aacagcggatcaaattcagtagtacttc

SEQ ID NO: 1280 - DNA, 42 bp, Artificial Sequence
  Synthetic Polynucleotide
agagacgtggtttatgcgggcggctagcatgtcaaataggaa

SEQ ID NO: 1281 - DNA, 35 bp, Artificial Sequence
  Synthetic Polynucleotide
tcacggtcgctgaggctgtcacccgcgattatgag

SEQ ID NO: 1282 - DNA, 41 bp, Artificial Sequence
  Synthetic Polynucleotide
tccagttaaaggacggataacctctgtgagagatagacaca

SEQ ID NO: 1283 - DNA, 46 bp, Artificial Sequence
  Synthetic Polynucleotide
taccgcttgccgttgcgggaggcgcagaagactttttcaatccgcc

SEQ ID NO: 1284 - DNA, 32 bp, Artificial Sequence
  Synthetic Polynucleotide
accttattagaaagcaactaatgcagatcttt

SEQ ID NO: 1285 - DNA, 35 bp, Artificial Sequence
  Synthetic Polynucleotide
aacgccaaaaggaattaaaaaacccggatatgatg

SEQ ID NO: 1286 - DNA, 42 bp, Artificial Sequence
  Synthetic Polynucleotide
cgcgtctatgggcgcatcgttcaactttattcaaaaataatt

SEQ ID NO: 1287 - DNA, 42 bp, Artificial Sequence
  Synthetic Polynucleotide
ttctcattttttaaccatcatatgggaagggctgcaagtcag

SEQ ID NO: 1288 - DNA, 42 bp, Artificial Sequence
  Synthetic Polynucleotide
aacttaaatttttgttaatcagaaattcaggtaacgccgctt

SEQ ID NO: 1289 - DNA, 32 bp, Artificial Sequence
  Synthetic Polynucleotide
ccattaaacgggtaaatgcgccgacaatgaca

SEQ ID NO: 1290 - DNA, 42 bp, Artificial Sequence
  Synthetic Polynucleotide
atacgtaatgccactacgaagaaacagcttgataccgatagt

SEQ ID NO: 1291 - DNA, 35 bp, Artificial Sequence
  Synthetic Polynucleotide
gcaccaacctaaaacgaaaaagaatacactaaaac

SEQ ID NO: 1292 - DNA, 35 bp, Artificial Sequence
  Synthetic Polynucleotide
aattgtatcggtttatctttcgaggtgaatttctt

SEQ ID NO: 1293 - DNA, 42 bp, Artificial Sequence
  Synthetic Polynucleotide
aaggctccaaaaggagcctttactcatctttgacccccagcg

SEQ ID NO: 1294 - DNA, 32 bp, Artificial Sequence
  Synthetic Polynucleotide
gaaaatctccaaaaaaattataccaagcgcga

SEQ ID NO: 1295 - DNA, 28 bp, Artificial Sequence
  Synthetic Polynucleotide
agatatataactatatataacaacgaat

SEQ ID NO: 1296 - DNA, 39 bp, Artificial Sequence
  Synthetic Polynucleotide
cagtatggaaggtaaatatatagcaatagactcctaacc

SEQ ID NO: 1297 - DNA, 28 bp, Artificial Sequence
  Synthetic Polynucleotide
gaatgagttaagcccaagacgggagcca

SEQ ID NO: 1298 - DNA, 21 bp, Artificial Sequence
  Synthetic Polynucleotide
tctagcaagaaacaatgtaaa

SEQ ID NO: 1299 - DNA, 45 bp, Artificial Sequence
  Synthetic Polynucleotide
tgaccgattgagggaggttagcaaggtctgatgaaaacaaaggaa

SEQ ID NO: 1300 - DNA, 42 bp, Artificial Sequence
  Synthetic Polynucleotide
gcccatatggtttaccaaaaagaaagcgtaacgatcagagtt

SEQ ID NO: 1301 - DNA, 35 bp, Artificial Sequence
  Synthetic Polynucleotide
taatcaaaaatgaaaatagagccttagttgctaga

SEQ ID NO: 1302 - DNA, 28 bp, Artificial Sequence
  Synthetic Polynucleotide
aagtttacagagagaataacgctactac

SEQ ID NO: 1303 - DNA, 31 bp, Artificial Sequence
  Synthetic Polynucleotide
aacagaccctcattttcccttttttattacg

SEQ ID NO: 1304 - DNA, 35 bp, Artificial Sequence
  Synthetic Polynucleotide
gaagcaagcctcagaacaatcctcaagagaaaaca

SEQ ID NO: 1305 - DNA, 35 bp, Artificial Sequence
  Synthetic Polynucleotide
aatatcggcattttcggctcagaaagccgcctctc

SEQ ID NO: 1306 - DNA, 42 bp, Artificial Sequence
  Synthetic Polynucleotide
gcagtaccgtccaccctgattagcacatgaaagtattagagt

SEQ ID NO: 1307 - DNA, 42 bp, Artificial Sequence
  Synthetic Polynucleotide
ccatcaccagtactcagtaccaggttcggaacctattataac

SEQ ID NO: 1308 - DNA, 48 bp, Artificial Sequence
  Synthetic Polynucleotide
cgattttttgaaaataatttgaagtaagaaccaagtaccgcactcgct

SEQ ID NO: 1309 - DNA, 42 bp, Artificial Sequence
  Synthetic Polynucleotide
acgctgaacacaagaataagtaagcagatagacgcaataaag

SEQ ID NO: 1310 - DNA, 35 bp, Artificial Sequence
  Synthetic Polynucleotide
gcccgcattataataagtaccgaagccctttcaaa

SEQ ID NO: 1311 - DNA, 42 bp, Artificial Sequence
  Synthetic Polynucleotide
agccatcgatcgacttgagacaaaagggcgatacataaagtg

SEQ ID NO: 1312 - DNA, 42 bp, Artificial Sequence
  Synthetic Polynucleotide
gccaccaccctcaatcttaccaattagcgtcagactgtagcg

SEQ ID NO: 1313 - DNA, 35 bp, Artificial Sequence
  Synthetic Polynucleotide
cccgaggttgaagccaggtcagtgccttgagtgcc

SEQ ID NO: 1314 - DNA, 21 bp, Artificial Sequence
  Synthetic Polynucleotide
ttgagccagttgtaattgttg

SEQ ID NO: 1315 - DNA, 42 bp, Artificial Sequence
  Synthetic Polynucleotide
aatcaatagctcatcgtaggaatccccatccaagtccttaat

SEQ ID NO: 1316 - DNA, 28 bp, Artificial Sequence
  Synthetic Polynucleotide
aggacaagcaagccgttgtagaaagcct

SEQ ID NO: 1317 - DNA, 49 bp, Artificial Sequence
  Synthetic Polynucleotide
catactaccgcgcctttatccctcagagccaccgcaatagattaattta

SEQ ID NO: 1318 - DNA, 35 bp, Artificial Sequence
  Synthetic Polynucleotide
tgactggtaataagtttttctgaaggggtttagcg

SEQ ID NO: 1319 - DNA, 28 bp, Artificial Sequence
  Synthetic Polynucleotide
tcgcacccagacgagcgtctttccagca

SEQ ID NO: 1320 - DNA, 42 bp, Artificial Sequence
  Synthetic Polynucleotide
accccaccagccgccaccctcagacgttttccagtagcaagg

SEQ ID NO: 1321 - DNA, 42 bp, Artificial Sequence
  Synthetic Polynucleotide
gttaaagtactgcaaatccaataaggcttagtaggcagaggg

SEQ ID NO: 1322 - DNA, 36 bp, Artificial Sequence
  Synthetic Polynucleotide
tcaggaggtttttgacagtcagagccgccacctcat

SEQ ID NO: 1323 - DNA, 28 bp, Artificial Sequence
  Synthetic Polynucleotide
attccagtataataacggaataccttaa

SEQ ID NO: 1324 - DNA, 35 bp, Artificial Sequence
  Synthetic Polynucleotide
acaaataagaagaacgcccaatcaataatcgatcg

SEQ ID NO: 1325 - DNA, 42 bp, Artificial Sequence
  Synthetic Polynucleotide
atatcaagtttgcctcaaatgacggaaattattcattagaca

SEQ ID NO: 1326 - DNA, 28 bp, Artificial Sequence
  Synthetic Polynucleotide
tcgatgaaacccccttattagcgtgcct

SEQ ID NO: 1327 - DNA, 21 bp, Artificial Sequence
  Synthetic Polynucleotide
ggtactggcatgattaagcta

SEQ ID NO: 1328 - DNA, 35 bp, Artificial Sequence
  Synthetic Polynucleotide
tccttaattttcccttagaatcctgagactaaggg

SEQ ID NO: 1329 - DNA, 42 bp, Artificial Sequence
  Synthetic Polynucleotide
ataacgtagaaaatacacattcaaattatcaccgtcacagca

SEQ ID NO: 1330 - DNA, 35 bp, Artificial Sequence
  Synthetic Polynucleotide
aatgattaagtgagaatagaaaggggattagcaga

SEQ ID NO: 1331 - DNA, 42 bp, Artificial Sequence
  Synthetic Polynucleotide
aataggtggcaacatatgcgccaaagccatttgggaatgtca

SEQ ID NO: 1332 - DNA, 46 bp, Artificial Sequence
  Synthetic Polynucleotide
atttgtactaatgcgaatatatcaagataatttgccagttacttta

SEQ ID NO: 1333 - DNA, 28 bp, Artificial Sequence
  Synthetic Polynucleotide
aattttttcacgttaactatcaacattt

SEQ ID NO: 1334 - DNA, 42 bp, Artificial Sequence
  Synthetic Polynucleotide
ttgcgaagaacaagcgccacctgagagccgccacctaagcgt

SEQ ID NO: 1335 - DNA, 35 bp, Artificial Sequence
  Synthetic Polynucleotide
actatagcgatagcttattatcaaaacccatccgt

SEQ ID NO: 1336 - DNA, 41 bp, Artificial Sequence
  Synthetic Polynucleotide
gagacgctgagataaagttttgtcctttcaacagtttctgc

SEQ ID NO: 1337 - DNA, 46 bp, Artificial Sequence
  Synthetic Polynucleotide
gtcttgttcagtcatcgcacaaattcttgtaaatgctgaaacggag

SEQ ID NO: 1338 - DNA, 32 bp, Artificial Sequence
  Synthetic Polynucleotide
cgagcattttatttaagcaaatcagatatatt

SEQ ID NO: 1339 - DNA, 35 bp, Artificial Sequence
  Synthetic Polynucleotide
agacttatccggtattcccttaaaaagtaccccat

SEQ ID NO: 1340 - DNA, 42 bp, Artificial Sequence
  Synthetic Polynucleotide
gatacagagaggctgagacaaataatatatatggcttttgat

SEQ ID NO: 1341 - DNA, 42 bp, Artificial Sequence
  Synthetic Polynucleotide
gtaatttaccgttccagagaaccagccaccccaataggaatc

SEQ ID NO: 1342 - DNA, 42 bp, Artificial Sequence
  Synthetic Polynucleotide
gggaatggaaagcgcaggccagcaagtaccgaacactgagtc

SEQ ID NO: 1343 - DNA, 28 bp, Artificial Sequence
  Synthetic Polynucleotide
tcgcaagacaaagataaatcgtcgctat

SEQ ID NO: 1344 - DNA, 34 bp, Artificial Sequence
  Synthetic Polynucleotide
acgcgagaaaattcaaagagtgaataaccttctg

SEQ ID NO: 1345 - DNA, 42 bp, Artificial Sequence
  Synthetic Polynucleotide
tatattttagttaatttcatcagtacataaatcaatatatgt

SEQ ID NO: 1346 - DNA, 34 bp, Artificial Sequence
  Synthetic Polynucleotide
ttctgacctaaaatggtattacctttttggaaac

SEQ ID NO: 1347 - DNA, 28 bp, Artificial Sequence
  Synthetic Polynucleotide
acaatttcatttgattgaaataccgacc

SEQ ID NO: 1348 - DNA, 30 bp, Artificial Sequence
  Synthetic Polynucleotide
ttttagacaggaacggtacgtatcggcctt

SEQ ID NO: 1349 - DNA, 27 bp, Artificial Sequence
  Synthetic Polynucleotide
ccagaacaatattaccgtagaaccctt

SEQ ID NO: 1350 - DNA, 42 bp, Artificial Sequence
  Synthetic Polynucleotide
gcgtaagaatacgtggcacagacaacagagaccagccactca

SEQ ID NO: 1351 - DNA, 35 bp, Artificial Sequence
  Synthetic Polynucleotide
gccacgctgagagccagcagcaaaggtcagtaatt

SEQ ID NO: 1352 - DNA, 37 bp, Artificial Sequence
  Synthetic Polynucleotide
atccgtagatacagtaccgggagctaaacaggaggcc

SEQ ID NO: 1353 - DNA, 30 bp, Artificial Sequence
  Synthetic Polynucleotide
gaaaccaccagaaggagcggattaacaccg

SEQ ID NO: 1354 - DNA, 21 bp, Artificial Sequence
  Synthetic Polynucleotide
atgaatatacagtatttcagg

SEQ ID NO: 1355 - DNA, 30 bp, Artificial Sequence
  Synthetic Polynucleotide
agttacaaaatcgcgcaaacattatcattt

SEQ ID NO: 1356 - DNA, 34 bp, Artificial Sequence
  Synthetic Polynucleotide
atatttgagtgaggcgacggattcgcctgattgc

SEQ ID NO: 1357 - DNA, 21 bp, Artificial Sequence
  Synthetic Polynucleotide
aatagattagagccttaggag

SEQ ID NO: 1358 - DNA, 30 bp, Artificial Sequence
  Synthetic Polynucleotide
gagctgaaaaggtggcatcattgcgggaga

SEQ ID NO: 1359 - DNA, 27 bp, Artificial Sequence
  Synthetic Polynucleotide
caacgcaaggataaaaacggagagggt

SEQ ID NO: 1360 - DNA, 42 bp, Artificial Sequence
  Synthetic Polynucleotide
agagatctacaaaggctatcaggtttaatgctttttagaata

SEQ ID NO: 1361 - DNA, 35 bp, Artificial Sequence
  Synthetic Polynucleotide
tgtaaacgttaatattttgttaaaggaagatccag

SEQ ID NO: 1362 - DNA, 37 bp, Artificial Sequence
  Synthetic Polynucleotide
gcacacgacgaggtggaacctgtttagctatattttc

SEQ ID NO: 1363 - DNA, 30 bp, Artificial Sequence
  Synthetic Polynucleotide
accgcttctggtgccggaaatgtataagca

SEQ ID NO: 1364 - DNA, 21 bp, Artificial Sequence
  Synthetic Polynucleotide
tgccaagctttcagttgtaaa

SEQ ID NO: 1365 - DNA, 30 bp, Artificial Sequence
  Synthetic Polynucleotide
gccatgtttaccagtcctcgcactccagcc

SEQ ID NO: 1366 - DNA, 34 bp, Artificial Sequence
  Synthetic Polynucleotide
gcgaggaagacggaattaccggaaacaatcggcg

SEQ ID NO: 1367 - DNA, 21 bp, Artificial Sequence
  Synthetic Polynucleotide
tctccgtgggaacaagtaaca

SEQ ID NO: 1368 - DNA, 30 bp, Artificial Sequence
  Synthetic Polynucleotide
gtcacaatcaatagaaaattagcaaaatca

SEQ ID NO: 1369 - DNA, 27 bp, Artificial Sequence
  Synthetic Polynucleotide
attaccattagcaaggccttttcataa

SEQ ID NO: 1370 - DNA, 42 bp, Artificial Sequence
  Synthetic Polynucleotide
ggaaccagagccaccaccggaaccttgccatcggaaactaga

SEQ ID NO: 1371 - DNA, 35 bp, Artificial Sequence
  Synthetic Polynucleotide
tcacaaacaaataaatcctcattaaggcaggatca

SEQ ID NO: 1372 - DNA, 37 bp, Artificial Sequence
  Synthetic Polynucleotide
ccgtacaaaccatagttacgcaaagacaccacggaat

SEQ ID NO: 1373 - DNA, 30 bp, Artificial Sequence
  Synthetic Polynucleotide
gtatagcccggaataggtgttcagacgatt

SEQ ID NO: 1374 - DNA, 21 bp, Artificial Sequence
  Synthetic Polynucleotide
ccacagacagcccttacaacg

SEQ ID NO: 1375 - DNA, 30 bp, Artificial Sequence
  Synthetic Polynucleotide
tctgtatgggattttgcgtgccgtcgagag

SEQ ID NO: 1376 - DNA, 34 bp, Artificial Sequence
  Synthetic Polynucleotide
tatcggataataaacaagtctttccagacgttag

SEQ ID NO: 1377 - DNA, 21 bp, Artificial Sequence
  Synthetic Polynucleotide
cagttaatgccccctaacagt

SEQ ID NO: 1378 - DNA, 11 bp, Artificial Sequence
  Synthetic Polynucleotide
tttgaatacca

SEQ ID NO: 1379 - DNA, 11 bp, Artificial Sequence
  Synthetic Polynucleotide
aaacgtacatt

SEQ ID NO: 1380 - DNA, 11 bp, Artificial Sequence
  Synthetic Polynucleotide
taaatgaatgc

SEQ ID NO: 1381 - DNA, 11 bp, Artificial Sequence
  Synthetic Polynucleotide
tgcggaacaag

SEQ ID NO: 1382 - DNA, 11 bp, Artificial Sequence
  Synthetic Polynucleotide
agctttccgtt

SEQ ID NO: 1383 - DNA, 11 bp, Artificial Sequence
  Synthetic Polynucleotide
ggttgatatag

SEQ ID NO: 1384 - DNA, 11 bp, Artificial Sequence
  Synthetic Polynucleotide
tttaacgtcaa

SEQ ID NO: 1385 - DNA, 11 bp, Artificial Sequence
  Synthetic Polynucleotide
acgacggccaa

SEQ ID NO: 1386 - DNA, 11 bp, Artificial Sequence
  Synthetic Polynucleotide
cctgtagcagc

SEQ ID NO: 1387 - DNA, 11 bp, Artificial Sequence
  Synthetic Polynucleotide
gattaaaggct

SEQ ID NO: 1388 - DNA, 11 bp, Artificial Sequence
  Synthetic Polynucleotide
gctggtaatgt

SEQ ID NO: 1389 - DNA, 11 bp, Artificial Sequence
  Synthetic Polynucleotide
ctgacctgaaa

SEQ ID NO: 1390 - DNA, 11 bp, Artificial Sequence
  Synthetic Polynucleotide
cctgcaacaat

SEQ ID NO: 1391 - DNA, 11 bp, Artificial Sequence
  Synthetic Polynucleotide
cactaacaaga

SEQ ID NO: 1392 - DNA, 11 bp, Artificial Sequence
  Synthetic Polynucleotide
atttggggcaa

SEQ ID NO: 1393 - DNA, 11 bp, Artificial Sequence
  Synthetic Polynucleotide
agcctttatat

SEQ ID NO: 1394 - DNA, 11 bp, Artificial Sequence
  Synthetic Polynucleotide
agctattttcc

SEQ ID NO: 1395 - DNA, 11 bp, Artificial Sequence
  Synthetic Polynucleotide
aatatttaacc

SEQ ID NO: 1396 - DNA, 11 bp, Artificial Sequence
  Synthetic Polynucleotide
acccgtcggtt

SEQ ID NO: 1397 - DNA, 11 bp, Artificial Sequence
  Synthetic Polynucleotide
aagtttattat

SEQ ID NO: 1398 - DNA, 11 bp, Artificial Sequence
  Synthetic Polynucleotide
ccagtagcaat

SEQ ID NO: 1399 - DNA, 11 bp, Artificial Sequence
  Synthetic Polynucleotide
tcaaaatcatg

SEQ ID NO: 1400 - DNA, 11 bp, Artificial Sequence
  Synthetic Polynucleotide
ggccttgattt

SEQ ID NO: 1401 - DNA, 11 bp, Artificial Sequence
  Synthetic Polynucleotide
gcccgtatagc

SEQ ID NO: 1402 - DNA, 46 bp, Artificial Sequence
  Synthetic Polynucleotide
tttttgctggcaagtgtagcggagcgggtcaaggtgccgtaaaacg

SEQ ID NO: 1403 - DNA, 31 bp, Artificial Sequence
  Synthetic Polynucleotide
tttttaaaaaccgtctacgctagggcttttt

SEQ ID NO: 1404 - DNA, 48 bp, Artificial Sequence; identical to SEQ ID NO: 440
  Synthetic Polynucleotide

SEQ ID NO: 1405 - DNA, 31 bp, Artificial Sequence; identical to SEQ ID NO: 441
  Synthetic Polynucleotide

SEQ ID NO: 1406 - DNA, 41 bp, Artificial Sequence
  Synthetic Polynucleotide
acttttctttacaccggaatcataattactagaaaattttt

SEQ ID NO: 1407 - DNA, 41 bp, Artificial Sequence
  Synthetic Polynucleotide
tttttggctggtaatgggtaaaggggtgtgttcagcttttt

SEQ ID NO: 1408 - DNA, 41 bp, Artificial Sequence; identical to SEQ ID NO: 443
  Synthetic Polynucleotide

SEQ ID NO: 1409 - DNA, 45 bp, Artificial Sequence
  Synthetic Polynucleotide
tttttcaacatgttttaaataatataatgcgaaccagaccggaaa

SEQ ID NO: 1410 - DNA, 31 bp, Artificial Sequence
  Synthetic Polynucleotide
ttttttcgagcttcaaagctgtagctttttt

SEQ ID NO: 1411 - DNA, 49 bp, Artificial Sequence
  Synthetic Polynucleotide
gactgaggacatcattacgaataagagtcaggacgttgggaagattttt

SEQ ID NO: 1412 - DNA, 31 bp, Artificial Sequence
  Synthetic Polynucleotide
tttttaagctgctcattcagtccaaatctac

SEQ ID NO: 1413 - DNA, 41 bp, Artificial Sequence
  Synthetic Polynucleotide
aggccggaactatgagccgggtcactgttgccctgcttttt

SEQ ID NO: 1414 - DNA, 41 bp, Artificial Sequence
  Synthetic Polynucleotide
tttttcctgctccatgttacttaggaaccgaactgattttt

SEQ ID NO: 1415 - DNA, 41 bp, Artificial Sequence
  Synthetic Polynucleotide
tttttaaaatctacgtttagtaagagcaacactatcttttt

SEQ ID NO: 1416 - DNA, 46 bp, Artificial Sequence
  Synthetic Polynucleotide
tttttgaaggaaaccgaggaaccgaacaagagagataacccaccct

SEQ ID NO: 1417 - DNA, 31 bp, Artificial Sequence
  Synthetic Polynucleotide
tttttagcgctaatatcaagttaccattttt

SEQ ID NO: 1418 - DNA, 48 bp, Artificial Sequence
  Synthetic Polynucleotide
gaaagaatcggacaaaaaacaacattccttatcattccaagaattttt

SEQ ID NO: 1419 - DNA, 31 bp, Artificial Sequence
  Synthetic Polynucleotide
tttttccagacgacgacaataggtaaagggg

SEQ ID NO: 1420 - DNA, 41 bp, Artificial Sequence
  Synthetic Polynucleotide
ccagcgttatctgataaattgtgtcgaaatccgcgattttt

SEQ ID NO: 1421 - DNA, 41 bp, Artificial Sequence
  Synthetic Polynucleotide
tttttagcctgtttagtatcatatacgctcaacagtttttt

SEQ ID NO: 1422 - DNA, 41 bp, Artificial Sequence
  Synthetic Polynucleotide
tttttcgggtattaaacgcgaggcgttttagcgaacttttt

SEQ ID NO: 1423 - DNA, 71 bp, Artificial Sequence
  Synthetic Polynucleotide
ggggtggtttgccccagcaggcgacagttaaaattctcattgcaatccaaataaagagggtaattgttttt

SEQ ID NO: 1424 - DNA, 71 bp, Artificial Sequence
  Synthetic Polynucleotide
cagacattgaatccccctcaaataatagtagtctaatctatgaaaatcctgtttcgtcaaagggcgttttt

SEQ ID NO: 1425 - DNA, 67 bp, Artificial Sequence
  Synthetic Polynucleotide
aggtacagccatattatttatcccactaatcttatgtagctttaaacagttcgcgttttaatttttt

SEQ ID NO: 1426 - DNA, 73 bp, Artificial Sequence
  Synthetic Polynucleotide
tttttaatcggccaacgtgctgcggccacaagttaaagattcgtcattgaagggcttaattgcaaagtcgaaa

SEQ ID NO: 1427 - DNA, 73 bp, Artificial Sequence
  Synthetic Polynucleotide
tttttataaccctcgttaacgtaacagtaatagtagtctacatctatggcaaatcgttaacgactccaagatg

SEQ ID NO: 1428 - DNA, 61 bp, Artificial Sequence
  Synthetic Polynucleotide
tttttctcccgacttgctaattctgttaatcttatgtaccaactttgaaatcaaatatcag

SEQ ID NO: 1429 - DNA, 19 bp, Artificial Sequence
  Synthetic Polynucleotide
caatgagaattttaactgt

SEQ ID NO: 1430 - DNA, 19 bp, Artificial Sequence
  Synthetic Polynucleotide
catagattagactactatt

SEQ ID NO: 1431 - DNA, 15 bp, Artificial Sequence
  Synthetic Polynucleotide
tacataagattagtg

SEQ ID NO: 1432 - DNA, 24 bp, Artificial Sequence
  Synthetic Polynucleotide
tcaatgacgaatctttaacttgtg

SEQ ID NO: 1433 - DNA, 24 bp, Artificial Sequence
  Synthetic Polynucleotide
gccatagatgtagactactattac

SEQ ID NO: 1434 - DNA, 12 bp, Artificial Sequence
  Synthetic Polynucleotide
tacataagatta

(19) United States
(12) Patent Application Publication
(10) Pub. No.: US 2017/0015698 A1
(43) Pub. Date: Jan. 19, 2017

(54) NUCLEIC ACID POLYHEDRA FROM SELF-ASSEMBLED VERTEX-CONTAINING FIXED-ANGLE NUCLEIC ACID STRUCTURES

(71) Applicant: President and Fellows of Harvard College, Cambridge, MA (US)

(72) Inventors: Ryosuke Iinuma, Tsukuba-city (JP); Yonggang Ke, Sandy Springs, GA (US); Ralf Jungmann, Munich (DE); Peng Yin, Brookline, MA (US)

(73) Assignee: President and Fellows of Harvard College, Cambridge, MA (US)

(21) Appl. No.: 15/124,066

(22) PCT Filed: Mar. 6, 2015
(86) PCT No.: PCT/US2015/019135

§ 371 Date: Sep. 7, 2016

Related U.S. Application Data
(60) Provisional application No. 61/950,098, filed on Mar. 8, 2014.

(51) Int. Cl. C07H 21/04

(52) U.S. Cl. CPC C07H 21/04

(57) ABSTRACT

Provided herein are compositions comprising nucleic acid structures comprising three or more arms arranged at fixed angles from each other, composites thereof such as DNA cages, and methods for their synthesis and use.

### RELATED APPLICATION

This application claims the benefit under 35 U.S.C. §119(e) of U.S. Provisional application No. 61/950,098, filed Mar. 8, 2014, which is incorporated by reference herein in its entirety.

### FEDERALLY SPONSORED RESEARCH

This invention was made with U.S. Government support under grant number N000141110914, N000141010827 and N00014130593, awarded by the Office of Naval Research; grant number W911NF1210238, awarded by the Army Research Office; grant numbers 1DP2OD007292, 1R01EB018659 and 5R21HD072481, awarded by the National Institutes of Health; and grant numbers CCF1054898, CCF1317291, CCF1162459 and CMM11333215, awarded by the National Science Foundation. The U.S. Government has certain rights in the invention.

### FIELD OF INVENTION

Provided herein are a novel compositions and methods for generating nucleic acid structures such as DNA cages.

### BACKGROUND OF INVENTION

DNA nanotechnology has produced a wide range of shape-controlled nanostructures (1-10). Hollow polyhedra (1, 5, 11-26) are particularly interesting, as they resemble natural structures such as viral capsids and promise applications for scaffolding and encapsulating functional materials. Previous work has constructed diverse polyhedra, such as tetrahedra (13, 16, 20, 24), cubes (1, 19, 23), bipyramids (15), truncated octahedra (11), octahedra (12), dodecahedra (16, 18), icosahedra (17, 21), nano-prisms (14, 22, 25, 26), and buckyballs (16), with sub-80 nm sizes and sub-5 megadalton (MD) molecular weights (e.g. structures 1-8 in FIG. 1A). Assembly strategies include step-wise synthesis (1, 11, 21, 22), folding of a long scaffold (12, 19, 20, 24, 25), cooperative assembly of individual strands (13-15, 18, 26), and hierarchical assembly of branched DNA tiles (16, 17, 23).

Another route to scaling up polyhedra is the hierarchical assembly of larger monomers. Previous work using small three-arm-junction (16, 21) (80 kD) and five-arm junction tiles (17) (130 kD) has produced several sub-5 MD polyhedra (e.g. structures 5-7 in FIG. 1A). Additionally, a 15 MD icosahedron (5) (FIG. 1A, structure 9) was assembled from three double-triangle shaped origami monomers. However, this icosahedron was generated in low yield (5) and this method has not been generalized to construct more complex polyhedra.

### SUMMARY OF INVENTION

The invention provides a novel, general strategy for, optionally, one-step self-assembly of wireframe DNA polyhedra that are larger than previous structures and that are produced at higher yield than previous structures. A stiff three-arm-junction tile motif, which can be made using for example DNA origami, with precisely controlled angles and arm lengths is used for hierarchical assembly of polyhedra. Using these methods, it was possible to construct a tetrahedron (20 megadaltons or MD), a triangular prism (30 MD), a cube (40 MD), a pentagonal prism (50 MD), and a hexagonal prism (60 MD) with edge widths of 100 nanometers. The structures were visualized by transmission electron microscopy and by three-dimensional DNA-PAINT super-resolution fluorescent microscopy of single molecules in solution.

Thus, in one aspect, provided herein is a nucleic acid structure comprising a first (x), a second (y), and a third (z) nucleic acid arm, each connected at one end to the other arms to form a vertex, and a first, a second, and a third nucleic strut, wherein the first nucleic acid strut connects the first (x) nucleic arm to the second (y) nucleic arm, the second nucleic acid strut connects the second (y) nucleic arm to the third (z) nucleic arm, and the third nucleic acid strut connects the third (z) arm to the first (x) nucleic acid strut.

In another aspect, provided herein is a nucleic acid structure comprising three nucleic acid arms radiating from a vertex at fixed angles.

In another aspect, provided herein is a nucleic acid structure comprising N nucleic acid arms radiating from a vertex, wherein N is the number of nucleic acid arms and is 3 or more, and M nucleic acid struts, each strut connecting two nucleic acid arms to each other, wherein M is the number of nucleic acid struts and is 3 or more. In some embodiments, N is equal to M. In some embodiments, N is less than M.

Embodiments relating to one or more of the foregoing aspects are now provided.

In some embodiments, the nucleic acid structure comprises 4 nucleic acids and at least 4 nucleic acid struts, or 5 nucleic acid arms and at 5 nucleic acid struts.

In some embodiments, the nucleic acid arms are equally spaced apart from each other (or the arms are separated from each other by the same angle). In some embodiments, the nucleic acid arms are not equally separated from each other (or the arms are separated from each other by different angles).

In some embodiments, the nucleic acid structure comprises three nucleic acid arms separated from each other by 60°-60°-60°. When four such structures are connected to each other at their free ends, they form a tetrahedron.

In some embodiments, the nucleic acid structure comprises three nucleic acid arms separated from each other by 60°-90°-90°. When six such structures are connected to each other at their free ends, they form a triangular prism.

In some embodiments, the nucleic acid structure comprises three nucleic acid arms separated from each other by 90°-90°-90°. When eight such structures are connected to each other at their free ends, they form a cube.

In some embodiments, the nucleic acid structure comprises three nucleic acid arms separated from each other by 108°-90°-90°. When ten such structures are connected to each other at their free ends, they form a pentagonal prism. In some instances, pentagonal prisms may be formed by connecting nucleic acid structures defined as 120°-90°-90°.

In some embodiments, the nucleic acid structure comprises three nucleic acid arms separated from each other by 120°-90°-90°. When twelve such structures are connected to each other at their free ends, they form a hexagonal prism. In some instances, pentagonal prisms may be formed by connecting nucleic acid structures defined as 140°-90°-90°. In some embodiments, the nucleic acid structure further comprises a vertex nucleic acid.

In some embodiments, the nucleic acid structure further comprises a connector nucleic acid.

In some embodiments, the nucleic acid arms, nucleic acid struts, and/or vertex nucleic acid are comprised of parallel double helices.

In some embodiments, nucleic acid arms are of identical length.

In some embodiments, the nucleic acid struts are of identical length. In some embodiments, the nucleic acid struts are of different lengths.

In some embodiments, at least one nucleic acid arm comprises a blunt end.

In some embodiments, at least one nucleic acid arm comprises a connector nucleic acid at its free (non-vertex) end that is up to 16 nucleotides in length. In some embodiments, at least one nucleic acid arm comprises a connector nucleic acid at its free (non-vertex) end, thereby comprising a 1 or 2 nucleotide overhang.

In some embodiments, the nucleic acid structure is up to 5 megadaltons (MD) in size.

In some embodiments, the nucleic acid arms are 50 nm in length.

In another aspect, provided herein is a composite nucleic acid structure comprising L nucleic acid structures selected from any of the foregoing nucleic acid structures, wherein L is an even number of nucleic acid structures, and wherein the L nucleic acid structures are connected to each other at free (non-vertex) ends of the nucleic acid arms.

In some embodiments, the two more nucleic acid structures are two, four, six, eight, ten, twelve or more nucleic acid structures.

In some embodiments, the composite nucleic acid structure is a tetrahedron, a triangular prism, a cube, a pentagonal prism, or a hexagonal prism.

In some embodiments, the composite nucleic acid structure is 20 megadaltons (MD), 30 MD, 40 MD, 50 MD, or 60 MD in size.

In some embodiments, the composite nucleic acid structure has edge widths, comprised of two nucleic acid arms from adjacent nucleic acid structures, of 100 nm.

In another aspect, provided herein are methods of synthesis of any of the foregoing nucleic acid structures and the composite nucleic acid structures. In some embodiments, the methods comprise combining a nucleic acid scaffold strand with nucleic acid staple strands in a reaction vessel, wherein the nucleic acid staple strands are selected to form any of the foregoing nucleic acid structures when hybridized to the nucleic acid scaffold strand. In some embodiments, the methods further comprise combining the nucleic acid scaffold strand, the nucleic acid staple strands, and nucleic acid connector strands, wherein when the nucleic acid scaffold strand, the nucleic acid staple strands, and nucleic acid connector strands are hybridized to each other, they form a composite nucleic acid structure, such as any of the foregoing composite nucleic acid structures.

These and other aspects and embodiments provided herein are described in greater detail herein.

### BRIEF DESCRIPTION OF DRAWINGS

FIGS. 1A-1B. DNA-origami polyhedra. (FIG. 1A) Polyhedra self-assembled from DNA tripods with tunable inter-arm angles, and comparison of their sizes and molecular weights with selected previous polyhedra (structures 1-9; see FIG. 5 for details). (FIG. 1B) Design diagram of a tripod. Cylinders represent DNA double helices. See FIG. 6 for details of the arm connection at the vertex. (FIG. 1C) Cylinder model illustrating the connection between two tripod monomers. (FIG. 1D and FIG. 1E) Connection schemes for assembling (FIG. 1E) the tetrahedron and (FIG. 1D) other polyhedra (represented here by the cube design).

FIGS. 2A-2F. Self-assembly of DNA tripods and polyhedra. (FIG. 2A) Gel electrophoresis and (FIG. 2B) TEM images of the 60°-60°-60° (lane 1 in the gel) and 90°-90°-90° (lane 2) tripods. Gel lane 3: 1 kb ladder. Gel electrophoresis: 1.5% native agarose gel, ice water bath. (FIGS. 2C and 2D) Two schemes of connector designs and corresponding gel electrophoresis results. For each scheme, the strand model depicts the connection between two pairs of DNA duplexes. The number above a gel lane denotes the number of connected helices between two adjacent arms. Lane L: 1 kb ladder. Lane S: scaffold. Arrowheads indicate the bands corresponding to assembled cubes. (FIG. 2C) Scheme i: long (30 nt) connector (colored red) including a 2 nt sticky end. The complete 30 nt connector is only shown on the left, with a 28 nt segment anchored on the left helices and a 2 nt exposed sticky end available for hybridization with the 90°-90°-90° right neighbor (dashed circle depicts hybridization site). (FIG. 2D) Scheme ii: short (11 nt) connector including a 2 nt sticky end. (FIG. 2E) Assembly yields of the cubes, calculated as intensity ratio between a cube band and the corresponding scaffold band. (FIG. 2F) Agarose gel electrophoresis of the polyhedra. Lane 1: 90°-90°-90° monomer. Lanes 2-6: polyhedra. Lane 7: assembly reaction containing tripods without struts. Lane 8: assembly reaction containing 90°-90°-90° tripods without vertex helices. Lane 9: 1 kb ladder. Gel bands corresponding to desired products are marked with arrowheads. Gel electrophoresis: 0.8% native agarose gel, ice water bath.

FIGS. 3A-3E. TEM images of polyhedra. The zoomed-in (columns 1 and 2) and zoomed-out (column 3) images are shown for the tetrahedron (FIG. 3A), the triangular prism (FIG. 3B), the cube (FIG. 3C), the pentagonal prism (FIG. 3D), and the hexagonal prism (FIG. 3E). Images of the tetrahedron, the triangular prism, and the cube were acquired from purified samples. Images of the pentagonal prism and hexagonal prism were collected from crude samples (denoted with “*”). Scale bars are 100 nm in the zoomed-in TEM images and 500 nm in the zoomed-out images. Note that aggregates are clearly visible for unpurified samples (e.g. in the rightmost panel of D).

FIGS. 4A1-4G. 3D DNA-PAINT super-resolution fluorescence imaging of polyhedra. (FIG. 4A1) Staple strands at the vertices of each polyhedron were extended with single-stranded docking sequences for 3D DNA-PAINT super-resolution imaging. (FIGS. 4A1-4E1) Schematics of polyhedra with DNA-PAINT sites highlighted. (FIGS. 4A2-4E2) 3D DNA-PAINT super-resolution reconstruction of typical polyhedra shown in the same perspective as depicted in A1-E1. (FIGS. 4A3-4E3) 2D x-y-projection. (FIGS. 4A4-4E4) 2D x-z-projection. (FIG. 2.4A5-4E5) Height measurements of the polyhedra obtained from the cross-sectional histograms in the x-z-projections. (FIG. 4F) A larger 2D super-resolution x-y-projection view of tetrahedra and drift markers (bright individual dots). The diffraction-limited image is super imposed on the super-resolution image in the upper half. (FIG. 4G) Tilted 3D view of a larger field of view image of the tetrahedron. Drift markers appear as bright individual dots. Scale bars: 200 nm. Color indicates height in the z direction.

FIG. 5.20-60 megadalton DNA polyhedra. 20-60 megadalton DNA wireframe polyhedra assembled from tunable DNA-origami tripods. Top, schematics showing the assembly process of tripod monomers and the polyhedra; middle, TEM images of polyhedra; bottom, super-resolution fluorescence images of polyhedra. These polyhedra are significantly larger than previous DNA polyhedra in FIG. 1A, including (1) a cube (1), a truncated octahedron (11), a tetrahedron (13), an octahedron (12), (2) a tetrahedron, a dodecahedron, and a buckyball assembled from three-arm DNA tiles (16), (3) a DNA-origami tetrahedron (24), and (4) an icosahedron assembled from three DNA-origami monomers (5).

FIG. 6. Connections at the vertex the three-arm monomer. Three layers of connections at the vertex: (1) the first-layer (innermost) connections are formed by the scaffold strand only. There are no extra bases between the duplexes. (2) the second-layer (middle) connections and (3) the third-layer (outmost) connections are DNA duplexes (i.e., the vertex helices) formed by staple strands and their complementary strands. Each polyhedron used different number of vertex helices with different lengths (see Table 2), which were estimated on the distances between the ends of the 16-helix arms at the vertexes. For detailed design and sequence information, refer to FIG. 8 to FIG. 13. The “*”s denote the helices where DNA handles were placed for DNA-PAINT.

FIGS. 7A-7C. Connection pattern. (FIG. 7A) A three-arm tripod monomer. (FIG. 7B) The cross-section of an arm of the three-arm monomer. The arrows in A and B indicate the same direction. The dotted line indicates the line of reflection symmetry. (FIG. 7C) The connection patterns that were implemented in FIG. 2B to FIG. 2E. See FIG. 8 to FIG. 13 for design and sequence details.

FIG. 8. Strand diagrams of the tetrahedron. The sequences used are provided in Table 4. The horizontal axis provides the position or length of the helix from the first base thereof. The vertical axis provides the helix number. As illustrated, there are three groupings of helices, each representing an arm. The 3 protrusions on the right side correspond to the 3 struts. The right end of the helices represents the free ends, while the left ends represent the ends at the vertex. Similarly renderings are provided in FIGS. 9-13.

FIG. 9. Strand diagrams of the triangular prism. The sequences used are provided in Table 5.

FIG. 10. Strand diagrams of the cube (short connectors). The sequences used are provided in Table 6.

FIG. 11. Strand diagrams of the cube (long connectors). The sequences used are provided in Table 7.

FIG. 12. Strand diagrams of the pentagonal prism. The sequences used are provided in Table 8.

FIG. 13. Strand diagrams of the hexagonal prism. The sequences used are provided in Table 9.

FIGS. 14A-14B. Schematics of nucleic acid structures having N arms, and N or more nucleic acid struts.

### DETAILED DESCRIPTION OF INVENTION

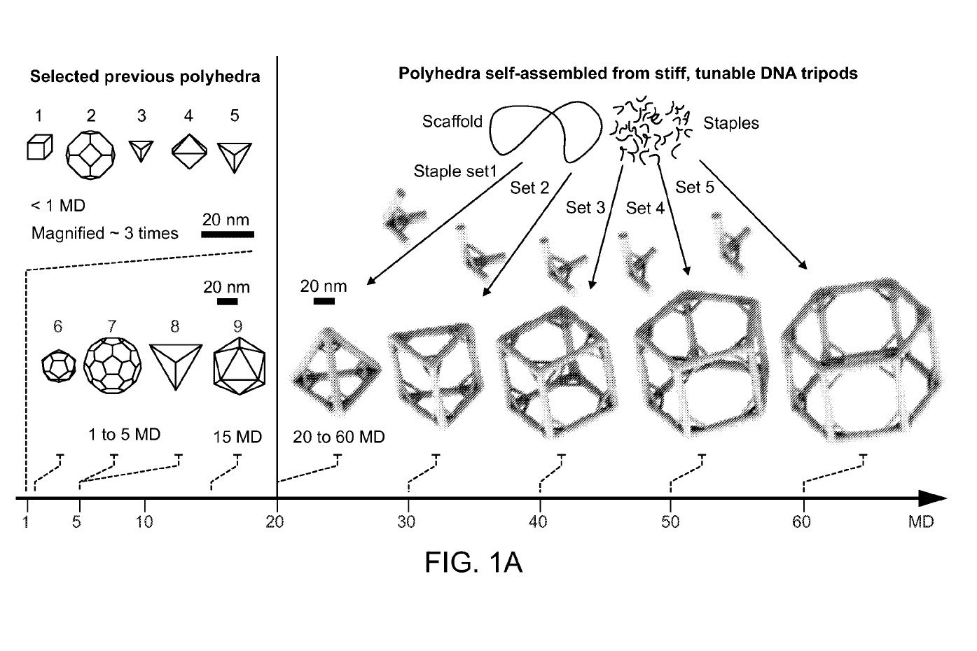

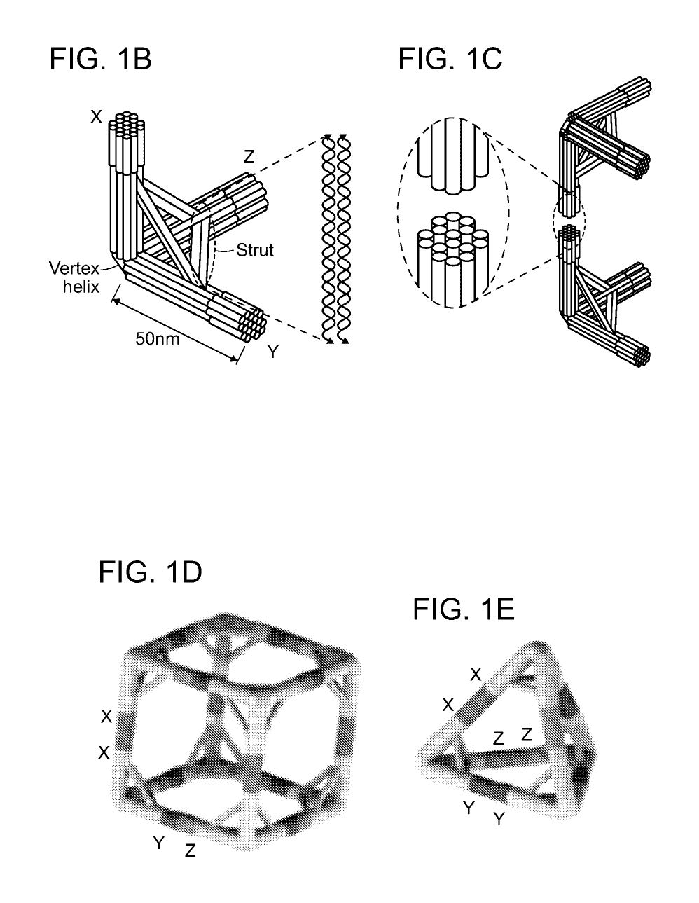

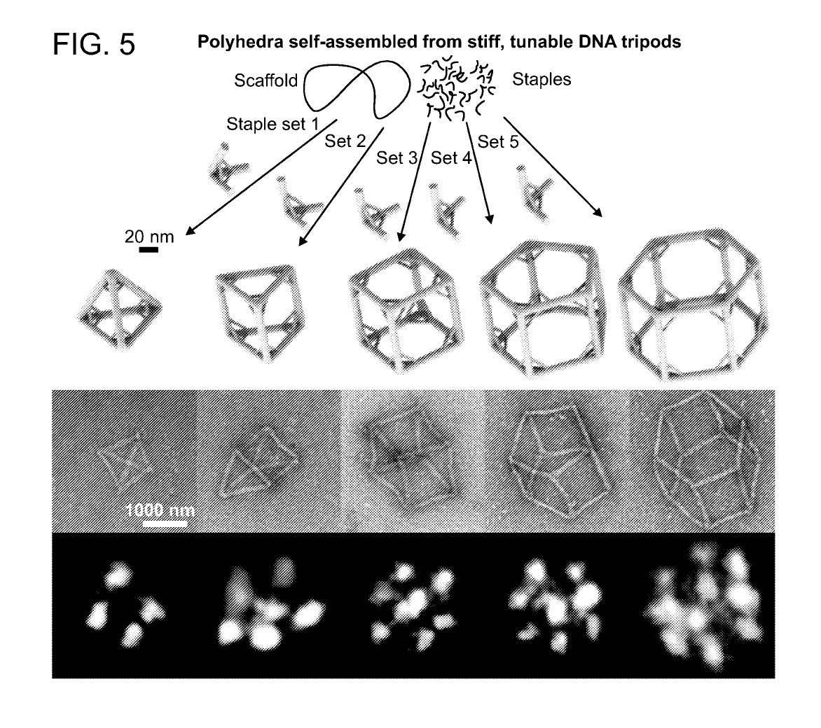

The invention is based, in part, on the discovery and development of a general strategy for hierarchical self-assembly of polyhedra from megadalton monomers using a DNA “tripod”, a 5 MD three-arm-junction origami tile that is 60 times more massive than previous three-arm tiles (16). The tripod motif features inter-arm angles controlled by supporting struts and strengthened by vertex helices. The invention further provides self-assembly of tripods into wireframe polyhedra using a dynamic connector design. Using this robust methodology, we constructed a tetrahedron (˜20 MD), a triangular prism (˜30 MD), a cube (˜40 MD), a pentagonal prism (˜50 MD), and a hexagonal prism (˜60 MD) (FIG. 1A and FIG. 5).

These structures have a variety of applications including but not limited to biological applications. For example, when generated having edges widths on the order of about 100 nm, these polyhedra have a size comparable to bacterial microcompartments such as carboxysomes. Additional applications include without limitation use in or as photonic devices, nanoelectronics and drug delivery systems.

To characterize the 3D single-molecule morphology of these polyhedra, we used a DNA-based super-resolution fluorescence imaging method (resolution below the diffraction limit) called DNA-PAINT (28, 29) (a variation of point accumulation for imaging in nanoscale topography (30)). Unlike traditional transmission electron microscopy (TEM) which images the samples in a vacuum under dried and stained conditions and thus may not render the structure in its native form, 3D DNA-PAINT introduces minimal distortion to the structures by rendering them in a more “native” hydrated imaging environment.

### General Tripod Design and Methodology

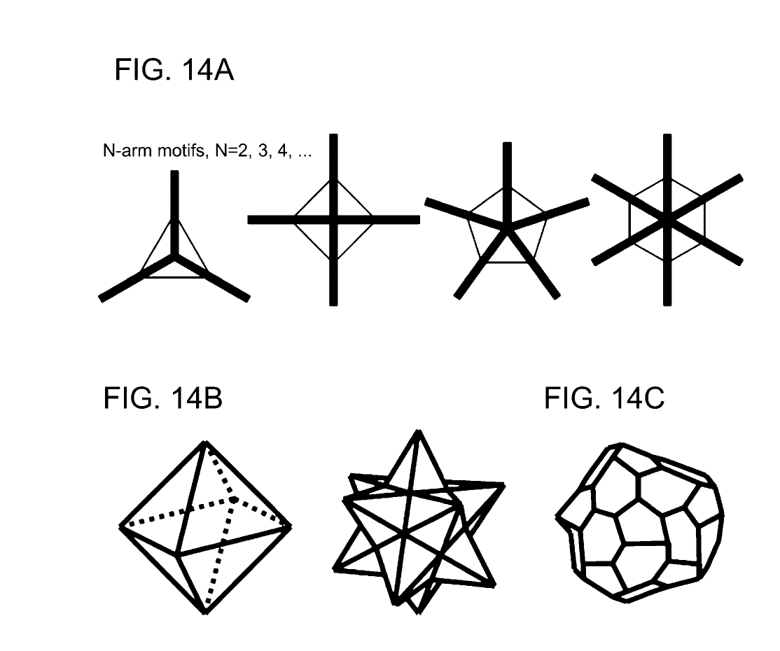

Disclosed herein are nucleic acid structures (alternatively referred to herein as structures) comprising at a minimum three nucleic acid arms (or arms). Such three arm structures are referred to herein as tripods. As will be understood, given the structure of a tripod, the three arms meet each other at a vertex and radiate outwards towards a free end on each arm. This disclosure contemplates and provides nucleic acid structures comprising more than three nucleic acid arms, including structures comprising four, five, six, seven, or more arms. Examples of such structures are provided in FIG. 14. In FIG. 14A, the longer thicker lines correspond to nucleic acid arms and the shorter thinner lines correspond to nucleic acid struts. In FIGS. 14B and C, only nucleic acid arms are illustrated but it is to be understood that such nucleic acid structures comprise nucleic acid struts also.

The nucleic acid arms within a structure (or within a composite structure) are typically of identical length. They are not however so limited and may differ in length depending on the embodiment.

Of particular significance and as provided herein, the nucleic acid arms exist at fixed angles with each other. This is achieved through the use of nucleic acids that are positioned between arms of a structure; these nucleic acids are referred to as nucleic acid struts (or struts). Each nucleic acid strut is connected to two nucleic acid arms in a single structure, thereby maintaining the angular distance between the two arms. The nucleic acid struts may be positioned anywhere along the length of the arms. The position of the strut along the length of the arm (from the vertex) and the length of the strut together can influence the angular distance between the arms. The angular distance between the arms can also be controlled in part by the vertex nucleic acids and other connections existing at the vertex including the nucleic acid connectors interactions. Examples of strut lengths and strut positions along an arm from the vertex are provided in Table 1 for a number of nucleic acid structures. As will be clear from the Table and from the remaining disclosure, struts in a structure (or within a composite structure) may be of identical length or of differing length.

It is to be understood nucleic acid structures may be produced having any particular defined angular distance between their arms, and any number of arms, based on the methodology provided herein. In this respect, the structures are considered to be “tunable” because an end user is able to modify the synthesis method in order to obtain structures of choice.

The arms of the structure may be referred to herein for clarity as the x, y and z arms, for example in the context of a tripod structure. In this structure, typically one (but optionally more than one) strut connects arms x and y, typically one (but optionally more than one) strut connects arms y and z, and typically one (but optionally more than one) strut connects arms z and x. These struts may be referred to, again for clarity, as the xy strut, the yz strut, and the zx strut. In the case of a tripod, each arm is connected to every other arm in the structure. In the case of a structure having more than three arms, all adjacent arms will typically be connected to each other by struts, and optionally non-adjacent arms may also be connected to each other by struts as well. It may be desirable to include struts between non-adjacent arms in order to provide greater structural integrity. As an example, in FIG. 14A, the second structure shown comprises four arms, and four struts between adjacent arms. This structure may also comprise additional struts between non-adjacent arms such as between the “north” and “south” arms and/or the “west” and “east” arms, imagining that the arms are directions on a compass for the sake of explanation.

Thus, the minimum number of arms is 3, and the minimum number of struts is 3. The disclosure contemplates structures having 3 or more arms and 3 or more struts. The number of struts is typically equal to or greater than the number of arms.

Accordingly, provided herein is a nucleic acid structure comprising a first (x), a second (y), and a third (z) nucleic acid arm, each connected at one end to the other arms to form a vertex, and a first, a second, and a third nucleic strut, wherein the first nucleic acid strut connects the first (x) nucleic arm to the second (y) nucleic arm, the second nucleic acid strut connects the second (y) nucleic arm to the third (z) nucleic arm, and the third nucleic acid strut connects the third (z) arm to the first (x) nucleic acid strut.

Provided herein is a nucleic acid structure comprising three nucleic acid arms radiating from a vertex at fixed angles. Such structures may have more than three arms, including 4, 5, 6, 7 or more arms.

Further provided herein is a nucleic acid structure comprising N nucleic acid arms radiating from a vertex, wherein N is the number of nucleic acid arms and is 3 or more, and M nucleic acid struts, each strut connecting two nucleic acid arms to each other, wherein M is the number of nucleic acid struts and is 3 or more. N may be equal to M or it may be less than M. Examples include a nucleic acid structure that comprises 4 nucleic acids and at least 4 nucleic acid struts, or a nucleic acid structure that comprises 5 nucleic acid arms and at 5 nucleic acid struts.

In some embodiments, nucleic acid arms (including adjacent arms) within a structure are equally spaced apart from each other. In other words, the arms are separated from each other by the same angle, or the angular distance between the arms is the same. An example of this is a three arm structure in which adjacent arms are separated from each other by a 60° C. angle. This tripod is referred to as 60° C.-60° C.-60° C. Tripods of this type, when connected to each other, will form a tetrahedron. Thus, it will be understood that the angular distance between the arms also dictates how to such structures will connect with each other and the ultimate 3D shape (or composite nucleic acid structure) to be formed. Another example is a three arm structure in which adjacent arms are separated from each other by a 90° C. angle. This tripod is referred to as 90° C.-90° C.-90° C. Tripods of this type, when connected to each other, will form a cube.

In some embodiments, nucleic acid arms (including adjacent arms) within a structure are not equally spaced apart from each other. In other words, the arms are separated from each other by a different angle, or the angular distance between the arms is different. An example of this is a three arm structure in which some adjacent arms are separated from each other by a 60° C. angle and other adjacent arms are separated from each other by a 90° C. angle. Such a tripod may be referred to as 90° C.-90° C.-60° C. Tripods of this type, when connected to each other, will form a triangular prism. Another example is a three arm structure in which some adjacent arms are separated from each other by a 108° C. angle and other adjacent arms are separated from each other by a 90° C. angle. This tripod is referred to as 90° C.-90° C.-108° C. Tripods of this type, when connected to each other, will form a pentagonal prism. Another example is a three arm structure in which some adjacent arms are separated from each other by a 120° C. angle and other adjacent arms are separated from each other by a 90° C. angle. This tripod is referred to as 90° C.-90° C.-120° C. Tripods of this type, when connected to each other, will form a hexagonal prism.

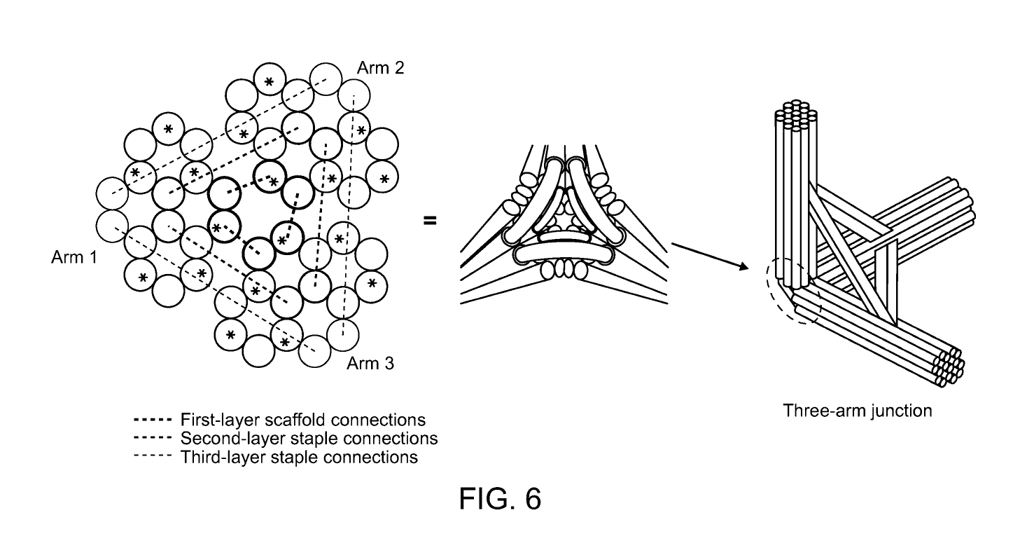

As will be understood based on this disclosure, the nucleic acid structures arrange their arms (three or more of their arms) so as to form a vertex. The arm ends that exist at the vertex may be connected to each other through nucleic acid helices or through nucleic acid connectors (or connector strands), or through a combination of helices and connector strands. Examples of this are illustrated in FIG. 6. The lengths of vertex helices in the first and second layers are provided in Table 2. Typically 0-6 vertex helices are present in a structure. Thus, the structures may further comprise vertex nucleic acids such as vertex helices. Some composite structures may not comprise vertex helices. An example is the tetrahedron which can be formed from the attachment of two tripod structures without vertex helices.

The structures may further comprise connector nucleic acids. These connector nucleic acids may be located at the vertex and/or at the free ends of arms. In the latter instance, such connector nucleic acids facilitate the attachment of two nucleic acid structures to each other, thereby forming a composite nucleic acid structure.

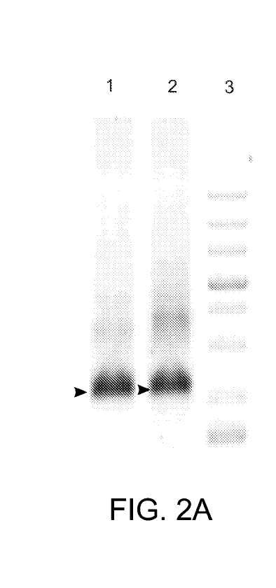

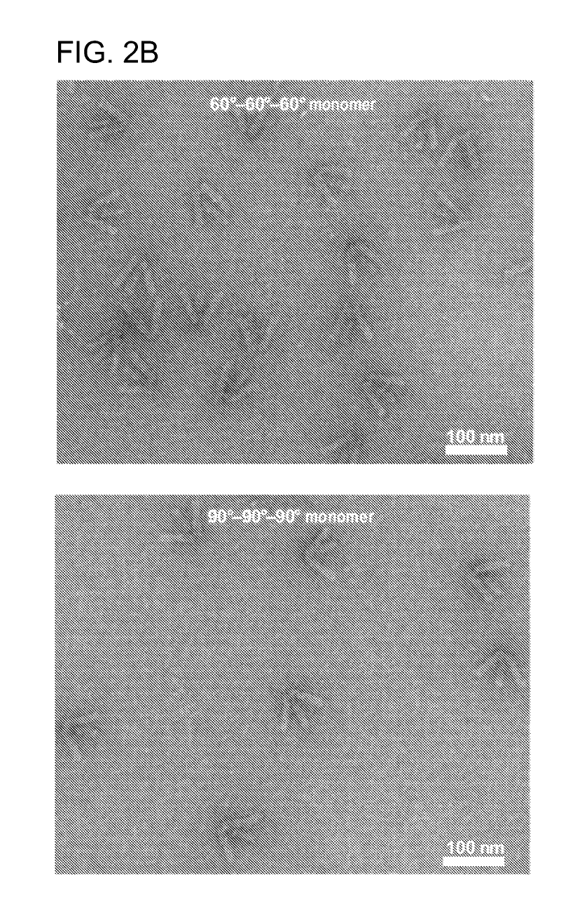

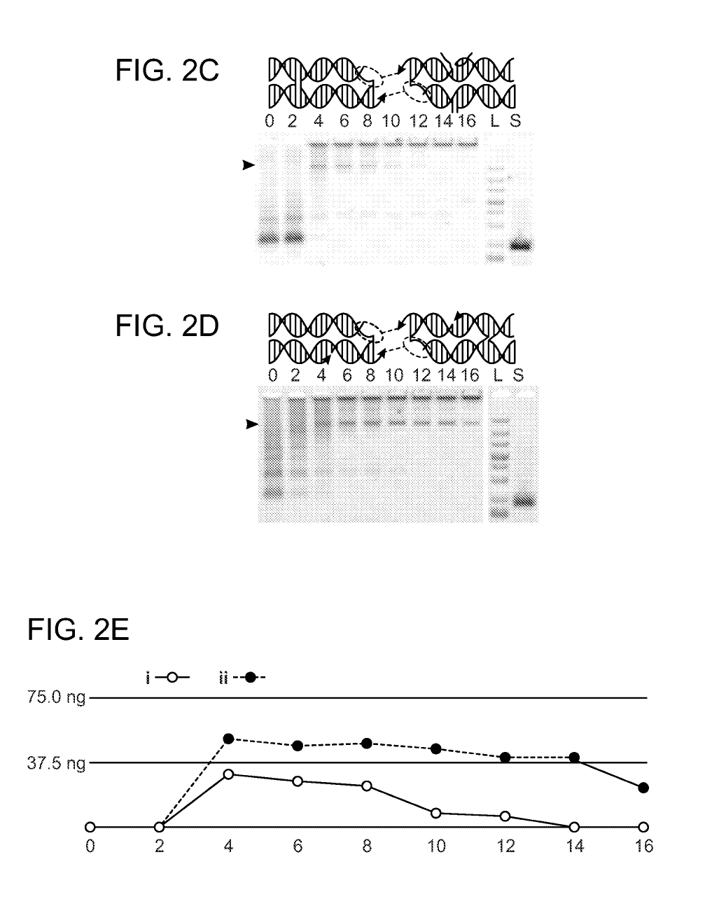

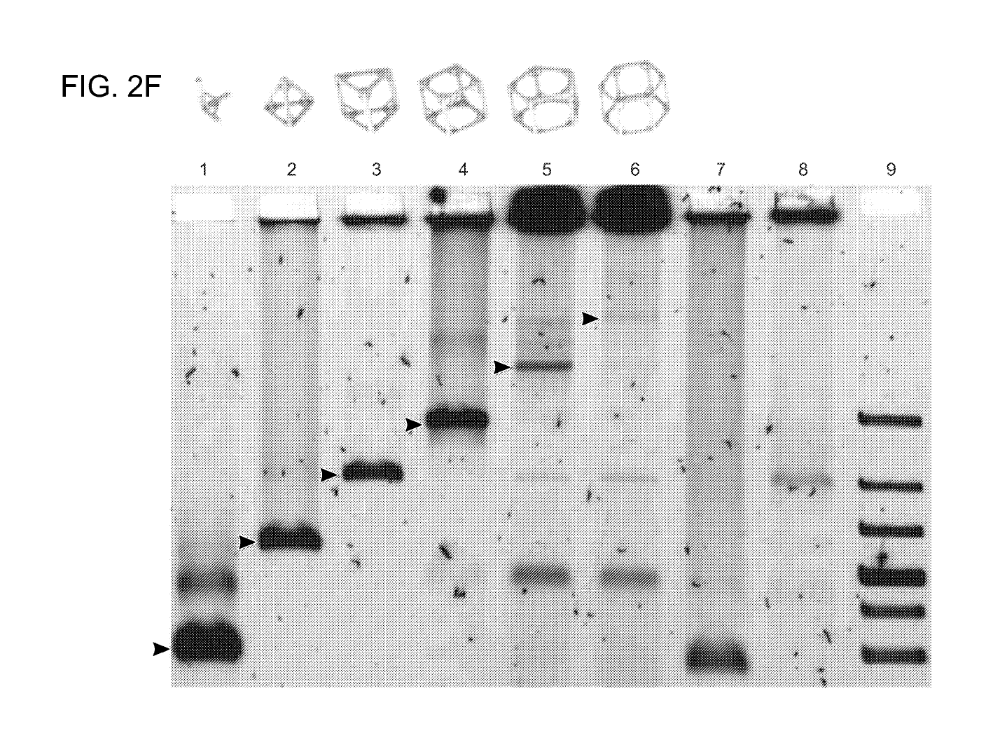

Each nucleic acid arm in a structure therefore typically has one end located at the vertex and one free end (i.e., an end not located at the vertex). The free end may be a blunt end, meaning that it lack any single stranded nucleic acid sequence. Alternatively it may be a sticky end, meaning that it comprises a single-stranded nucleic acid sequence. That sequence, referred to as an overhang, may be 1 or 2 nucleotides in length. It may be longer, although 1-2 nucleotides are suitable and in some instances may result in more efficient synthesis of composite nucleic acids (and thus greater yields of such composites). The overhang may be provided by connector nucleic acids. Such connector nucleic acids may be present in the initial hybridization reaction or they may be added post-synthesis of the nucleic acid structures, with or without purification of the synthesized structures. The connector nucleic acids (also referred to herein as connector strands) may be of any length although it has been found that shorter lengths result in higher composite nucleic acid structure yields. FIG. 2 C provides a schematic of a longer connector strand (on the order of 30 nucleotides with a 2 nucleotide overhang). FIG. 2D provides a schematic of a shorter connector strand (on the order of 11 nucleotides with a 2 nucleotide overhang). The structures of FIGS. 2C and 2D were used to form composite nucleic acid structures that are cubes. The yields of such cubes are shown in FIG. 2E. The top line corresponds to the shorter connector and the bottom line corresponds to the longer connector. Thus, the shorter connector led to higher yield of its composite cube. Although not intending to be bound by any theory, the lower yields using the longer connector strands may be because mismatched composites (or mismatched composite intermediates) comprising longer connector strands may be more stable while mismatched composites (or mismatched composite intermediates) comprising shorter connectors may be less stable and therefore more likely to dissociate and re-associate to form properly matched composite and composite intermediates. As used herein, a composite intermediate comprises a subset of the nucleic acid structures needed to form a composite structure. For example, if the desired composite is a cube (which requires 4 structures), then an intermediate may consist of 2 or 3 structures.

The disclosure contemplates that the connector may be of any length, including lengths of 50 or fewer nucleotides, 40 or fewer nucleotides, 30 or fewer nucleotides, 25 or fewer nucleotides, 20 or fewer nucleotides, 15 or fewer nucleotides, 10 or fewer nucleotides, or 5 or fewer nucleotides. The connector may be 5, 6, 7, 8, 9, 10, 11, 12, 13, 14, 15, 16, 17, 18, 19, 20 or more nucleotides.

The nucleic acid structures may be of any size although typically they are in the range of up to about 5 megadaltons (MD). Thus, they may be 3, 4, 5, or 6 MD in some embodiments. The length of the nucleic acid arms is dictated by the desired rigidity and by their method of synthesis. For example, the structures described herein have arms made of 16 parallel double helices. Since they were made using DNA origami techniques starting with the M13 scaffold strand, the length of the arms is typically about 50 nm. It is to be understood that if a scaffolds of a different length was used, or if the arms were designed to have a different number of double helices (for example if more or less rigidity and strength was desired), then the length of the arm could vary from that described herein. Assuming the nucleic acid structures have arms of 50 nm, and assuming all arms are of equal length, then it will be understood that composite nucleic acid structures will have edges widths on the order of 100 nm. Thus the composites that may be generated according to this disclosure may be defined as having edge widths that are at least 100 nm, including 120, 140, 160, 180, 200, or more nm. In some instances, the composites may have edge widths of 80 nm or more.

The nucleic acid arms, nucleic acid struts and vertex nucleic acids may be comprised of double helices such as parallel double helices. Illustrated herein are arms comprised of 16 parallel double helices each, struts comprised of 2 parallel double helices each, and vertex nucleic acids comprised of a single double helix each. When more than one double helix is present, there typically be cross-over strands that hybridize to parallel helices and thereby promote the proximity of the helices and ultimately rigidity thereof.

It is to further understood that the nucleic acid structures disclosed herein may be synthesized using any number of nucleic acid nanostructure synthesis methods including without limitation DNA origami and DNA single stranded tiles (SST). These techniques are known in the art, and are described in greater detail in U.S. Pat. Nos. 7,745,594 and 7,842,793; U.S. Patent Publication No. 2010/00696621; and Goodman et al. Nature Nanotechnology.

The nucleic acid structures may be used to generate larger structures referred to herein as composite nucleic acid structures (or composites or composite structures). Composite structures are formed through the connection of nucleic acid structures to each other. Typically the nucleic acid structures are identical in terms of length and angle definition. Thus a plurality of identical nucleic acid structures are combined in a single reaction vessel, and allowed to attached to each other to form larger 3D structures via connections of their free arm ends. Such connections may be facilitated by the presence (or inclusion) of connector strands, although the synthesis method is not so limited.

Therefore, disclosed and provided herein is a composite nucleic acid structure comprising L nucleic acid structures, wherein L is the number of nucleic acid structures, and wherein the L nucleic acid structures are connected to each other at free (non-vertex) ends of the nucleic acid arms. The number of structures needed to make a composite will depend on the composite structure desired and the structures used as components. In some instances, the composite structure may comprise two, four, six, eight, ten, twelve or more nucleic acid structures each of which has three arms. As illustrated throughout, this methodology may be used to generate composite nucleic acid structures that are tetrahedrons, triangular prisms, cubes, pentagonal prisms, or hexagonal prisms. It is to be understood that any arbitrary composite structure may be made using the methodology provided herein. These composites may be of virtually any size, including but not limited to. Illustrated herein are composite nucleic acid structures that are 20 megadaltons (MD), 30 MD, 40 MD, 50 MD, and 60 MD in size.

The composites may be generated immediately following the generation of the nucleic acid structures and thus in the same vessel as the structures. Connector strands, if used, may be present at the beginning of the hybridization reaction or may be added once the structures are formed and prior to formation of the composites. Such single reaction vessel synthesis is referred to as “one-pot” annealing.

Below are more detailed and exemplary descriptions of the particular nucleic acid structures, and particular composite nucleic acid structures, and their methods of synthesis.

These descriptions are meant to be exemplary and not limiting as to the breadth of this disclosure. For example, it is to be understood that although much of the following description and exemplification involves 3-arm “tripod” nucleic acid structures, the teachings may be generalized to structures of any number of arms as described herein.

### Exemplary Tripod Design and Methodology

### Assembly Strategy of Polyhedra and Design Features of Tripods.

In one-pot annealing, the scaffold and staple strands first assemble into a tripod origami monomer, and then the tripods (without intermediate purification) assemble into the polyhedron (FIG. 1A). It is also contemplated that the tripod monomers may be purified prior to the final assembly into composite nucleic acid structures. Diverse polyhedra can be constructed by using tripods with different designed inter-arm angles. The tripod has three typically equal-length (e.g., ˜50 nm) stiff arms connected at the vertex (see FIG. 6 for connection details) with controlled inter-arm angles (FIG. 1B). To ensure stiffness, each arm contains a sufficient number (e.g., 16) of parallel double-helices packed on a honeycomb lattice (5) with twofold rotational symmetry. A supporting “strut” consisting of two double-helices controls the angle between the two arms. The tripod is named according to its three inter-arm angles (e.g. the tetrahedron and the cube are respectively assembled from 60°-60°-60° and 90°-90°-90° tripods). To avoid potential unwanted aggregation resulting from blunt-end stacking of DNA helices (5), up to six short DNA double-helices (denoted “vertex helices”) are included at the vertex to partially conceal its blunt duplex ends (FIG. 1B; the number of helices and their lengths vary for different polyhedra, see FIG. 6 and Table 2 for details). Additionally, the vertex helices are expected to help maintain inter-arm angles by increasing rigidity of the vertices. Two connection strategies are used to assemble tripods into polyhedra. To facilitate exposition, the three arms are denoted as X-arm, Y-arm, and Z-arm (FIG. 1C). Connecting X-arm to X-arm and Y-arm to Z-arm produces polyhedra (such as a cube; FIG. 1D) other than the tetrahedron, which is assembled by connecting X to X, Y to Y, and Z to Z (FIG. 1E).

Tripod Conformation Control with Struts.

First, we verified that the inter-arm angle was controlled by the length of the supporting strut. Gel electrophoresis of 60°-60°-60° and 90°-90°-90° tripods revealed a dominant band for each tripod (FIG. 2A), confirming their correct formation. Consistent with its more compact designed conformation, the 60°-60°-60° tripod migrated slightly faster than the 90°-90°-90° one. The two tripod bands were each purified, imaged by TEM, and showed designed tripod-like morphologies (FIG. 2B). The measured inter-arm angles were slightly smaller than designed (53±5° [SD, n=60] for 60°-60°-60° tripods; 87±4° [SD, n=60] for 90°-90°-90° tripods), possibly reflecting a small degree of strut bending.

### Connector Designs.

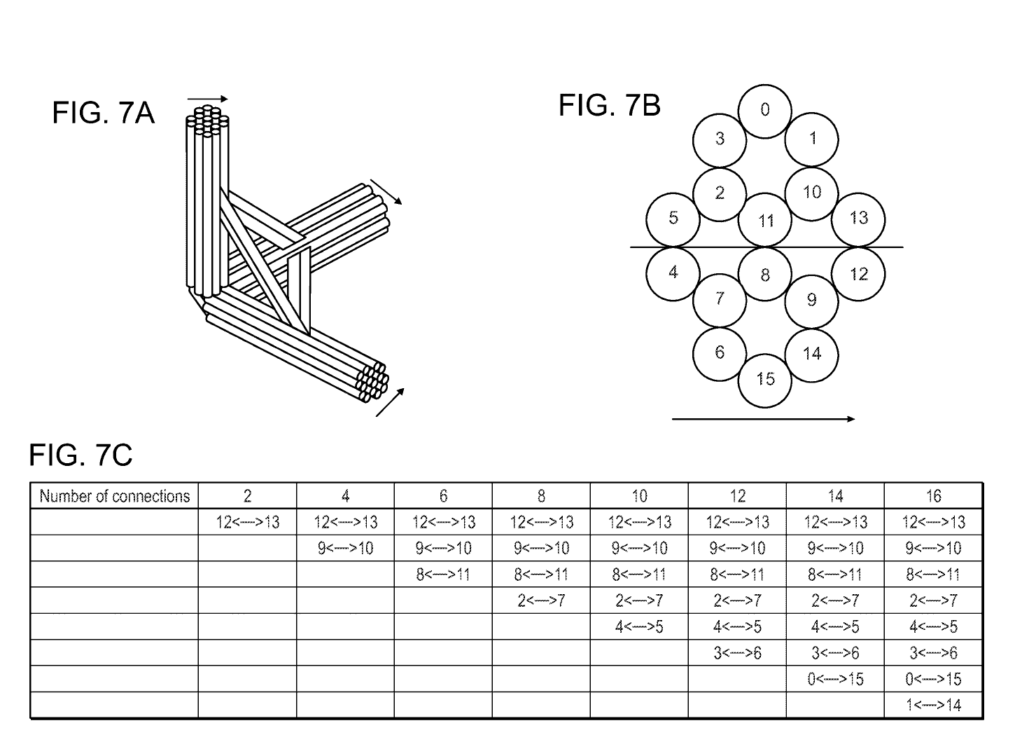

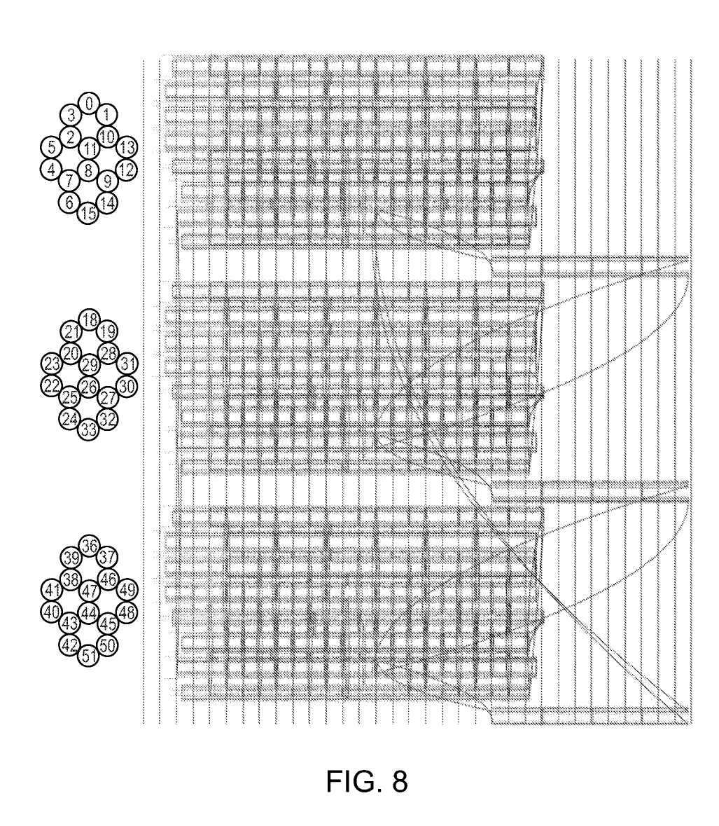

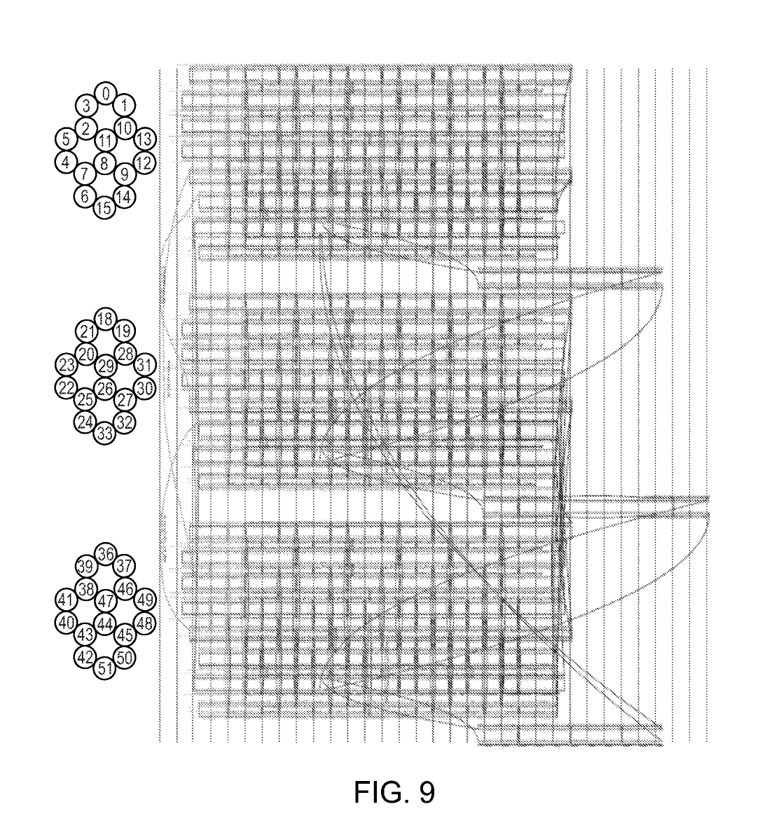

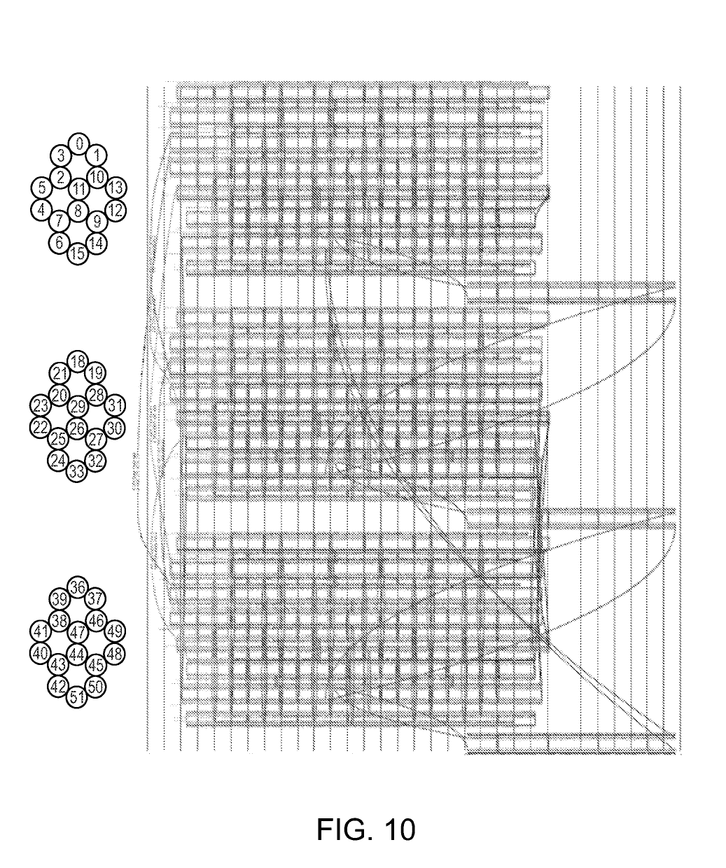

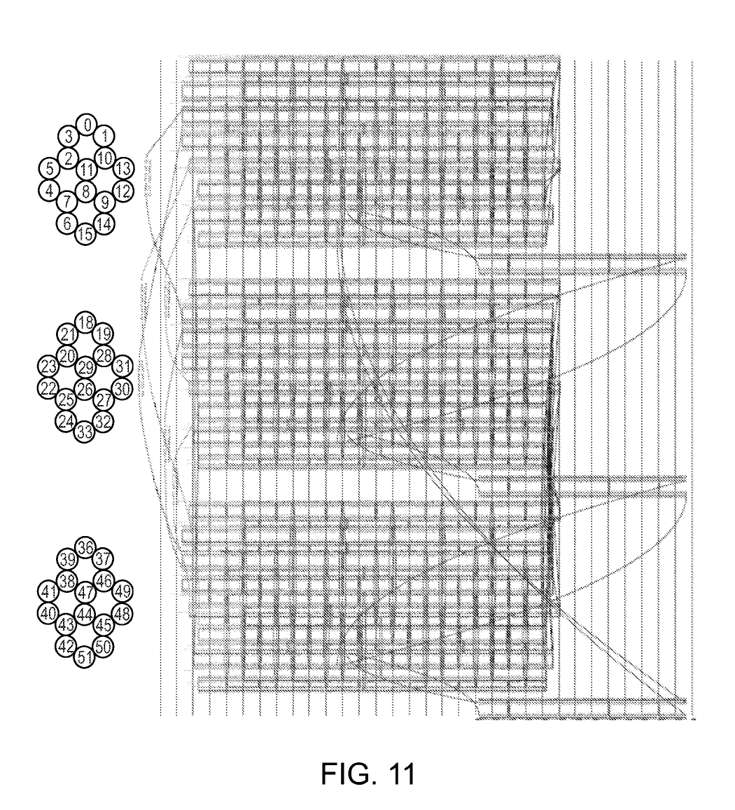

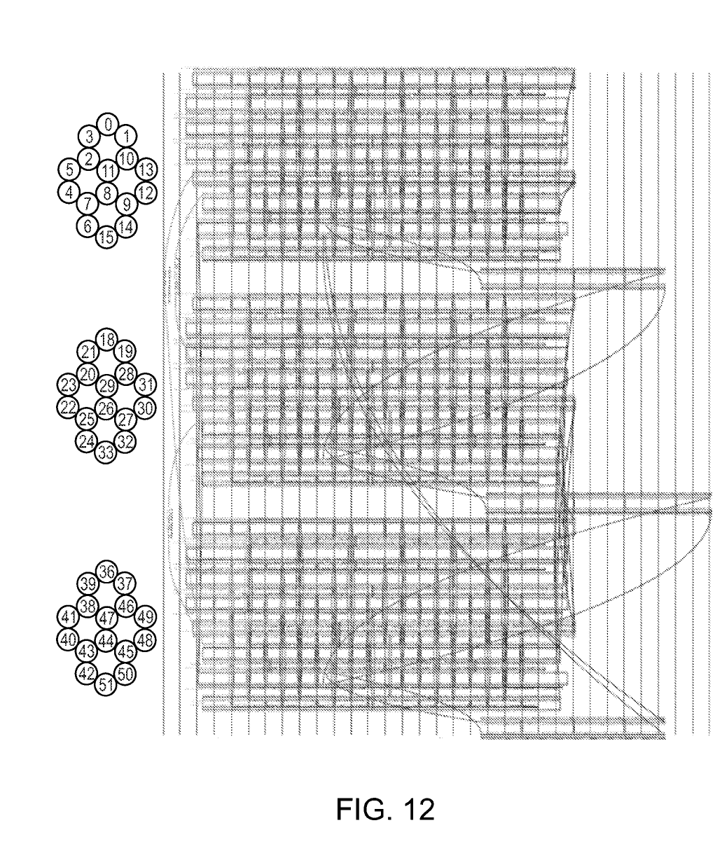

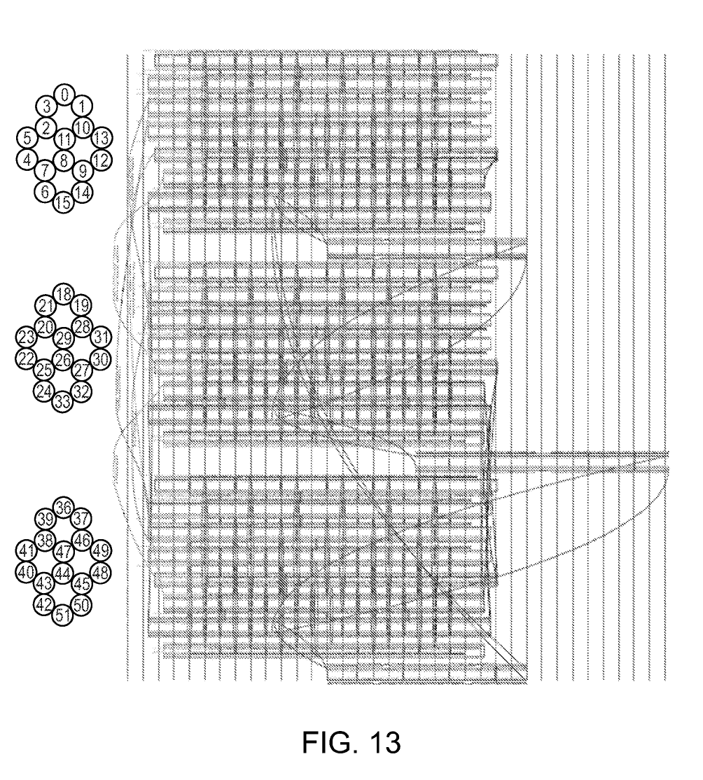

The strands connecting the tripods are called “connectors.” Connector designs affected the polyhedra assembly yields. Two designs were tested for the cube. In scheme i, each 30-base connector spanned two adjacent tripods, with a 28-base segment anchored on one tripod and another 2-base (sticky end) on the other (FIG. 6; see FIG. 7 for details). Gel electrophoresis (quantified in FIG. 2E) revealed that the assembly yield was affected by the number of connected helices (n): a product band was only observed for 4≦ n≦12; for n<4, the dominant band were monomers, likely reflecting overly weak inter-monomer connections; for n>12, aggregations dominated.

In scheme i, the connectors were stably anchored (forming 28 base pairs) on tripods before inter-monomer connection occurred. In scheme ii, the connector was shortened from 30 to 11 bases so that it should only be anchored to two adjacent tripods by 9-base and 2-base segments in the assembled cube (FIG. 2D), and only dynamically binds to a monomeric tripod. Compared with the stably attached connector design, the dynamic connector design is expected to reduce inter-monomer mismatches that may occur during the assembly, as such mismatches would be less likely frozen in a kinetic trap. Indeed, scheme ii showed substantially increased assembly yield (FIG. 2E). It was thus used for subsequent polyhedra designs, except for the tetrahedron, where scheme i produced sufficient yield for this relatively simple structure. The assembly yields were estimated from the gel (FIG. 2F). The 90°-90°-90° monomer sample (FIG. 2F, lane 1) showed a strong monomer band and a putative dimer band (not studied by TEM, ˜27% intensity compared to the monomer). We define the assembly yield of a polyhedron as the ratio between its product band intensity and the combined intensity of the 90°-90°-90° monomer and dimer bands (lane 1), and obtained yields of 45%, 24%, 20%, 4.2%, and 0.11% for the tetrahedron, the triangular prism, the cube, the pentagonal prism, and the hexagonal prism, respectively (FIG. 2F).

### Polyhedra Assembly.

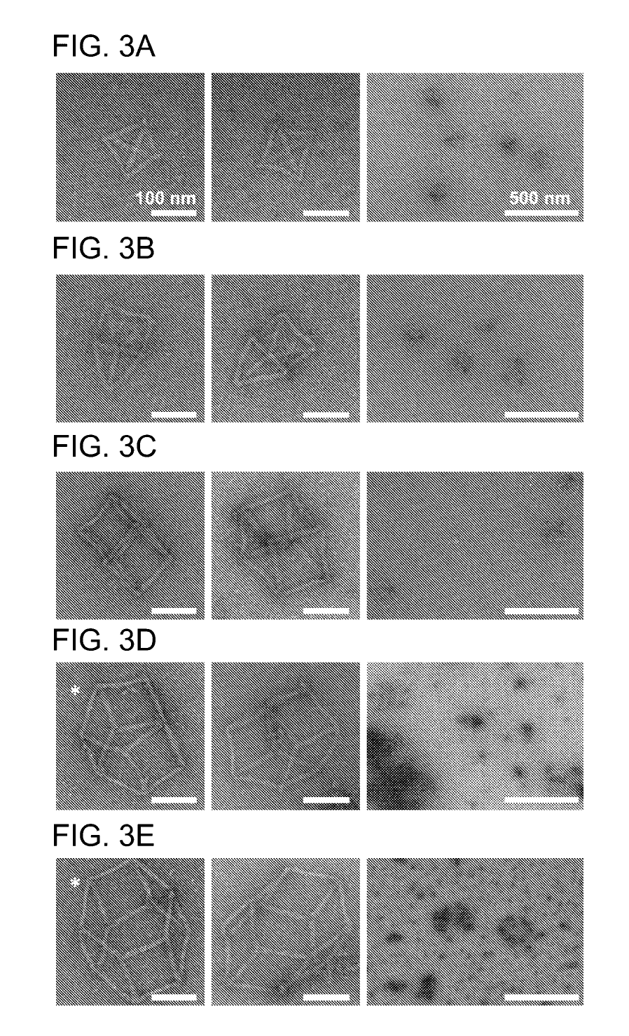

The lengths and the attachment points of the struts varied for each polyhedron (Table 1). The tetrahedron, the triangular prism, the cube, the pentagonal prism, and the hexagonal prism should be assembled from monomers with designed 60°-60°-60°, 90°-90°-60°, 90°-90°-90°, 90°-90°-108°, and 90°-90°-120° angles, respectively (FIG. 1B). The first three monomers indeed produced tetrahedra, triangular prisms, and cubes [verified by gel electrophoresis (FIG. 2F) and TEM imaging (FIG. 3, A to C)], suggesting accurate control for angles within 90°. However, the pentagonal prism was assembled from monomers with designed angles of 90°-90°-120° (instead of)90°-90°-108°, and the hexagonal prism from 90°-90°-140° (instead of)90°-90°-120°. Thus the assembly of these two polyhedra requires monomers with designed Y-Z angles greater than the design criteria. This requirement likely reflects slight bending of the relevant struts, which could be compensated by using longer struts.

### Effects of Struts and Vertex Helices on Polyhedra Assembly.

We next verified that both the struts and the vertex helices were required for the tripods to assemble into the designed polyhedron. Three samples were prepared for cube assembly using tripods that contain (i) both the struts and the vertex helices (FIG. 2F, lane 4), (ii) the vertex helices but not the struts (lane 7), and (iii) the struts but not the vertex helices (lane 8; the samples were subjected to gel electrophoresis after annealing). The first sample showed a sharp strong band corresponding to the cube (verified by TEM, FIG. 3B). The second failed to produce any clear product band. The third produced substantial aggregates, and a clear but weak band with mobility comparable to the triangular prism. This band may correspond to a hexamer, but its molecular morphology was not investigated. Based on the above experiments, we included both the struts and the vertex helices in the tripods for subsequent polyhedra assembly.

### TEM Characterization.

Product bands were purified and imaged under TEM. For the tetrahedron, the triangular prism, and the cube, most structures appeared as intact polyhedra; a small fraction of broken structures (<20%) were likely ruptured during the purification and imaging (FIG. 3, A to C). In contrast, few intact structures were observed for the purified pentagonal and hexagonal prisms (data not shown). Thus, unpurified samples for these two were directly imaged and the expected molecular morphologies were observed (FIGS. 3, D and E, for exemplary images, further images available but not shown). The struts are clearly visible in many images.

### 3D DNA-PAINT Super-Resolution Microscopy.

Localization-based 3D super-resolution fluorescence microscopy (31-33) offers a minimally invasive way to obtain true single molecule 3D images of DNA nanostructures in their “native” hydrated environment. In stochastic reconstruction microscopy (34), most molecules are switched to a fluorescent dark (OFF) state, and only a few emit fluorescence (ON state). Each molecule is localized with nanometer precision by fitting its emission to a 2D Gaussian function. In DNA-PAINT, the “switching” between ON- and OFF-states is facilitated by repetitive, transient binding of fluorescently labeled oligonucleotides (“imager” strands) to complementary “docking” strands (24, 28, 29, 35).

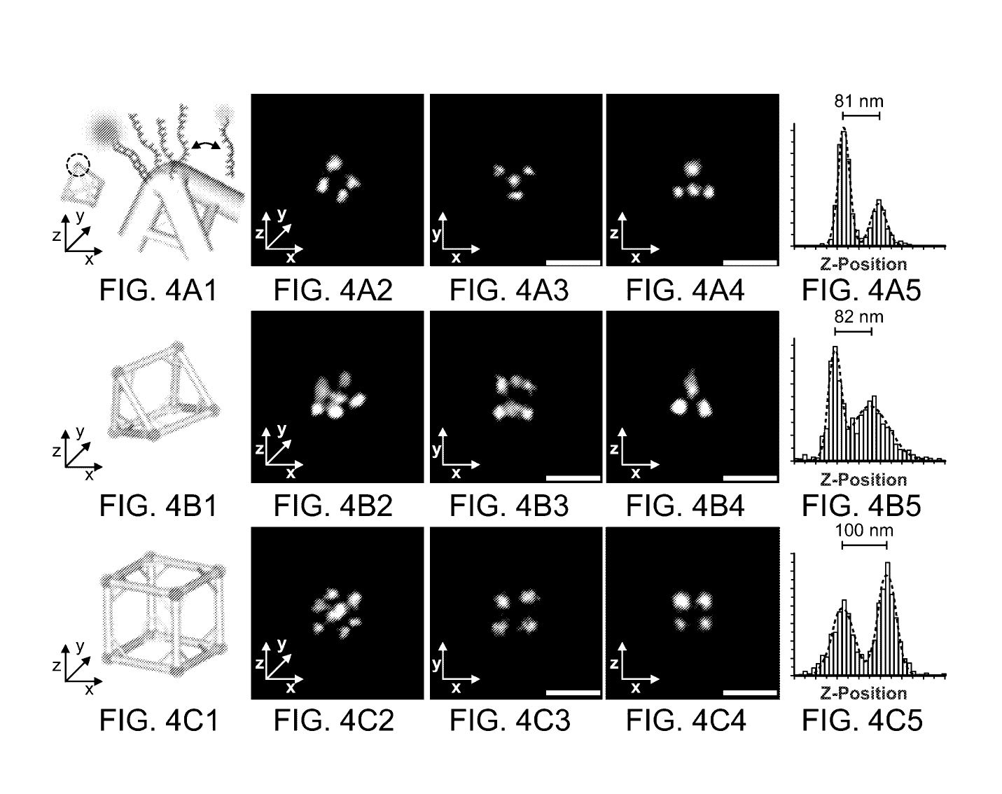

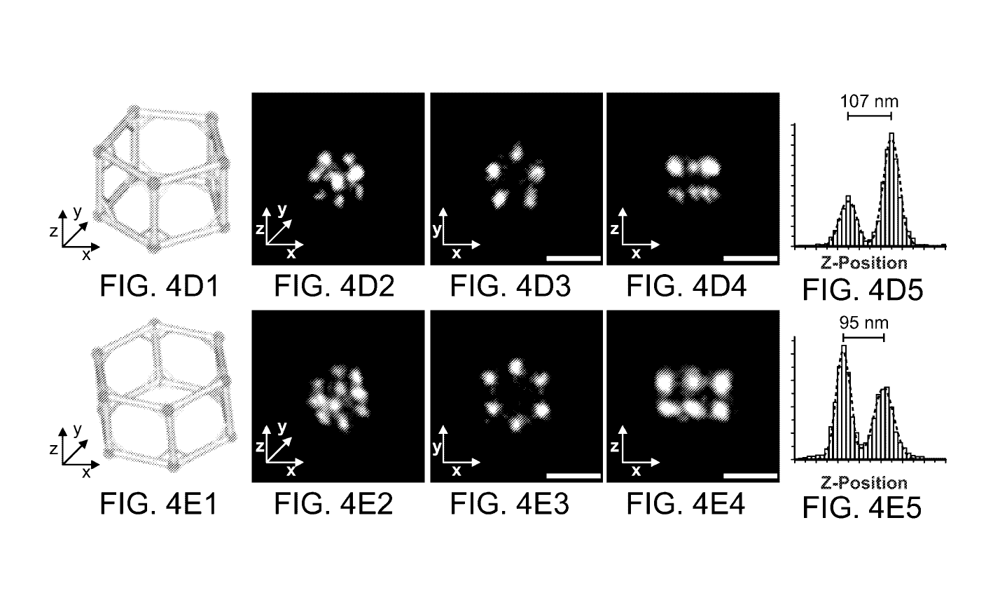

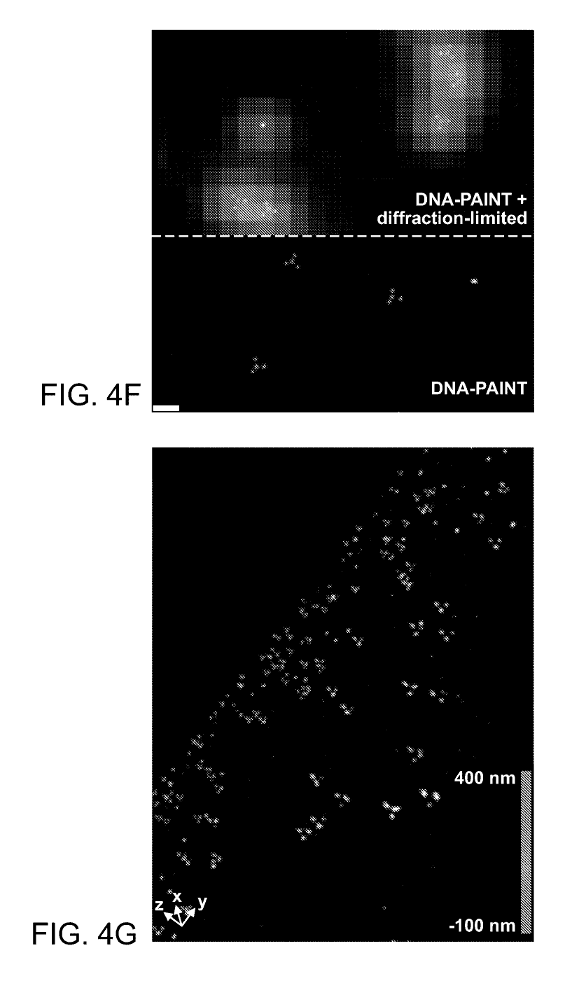

We extended DNA-PAINT to 3D imaging (29) by using optical astigmatism (31, 36), in which a cylindrical lens used in the imaging path “converts” the spherical point spread function (PSF) of a molecule to an elliptical PSF when imaged out of focus. The degree and orientation of the elliptical PSF depends on the displacement and direction of the point source from the current focal imaging plane, and is used to determine its z position (31, 36). We applied 3D DNA-PAINT to obtain sub-diffraction-resolution single-molecule images of the polyhedra. To ensure all the vertices of a polyhedron will be imaged, each vertex is modified with multiple (about eighteen) 9-nt docking strands (Staple-TTATCTACATA-3′; SEQ ID NO: 1) (FIG. 4A1) in a symmetric arrangement (FIG. 6). For surface immobilization, a subset of strands along the polyhedron edges were modified with 21-nt extensions (Staple-TTCGGTTGTACTGTGACCGATTC-3′; SEQ ID NO: 2), which were hybridized to biotinylated complementary strands attached to a streptavidin covered glass slide (Biotin-GAATCGGTCACAGTACAACCG-3′; SEQ ID NO: 3).

Using 3D DNA-PAINT microscopy, all five polyhedra showed designed 3D patterns of vertices (FIG. 4, columns 1-4) with expected heights (FIG. 4, A5-E5), suggesting that the solution shape of the structures is maintained during surface immobilization and imaging. We quantified the tetrahedra formation and imaging yields (FIGS. 4, F and G). 253 out of 285 structures (89%) contained 4 spots in the expected tetrahedral geometry. Height measurement yielded 82±15 nm, consistent with the designed value (82 nm). Single DNA-PAINT binding events were localized with an accuracy of 5.4 nm in x-y and 9.8 nm in z [see below for how localization accuracy was determined]. This z localization accuracy almost completely accounts for the 15 nm spread in the height measurement distribution. The calculated localization precisions translate to an obtainable resolution of ˜13 nm in x and y, and ˜24 nm in z.

Previous work demonstrated diverse DNA polyhedra self-assembled from small 3-arm-junction tiles (˜80 kD) (16), which consist of three double-helix arms connected by flexible single-stranded hinges. However, straightforward implementation of megadalton 3-arm origami tiles using similar flexible inter-arm hinges (i.e. tripods with no struts or vertex helices) failed to produce well-formed polyhedra (FIG. 2B, lane 7). An origami tripod contains 50 times more distinct strands than previous 3-arm-junction tiles (formed from 3 distinct strands) and is 60 times more massive in molecular weight. Apart from the challenges associated with the more error-prone construction of the more complex monomers from individual strands, successful hierarchical assembly of such large monomers into polyhedra also needs to overcome much slower reaction kinetics, caused by the larger size and lower concentration of the tripod monomers. The stiff DNA tripods, with rationally designed inter-arm angles controlled by supporting struts and vertex helices, lead to successful construction of diverse polyhedra, suggesting that conformation control of branched megadalton monomers can facilitate their successful assembly into higher order structures.

The design principles of DNA tripods may be extended to stiff megadalton n-arm (n>4) branched motifs with controlled inter-arm angles. Self-assembly with such n-arm motifs could be used to construct more sophisticated polyhedra, and potentially extended 2D and 3D lattices with sub-100 nm tunable cavities.

Such structures could potentially be used to template guest molecules for diverse applications, e.g. spatially arranging multiple enzymes into efficient reaction cascades (37) or nanoparticles to achieve useful photonic properties (38, 39). Furthermore, the DNA polyhedra constructed here, with a size comparable to bacterial microcompartments, may potentially be used as skeletons for making compartments with precisely controlled dimensions and shapes by wrapping lipid membranes around their outer surfaces (40). Such membrane-enclosed microcompartments could potentially serve as bioreactors for synthesis of useful products or as delivery vehicles for therapeutic cargo (25).

For 3D characterization of DNA nanostructures, super-resolution fluorescence microscopy (e.g. 3D DNA-PAINT) provides complementary capabilities to present electron microscopy (e.g. cryo-EM (12, 16, 17, 23)). While cryo-EM offers higher spatial resolution imaging of unlabeled structures, DNA-PAINT is less technically involved to implement, obtains true single molecule images of individual structures (rather than relying on class averaging), and preserves the multi-color capability of fluorescence microscopy (29). Additionally, DNA-PAINT in principle allows for observation of dynamic structural changes of nanostructures in their “native” hydrated environment, currently suitable for slow changes on the minutes timescale (e.g. locomotion of synthetic DNA walkers) and potentially for faster motions with further development.

TABLE 1
Strut designs of the polyhedra. All units are nanometers. Designed
length of the strut connecting (i) Y-arm and Z-arm, (ii) X-arm
and Z-arm, or (iii) X-arm and Y-arm. Designed distance from the
vertex to the strut attachment point on (iv) X-, (v) Y-, or (vi) Z-arm.

| | i | ii | iii | iv | v | vi |
|---|---|---|---|---|---|---|
| Tetrahedron | 28 | 28 | 28 | 29 | 29 | 29 |
| Triangular prism | 18 | 26 | 26 | 18 | 18 | 18 |
| Cube | 30 | 30 | 30 | 21 | 21 | 21 |
| Pentagonal prism | 32 | 26 | 26 | 19 | 18 | 18 |
| Hexagonal prism | 37 | 28 | 28 | 20 | 20 | 20 |

TABLE 2

| | Number | | | Length |
|---|---|---|---|---|
| | of 1^{st}- | length of 1^{st}- | Number of 2^{nd}- | of 2^{nd}- |
| | layer helices | layer helices | layer helices | layer helices |
| Tetrahedron | 0 | n/a | 0 | n/a |
| Triangular | 3 | 15 bp, 15 bp, | 0 | n/a |
| prism | | 18bp | | |
| Cube | 3 | 15 bp, 15 bp, | 3 | 15 bp, 15 bp, |
| | | 15bp | | 15bp |
| Pentagonal | 3 | 15 bp, 15 bp, | 0 | n/a |
| prism | | 12bp | | |
| Hexagonal | 3 | 24 bp, 24 bp, | 3 | 19 bp, 19 bp, |
| prism | | 12bp | | 15bp |

### Nucleic Acid Nanostructure Methodology Generally

The nucleic acid structures provided herein may be formed using any nucleic acid folding or hybridization approach. One such approach is DNA origami (Rothemund, 2006, Nature, 440:297-302, incorporated herein by reference in its entirety). In a DNA origami approach, a structure is produced by the folding of a longer “scaffold” nucleic acid strand through its hybridization to a plurality of shorter “staple” oligonucleotides, each of which hybridize to two or more non-contiguous regions within the scaffold strand. In some embodiments, a scaffold strand is at least 100 nucleotides in length. In some embodiments, a scaffold strand is at least 500, at least 1000, at least 2000, at least 3000, at least 4000, at least 5000, at least 6000, at least 7000, or at least 8000 nucleotides in length. The scaffold strand may be naturally or non-naturally occurring. The scaffold typically used in the M13 mp18 viral genomic DNA, which is approximately 7 kb. Other single stranded scaffolds may be used including for example lambda genomic DNA. Staple strands are typically less than 100 nucleotides in length; however, they may be longer or shorter depending on the application and depending upon the length of the scaffold strand. In some embodiments, a staple strand may be about 15 to about 100 nucleotides in length. In some embodiments the staple strand is about 25 to about 50 nucleotides in length.

In some embodiments, a nucleic acid structure may be assembled in the absence of a scaffold strand (e.g., a scaffold-free structure). For example, a number of oligonucleotides (e.g., <200 nucleotides or less than 100 nucleotides in length) may be assembled to form a nucleic acid nanostructure. This approach is described in WO 2013/022694 and WO 2014/018675, each of which is incorporated herein by reference in its entirety.

Other methods for assembling nucleic acid structures are known in the art, any one of which may be used herein. (See for example Kuzuya and Komiyama, 2010, Nanoscale, 2:310-322. It is also to be understood that a combination or hybrid of these methods may also be used to generate the nucleic acid structures disclosed herein. These methods may be modified based on the teaching provided herein in order to obtain the fixed-angle nucleic acid structures of this disclosure.

### Nucleic Acids

The nucleic acid structures may comprise naturally occurring and/or non-naturally occurring nucleic acids. If naturally occurring, the nucleic acids may be isolated from natural sources or they may be synthesized apart from their naturally occurring sources. Non-naturally occurring nucleic acids are synthetic.

The terms “nucleic acid”, “oligonucleotide”, and “strand” are used interchangeably to mean multiple nucleotides attached to each other in a contiguous manner. A nucleotide is a molecule comprising a sugar (e.g. a deoxyribose) linked to a phosphate group and to an exchangeable organic base, which is either a pyrimidine (e.g., cytosine (C), thymidine (T) or uracil (U)) or a purine (e.g., adenine (A) or guanine (G)). In some embodiments, the nucleic acid may be L-DNA. In some embodiments, the nucleic acid is not RNA or an oligoribonucleotide. In these embodiments, the nucleic acid structure may be referred to as a DNA structure. A DNA structure however may still comprise base, sugar and backbone modifications.

### Modifications

A nucleic acid structure may be made of DNA, modified DNA, and combinations thereof. The oligodeoxyribonucleotides (also referred to herein as oligonucleotides, and which may be staple strands, connector strands, and the like) that are used to generate the nucleic acid structure or that are present in the nucleic acid structure may have a homogeneous or heterogeneous (i.e., chimeric) backbone. The backbone may be a naturally occurring backbone such as a phosphodiester backbone or it may comprise backbone modification(s). In some instances, backbone modification results in a longer half-life for the oligonucleotides due to reduced nuclease-mediated degradation. This is turn results in a longer half-life. Examples of suitable backbone modifications include but are not limited to phosphorothioate modifications, phosphorodithioate modifications, p-ethoxy modifications, methylphosphonate modifications, methylphosphorothioate modifications, alkyl- and aryl-phosphates (in which the charged phosphonate oxygen is replaced by an alkyl or aryl group), alkylphosphotriesters (in which the charged oxygen moiety is alkylated), peptide nucleic acid (PNA) backbone modifications, locked nucleic acid (LNA) backbone modifications, and the like. These modifications may be used in combination with each other and/or in combination with phosphodiester backbone linkages.

Alternatively or additionally, the oligonucleotides may comprise other modifications, including modifications at the base or the sugar moieties. Examples include nucleic acids having sugars which are covalently attached to low molecular weight organic groups other than a hydroxyl group at the 3′ position and other than a phosphate group at the 5′ position (e.g., a 2′-O-alkylated ribose), nucleic acids having sugars such as arabinose instead of ribose. Nucleic acids also embrace substituted purines and pyrimidines such as C-5 propyne modified bases (Wagner et al., Nature Biotechnology 14:840-844, 1996). Other purines and pyrimidines include but are not limited to 5-methylcytosine, 2-aminopurine, 2-amino-6-chloropurine, 2,6-diaminopurine, hypoxanthine. Other such modifications are well known to those of skill in the art.

Modified backbones such as phosphorothioates may be synthesized using automated techniques employing either phosphoramidate or H-phosphonate chemistries. Aryl-and alkyl-phosphonates can be made, e.g., as described in U.S. Pat. No. 4,469,863, and alkylphosphotriesters (in which the charged oxygen moiety is alkylated as described in U.S. Pat. No. 5,023,243 and European Patent No. 092574) can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

Nucleic acids can be synthesized de novo using any of a number of procedures known in the art including, for example, the b-cyanoethyl phosphoramidite method (Beaucage and Caruthers Tet. Let. 22:1859, 1981), and the nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated nucleic acid synthesizers available in the market. These nucleic acids are referred to as synthetic nucleic acids. Modified and unmodified nucleic acids may also be purchased from commercial sources such as IDT and Bioneer.

An isolated nucleic acid generally refers to a nucleic acid that is separated from components with which it normally associates in nature. As an example, an isolated nucleic acid may be one that is separated from a cell, from a nucleus, from mitochondria, or from chromatin.

The nucleic acid structures and the composite nucleic acid structures may be isolated and/or purified. Isolation, as used herein, refers to the physical separation of the desired entity (e.g., nucleic acid structures, etc.) from the environment in which it normally or naturally exists or the environment in which it was generated. The isolation may be partial or complete.

Isolation of the nucleic acid structure may be carried out by running a hybridization reaction mixture on a gel and isolating nucleic acid structures that migrate at a particular molecular weight and are thereby distinguished from the nucleic acid substrates and the spurious products of the hybridization reaction. As another example, isolation of nucleic acid structures may be carried out using a buoyant density gradient, sedimentation gradient centrifugation, or through filtration means.

### Agents

The composite nucleic acid structures may contain an agent that is intended for use in vivo and/or in vitro, in a biological or non-biological application. For example, an agent may be any atom, molecule, or compound that can be used to provide benefit to a subject (including without limitation prophylactic or therapeutic benefit) or that can be used for diagnosis and/or detection (for example, imaging) in vivo, or that may be used for effect in an in vitro setting (for example, a tissue or organ culture, a clean-up process, and the like). The agents may be without limitation therapeutic agents and diagnostic agents. Examples of agents for use with any one of the embodiments described herein are described below.

In some aspects, the composite nucleic acid structures are used to deliver agent either systemically or to localized regions, such as for example tissues or cells. Any agent may be delivered using the methods of the invention provided that it can be loaded into the composite structure.

The agent may be without limitation a chemical compound including a small molecule, a protein, a polypeptide, a peptide, a nucleic acid, a virus-like particle, a steroid, a proteoglycan, a lipid, a carbohydrate, and analogs, derivatives, mixtures, fusions, combinations or conjugates thereof. The agent may be a prodrug that is metabolized and thus converted in vivo to its active (and/or stable) form. The invention further contemplates the loading of more than one type of agent in a composite structure and/or the combined use of composite structures comprising different agents.

One class of agent is peptide-based agents such as (single or multi-chain) proteins and peptides. Examples of peptide-based agents include without limitation antibodies, single chain antibodies, antibody fragments, enzymes, co-factors, receptors, ligands, transcription factors and other regulatory factors, some antigens (as discussed below), cytokines, chemokines, hormones, and the like.

Another class of agents includes chemical compounds that are non-naturally occurring.

A variety of agents that are currently used for therapeutic or diagnostic purposes include without limitation imaging agents, immunomodulatory agents such as immunostimulatory agents and immunoinhibitory agents (e.g., cyclosporine), antigens, adjuvants, cytokines, chemokines, anti-cancer agents, anti-infective agents, nucleic acids, antibodies or fragments thereof, fusion proteins such as cytokine-antibody fusion proteins, Fc-fusion proteins, analgesics, opioids, enzyme inhibitors, neurotoxins, hypnotics, anti-histamines, lubricants, tranquilizers, anti-convulsants, muscle relaxants, anti-Parkinson agents, anti-spasmodics, muscle contractants including channel blockers, miotics and anti-cholinergics, anti-glaucoma compounds, modulators of cell-extracellular matrix interactions including cell growth inhibitors and anti-adhesion molecules, vasodilating agents, inhibitors of DNA, RNA or protein synthesis, anti-hypertensives, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, targeting agents, neurotransmitters, proteins, cell response modifiers, and vaccines.

In some embodiments, an agent is a diagnostic agent such as an imaging agent. As used herein, an imaging agent is an agent that emits signal directly or indirectly thereby allowing its detection in vivo. Imaging agents such as contrast agents and radioactive agents can be detected using medical imaging techniques such as nuclear medicine scans and magnetic resonance imaging (MRI). Imaging agents for magnetic resonance imaging (MRI) include Gd(DOTA), iron oxide or gold nanoparticles; imaging agents for nuclear medicine include ²⁰¹Tl, gamma-emitting radionuclide 99 mTc; imaging agents for positron-emission tomography (PET) include positron-emitting isotopes, (18)F-fluorodeoxyglucose ((18)FDG), (18)F-fluoride, copper-64, gadoamide, and radioisotopes of Pb(II) such as 203Pb, and 11In; imaging agents for in vivo fluorescence imaging such as fluorescent dyes or dye-conjugated nanoparticles.

The present disclosure further provides the following numbered embodiments:

1. A nucleic acid structure comprising

a first (x), a second (y), and a third (z) nucleic acid arm, each connected at one end to the other arms to form a vertex, and

a first, a second, and a third nucleic strut, wherein the first nucleic acid strut connects the first (x) nucleic arm to the second (y) nucleic arm, the second nucleic acid strut connects the second (y) nucleic arm to the third (z) nucleic arm, and the third nucleic acid strut connects the third (z) arm to the first (x) nucleic acid strut.

2. A nucleic acid structure comprising

three nucleic acid arms radiating from a vertex at fixed angles.

3. A nucleic acid structure comprising

N nucleic acid arms radiating from a vertex, wherein N is the number of nucleic acid arms and is 3 or more, and

M nucleic acid struts, each strut connecting two nucleic acid arms to each other, wherein M is the number of nucleic acid struts and is 3 or more.

4. The nucleic acid structure of embodiment 3, wherein N is equal to M.

5. The nucleic acid structure of embodiment 3, wherein N is less than M.

6. The nucleic acid structure of any one of embodiments 1-5, wherein the nucleic acid structure comprises 4 nucleic acids and at least 4 nucleic acid struts, or 5 nucleic acid arms and at 5 nucleic acid struts.

7. The nucleic acid structure of any one of embodiments 1-6, wherein the nucleic acid arms are equally spaced apart from each other (or the arms are separated from each other by the same angle).

8. The nucleic acid structure of any one of embodiments 1-7, wherein the nucleic acid arms are not equally separated from each other (or the arms are separated from each other by different angles).

9. The nucleic acid structure of any one of embodiments 1-8, further comprising a vertex nucleic acid.

10. The nucleic acid structure of any one of embodiments 1-9, further comprising a connector nucleic acid.

11. The nucleic acid structure of any one of embodiments 1-10, wherein the nucleic acid arms, nucleic acid struts, and/or vertex nucleic acid are comprised of parallel double helices.

12. The nucleic acid structure of any one of embodiments 1-11, wherein nucleic acid arms are of identical length.

13. The nucleic acid structure of any one of embodiments 1-12, wherein the nucleic acid struts are of identical length.

14. The nucleic acid structure of any one of embodiments 1-13, wherein the nucleic acid struts are of different lengths.

15. The nucleic acid structure of any one of embodiments 1-14, wherein at least one nucleic acid arm comprises a blunt end.

16. The nucleic acid structure of any one of embodiments 1-15, wherein at least one nucleic acid arm comprises a connector nucleic acid at its free (non-vertex) end that is up to 16 nucleotides in length.

17. The nucleic acid structure of any one of embodiments 1-16, wherein at least one nucleic acid arm comprises a connector nucleic acid at its free (non-vertex) end, thereby comprising a 1 or 2 nucleotide overhang.

18. The nucleic acid structure of any one of embodiments 1-17, wherein the nucleic acid structure is up to 5 megadaltons (MD) in size.

19. The nucleic acid structure of any one of embodiments 1-18, wherein the nucleic acid arms are 50 nm in length.

20. The nucleic acid structure of any one of embodiments 1-19, wherein the nucleic acid structure comprises three nucleic acid arms separated from each other by 60°-60°-60° (tetrahedron).

21. The nucleic acid structure of any one of embodiments 1-20, wherein the nucleic acid structure comprises three nucleic acid arms separated from each other by 60°-90°-90° (triangular prism).

22. The nucleic acid structure of any one of embodiments 1-21, wherein the nucleic acid structure comprises three nucleic acid arms separated from each other by 90°-90°-90° (cube).

23. The nucleic acid structure of any one of embodiments 1-22, wherein the nucleic acid structure comprises three nucleic acid arms separated from each other by 108°-90°-90° (pentagonal prism).

24. The nucleic acid structure of any one of embodiments 1-23, wherein the nucleic acid structure comprises three nucleic acid arms separated from each other by 120°-90°-90° (hexagonal prism).

25. A composite nucleic acid structure comprising L nucleic acid structures selected from the nucleic acid structures of any one of embodiments 1-24, wherein L is an even number of nucleic acid structures, and wherein the L nucleic acid structures are connected to each other at free (non-vertex) ends of the nucleic acid arms.

26. The composite nucleic acid structure of embodiment 25, wherein the two more nucleic acid structures are two, four, six, eight, ten, twelve or more nucleic acid structures.

27. The composite nucleic acid structure of embodiment 25 or 26, wherein the composite nucleic acid structure is a tetrahedron, a triangular prism, a cube, a pentagonal prism, or a hexagonal prism.

28. The composite nucleic acid structure of any one of embodiments 25-27, wherein the composite nucleic acid structure is 20 megadaltons (MD), 30 MD, 40 MD, 50 MD, or 60 MD in size.

29. The composite nucleic acid structure of any one of embodiments 25-28, wherein the composite nucleic acid structure has edge widths, comprised of two nucleic acid arms from adjacent nucleic acid structures, of 100 nm.

### EXAMPLES

### Materials and Sample Preparation.

DNA strands were synthesized by Integrated DNA Technology, Inc. or Bioneer Corporation. To assemble the structures, unpurified 100 μM DNA strands were mixed with p8064 scaffold in a molar stoichiometric ratio of 10:1 in 0.5× TE buffer (5 mM Tris, pH 7.9, 1 mM EDTA) supplemented with 12 mM MgCl₂. The final concentration of p8064 scaffold was adjusted to 10 nM. Cy3b-modified DNA oligonucleotides were purchased from Biosynthesis (Lewisville, Tex.) (5′-TATGTAGATC-Cy3b; SEQ ID NO: 4). Streptavidin was purchased from Invitrogen (S-888, Carlsbad, Calif.). Bovine serum albumin (BSA), and BSA-Biotin was obtained from Sigma Aldrich (A8549, St. Louis, Mo. Glass slides and coverslips were purchased from VWR (Radnor, Pa.). Two buffers were used for sample preparation and imaging for super-resolution DNA-PAINT imaging: Buffer A (10 mM Tris-HCl, 100 mM NaCl, 0.05% Tween-20, pH 7.5), buffer B (5 mM Tris-HCl, 10 mM MgCl₂, 1 mM EDTA, 0.05% Tween-20, pH 8).

### Annealing Ramps.

The strand mixture was then annealed in a PCR thermo cycler using a fast linear cooling step from 80° C. to 65° C. over 1 hour, then a 42 hour linear cooling ramp from 64° C. to 24° C.

### Agarose Gel Electrophoresis.

Annealed samples were subjected to gel electrophoresis in 0.5% TBE buffer that includes 10 mM of MgCl₂, at 90V for 3 hours in an ice-water bath. Gels were stained with Syber® Safe before imaging.

### TEM Imaging.

For imaging, 2.5 μL of annealed sample were adsorbed for 2 minutes onto glow-discharged, carbon-coated TEM grids. The grids were then stained for 10 seconds using a 2% aqueous uranyl formate solution containing 25 mM NaOH. Imaging was performed using a JEOL JEM-1400 TEM operated at 80 kV.

### Super-Resolution Imaging.

Fluorescence imaging was carried out on an inverted Nikon Eclipse Ti microscope (Nikon Instruments, Melville, N.Y.) with the Perfect Focus System, applying an objective-type TIRF configuration using a Nikon TIRF illuminator with an oil-immersion objective (CFI Apo TIRF 100, NA 1.49, Oil). For Cy3b excitation a 561 nm laser (200 mW nominal, Coherent Sapphire) was used. The laser beam was passed through cleanup filters (ZET561/10, Chroma Technology, Bellows Falls, Vt.) and coupled into the microscope objective using a multi-band beam splitter (ZT488rdc/ZT561rdc/ZT640rdc, Chroma Technology). Fluorescence light was spectrally filtered with an emission filter (ET600/50m, Chroma Technology) and imaged on an EMCCD camera (iXon X3 DU-897, Andor Technologies, North Ireland). Imaging was performed without additional magnification in the detection path, yielding 160 nm pixel size.

### Sample Preparation and Imaging.

For sample preparation, a piece of coverslip (No. 1.5, 18×18 mm², 0.17 mm thick) and a glass slide (3×1 inch², 1 mm thick) were sandwiched together by two strips of double-sided tape to form a flow chamber with inner volume of 20 μL. First, 20 μL of biotin-labeled bovine albumin (1 mg/mL, dissolved in buffer A) was flown into the chamber and incubated for 2 min. The chamber was then washed using 40 μL of buffer A. 20 μL of streptavidin (0.5 mg/mL, dissolved in buffer A) was then flown through the chamber and allowed to bind for 2 min. After washing with 40 μL of buffer A and subsequently with 40 μL of buffer B, 20 μL of biotin-labeled microtubule-like DNA structures (≈300 pM monomer concentration) and DNA origami drift markers (≈100 pM) in buffer B were finally flown into the chamber and incubated for 5 min. The chamber was washed using 40 μL of buffer B. The final imaging buffer solution contained 3 nM Cy3b-labeled imager strands in buffer B. The chamber was sealed with epoxy before subsequent imaging. The CCD readout bandwidth was set to 3 MHz at 14 bit and 5.1 pre-amp gain. No EM gain was used. Imaging was performed using inclined illumination with an excitation intensity of ˜200 W/cm² at 561 nm. 3D images were acquired with a cylindrical lens in the detection path (Nikon). All images were reconstructed from 5000 frame long time-lapsed movies acquired with 200 ms integration time, resulting in ≈17 min imaging time.

### Image Processing and Drift Correction.

Super-resolution DNA-PAINT images were reconstructed using spot-finding and 2DGaussian fitting algorithms programmed in LabVIEW (Jungmann, R., et al. Nature Methods, advance online publication, 2014). A simplified version of this software is available for download at the “dna-paint” website. The N-STORM analysis package for NIS Elements (Nikon) was used for data processing. 3D calibration was carried out according to the manufacturer's instructions. DNA origami drift markers (Lin, C., et al. Nature Chemistry 4, 832-839, 2012) were used as fiducial markers. The high binding site density increases the probability to observe one bound imager strand per structure in each image frame. Furthermore, the fluorescence intensity of the origami drift markers is similar to single imager strand binding events and the markers never “bleach”. These properties render DNA origami structures as ideal drift markers. Drift correction was performed by tracking the position of each origami drift marker structure throughout the duration of each movie. The trajectories of all detected drift markers were then averaged and used to correct the drift in the final super-resolution reconstruction.

### Determination of Localization Accuracy.

Fitting a 1D-Gaussian function to the distribution of z localizations from DNA origami drift markers and calculating the standard deviation was used to determine the localization accuracy in z. As origami drift markers are 2D structures, all binding events occur in a 2D plane on the surface, and thus at the same z location. Localization accuracy in x and y was determined by calculating the average separation of single-molecule localizations in neighboring frames, which can be attributed to an imager strand binding to a single docking strand. As multiple docking strands are used in each vertex of the polyhedral (˜18 strands per vertex), one cannot fit the distribution of binding events per vertex, as this would result in an overestimation of the localization accuracy. The measured value per vertex would represent a convolution of the actual localization accuracy with the spatial extent of the binding sites in this vertex.

Spatial vs. Temporal Imaging Resolution.

In stochastic super-resolution microscopy such as DNA-PAINT, one can generally make the statement that there is a tradeoff between spatial and temporal resolution. Higher spatial resolution can be obtained by collecting a larger amount of photons per binding or photoswitching event. This can be achieved by increasing fluorescence ON times and matching the camera integration time to these ON times. In DNA-PAINT imaging, this can be accomplished by increasing the binding stability of the imager/docking complex (i.e. going from a 9 to a 10-nt interaction region) and increasing the camera integration time to match the longer binding time, which in turn results in a longer image acquisition time. Higher temporal resolution can be obtained by reducing the binding stability of the imager/docking complex (i.e. going from a 9 to a 8-nt interaction region) and decreasing the camera integration time to match the shorter binding time.

TABLE 3
Sequences for super-resolution DNA-PAINT imaging.

| Description | Sequence |
|---|---|
| Cy3b imager strand | 5′-TATGTAGATC-Cy3b (SEQ ID NO: 1) |
| 9 nt docking site for P2 imager | Staple-TTATCTACATA-3′ (SEQ ID NO: 2) |
| Biotinylated surface strand for | Biotin-GAATCGGTCACAGTACAACCG-3′ |
| structure immobilization | |
| Handle strand on the DNA structure for | Staple-TTCGGTTGTACTGTGACCGATTC- |
| surface immobilization; 7 staples (5′ | 3′ (SEQ ID NO: 4) |
| ends are 48[69], 43[130], 27[129], | |
| 11[88], 9[130], 26[65]) are | |
| modified. See Table 4 for sequence | |
| details. | |

TABLE 4
Sequences of the tetrahedron.

| 5′-end | Sequence | Note | SEQ ID NO: |
|---|---|---|---|
| 1[84] | TGAGGCCAACGCTCATGGACGTACTATGGTTTTTACAGCCTCCGGA | Core staple | 5 |
| 0[54] | ACGTATTACGCCACCAAACATCCCTTAGCCAGCGAAAG | Core staple | 6 |
| 3[102] | TCGATTGCAACAGGAAAACCGAGTGTTTTTTTGGT | Core staple | 7 |
| 3[144] | CACTCGGCCTTGCTGGTAGCAATATAATTACATTTATGTATT | Core staple | 8 |
| 2[44] | AACATAAATCAAAAGAAGCAGCAAGTTTTTCTCCA | Core staple | 9 |
| 2[51] | ATTGTGCCGGCACTGCGGCACGCGGTCATAGCTGTTTCCATA | Core staple | 10 |
| 2[72] | AGTGACGGATTCGCCTGTCGCTGGTAATCAG | Core staple | 11 |
| 2[93] | ATGTGAATACACCTTTTTGATCAATATAATCTTTC | Core staple | 12 |
| 2[107] | GACCATCGCCATTAAAAATGAAAATGGTCAGTACA | Core staple | 13 |
| 2[114] | TGGGCGCAGAAGATGAATTTGGATTCCTGATTATCAGAATTA | Core staple | 14 |
| 2[135] | ACCTTCAATTTAGATTTATGGAAGGGAGCGGAATTATCTTAT | Core staple | 15 |
| 5[39] | CTTGTGGACTCGTAACCTTTCCTCGTTAGAAAGGG | Core staple | 16 |
| 5[60] | CCGAAGAGTCGCTTAATTGACGAGC | Core staple | 17 |
| 5[123] | CGAGTAAGAATTTACATAGAACAATATTACCATCACGCCCGT | Core staple | 18 |
| 4[83] | CCCTTCAGTTAATGGTCTTTGCGAATACCTACATTTTGACGCTTGA | Core staple | 19 |
| 7[32] | TATGCCAGCTATACGAGCCGGAAGCTGTGTGGGGGGTTTAAT | Core staple | 20 |
| 7[74] | GCACGTTGCGTGAGTGAGCTAACTGGGTACCAGCCTCCCAAA | Core staple | 21 |
| 7[81] | CTGGAGAAACAATAACGGTCCGTGGAGCTCGAATTCGTTGCC | Core staple | 22 |
| 7[91] | ATCAAACATTAGACTTTACCATTAATTGACAG | Core staple | 23 |
| 7[109] | ATCATCTAAAGCATCACCCTAAAAAATATTTTCAA | Core staple | 24 |
| 6[51] | GTCTGTAAAGCCTGGGGAATCATGTGCC | Core staple | 25 |
| 6[114] | TTTCCTTTGCCCGAACGATCATATTATACTTAAAT | Core staple | 26 |
| 8[44] | TGTCAGGGTGGCGGTCCACGCTGGATCC | Core staple | 27 |
| 8[65] | AGCCAGTGAGGCCCTGAGAGAGTTTAGC | Core staple | 28 |
| 9[60] | TGTCCAACGCATAACGGAACGTGCCGGC | Core staple | 29 |
| 9[130] | ATATCAGGTTATCAACAAGAGCCAGCAGCAAATAC | Core staple | 30 |
| 11[88] | CTTGCTATTACGCGAACTGATAGCCTTGCTGAACCTTG | Core staple | 31 |
| 11[130] | CATTGAAAGCACGAACCACCAGCACACGCTGGTTG | Core staple | 32 |
| 10[37] | GGTTTAGACAGGAACGGAACGTGCACCACACCCGCCGCCACT | Core staple | 33 |
| 10[58] | CATGAATCCTGAGAAGTGTTGCTTGCGCCGCTACAGGGTTCC | Core staple | 34 |
| 10[65] | CAGTGCATCATTGGAACAGATAGGGTTGAGTCCGCCTGACGG | Core staple | 35 |
| 10[100] | TCCAAAAGAGTCTGTCCGCCAGCCTCTGAAATGGATTATACG | Core staple | 36 |
| 10[114] | TCCGGGTAAACGCTATTAATTAATCTGATTGTATACAGCAAT | Core staple | 37 |
| 10[121] | TTGAAATTAACCGTTGTAATATCCTGGCAGATTCACCATCTG | Core staple | 38 |
| 13[74] | CTTTTACCAGTATAAAGTCTTCGCATCC | Core staple | 39 |
| 13[95] | GCTTCATATGCGTTATATCACAGTACATCGGATCAAAT | Core staple | 40 |
| 12[37] | TGAAGGTTTCTTTGCTCGTCATTCTCAACAGTAGGGCTTCTGCCACGCC | Core staple | 41 |
| 12[79] | TTCGTAGAACGTCAGCGCGTCTCGATTG | Core staple | 42 |
| 12[100] | CCTGCTTTAGTGATGAAGGCAAACCAAAATCCACA | Core staple | 43 |
| 12[121] | CGTGTTAAACGAACAATTTCATTTAACCTTGCTTCTGTCTGA | Core staple | 44 |
| 15[46] | AAGGGGAAACCTGTCGTTGGGCGCGCACTCTACCTGCACACT | Core staple | 45 |
| 15[67] | TAACTCACTGCCCGCTTTTTTCACGCAGTGTTGCCCCCAGCA | Core staple | 46 |
| 15[88] | ACAATTCGACAACTCGTTGATGGCAATTCAGGATCCCCCAAA | Core staple | 47 |
| 15[109] | AATGAGGATTTAGAAGTCCTCAATTAACAGTCAAGTTAGCGG | Core staple | 48 |
| 15[130] | TAACCGTCAATAGATAATTGGCAATAACGTCGGCGAATCTGA | Core staple | 49 |
| 17[147] | GTCTGGTCAGCAGCAACCGCAAAAAAAAGCCGCACAGGCGGC | Core staple | 50 |
| 16[188] | ATCGACATAAAAAAATCCCGTAGAATGCCAACGGCAGCACCG | Core staple | 51 |
| 16[209] | AGCAGTTGGGCGGTTGTGTACTCGGTGGTGCCATCCCACGCA | Core staple | 52 |
| 16][229] | ATTTCTGCTCATTTGCCGCCACCAGCTTACGGCTGGAGGT | Core staple | 53 |
| 19[53] | GAACTGACCAACTTTGAATCAAGATAAT | Core staple | 54 |
| 19[84] | CATTTCGAGCTAAATCGGTGAGCTTAATTTGACCAAGAG | Core staple | 55 |
| 19[116] | ATAAGCAGCGCCGCTTTAGAAACAGCGGATCGGAAGATTATT | Core staple | 56 |
| 18[44] | CATCTCCTTTTGATAAGCGCGTTTGTAA | Core staple | 57 |
| 18[65] | GAATTTTGCGGATGGCTAGCC | Core staple | 58 |
| 21[39] | TTGGTTTTAAATATGCATATAACACAGATGAACGG | Core staple | 59 |
| 21[102] | GTAGCCTCAGAGCATAACAAATGGAACG | Core staple | 60 |
| 21[144] | AAATCATACAGGCAAGGGCGAGCTCGGCGAAACGTAGTCAGT | Core staple | 61 |
| 20[44] | TCGTCAGAAGCAAAGCGCCCCCTCGTAATAGGCAA | Core staple | 62 |
| 20[65] | CTTTCAAAAAGATTAAGCGTCATATGGATAGGAAT | Core staple | 63 |
| 20[72] | CGATAATTAAGTTGGGTCGGCTACTTAGATA | Core staple | 64 |
| 20[93] | ATCGGGTTTTGCGAAAGTTGTATCGGCCTCAAAAC | Core staple | 65 |
| 20[107] | CCGTAATGCCGGAGAGGGCATGTCGTATAAGAAAA | Core staple | 66 |
| 20[114] | AGATGTAAAATCTTCGCCGCACTCTCTGCCAGTTTGAGTGAG | Core staple | 67 |
| 20[135] | AGGAAGCTTTGAAGGGCGCACCGCTGGGCGCATCGTAAGATT | Core staple | 68 |
| 23[60] | GCACAAATATAGGTCATTATAATGCTGTAGCCTGC | Core staple | 69 |
| 23[123] | CTATCAAAAGGAAGCCTTTAGCAAAATTAAGAGCT | Core staple | 70 |
| 22[97] | CGGTTGATAATCCTGCGGAATAGATATTCAACCGTTCTAGCT | Core staple | 71 |
| 25[32] | AAGTTTACCAAGAAAGATTCATCATTAATAAATTGGGCGTTG | Core staple | 72 |
| 25[60] | ATGCAAATCATGACAAGCTAAAGACGAGTAGATTTAGTTGCT | Core staple | 73 |
| 24[51] | CACTTTAGGAATACCACCGTTGGGTTTCAACGCA | Core staple | 74 |
| 24[72] | TACTAATGCAGATACATGGCTCATATTACCTGGGG | Core staple | 75 |
| 24[90] | GCCAGCGCCAAAAGCGTCCAATGCTGCAAGGCGTTATTG | Core staple | 76 |
| 24[114] | TAAGTAACAACCCGTCGCCGTGCACAGCCAGGAGA | Core staple | 77 |
| 26[44] | CTGAGAGGGGAAATGCTTTAAACAATTATAGAGCTTCATTAA | Core staple | 78 |
| 26[65] | ACCTTTAGACAATATTCATTGAATGATT | Core staple | 79 |
| 26[86] | ATGTAAGAAAAGCCCCATCCTGTA | Core staple | 80 |
| 26[107] | ACGGAAGATTAATCATATGTACCCGATAAATGAGACAGCCCT | Core staple | 81 |
| 27[74] | TGATATACCAGTCAGGAATTCAACGAGGCATAGTAAGATAAA | Core staple | 82 |
| 27[129] | TCCGGATCGGTTTAAATTTAATCGTAAAACTAGTAG | Core staple | 83 |
| 29[39] | TTCAAGAGGAGTTGATTCCCAATTTCAA | Core staple | 84 |
| 29[53] | TCTACGTAACGGTTTAAAAGAAAAATCTACGGTTG | Core staple | 85 |
| 29[88] | CCAACCATCAATATGGATATGTACCAAAAACATTATGATCAA | Core staple | 86 |
| 29[102] | GTCGCATCGGTCAATAACCTGTTTCAATAAAATACTTTTGCGGGAGGTG | Core staple | 87 |
| 29[130] | GCCTAAAGATTTTTTGAGAGATCTTGAACGGGTAA | Core staple | 88 |
| 28[72] | GCTTCCATTATTGCAGGCGCTTTCTTTAATCCATT | Core staple | 89 |
| 28[93] | AGGGTAATGCAGTCCAGCATCAGCTATGCGAGGGG | Core staple | 90 |
| 28[121] | CTCTTTTCATTTGGGGCCAAAGAATTATTTCAACGCAAGTGT | Core staple | 91 |
| 30[37] | CGGATCATAAGGGAACCGAACTTTATCCGCCGGGCGCGTTGAGATAAAG | Core staple | 92 |
| 30[59] | CTCATTCATGAGGAAGTTTTGAGGAAACCGGAAAGA | Core staple | 93 |
| 30[79] | TCAAACGGGTAAAATACGTAGCAAAACG | Core staple | 94 |
| 30[100] | TTACAGGGAGTTAAAGGAAAGACAACGACGTAAGG | Core staple | 95 |
| 30[121] | CGCTGCGGGATCCAGCGCCATGTTCTCTCACGGAAAAACTT | Core staple | 96 |
| 33[46] | AGATATCATAACCCTCGTTTTGCCCTCATTCGACC | Core staple | 97 |
| 33[91] | ATCAACATTAAATGGGGACGACGACATTAAGAACTAACTTTC | Core staple | 98 |
| 33[109] | CGATTCGCGTCTGGCCTAAAACAGCCAGCTGCCCA | Core staple | 99 |
| 33[130] | CTCTAGGAACGCCATCACAAATATGCGGGCCCGACGGCCACC | Core staple | 100 |
| 35[147] | ACTACGAAGGCACCAACCTAATATTCGGTCGCTGAGGCTTGC | Core staple | 101 |
| 34[188] | ATCGCCCACGCATAACCGATAAACGAAAGAGGCAAAAGAATA | Core staple | 102 |
| 34[209] | GCGCCGACAATGACAACAACCCACTAAAACACTCATCTTTGA | Core staple | 103 |
| 34[229] | ACAGCTTGATACCGATAGTTCCCCCAGCGATTATACCAAG | Core staple | 104 |
| 37[53] | TATAATAAGAGAATATAATGTTCAAGCA | Core staple | 105 |
| 37[84] | GGTTTACCAAGGCCGGAAACTG | Core staple | 106 |
| 37[116] | TTCTAACTATAACCTCCGCTTTCGAGGTGAACGCCACCAACT | Core staple | 107 |
| 36[44] | TTACCGAGGAAACGCAAATGAAATGCTAATGTCCT | Core staple | 108 |
| 36[65] | GACGGAATACCCAAAAGCAAT | Core staple | 109 |
| 36[75] | GCATGATAGAAAAAGAACGCTTCATCTAGATTTG | Core staple | 110 |
| 39[39] | AAAGCAAACGTAGAAAAACGCAAAGACAAAAAGGC | Core staple | 111 |
| 39[102] | GCAACCATTACCATTAGCAGCGCCGCAAATCAATGGTTACGCGAA | Core staple | 112 |
| 39[144] | GCGTTGAGCCATTTGGGGGGAAGGACAACTAAAGGATGTCTG | Core staple | 113 |
| 38[44] | ATATAATATCAGAGAGAAATAACACCCAATCAATT | Core staple | 114 |
| 38[65] | GCACAAGAATTGAGTTAAATAGCATTTTTTGTGCT | Core staple | 115 |
| 38[72] | AATTTTTAGCGTAACGAAAGACAATTCATAT | Core staple | 116 |
| 38[83] | GGAACCCAACGTCACCAATGAAACCATCCCAG | Core staple | 117 |
| 38[93] | AGCTTTTGTCTAGCATTACGAGGTTTAGTACTTTC | Core staple | 118 |
| 38[107] | ATCGAACCGCCACCCTCTATTCACACCGTTCCAGT | Core staple | 119 |
| 38[114] | AATTAGTAAACAGTACACTCAGAACGGAATAGGTGTATATTA | Core staple | 120 |
| 38[135] | TAGGGGATTTCGTAACAACCGCCAAGGGTTGATATAAGAAGA | Core staple | 121 |
| 41[60] | CCAAGAAACATAATAACTCCTTATTACGCAGAGTT | Core staple | 122 |
| 41[123] | CCACATCTTTAGCGACAGCCAGCAAAATCACGACA | Core staple | 123 |
| 40[97] | TCATTAAAGCCAAAAAATGAAAGCGCCTCCCTCAGAGCCGCC | Core staple | 124 |
| 43[32] | ACAAACGCTAGAACGCGAGGCGTTAAGCAAAGTCTTTCTCCG | Core staple | 125 |
| 43[60] | TAAAGATAAGCAGAACGCTTTTTCTTTGTCACAATCAATTAA | Core staple | 126 |
| 43[130] | ATAACGATTGGCCTTGAAGAG | Core staple | 127 |
| 42[51] | TTAACCTCCCGACTTGCATCATTAAACGGGTGCCT | Core staple | 128 |
| 42[72] | ATTTTTGAAGCCTTAAAGTTTTTACGCACTCACAA | Core staple | 129 |
| 42[90] | CCTATAAGATTAGTTTTAACGCAGCCCTCATAGATCAAG | Core staple | 130 |
| 42[114] | TAAGGCTGAGACTCCTCTATAGCCCCGCCACTCAGCTTGGCTTAG | Core staple | 131 |
| 44[51] | GAATTCCAAGCCGCGCCCAATAGCTTAG | Core staple | 132 |
| 44[107] | ACATGAATTTAAACAAATAAATCCACCCTCAACCGGAAGATA | Core staple | 133 |
| 45[46] | TCACAAGAAATATTTATTAAAAACAGGGAAGTGAGCGCGCTATCTAAGG | Core staple | 134 |
| 45[74] | TACTTTTCATCGTAGGAGGGAGGTTTGCACCCAGCTACCAAA | Core staple | 135 |
| 47[39] | AACAAGTACCGACACCACGGAATATATG | Core staple | 136 |
| 47[102] | TTCTGCTGATAAAGACAAAAGGGCCAGTAGCGCACCGTAATCAGTTCAT | Core staple | 137 |
| 47[130] | TATCGTTTGCCCACCCTCAGAGCCAGGTCAGCATGGCTGAGT | Core staple | 138 |
| 46[121] | ATAAACCGATTGAGGGAAATTAGAGAATCAAGTTTGCCTTAT | Core staple | 139 |
| 49[126] | GTATTGCGAATAATATTGTATCGGTTTACCTCAGACTGAGTTCGTC | Core staple | 140 |
| 48[37] | CGAGGCATTTTCGAGCCAGTAAATAAATTGTGTCGAAACTTA | Core staple | 141 |
| 48[58] | GATATATTTTAGTTAATGAGAAAACGCCTGTAAGA | Core staple | 142 |
| 48[69] | TATCATCATTAAACCAACAATGAAACGAGCCTTTACAGAGAGTAAC | Core staple | 143 |
| 48[79] | CGGTCTGACCTAAATTTCAATCGCTCTAAAGCACCACC | Core staple | 144 |
| 48[90] | ACAAAGTATCGAGACCACAGATCGAATGGAAAGCGTTCGGAA | Core staple | 145 |
| 48[100] | TTATAGACTACCTTTTTATGTAAACAGACGTCAAA | Core staple | 146 |
| 50[104] | CACCGTACTCAGAAGCAAGCCTCTATTCTGAAACATGAAAGT | Core staple | 147 |
| 51[46] | CGATCCTGAATCTTACCGCCATATAATAATAAAAC | Core staple | 148 |
| 51[109] | AGATGCCCCCTGCCTATCAGTCTCACGCCTGGTCT | Core staple | 149 |
| 51[130] | GAAAGTGCCCGTATAAACAGTAAGTCGTCACTGAATTTGGTT | Core staple | 150 |
| 53[147] | GAAATACCGACCGTGTGATAATATCAAAATCATAGGTCTGAG | Core staple | 151 |
| 52[188] | GAGAAGAGTCAATAGTGAATTATAAGGCGTTAAATAAGAATA | Core staple | 152 |
| 52[209] | GATAGCTTAGATTAAGACGCTAACACCGGAATCATAATTACT | Core staple | 153 |
| 52[229] | AGAATCCTTGAAAACATAGCAGAAAAAGCCTGTTTAGTAT | Core staple | 154 |
| 7[137] | AAAATTAGAGTTTTAAAAGTTTGAACCAGAAGGTTAGAAGTG | Core staple | 155 |
| 7[151] | AGGGCCTGCAACAGTGCGAAGATAGAACCCTGTCA | Core staple | 156 |
| 6[146] | CTAATAGGGAATTGAATTGCGACCTGAGACAA | Core staple | 157 |
| 12[142] | AATGAATTACCTTTTTTCAAGAAACAAA | Core staple | 158 |
| 25[137] | ACGTAACCAACGTGGGAACAAACGGTGTAGATTCTGGTGGGA | Core staple | 159 |
| 25[151] | TTAAACAAGAGAATCGAACAAAGGGAGTAATGGAT | Core staple | 160 |
| 24[146] | CATTTTTTTAATATCTGTTGGCAGAGGTAAAC | Core staple | 161 |
| 30[142] | TAGTACCAGTCCCGGAATCACCGGGGAG | Core staple | 162 |
| 43[151] | AGGCAGGAGGTTGAGGCGCCACCAAGCCCCCTTTA | Core staple | 163 |
| 42[135] | AACGGATTAGGATTAGCCGTCGAGCCCTCAGGCCT | Core staple | 164 |
| 42[146] | GTGCCTTTTTGATGCATGTACTGCTAAAGAAA | Core staple | 165 |
| 48[142] | TTAAATTTTTTCACGTTGAGAATACAAC | Core staple | 166 |
| 0[166] | GAGTAGAAGAACTAATAACATCACTTGCGC | Connector staple | 167 |
| 2[163] | TCTGGCCAACAGATGATGAGC | Connector staple | 168 |
| 4[163] | TATTAACACCTTATCTAAAATAAT | Connector staple | 169 |
| 6[163] | TTTAGGAGCATATCATTTTCT | Connector staple | 170 |
| 8[166] | ACGTAAAACAGAAATATCAAAATTATTTAA | Connector staple | 171 |
| 11[151] | AGAAGAGATAAAACAGAGGTGAGGCGGTCAG | Connector staple | 172 |
| 10[142] | AATCTTCTTTGATTAGTCAAACTAGACCAGTAATAAAAGGGACTC | Connector staple | 173 |
| 10[160] | CAAACATAATGGAAACAGTAC | Connector staple | 174 |
| 12[163] | ATAAATCAATATATGTGACCTACCATAAAGAAGGA | Connector staple | 175 |
| 14[160] | GGAACAAAGAAACCGTAACATCTAACAA | Connector staple | 176 |
| 18[166] | TAGCATTAACATCAATTCTACTAATAGTGG | Connector staple | 177 |
| 20[163] | TTTTAAATGCCCACGGGAAAT | Connector staple | 178 |
| 22[163] | GTCTGGAGCAAAATTCGCATTATA | Connector staple | 179 |
| 24[163] | TTTTTGTTAAGACCGTAATAG | Connector staple | 180 |
| 26[166] | TCGCCATTCAGGCACCAGGCAAAGCGCCCG | Connector staple | 181 |
| 29[151] | CCGAATGCCTCTATCAGGTCATTGCCTGAGA | Connector staple | 182 |
| 28[142] | AATGAAAAGGTGGCATCCAATAAAAATTTTTAGAACCCTCATAAA | Connector staple | 183 |
| 28[160] | GATAACCTTTGTGAGAGATAG | Connector staple | 184 |
| 30[163] | ACTTTCTCCGTGGTGAAGCCGGAATGCGCAATTTG | Connector staple | 185 |
| 32[160] | GATAGGTCACGTTGGCGGATTATCAGCT | Connector staple | 186 |
| 36[166] | GAATTATCACCGTAATTATTCATTAAAGCC | Connector staple | 187 |
| 38[163] | TCGGCATTTTCAACAGTTTGA | Connector staple | 188 |
| 40[163] | CCAGCATTGAAGTGTACTGGTACA | Connector staple | 189 |
| 42[163] | AAGTTTTAACTGCTCAGTAGT | Connector staple | 190 |
| 44[166] | TAGCAAGCCCAATACCCTCATTTTCAGGCA | Connector staple | 191 |
| 47[151] | TTTCGGTCATGAACCACCACCAGAGCCGCCG | Connector staple | 192 |
| 46[142] | GGATAAATATTGACGGACACCGACTCAGACTGTAGCGCGTTTTAT | Connector staple | 193 |
| 46[160] | GCGGAGTGAAAATCTCCAAAA | Connector staple | 194 |
| 48[163] | AAAAGGCTCCAAAAGGAAGCCACCAGGAACCATAC | Connector staple | 195 |
| 50[160] | AGGCGGATAAGTGCGGGGTTTGGGGTCA | Connector staple | 196 |
| 1[12] | ACAGGAGGCCGATTAATCAGAGCGCGGTCACGCTGCGCCAA | Vertex staple | 197 |
| 1[32] | ATTGTGTTCATGGGTAAGAATCGCCATATTTAACAACG | Vertex staple | 198 |
| 3[9] | TATCAAAGTGTAGGGAGCTAA | Vertex staple | 199 |
| 2[30] | CGTCCGGGTTGTGGTGCTCATACCAAATTGTTATCCGCTCACA | Vertex staple | 200 |
| 5[9] | TTGATGGTGGTTCGAAAAACCGTC | Vertex staple | 201 |
| 7[9] | CGCGCGGGGAGAAGAATGCGG | Vertex staple | 202 |
| 9[12] | CGGGCCGTTTTCACGGTGCGGCCGGCGGTTCAGCAGGCGAAAATCCTGT | Vertex staple | 203 |
| 11[16] | CGGCATCAGATGCAAAGGGCCGAAATCGGCAAATTTGCCCTGCG | Vertex staple | 204 |
| 13[14] | CCTGCGGCTGGTAAGCAAATCGTTAA | Vertex staple | 205 |
| 15[16] | ATTCCACACAACGCATTAATGAATCGGCCAA | Vertex staple | 206 |
| 19[12] | TGGAAGTTTCATTCCAACTAAAGATTAGAGAGTACCTAAG | Vertex staple | 207 |
| 21[9] | CAACAGGTCAGGTACGGTGTC | Vertex staple | 208 |
| 20[31] | CGAAGCTGGCTAGTGAATGTAGTAAAACGAACTAACGGAACAAC | Vertex staple | 209 |
| 23[9] | TCAAAAATCAGGGGAAGCAAACTC | Vertex staple | 210 |
| 25[9] | ATAGCGAGAGGCGCCCTGACG | Vertex staple | 211 |
| 27[12] | AGAAACACCAGAACGAAAGGCTTTTTTGCAAAACGAGAATGACCATAAA | Vertex staple | 212 |
| 29[16] | CCAGGCGCATAGCCAGACCTCTTTACCCTGACTGTTCAGAAAAG | Vertex staple | 213 |
| 31[14] | GGAACGAGGCGCAGACGGTGTACAGA | Vertex staple | 214 |
| 31[32] | TCATATGAGCCGGGTCACTGTTGC | Vertex staple | 215 |
| 33[16] | ATTATTACAGGTGACGACGATAAAAACCAAA | Vertex staple | 216 |
| 37[12] | GCAACATATAAAAGAATACATACAACAAAGTTACCAGTACC | Vertex staple | 217 |
| 39[9] | AGCAGATAGCCGATAAAGGTG | Vertex staple | 218 |
| 38[30] | GAACGACAATTCCCATCATCGGCTTCAGATATAGAAGGCTTAT | Vertex staple | 219 |
| 41[9] | CACCCTGAACAATTAAGAAAAGTA | Vertex staple | 220 |
| 43[9] | CTAATTTGCCAGACGAGCATG | Vertex staple | 221 |
| 45[12] | TAGAAACCAATCAATACTAATTTTTACAAAGACGGGAGAATTAACTGAA | Vertex staple | 222 |
| 47[16] | CTGTCCAGACGAGCCCTTTAGTCAGAGGGTAATCGCATTAATAA | Vertex staple | 223 |
| 49[14] | CCAACATGTAATTTGGTAAAGTAATT | Vertex staple | 224 |
| 49[32] | AGACCTGCTCCATGTTACTTAGCC | Vertex staple | 225 |
| 51[16] | CCGGTATTCTAAACGAGCGTCTTTCCAGAGC | Vertex staple | 226 |

TABLE 5
Sequences of the triangular prism.

| | | | SEQ ID |
|---|---|---|---|
| 5′-end | Sequence | Note | NO: |
| 1[53] | CGCCAACCGCAAGAAAAGTTACCTGTCC | Core staple | 227 |
| 1[84] | AGTGAGGAAAACGCTCATGCGCGTACTAGTGTTTTTGGT | Core staple | 228 |
| 0[44] | CGTCCACCACACCCGCCAACAAGAGCAG | Core staple | 229 |
| 3[102] | AATCCATTGCAACAGGACCACCGACGGACTTGCGGTCCCTTAGAA | Core staple | 230 |
| 3[144] | CACTATCGGCCTTGCTGGTAGCAAATTAATTACATTGCATTA | Core staple | 231 |
| 2[44] | ACTAAAATCCCTTATAATGAGAGACGCCAGGCTGC | Core staple | 232 |
| 2[65] | TCCGAATAGCCCGAGATTTGCCCTCACC | Core staple | 233 |
| 2[72] | GTGCCAACGGATTCGCCGTCAGCGTATAATC | Core staple | 234 |
| 2[93] | GAATTTGAATGTACCTTTCTCATCAATATAAATTT | Core staple | 235 |
| 2[107] | CAGAACATCGCCATTAAAAATGAATCTGGTCAATA | Core staple | 236 |
| 2[114] | CGTTCGCGCATCAGATGTGTTTGGATTCCTGATTATCAGTAT | Core staple | 237 |
| 2[135] | TGAATTTCAACGTAGATTAATGGAAAGGAGCGGAATTACGTT | Core staple | 238 |
| 5[60] | AAAAGTTTGGGCGCTTATTTGACGAGCACGTGGTA | Core staple | 239 |
| 5[123] | ACCGCGTAAGTATTTACCCAGAACAATATTACCATCACCATC | Core staple | 240 |
| 4[41] | CAAGCGGAATCGGCATTAAAGCGCGTAAGCTTTCC | Core staple | 241 |
| 4[97] | ACCTTGCTGAACAACAGCTGAAGTTTAATGCGCGAACTGATA | Core staple | 242 |
| 4[135] | CGCCAGTTGAAGATTAGAATTTTAAAAGTTTCCAC | Core staple | 243 |
| 7[32] | GCGAACCTGTTCCACACAACATACTAGCTGTCGGTCATTGAG | Core staple | 244 |
| 7[60] | TTTACGATCCGCGGTGCTCAG | Core staple | 245 |
| 7[74] | AGTACATTAAGGGTGCCTAATGAGGAGGATCCGCGTCCAAAC | Core staple | 246 |
| 7[109] | ATAAAATCTAAAGCATCGCCCTAAACAATATGCTC | Core staple | 247 |
| 6[51] | CCGAAGCATAAAGTGTATCGAATTCCAG | Core staple | 248 |
| 6[90] | ACTTTAGCTAACTCGAGACGGGGGAGAAACAATCTTGTTCTTCCCGG | Core staple | 249 |
| | GT | | |
| 6[114] | CATATCCTTTGCCCGAATCATCATATTATACGTAA | Core staple | 250 |
| 8[65] | CAGTTCTTTTTCACCGCCTGGCCCATCA | Core staple | 251 |
| 9[60] | CACCGCTCAACACCGTCGGTGATGGGTCTGGCGGTGCCTTGT | Core staple | 252 |
| 9[130] | GAATTTCAGGAAATCAATGAGAGCCAGCAGCAAAT | Core staple | 253 |
| 11[39] | CGGACATCCCTTTTAGACAGGAACATAA | Core staple | 254 |
| 11[53] | CCAAGCGCAGGTTTCTGCGTAATCATGGTCAGAGC | Core staple | 255 |
| 11[88] | TGCTGGCTATTAGTCGGGGGAAATACCTACATTTTGACTTTT | Core staple | 256 |
| 11[130] | TTCCCTGAAAGAACGAACCACCAGGCCA | Core staple | 257 |
| 10[58] | CAGCAGAATCCTGAGAATGGTTGCATGCGCCGCTACAGTTGA | Core staple | 258 |
| 10[72] | GCTCTGATTGCCGTTCCGGCAAACGTAGAACTGAT | Core staple | 259 |
| 10[100] | TGCGTAAAAGAGTCTGTCCGCCAGCGTCTGAAATGGATAATA | Core staple | 260 |
| 10[114] | CTCTCGCTGGGTCGCTATTAATTATCCTGATAATATACATCA | Core staple | 261 |
| 10[121] | GCAGCAAATTAACCGTTGTAATATATTGGCAGATTCACCTTC | Core staple | 262 |
| 12[37] | AATGCTCGTCATTGCCAACGGCAGCAGTAGG | Core staple | 263 |
| 12[48] | GCTTAATACCGGGGTGTCACTTATTGGGGTTGCAG | Core staple | 264 |
| 12[79] | ATAGCGATAGCTTACAAGCGTGCCGCAT | Core staple | 265 |
| 12[90] | TCCTTGAGTGAGCCTTACATCGCCTCAAATATCAAGTATTAG | Core staple | 266 |
| 12[100] | TCCGTTTTTTCGTCTCGATAACGGTACAAAAGGCA | Core staple | 267 |
| 12[121] | ATCCAGCCTCCGTAACAATTTCATATAACCTTGCTTCTTTCT | Core staple | 268 |
| 14[69] | ACCGAGCAAGCCTGTTGCGTTGCGCTCAGTGG | Core staple | 269 |
| 15[46] | CGGCTTTCCAGTCGGGAGTTTGCGGCGCGCCATGC | Core staple | 270 |
| 15[98] | ACAACTCGATGATGGCAATCTCACAGTTTGACAAACAATTCG | Core staple | 271 |
| 15[109] | TAATTGAGGATTTAGAAACCCTCAAGTAACAACCAAGTAACG | Core staple | 272 |
| 15[130] | ATTAGCCGTCAATAGATAGTTGGCTTTAACGGAGGCGACAGA | Core staple | 273 |
| 17[130] | GTGCCATCCCACGCAACAAGGGTAAAGTTAAACG | Core staple | 274 |
| 16[167] | CACAGGCGGCCTTTAGTGATGCAGCTTACGGCTGGAGGTGTC | Core staple | 275 |
| 16[188] | AAAATCCCGTAAAAAAAGCCGCAGCATCAGCGGGGTCATTGC | Core staple | 276 |
| 16[205] | GTGTACATCGACATAAAAGGCGCTTTCGCACTCA | Core staple | 277 |
| 19[53] | GAGCACCAACCTAAAGAAGAGTAATCGA | Core staple | 278 |
| 19[84] | TCGCAAAAAATCGGTTGTATTAATTGCTCCATTAGTACG | Core staple | 279 |
| 18[44] | TTTTTTTGATAAGAGGTTTTTAATTCTT | Core staple | 280 |
| 21[102] | TACCAGAGCATAAAGCTTGGTCAAGTTTCCAACAGCATTCTGCTC | Core staple | 281 |
| 21[144] | ATTACAGGCAAGGCAAAGCTGAAAGAAACGTACAGCTTGCCA | Core staple | 282 |
| 20[44] | GCTAAGCAAAGCGGATTCTCAAATTAGTAAACACT | Core staple | 283 |
| 20[65] | AAAAAAGATTAAGAGGAATAAATATAGC | Core staple | 284 |
| 20[72] | AGACAAGTTGGGTAACGGGTAAAAATACATT | Core staple | 285 |
| 20[93] | CCATTTCCCAAAGGGGGAACGGCCTCAGGAATTAA | Core staple | 286 |
| 20[107] | AGAGCCGGAGAGGGTAGGTCAATCAAGCAAATAAT | Core staple | 287 |
| 20[114] | AGGAAACGACCGCTATTCTCCAGCCCAGTTTGAGGGGACGAG | Core staple | 288 |
| 20[135] | AAATTTCAGAGGCGATCCGCTTCTCGCATCGTAACCGTCTCC | Core staple | 289 |
| 23[60] | CAATATCGCGCATTTTTATGCTGTAGCTCAAGAAC | Core staple | 290 |
| 23[123] | TTTAAGGGTGCCTTTATCAAAATTAAGCAATATATTTTTAAA | Core staple | 291 |
| 22[41] | ACAGTTCTAGTCAGTCAAAGCTTGCTCCTAAATAT | Core staple | 292 |
| 22[97] | TGATAATCAGAAGGAATCGTCAGTCAACCGTTCTAGCTGATA | Core staple | 293 |
| 22[135] | AATACGTTAACAATAGGGGAACAAACGGCGGAGAT | Core staple | 294 |
| 25[32] | TTTCCAGACGAGATTCATCAGTTGTAAAACGGGCTTGAGAGC | Core staple | 295 |
| 25[60] | TTATCAACGTAAGAACCACGA | Core staple | 296 |
| 25[74] | GTCTACGAGGGCAGATACATAACGCATTATACCTTATGGCCA | Core staple | 297 |
| 24[51] | ATCGGAATACCACATTCGGGAAGAAACT | Core staple | 298 |
| 24[90] | GCTTTAAAAGGAATCAATACTGCAAGGCGATTATTTGAATTACCAGT | Core staple | 299 |
| | CA | | |
| 24[114] | TCGCAACCCGTCGGATTGCATCTGCAGCTTTCGCA | Core staple | 300 |
| 26[65] | AAAGACTGGATTCATTGAATCCCCGCAT | Core staple | 301 |
| 26[107] | CAGATTGTATATATGTACCCCGGTAATTAATCAGTCAAGTAA | Core staple | 302 |
| 27[60] | TTACGCCGGGAAAGAATACACGATTGCCACTGGATATTCTTC | Core staple | 303 |
| 27[129] | GCACGGTGCGGATTGTAACGTAAAACTAGCATCTAT | Core staple | 304 |
| 29[39] | TCAGGACAGAATTCCCAATTCTGCCATG | Core staple | 305 |
| 29[53] | GACAACAAAGTAATTTCAAAATCTACGTTAAAGAT | Core staple | 306 |
| 29[88] | GGTTCAATATGATATCCGCCCAAAAACATTATGACCCTATCA | Core staple | 307 |
| 29[130] | AGCGATTCAATGAGAGATCTACAACGGT | Core staple | 308 |
| 28[58] | AGGTAGATTTAGTTTGAGAATATAGCGGATGGCTTAGACGAA | Core staple | 309 |
| 28[72] | TAACGTCACCCTCAGCAGCGAAAGTTAAACGCCAG | Core staple | 310 |
| 28[100] | GAATAACCTGTTTAGCTAAAGCCTTTTTGCGGGAGAAGAGAA | Core staple | 311 |
| 28[114] | GACCAACGGCACAGCGGATCAAACGATCGCAACGC | Core staple | 312 |
| 28[121] | GACCATTTGGGGCGCGAGAATTAGTTCAACGCAAGGATAGGT | Core staple | 313 |
| 30[37] | CGGACTTTGAAAACGAAAGAGGCACGCGGTT | Core staple | 314 |
| 30[48] | GCGGTATGATGGTTCTGCTCAGGGGTAAGCTTTAA | Core staple | 315 |
| 30[79] | GCAGTTGGGCGGTTATCATCATTGACCC | Core staple | 316 |
| 30[90] | ATTTGCCCGATTTTATGTGCTGCAAGCCCCAAAAAGTAGCCA | Core staple | 317 |
| 30[100] | ATTCGGAACGAGGGTAGTTTTTCACGTTGTACCGG | Core staple | 318 |
| 30[121] | GAATACAGAGGCGCCATGTTTACCCACGGAAAAAGAGACCG | Core staple | 319 |
| 32[69] | GGACGTTAACTAATCATAGTAAGAGCAAATGT | Core staple | 320 |
| 33[46] | TTAATAACCCTCGTTTAGCCAGAGTTCAGTGTTCA | Core staple | 321 |
| 33[98] | ATGTGAGCGACGACAGTATGAACTGGCTCCCATCAACATTAA | Core staple | 322 |
| 33[109] | TAACGTCTGGCCTTCCTCAGGAAGCTGGCGAGTCACGATGAG | Core staple | 323 |
| 33[130] | GTGAACGCCATCAAAAATATTTAAGCCTCTTGGCCAGTTGAG | Core staple | 324 |
| 35[132] | TAAAACACTCATCTTAGGCCGCTTTTGCGG | Core staple | 325 |
| 34[224] | TAGTTGCGCCGACAATAAATTGTGTCGAAA | Core staple | 326 |
| 37[53] | CACCGACCGTGTGATCAGACGACACAAG | Core staple | 327 |
| 37[84] | AATAGAAGCACCATTACCAGGAATACCCATTTTGTAAAT | Core staple | 328 |
| 36[44] | CTTAGTTACCAGAAGGAATAAGAGATAA | Core staple | 329 |
| 36[65] | GAAGAAACGCAATAATAAGAA | Core staple | 330 |
| 39[102] | AATCAAAATCACCAGTAAATTCATGTTAATTTGTAAATCGAGGTG | Core staple | 331 |
| 39[144] | ATCTATCACCGTCACCGTCAACCGGTGAGAATAGAAACGTTA | Core staple | 332 |
| 38[44] | AAAGAGGGTAATTGAGCCAGCCTTCAGCCATTTTT | Core staple | 333 |
| 38[65] | AAGTCAGAGAGATAACCTAACGTCTCCA | Core staple | 334 |
| 38[72] | TTGTGCAGACAGCCCTCCTGACCTCACAATC | Core staple | 335 |
| 38[93] | AAAGCGTAACCAAACTAACGTATCACCGTACTTGC | Core staple | 336 |
| 38[107] | TCTAGAGCCGCCACCCTAGACGATCGCAGTCACAG | Core staple | 337 |
| 38[114] | TTTTCGTCTTCACTGAGGTTTAGTTGATATAAGTATAGTCTG | Core staple | 338 |
| 38[135] | GTCAATGAATATAGGAAAACCGCCGATAAGTGCCGTCGGAGG | Core staple | 339 |
| 41[60] | ATACCCAATAAACCGAGCTGGCATGATTAAGAAGA | Core staple | 340 |
| 41[123] | ACCCCTTATTCAGCACCCCATTTGGGAATTACCAAAGAAACT | Core staple | 341 |
| 40[41] | AGAATAAAAAGTCACAATGAACGAACAAATTACGC | Core staple | 342 |
| 40[97] | ACAAACAAATAATTTTTTGTTCAGAGCCACCACCGGAACCGC | Core staple | 343 |
| 40[135] | GGATCCAGTAACGGGGTAGACTCCTCAAGAGCCAG | Core staple | 344 |
| 43[32] | GCCTATCCTGTTATCCGGTATTCTTACCGCGCAATCAAAGCC | Core staple | 345 |
| 43[60] | TTTCCTGTTTACATGTTGAAA | Core staple | 346 |
| 43[74] | AATTTAAATCCCGACTTGCGGGAGCGAGAACGTATTAATAAA | Core staple | 347 |
| | TT | | |
| 27[12] | TTTTTACACCAGAACGAGTAGCTTGCCCGCA | Vertex staple | 448 |
| 31[14] | TTTTTATAAGGGAACCGAATGTACAGACCAGTTTTT | Vertex staple | 449 |
| 33[16] | TTTTTTTACAGGTAGAAACGATAAAAACCAAAATAGTTTTT | Vertex staple | 450 |
| 37[12] | TTTTTTACATACATAAAGGTGTAGCAAAAGTAAGCAGATAGCATAG | Vertex staple | 451 |
| 36[34] | AGTATGTGCAACATGAGAATAAGAGGCAACGAGGCGCAGACGGTCA | Vertex staple | 452 |
| | ATCTTTTT | | |
| 39[9] | TTTTTCTTTTTAAGAAACGTAGAAAATTTTT | Vertex staple | 453 |
| 38[30] | CAAAATTCTGAACAAGATAGAAACCCCAATAGCAAGCAAATCATTTT | Vertex staple | 454 |
| | T | | |
| 45[12] | TTTTTCTAATTTACGAGCATGAAAATAAGAG | Vertex staple | 455 |
| 49[14] | TTTTTCATGTAATTTAGGCTAAAGTACCGACTTTTT | Vertex staple | 456 |
| 51[16] | TTTTTGATATAGAAGGCAATCTTACCAACGCTAACGTTTTT | Vertex staple | 457 |
| 5[9] | TTTTTAAAATCCTGTTTCGTCAAAGGGCGTTTTT | Vertex staple | 458 |
| 7[24] | GGGGTGGTTTGCCCCAGCAGGCGTTTTT | Vertex staple | 459 |
| 23[9] | TTTTTAAATCAGGTCTTGCAAACTCCAACTTTTT | Vertex staple | 460 |
| 25[24] | AAAGGAGAATGACCATAAATCAATTTTT | Vertex staple | 461 |
| 41[9] | TTTTTGGGAGAATTAACCTTACCGAAGCCTTTTT | Vertex staple | 462 |
| 43[24] | CCTAACAGGGAAGCGCATTAGACTTTTT | Vertex staple | 463 |
| 7[9] | TTTTTAATCGGCCAACGTGCTGCGGCTTCACTAATCTGATGAAAAGG | Vertex bundle | 464 |
| | TAAAGTTAGCTATTGAA | strand | |
| 25[9] | TTTTTCGAGAGGCTTTTTGACGAGAAGCAAAATTCTCATTGAAATCGT | Vertex bundle | 465 |
| | TAACGACTCCAAGATG | strand | |
| | TTTTTAGCGTCTTTCCATATCCCATCTTCACTAATCTTATGTACT | | 466 |
| 43[9] | GCGCATAGGCTGACCGGAATACC | Vertex bundle strand | 467 |
| | CATCAGATTAGTGAA | Vertex bundle | 468 |
| | | strand | |
| | | (complementary) | |
| | CAATGAGAATTTTGC | Vertex bundle | 469 |
| | | strand | |
| | | (complementary) | |
| | AGTACATAAGATTAGTGAA | Vertex bundle | 470 |
| | | strand | |
| | | (complementary) | |

TABLE 6
Sequences of the cube with long connector staples.

| | | | SEQ ID |
|---|---|---|---|
| 5′-end | Sequence | Note | NO: |
| 1[84] | AACGGTATATCCAGAACAAACCACCACAGGATTTTAACGGAATGGT | Core staple | 471 |
| 0[54] | GCGCCGTAAACAGAGTGCTCGTCATAAGTTACCTGTCC | Core staple | 472 |
| 3[102] | GGAGGCCTTGCTGGTAACGCCAGACCGGCCAAGTT | Core staple | 473 |
| 3[144] | GTCAGTAATAACATCACCGAGTAAGCAAAAGAAGATTCTGCT | Core staple | 474 |
| 2[44] | ACTAAAATCCCTTATAATGAGAGACGCCAGGCTGC | Core staple | 475 |
| 2[51] | AGAGCAGCCAAGCGCAGGTTTCTGCGTAATCATGGTCAGAGC | Core staple | 476 |
| 2[72] | GTGCCTATACAGTAACATCCTCATAGACAGG | Core staple | 477 |
| 2[93] | CTGTTACATCGATTTTCTCAATTATCATCATTGAA | Core staple | 478 |
| 2[107] | AGATGGCTATTAGTCTTACACCGCACCTTGCGAGC | Core staple | 479 |
| 2[114] | CAGCGGATTCCAGAAATATTATCAAACAAAGAAACCACTTTA | Core staple | 480 |
| 2[135] | TAAAATACCACAAAATTATCAATAAGTAACATTATCATAAAC | Core staple | 481 |
| 5[25] | GTGGTTCCGATCCACGCAGAG | Core staple | 482 |
| 5[60] | AAAAGTTTGGGTGTAGCCGCTTAAT | Core staple | 483 |
| 5[123] | GCGATTCTGGAATACCTAGTAGAAGAACTCATTTTATATCGT | Core staple | 484 |
| 4[41] | CAAGCGGAATCGGCATTAAAGCGGGCGCGCGCGTA | Core staple | 485 |
| 4[83] | CAGCTGAAGTACGTAAGAAGGTATATTACCGCCAGCCATTGCTGAC | Core staple | 486 |
| 7[32] | GCGAACCTGTTCCACACAACATACTAGCTGTCGGTCATAGTA | Core staple | 487 |
| 7[74] | AGTACATTAAGGGTGCCTAATGAGGAGGATCCGCGTCCATCG | Core staple | 488 |
| 7[81] | CGGACGTCAGATGAACTTGTTCTTCCCGGGTACCGAGCAAGC | Core staple | 489 |
| 7[91] | AAATGAATAGAGCCGTCAAAGCTAACTCGAGA | Core staple | 490 |
| 7[109] | ATCCTGCAACAGTGCCATTTTGAAACCCTTCAACA | Core staple | 491 |
| 6[51] | CCGAAGCATAAAGTGTATCGAATTCCAG | Core staple | 492 |
| 6[114] | ACTGTATTAGACTTTACTTTGCGGGATGATGACAT | Core staple | 493 |
| 8[65] | CAGTTCTTTTTCACCGCCTGGCCCATCA | Core staple | 494 |
| 9[60] | CACTGCGTTACGTCAGCGTGGTGCCGTG | Core staple | 495 |
| 9[130] | TTCATTTGCACAAATATGGCGGTCAGTATTATAAT | Core staple | 496 |
| 11[88] | CTTAAAGCGTGGCACAGACAATATCGCTGAGAGCCAAA | Core staple | 497 |
| 11[130] | TTGAAGGGACCGAACTGATAGCCCGAGGTGACAAA | Core staple | 498 |
| 10[37] | CCCATCAGAGCGGGAGCCTACAGGTAGGGCGCTGGCAAAACA | Core staple | 499 |
| 10[58] | TGTGAGGCCGATTAAAGCCCGCCGGGTCACGCTGCGCGTTGA | Core staple | 500 |
| 10[65] | CCGCGGTGCCTTGTTCCGAATAGCCCGAGATTTGCCCTCACC | Core staple | 501 |
| 10[100] | CCTATCCTGAGAAGTGTAACTATCAAAACGCTCATGGACCAA | Core staple | 502 |
| 10[114] | CTCGTTCCGGTCAATATATGTGAGATTCCTGAAAGAAAAAGC | Core staple | 503 |
| 10[121] | TTTATCAGTGAGGCCACTTGCCTGACATTTTGACGCTCGTAA | Core staple | 504 |
| 13[74] | CTGGTGATGAAGGGTAAGAGCACAGTAC | Core staple | 505 |
| 13[95] | AAACCTTGCTTCTGTAAGTGAGCCAGGTTTAGCGCAGC | Core staple | 506 |
| 12[37] | TAATAATGGGTAAAGGTTTCTTAATACAAAT | Core staple | 507 |
| 12[48] | TCTTACCACCGGGGTGTCACTTATTGGGGTTGCAG | Core staple | 508 |
| 12[79] | TCGCTTTTAGTATCATAGCGTGCCGCAT | Core staple | 509 |
| 12[100] | TAACGATGCTGATTGCCGTCGCTGACAATAAAGAT | Core staple | 510 |
| 12[121] | AAACAAACGCGGGATGAAACAAACTTAATGGAAACAGTGCAA | Core staple | 511 |
| 15[46] | CGGCTTTCCAGTCGGGAGTTTGCGGCGCGCCATGCCGGACAT | Core staple | 512 |
| 15[67] | CTGTTGCGTTGCGCTCAGTGGTTTACGATCCGCGGTGCGACT | Core staple | 513 |
| 15[88] | GATAATACATTTGAGGACAGAAGGAGCGGCTCACAGTTTGTA | Core staple | 514 |
| 15[109] | GAAAACAACTAATAGATAAATCTATTGCGTAGGGAGAAGCAG | Core staple | 515 |
| 15[130] | AATTAAAATATCTTTAGTGAACCTCGTAAAAGCCTGATCGTT | Core staple | 516 |
| 17[134] | CAGCAGCAACCGCGGCGGCCTTTAGT | Core staple | 517 |
| 16[167] | TCCCGTAAAAAAAGCCGCACAAAGAATGCCAACGGCAGCACC | Core staple | 518 |
| 16[188] | GTGTACATCGACATAAAAAAAGTCGGTGGTGCCATCCCACGC | Core staple | 519 |
| 16[209] | GCCGCCAGCAGTTGGGCGGTTAACCAGCTTACGGCTGGAGGT | Core staple | 520 |
| 16[221] | TTCTGCTCATTTGTCCAGCATCAG | Core staple | 521 |
| 19[53] | CAGTTAATCATAAGGGAGCATAGGAGAC | Core staple | 522 |
| 19[84] | TTTAGTTAATAAAGCCTCATCATTTTTGTGCGAACAAGA | Core staple | 523 |
| 19[116] | GGTTCGGAACTCACCCTTCTCACGGAAAAAGCGACGACATCG | Core staple | 524 |
| 18[44] | AATTTAGAGAGTACCTTGCCCGAACTGG | Core staple | 525 |
| 18[65] | TGGTCCTTTTGATAAGACATC | Core staple | 526 |
| 21[102] | ACCTAGCAAAATTAAGCTGACCATCTAC | Core staple | 527 |
| 21[144] | CTTTAGCATTAACATCCGCTATATATAACCTCACCGAACGAC | Core staple | 528 |
| 20[44] | TTCCTTTACCCTGACTAGTCATAAAAGAAGTAATT | Core staple | 529 |
| 20[65] | TTACAGAAGCAAAGCGGAGCGTCCTAATAGTCAGA | Core staple | 530 |
| 20[72] | AAATAGGGGGATGTGCTAGGACTAGAGTAGA | Core staple | 531 |
| 20[93] | GAAGATTAAGCTTCGCTTTAGTTTGAGGGGAAGAC | Core staple | 532 |
| 20[107] | ATTAACCGTTCTAGCTGGAACGGTGCCCCAAAACC | Core staple | 533 |
| 20[114] | GGTGGTTTTCAAGGGCGAGTATCGGGGCGCATCGTAACGCTT | Core staple | 534 |
| 20[135] | GCAGTAAAACTCAGGCTGCACTCCATAGGTCACGTTGGGAGC | Core staple | 535 |
| 23[25] | TAAATCAAAACCCCTCAAATA | Core staple | 536 |
| 23[60] | AGTAGAGGAATAATTGCCTTAGAGCTTAATTATAA | Core staple | 537 |
| 23[123] | ATTAGTAATGCCTGTAACATACAGGCAAGGCAAAT | Core staple | 538 |
| 22[41] | TTGAATCATCAGGTAAATATCGTCAGGAATAATGC | Core staple | 539 |
| 22[97] | CATGTCAATCATAGACTGGATATGTCAAATCACCATCAATAT | Core staple | 540 |
| 25[32] | GCGCAACACTGGAACAACATTATTGTTGGGAAACACCAGCCG | Core staple | 541 |
| 25[60] | CCAAGAACCGACCTTCAAGGAAGTTTGATTCCCAATTCCGGA | Core staple | 542 |
| 24[51] | ACGGAAAGATTCATCAGGCTCATTTTGGGCTAGG | Core staple | 543 |
| 24[72] | TACTTAGGAATACCACACTTATGCTTCAACTAACT | Core staple | 544 |
| 24[90] | TCGCGCAACTAATGAAAATGTCAGCTGGCGAAAATGTTT | Core staple | 545 |
| 24[114] | AATTCAACATTAAATGTTGTAGATGCCTCAGGGAT | Core staple | 546 |
| 26[65] | ACAGAGGGGGAATACTGCGGAATCTTAT | Core staple | 547 |
| 26[86] | CGCTTATGTACCCCGGTAAATAAT | Core staple | 548 |
| 26[107] | GTGCAGAAAAAATCGTAAAACTAGGATATTCCAAAAGGTTGT | Core staple | 549 |
| 27[74] | AATGATTTTAAGAACTGTTGAGATATAACGCCAAAAGGTTTG | Core staple | 550 |
| 27[129] | GATCGCGCAACAAGATTGACAAGAGAATCGATATAA | Core staple | 551 |
| 29[39] | GGCACCGAACAAGTTTCATTCCATGCTG | Core staple | 552 |
| 29[53] | CTGGATATTCTAGTAAAATACCAGTCAGGACACAG | Core staple | 553 |
| 29[88] | GGCAGGCCGGAGACATGGGGAGCATAAAGCTAAATCGGGTGA | Core staple | 554 |
| 29[102] | GTAGCAACGGTAGATACATTTCGCAAAGAATAAAAACATTATGACTGT | Core staple | 555 |
| | A | | |
| 29[130] | GTTATGCCTGAATGCCGGAGAGGGGGAGCAATATA | Core staple | 556 |
| 28[72] | CTTATACGTAATTGCAGGGAGTTAGGCTTTGGCAA | Core staple | 557 |
| 28[93] | AGAAAGGCCGGAAACAGCGGATCATTAATCAATTA | Core staple | 558 |
| 28[121] | GCACAATAACCTGTTTAAATAAATTACTTTTGCGGGAGAAAT | Core staple | 559 |
| 30[37] | GGCGAACGAGGCGCAGACGGTCCCTTCGCAC | Core staple | 560 |
| 30[48] | TCAATCCGAACGAGATTACCCTTTGCAAATATTCA | Core staple | 561 |
| 30[59] | CGCTATTAAACGGGTAAATTTCATGTCAAGAGAAGA | Core staple | 562 |
| 30[79] | TAAATCGGGGTCATTGCTGAGATGCTTG | Core staple | 563 |
| 30[100] | GCACTTTTGCGGGATCGGAGGGTAACGCCAGAAAG | Core staple | 564 |
| 30[121] | AGCCAGCAGCGAGAAACAATCGGCTCTCCGTGGTGAAGGAA | Core staple | 565 |
| 33[46] | GTAAGGCATAGTAAGAGAGAGGCTAAATCAAACCA | Core staple | 566 |
| 33[91] | CCTTCCTGTAGCCACGTGCATCTGCCGTGAATTACTTTCTGG | Core staple | 567 |
| 33[109] | TCAAGGAACGCCATCAATGATAATCGGGCCTTTGG | Core staple | 568 |
| 33[130] | GAGTCAGCTCATTTTTTAAACAGGTGTTGGGCCAGTCAGACA | Core staple | 569 |
| 35[134] | GCCACTACGAAGGGGTCGCTGAGGCT | Core staple | 570 |
| 34[167] | CCACGCATAACCGATATATTCCACCAACCTAAAACGAAAGAG | Core staple | 571 |
| 34[188] | GACAATGACAACAACCATCGCGCAAAAGAATACACTAAAACA | Core staple | 572 |
| 34[209] | CTTGATACCGATAGTTGCGCCCTCATCTTTGACCCCCAGCGA | Core staple | 573 |
| 34[221] | TTTCTTAAACAGTTATACCAAGCG | Core staple | 574 |
| 37[53] | AAGTTATTTAGGCAGAGAATTCTGCCCA | Core staple | 575 |
| 37[84] | ATTTTGTCAAAATCACCAGAAC | Core staple | 576 |
| 37[116] | TTTATGTAAAGGCTTAGGAGCCTTTAATTGTGTGTATCACCG | Core staple | 577 |
| 36[44] | CATAGATAGCCGAACAAAGTTAAGTCCAGACGAAC | Core staple | 578 |
| 36[65] | CGGAGAAGGAAACCGAGAGAG | Core staple | 579 |
| 36[75] | GCAATACACGGAAGAGAAAATCTGACCTATCATA | Core staple | 580 |
| 39[102] | CCGGGAATTAGAGCCAGCACAATCCAATCGCGAGACTATATCAGC | Core staple | 581 |
| 39[144] | TCACATTAAAGGTGAATCAAAAGGACAGTTTCAGCGTATCGT | Core staple | 582 |
| 38[44] | ATACCTGAACAAAGTCAAAAAATGAGTTACAAAGA | Core staple | 583 |
| 38[65] | ACAATTGAGCGCTAATAAACGATTATTATTTGAGG | Core staple | 584 |
| 38[72] | ATAACCCTGTAGCATTCAGAACGCTAAGTTT | Core staple | 585 |
| 38[83] | ATCAAAGGATAGCACCATTACCATTAGCGCCA | Core staple | 586 |
| 38[93] | TCTAGCCCTCTTTCGTCGTAGCCCGGAATAGATCG | Core staple | 587 |
| 38[107] | ATTGAACCGCCTCCCTCGGTTGAGGCCAGAACAGT | Core staple | 588 |
| 38[114] | CCCGATCTAACCCATGTACCGTACGCCGTCGAGAGGGTTCGG | Core staple | 589 |
| 38[135] | CATTCCAGACGGATAGCACCGCCACTCAGTACCAGGCGCATG | Core staple | 590 |
| 41[25] | GAGAATTAACTACAGAGCTTT | Core staple | 591 |
| 41[60] | GTAAGAATTGAGTTACCAATACCCAAAAGAAATAA | Core staple | 592 |
| 41[123] | CCGTTCGGTCGAAACCAGTCACCGACTTGAGATGG | Core staple | 593 |
| 40[41] | CAGCCTTTGAACACATAAGAGAGTAAGCGATTAAG | Core staple | 594 |
| 40[97] | TGGCCTTGATATCAAATAAGATCAATCACCGGAACCAGAGCC | Core staple | 595 |
| 43[32] | CCACCCAGCTCAGATATAGAAGGCATCGTAGGAGCATGCCTG | Core staple | 596 |
| 43[60] | AAATAATGCAGACGACAAAATATAAAACGCAAAGACACATAA | Core staple | 597 |
| 43[130] | GTCCAGCATTGACAGGAAGAG | Core staple | 598 |
| 42[51] | TTAGTATTCTAAGAACGAAGCAAGTAATCGGCAAC | Core staple | 599 |
| 42[72] | TTTTTTTAGCGAACCTCAGTACCGCATTCCACGAGGTGAACGAAA | Core staple | 600 |
| 42[90] | AACAGGACTTGCGGATCCCAACAAACTACAACGATTCCT | Core staple | 601 |
| 42[114] | GCCCTATTATTCTGAAAGATAAGTTCAGGAGCCAAAAGGTTGGGT | Core staple | 602 |
| 44[51] | GCGCAATCAACCGTTTTTATTTTCTTAT | Core staple | 603 |
| 44[107] | TAACATTAAAGCAGGTCAGACGATACCACCGAGCGTTTAAGG | Core staple | 604 |
| 45[74] | TATCACTCATCGAGAACCGAGGCGTGAAGCCTTAAATCAAAT | Core staple | 605 |
| 47[39] | AGTGCATTTTAAAGGTGGCAACATCTGG | Core staple | 606 |
| 47[102] | TTAGCAAATCAATAGAAAATTCATCCATTTGGAAACGTCACCAATATAG | Core staple | 607 |
| 47[130] | CTTCGGCATTCCACCCTCAGAACCCCGCCGCTCTGAATGGTA | Core staple | 608 |
| 46[121] | TATACCAGCGCCAAAGATATCACCTCGATAGCAGCACCTTTT | Core staple | 609 |
| 49[84] | GGTCTGAAAGACAACACAGACTTTCATA | Core staple | 610 |
| 49[126] | TAGAGTGAGAATAGCCAAAAAAAAGGCTGTTTAGTAAGCCCACGCA | Core staple | 611 |
| 48[37] | ATATTAACAACGCCAACATGTATTGATTTGT | Core staple | 612 |
| 48[48] | ATCATCGTAGAAACCCTGTTTATTTGCCAAAATAG | Core staple | 613 |
| 48[58] | GGAAGTTAATTTCATCTCTTTTTCATAAACAACCC | Core staple | 614 |
| 48[69] | CAAAGTACTGTCTTGTTCAGCCAGCCATTTTTGTTTAACGTCGAGG | Core staple | 615 |
| 48[90] | TTGCTTTAGAACGGACCAGTATCTCACAAACAAATCCGTATA | Core staple | 616 |
| 48[100] | GTTCCTTTTTAACCTCCTGCTGATGCGTAACCCTT | Core staple | 617 |
| 50[104] | TGATATAAGTATATTAAACCACCTTAATGCCCCCTGCCTATT | Core staple | 618 |
| 51[46] | CCGGTTGCTATTTTGCAGAGCCTAATCAACAGTAA | Core staple | 619 |
| 51[109] | AACTTGAGTAACAGTGCAAATCCTCACTGAGATAG | Core staple | 620 |
| 51[130] | AAAAGTTTTAACGGGGTTGGAAAGATAGGAAAGTTTTGTAAC | Core staple | 621 |
| 53[134] | AATTTAATGGTTTGAATTTATCAAAA | Core staple | 622 |
| 52[167] | ACGCTGAGAAGAGTCAATAGTGAAATACCGACCGTGTGATAA | Core staple | 623 |
| 52[188] | ATAGCGATAGCTTAGATTAAGATAAGGCGTTAAATAAGAATA | Core staple | 624 |
| 52[209] | TCCCTTAGAATCCTTGAAAACAACACCGGAATCATAATTACT | Core staple | 625 |
| 52[221] | ATTAATTAATTTAGAAAAAGCCTG | Core staple | 626 |
| 7[137] | CCCGGTTATCTCGACAACTCGTATAAGTTTGTAATCCTACCT | Core staple | 627 |
| 7[151] | CTGCAGAAGATAAAACATAAAACAACGACCAAATC | Core staple | 628 |
| 6[146] | TGAGGAATCAATCAACCATATAGTTACATACCTGAAAGAGTC | Core staple | 629 |
| 12[142] | TTTATCAAGAAAACAAATTTCAATAAATCGCCAGTCAC | Core staple | 630 |
| 12[163] | ACAATTTCATTTGAATTGATTGTTAGAACCTATAT | Core staple | 631 |
| 14[160] | GTTATTAATTTTAATAAATCCAAGGAAT | Core staple | 632 |
| 25[137] | AGCTGTTAAATAACAACCCGTCGGTAATGGGAGCCAGCTAGA | Core staple | 633 |
| 25[151] | TTGTTGCCTGAGAGTCTTAGCTATATATTTTAAGC | Core staple | 634 |
| 24[146] | AAATTTTAAATATTTCGCCATGACGGCCGGAACGGTTTCATT | Core staple | 635 |
| 30[142] | CTTGAAACGTACAGCGCCGCCACGAGTGCCACCCTCAT | Core staple | 636 |
| 30[163] | CCGGAATTTGTGAGAGATTTCCGGGCGCCATTAAA | Core staple | 637 |
| 32[160] | CGGCGGATTGACCGATTCTCCTCGCATT | Core staple | 638 |
| 43[151] | GTAAACCACCACCAGAGGCCACCCTAGCGCGGTAA | Core staple | 639 |
| 42[135] | ATAGTATTAAGAGGCTGGGTTTTGCCCTCAGAAAA | Core staple | 640 |
| 42[146] | GTGTACTTTACCGTTTTTCAGGTTAGTAACTTTCAGCGACAT | Core staple | 641 |
| 48[142] | TCTAAAGGAACAACTAACTAAACAAATGAATCAGACTG | Core staple | 642 |
| 48[163] | ATAATTTTTTCACGTTGAACCGCCACCCTCATCCA | Core staple | 643 |
| 50[160] | ATTAGGATTAGCGGAGACTCCTACAGGA | Core staple | 644 |
| 10[160] | TTATTCAATTAATTACATTTA | Connector staple | 645 |
| 28[160] | GTGGAGCCATGTTTACCAGTA | Connector staple | 646 |
| 46[160] | GATTTTGAGGAATTGCGAATC | Connector staple | 647 |
| 8[166] | TAATGGAAGGGTTTGGATTATACTTCTGAA | Connector staple | 648 |
| 26[166] | GAAACCAGGCAAACACCGCTTCTGGTGCGG | Connector staple | 649 |
| 44[166] | CCTCAGAGCCACCACCCTCAGAACCGCCAG | Connector staple | 650 |
| 2[163] | GCAGATTCACGCAGAGGCGAA | Connector staple | 651 |
| 20[163] | ATTTTTAGAAAGCTTTCAGAC | Connector staple | 652 |
| 38[163] | CCTTTAGCGTTTTCTGTATCG | Connector staple | 653 |
| 4[163] | GAACCACCAGGTCAGTTGGCAATG | Connector staple | 654 |
| 22[163] | TATCAGGTCATAAACGTTAATATG | Connector staple | 655 |
| 40[163] | CCGCCACCAGAGCGTCATACATAA | Connector staple | 656 |
| 5[147] | TCGCCATTAAAAATACCGAAC | Connector staple | 657 |
| 23[147] | TTTTGAGAGATCTACAAAGAG | Connector staple | 658 |
| 41[147] | TCAGAGCCACCACCCTCAGGC | Connector staple | 659 |
| 1[147] | TGTCCATTTTGATTTGAAATGGATTATTTACATAT | Connector staple | 660 |
| 19[147] | TGGGGCGATAGTAGTATTTCAACGCAAGGATAAGG | Connector staple | 661 |
| 37[147] | TCAACCGAATTATTGTAGCGACAGAATCAAGTTTT | Connector staple | 662 |
| 6[163] | CAACAGTTGATTTGCCCGATT | Connector staple | 663 |
| 24[163] | TTGTTAAAATGTGGGAACAGT | Connector staple | 664 |
| 42[163] | CTTTTGATGATCAAGAGAAGC | Connector staple | 665 |
| 0[166] | GTAGCAATACTTCCACGCAAATTAACCGAC | Connector staple | 666 |
| 18[166] | ATCAATTCTACTACGAGCTGAAAAGGTGGG | Connector staple | 667 |
| 36[166] | AAATATTGACGGAATTGAGGGAGGGAAGAA | Connector staple | 668 |
| 9[12] | TTTTTCAGAATGCGGCGGGCCTCTGTGGCGC | Vertex staple | 669 |
| 15[16] | TTTTTTCCGCTCACAATCGTGCCAGCTGCATTAATGTTTTT | Vertex staple | 670 |
| 38[30] | AAAACAAAAGATAGATAAATTTACGAATCATTACCGCGCCCAATTTTT | Vertex staple | 671 |
| 36[34] | ACTCCTTCATACATCGAGCCAGCCATATAATTGTGTCGAAATCCGCGAC | Vertex staple | 672 |
| | TTTTT | | |
| 49[14] | TTTTTCTTAATTGAGAATCGTAATAAGAGAATTTTT | Vertex staple | 673 |
| 45[12] | TTTTTAATAATATCCCATCCTAGTCCTGCGA | Vertex staple | 674 |
| 51[16] | TTTTTTAGCAAGCAAATACAATTTTATCCTGAATCTTTTTT | Vertex staple | 675 |
| 37[12] | TTTTTGCAAACGTAGAAAATAATTACGCCCCTTTTTAAGAAACAAG | Vertex staple | 676 |
| 39[9] | TTTTTATCTTACCGAAGAGTATGTTATTTTT | Vertex staple | 677 |
| 20[31] | TTTTTGTACAGCGTAACAGACGAGAAGAAAAATCTACGTTAATATTTTT | Vertex staple | 678 |
| 18[34] | TGTAGCTTGTCTGGTGACCAATTAGCCGGCGGTTGCGGTATGAGCCGGG | Vertex staple | 679 |
| | TTTTT | | |
| 31[14] | TTTTTCTGCTCCATGTTACCTTTGAAAGAGGTTTTT | Vertex staple | 680 |
| 27[12] | TTTTTGAATAAGGCTTGCCCTAAGCTGCAAA | Vertex staple | 681 |
| 33[16] | TTTTTAAACGAACTAACATCATAACCCTCGTTTACCTTTTT | Vertex staple | 682 |
| 19[12] | TTTTTTGCAACTAAAGTACGGCAACATGGCAAACTCCAACAGGCG | Vertex staple | 683 |
| 1[12] | TTTTTTATAACGTGCTTTCCTTGCTTTGTCAAGCGAAAGGAGAACG | Vertex staple | 684 |
| 21[9] | TTTTTACCAGACCGGAATTTTAAATATTTTT | Vertex staple | 685 |
| 2[30] | TGGGCATCAGTGTGCACGTTTTCATTCCTGTGTGAAATTGTTATTTTT | Vertex staple | 686 |
| 0[34] | CTATGGTCGTTAGATTACACTCGGCTGGAGCCAACGCTCAACAGTAGG | Vertex staple | 687 |
| | GTTTTT | | |
| 13[14] | TTTTTTCACTGTTGCCCTGGGTGTGTTCAGCTTTTT | Vertex staple | 688 |
| 3[9] | TTTTTAAAAACCGTCTAACGAGCACGTTTTT | Vertex staple | 689 |
| 7[24] | GGGGTGGTTTGCCCCAGCAGGCGTTCACTAATCTGATGGAAGCGCATTA | Vertex bundle | 690 |
| | GATAGCAATAGCTTTTTT | strand | |
| 25[24] | CCAAAATGCTTTAAACAGTTCAGGCAAAATTCTCATTGAAAATCCTGTT | Vertex bundle | 691 |
| | TCGTCAAAGGGCGTTTTT | strand | |
| 43[24] | GCGTAGAATAACATAAAAACAGGAATGTCGATATCTAGAAAACGAGAA | Vertex bundle | 692 |
| | TGGCTTCAAAGCGATTTTT | strand | |
| 7[9] | TTTTTAATCGGCCAACGTGCTGCGGCTTCACTAATCTGATGTATAAAGT | Vertex bundle | 693 |
| | ACCGCAATGAAACGG | strand | |
| 25[9] | TTTTTAGACGACGATAATCATTCAGTGCAAAATTCTCATTGAAATCGTT | Vertex bundle | 694 |
| | AACGACTCCAAGATG | strand | |
| 43[9] | TTTTTTACCAACGCTAAAACAAGAAAAATGTCGATATCTAGACAGATG | Vertex bundle | 695 |
| | AACGGAATTCGAACCA | strand | |
| | CATCAGATTAGTGAA | Vertex bundle | 696 |
| | | strand | |
| | | (complementary) | |
| | CAATGAGAATTTTGC | Vertex bundle | 697 |
| | | strand | |
| | | (complementary) | |
| | CTAGATATCGACATT | Vertex bundle | 698 |
| | | strand | |
| | | (complementary) | |

TABLE 7
Sequences of the cube with short connector staples.

| | | | SEQ ID |
|---|---|---|---|
| 5′-end | Sequence | Note | NO: |
| 1[84] | AACGGTATATCCAGAACAAACCACCACAGGATTTTAACGGAATGGT | Core staple | 699 |
| 0[54] | GCGCCGTAAACAGAGTGCTCGTCATAAGTTACCTGTCC | Core staple | 700 |
| 3[102] | GGAGGCCTTGCTGGTAACGCCAGACCGGCCAAGTT | Core staple | 701 |
| 3[144] | GTCAGTAATAACATCACCGAGTAAGCAAAAGAAGATTCTGCT | Core staple | 702 |
| 2[44] | ACTAAAATCCCTTATAATGAGAGACGCCAGGCTGC | Core staple | 703 |
| 2[51] | AGAGCAGCCAAGCGCAGGTTTCTGCGTAATCATGGTCAGAGC | Core staple | 704 |
| 2[72] | GTGCCTATACAGTAACATCCTCATAGACAGG | Core staple | 705 |
| 2[93] | CTGTTACATCGATTTTCTCAATTATCATCATTGAA | Core staple | 706 |
| 2[107] | AGATGGCTATTAGTCTTACACCGCACCTTGCGAGC | Core staple | 707 |
| 2[114] | CAGCGGATTCCAGAAATATTATCAAACAAAGAAACCACTTTA | Core staple | 708 |
| 2[135] | TAAAATACCACAAAATTATCAATAAGTAACATTATCATAAAC | Core staple | 709 |
| 5[25] | GTGGTTCCGATCCACGCAGAG | Core staple | 710 |
| 5[60] | AAAAGTTTGGGTGTAGCCGCTTAAT | Core staple | 711 |
| 5[123] | GCGATTCTGGAATACCTAGTAGAAGAACTCATTTTATATCGT | Core staple | 712 |
| 4[41] | CAAGCGGAATCGGCATTAAAGCGGGCGCGCGCGTA | Core staple | 713 |
| 4[83] | CAGCTGAAGTACGTAAGAAGGTATATTACCGCCAGCCATTGCTGAC | Core staple | 714 |
| 7[32] | GCGAACCTGTTCCACACAACATACTAGCTGTCGGTCATAGTA | Core staple | 715 |
| 7[74] | AGTACATTAAGGGTGCCTAATGAGGAGGATCCGCGTCCATCG | Core staple | 716 |
| 7[81] | CGGACGTCAGATGAACTTGTTCTTCCCGGGTACCGAGCAAGC | Core staple | 717 |
| 7[91] | AAATGAATAGAGCCGTCAAAGCTAACTCGAGA | Core staple | 718 |
| 7[109] | ATCCTGCAACAGTGCCATTTTGAAACCCTTCAACA | Core staple | 719 |
| 6[51] | CCGAAGCATAAAGTGTATCGAATTCCAG | Core staple | 720 |
| 6[114] | ACTGTATTAGACTTTACTTTGCGGGATGATGACAT | Core staple | 721 |
| 8[65] | CAGTTCTTTTTCACCGCCTGGCCCATCA | Core staple | 722 |
| 9[60] | CACTGCGTTACGTCAGCGTGGTGCCGTG | Core staple | 723 |
| 9[130] | TTCATTTGCACAAATATGGCGGTCAGTATTATAAT | Core staple | 724 |
| 11[88] | CTTAAAGCGTGGCACAGACAATATCGCTGAGAGCCAAA | Core staple | 725 |
| 11[130] | TTGAAGGGACCGAACTGATAGCCCGAGGTGACAAA | Core staple | 726 |
| 10[37] | CCCATCAGAGCGGGAGCCTACAGGTAGGGCGCTGGCAAAACA | Core staple | 727 |
| 10[58] | TGTGAGGCCGATTAAAGCCCGCCGGGTCACGCTGCGCGTTGA | Core staple | 728 |
| 10[65] | CCGCGGTGCCTTGTTCCGAATAGCCCGAGATTTGCCCTCACC | Core staple | 729 |
| 10[100] | CCTATCCTGAGAAGTGTAACTATCAAAACGCTCATGGACCAA | Core staple | 730 |
| 10[114] | CTCGTTCCGGTCAATATATGTGAGATTCCTGAAAGAAAAAGC | Core staple | 731 |
| 10[121] | TTTATCAGTGAGGCCACTTGCCTGACATTTTGACGCTCGTAA | Core staple | 732 |
| 13[74] | CTGGTGATGAAGGGTAAGAGCACAGTAC | Core staple | 733 |
| 13[95] | AAACCTTGCTTCTGTAAGTGAGCCAGGTTTAGCGCAGC | Core staple | 734 |
| 12[37] | TAATAATGGGTAAAGGTTTCTTAATACAAAT | Core staple | 735 |
| 12[48] | TCTTACCACCGGGGTGTCACTTATTGGGGTTGCAG | Core staple | 736 |
| 12[79] | TCGCTTTTAGTATCATAGCGTGCCGCAT | Core staple | 737 |
| 12[100] | TAACGATGCTGATTGCCGTCGCTGACAATAAAGAT | Core staple | 738 |
| 12[121] | AAACAAACGCGGGATGAAACAAACTTAATGGAAACAGTGCAA | Core staple | 739 |
| 15[46] | CGGCTTTCCAGTCGGGAGTTTGCGGCGCGCCATGCCGGACAT | Core staple | 740 |
| 15[67] | CTGTTGCGTTGCGCTCAGTGGTTTACGATCCGCGGTGCGACT | Core staple | 741 |
| 15[88] | GATAATACATTTGAGGACAGAAGGAGCGGCTCACAGTTTGTA | Core staple | 742 |
| 15[109] | GAAAACAACTAATAGATAAATCTATTGCGTAGGGAGAAGCAG | Core staple | 743 |
| 15[130] | AATTAAAATATCTTTAGTGAACCTCGTAAAAGCCTGATCGTT | Core staple | 744 |
| 17[134] | CAGCAGCAACCGCGGCGGCCTTTAGT | Core staple | 745 |
| 16[167] | TCCCGTAAAAAAAGCCGCACAAAGAATGCCAACGGCAGCACC | Core staple | 746 |
| 16[188] | GTGTACATCGACATAAAAAAAGTCGGTGGTGCCATCCCACGC | Core staple | 747 |
| 16[209] | GCCGCCAGCAGTTGGGCGGTTAACCAGCTTACGGCTGGAGGT | Core staple | 748 |
| 16[221] | TTCTGCTCATTTGTCCAGCATCAG | Core staple | 749 |
| 19[53] | CAGTTAATCATAAGGGAGCATAGGAGAC | Core staple | 750 |
| 19[84] | TTTAGTTAATAAAGCCTCATCATTTTTGTGCGAACAAGA | Core staple | 751 |
| 19[116] | GGTTCGGAACTCACCCTTCTCACGGAAAAAGCGACGACATCG | Core staple | 752 |
| 18[44] | AATTTAGAGAGTACCTTGCCCGAACTGG | Core staple | 753 |
| 18[65] | TGGTCCTTTTGATAAGACATC | Core staple | 754 |
| 21[102] | ACCTAGCAAAATTAAGCTGACCATCTAC | Core staple | 755 |
| 21[144] | CTTTAGCATTAACATCCGCTATATATAACCTCACCGAACGAC | Core staple | 756 |
| 20[44] | TTCCTTTACCCTGACTAGTCATAAAAGAAGTAATT | Core staple | 757 |
| 20[65] | TTACAGAAGCAAAGCGGAGCGTCCTAATAGTCAGA | Core staple | 758 |
| 20[72] | AAATAGGGGGATGTGCTAGGACTAGAGTAGA | Core staple | 759 |
| 20[93] | GAAGATTAAGCTTCGCTTTAGTTTGAGGGGAAGAC | Core staple | 760 |
| 20[107] | ATTAACCGTTCTAGCTGGAACGGTGCCCCAAAACC | Core staple | 761 |
| 20[114] | GGTGGTTTTCAAGGGCGAGTATCGGGGCGCATCGTAACGCTT | Core staple | 762 |
| 20[135] | GCAGTAAAACTCAGGCTGCACTCCATAGGTCACGTTGGGAGC | Core staple | 763 |
| 23[25] | TAAATCAAAACCCCTCAAATA | Core staple | 764 |
| 23[60] | AGTAGAGGAATAATTGCCTTAGAGCTTAATTATAA | Core staple | 765 |
| 23[123] | ATTAGTAATGCCTGTAACATACAGGCAAGGCAAAT | Core staple | 766 |
| 22[41] | TTGAATCATCAGGTAAATATCGTCAGGAATAATGC | Core staple | 767 |
| 22[97] | CATGTCAATCATAGACTGGATATGTCAAATCACCATCAATAT | Core staple | 768 |
| 25[32] | GCGCAACACTGGAACAACATTATTGTTGGGAAACACCAGCCG | Core staple | 769 |
| 25[60] | CCAAGAACCGACCTTCAAGGAAGTTTGATTCCCAATTCCGGA | Core staple | 770 |
| 24[51] | ACGGAAAGATTCATCAGGCTCATTTTGGGCTAGG | Core staple | 771 |
| 24[72] | TACTTAGGAATACCACACTTATGCTTCAACTAACT | Core staple | 772 |
| 24[90] | TCGCGCAACTAATGAAAATGTCAGCTGGCGAAAATGTTT | Core staple | 773 |
| 24[114] | AATTCAACATTAAATGTTGTAGATGCCTCAGGGAT | Core staple | 774 |
| 26[65] | ACAGAGGGGGAATACTGCGGAATCTTAT | Core staple | 775 |
| 26[86] | CGCTTATGTACCCCGGTAAATAAT | Core staple | 776 |
| 26[107] | GTGCAGAAAAAATCGTAAAACTAGGATATTCCAAAAGGTTGT | Core staple | 777 |
| 27[74] | AATGATTTTAAGAACTGTTGAGATATAACGCCAAAAGGTTTG | Core staple | 778 |
| 27[129] | GATCGCGCAACAAGATTGACAAGAGAATCGATATAA | Core staple | 779 |
| 29[39] | GGCACCGAACAAGTTTCATTCCATGCTG | Core staple | 780 |
| 29[53] | CTGGATATTCTAGTAAAATACCAGTCAGGACACAG | Core staple | 781 |
| 29[88] | GGCAGGCCGGAGACATGGGGAGCATAAAGCTAAATCGGGTGA | Core staple | 782 |
| 29[102] | GTAGCAACGGTAGATACATTTCGCAAAGAATAAAAACATTATGACTGTA | Core staple | 783 |
| 29[130] | GTTATGCCTGAATGCCGGAGAGGGGGAGCAATATA | Core staple | 784 |
| 28[72] | CTTATACGTAATTGCAGGGAGTTAGGCTTTGGCAA | Core staple | 785 |
| 28[93] | AGAAAGGCCGGAAACAGCGGATCATTAATCAATTA | Core staple | 786 |
| 28[121] | GCACAATAACCTGTTTAAATAAATTACTTTTGCGGGAGAAAT | Core staple | 787 |
| 30[37] | GGCGAACGAGGCGCAGACGGTCCCTTCGCAC | Core staple | 788 |
| 30[48] | TCAATCCGAACGAGATTACCCTTTGCAAATATTCA | Core staple | 789 |
| 30[59] | CGCTATTAAACGGGTAAATTTCATGTCAAGAGAAGA | Core staple | 790 |
| 30[79] | TAAATCGGGGTCATTGCTGAGATGCTTG | Core staple | 791 |
| 30[100] | GCACTTTTGCGGGATCGGAGGGTAACGCCAGAAAG | Core staple | 792 |
| 30[121] | AGCCAGCAGCGAGAAACAATCGGCTCTCCGTGGTGAAGGAA | Core staple | 793 |
| 33[46] | GTAAGGCATAGTAAGAGAGAGGCTAAATCAAACCA | Core staple | 794 |
| 33[91] | CCTTCCTGTAGCCACGTGCATCTGCCGTGAATTACTTTCTGG | Core staple | 795 |
| 33[109] | TCAAGGAACGCCATCAATGATAATCGGGCCTTTGG | Core staple | 796 |
| 33[130] | GAGTCAGCTCATTTTTTAAACAGGTGTTGGGCCAGTCAGACA | Core staple | 797 |
| 35[134] | GCCACTACGAAGGGGTCGCTGAGGCT | Core staple | 798 |
| 34[167] | CCACGCATAACCGATATATTCCACCAACCTAAAACGAAAGAG | Core staple | 799 |
| 34[188] | GACAATGACAACAACCATCGCGCAAAAGAATACACTAAAACA | Core staple | 800 |
| 34[209] | CTTGATACCGATAGTTGCGCCCTCATCTTTGACCCCCAGCGA | Core staple | 801 |
| 34[221] | TTTCTTAAACAGTTATACCAAGCG | Core staple | 802 |
| 37[53] | AAGTTATTTAGGCAGAGAATTCTGCCCA | Core staple | 803 |
| 37[84] | ATTTTGTCAAAATCACCAGAAC | Core staple | 804 |
| 37[116] | TTTATGTAAAGGCTTAGGAGCCTTTAATTGTGTGTATCACCG | Core staple | 805 |
| 36[44] | CATAGATAGCCGAACAAAGTTAAGTCCAGACGAAC | Core staple | 806 |
| 36[65] | CGGAGAAGGAAACCGAGAGAG | Core staple | 807 |
| 36[75] | GCAATACACGGAAGAGAAAATCTGACCTATCATA | Core staple | 808 |
| 39[102] | CCGGGAATTAGAGCCAGCACAATCCAATCGCGAGACTATATCAGC | Core staple | 809 |
| 39[144] | TCACATTAAAGGTGAATCAAAAGGACAGTTTCAGCGTATCGT | Core staple | 810 |
| 38[44] | ATACCTGAACAAAGTCAAAAAATGAGTTACAAAGA | Core staple | 811 |
| 38[65] | ACAATTGAGCGCTAATAAACGATTATTATTTGAGG | Core staple | 812 |
| 38[72] | ATAACCCTGTAGCATTCAGAACGCTAAGTTT | Core staple | 813 |
| 38[83] | ATCAAAGGATAGCACCATTACCATTAGCGCCA | Core staple | 814 |
| 38[93] | TCTAGCCCTCTTTCGTCGTAGCCCGGAATAGATCG | Core staple | 815 |
| 38[107] | ATTGAACCGCCTCCCTCGGTTGAGGCCAGAACAGT | Core staple | 816 |
| 38[114] | CCCGATCTAACCCATGTACCGTACGCCGTCGAGAGGGTTCGG | Core staple | 817 |
| 38[135] | CATTCCAGACGGATAGCACCGCCACTCAGTACCAGGCGCATG | Core staple | 818 |
| 41[25] | GAGAATTAACTACAGAGCTTT | Core staple | 819 |
| 41[60] | GTAAGAATTGAGTTACCAATACCCAAAAGAAATAA | Core staple | 820 |
| 41[123] | CCGTTCGGTCGAAACCAGTCACCGACTTGAGATGG | Core staple | 821 |
| 40[41] | CAGCCTTTGAACACATAAGAGAGTAAGCGATTAAG | Core staple | 822 |
| 40[97] | TGGCCTTGATATCAAATAAGATCAATCACCGGAACCAGAGCC | Core staple | 823 |
| 43[32] | CCACCCAGCTCAGATATAGAAGGCATCGTAGGAGCATGCCTG | Core staple | 824 |
| 43[60] | AAATAATGCAGACGACAAAATATAAAACGCAAAGACACATAA | Core staple | 825 |
| 43[130] | GTCCAGCATTGACAGGAAGAG | Core staple | 826 |
| 42[51] | TTAGTATTCTAAGAACGAAGCAAGTAATCGGCAAC | Core staple | 827 |
| 42[72] | TTTTTTTAGCGAACCTCAGTACCGCATTCCACGAGGTGAACGAAA | Core staple | 828 |
| 42[90] | AACAGGACTTGCGGATCCCAACAAACTACAACGATTCCT | Core staple | 829 |
| 42[114] | GCCCTATTATTCTGAAAGATAAGTTCAGGAGCCAAAAGGTTGGGT | Core staple | 830 |
| 44[51] | GCGCAATCAACCGTTTTTATTTTCTTAT | Core staple | 831 |
| 44[107] | TAACATTAAAGCAGGTCAGACGATACCACCGAGCGTTTAAGG | Core staple | 832 |
| 45[74] | TATCACTCATCGAGAACCGAGGCGTGAAGCCTTAAATCAAAT | Core staple | 833 |
| 47[39] | AGTGCATTTTAAAGGTGGCAACATCTGG | Core staple | 834 |
| 47[102] | TTAGCAAATCAATAGAAAATTCATCCATTTGGAAACGTCACCAATATAG | Core staple | 835 |
| 47[130] | CTTCGGCATTCCACCCTCAGAACCCCGCCGCTCTGAATGGTA | Core staple | 836 |
| 46[121] | TATACCAGCGCCAAAGATATCACCTCGATAGCAGCACCTTTT | Core staple | 837 |
| 49[84] | GGTCTGAAAGACAACACAGACTTTCATA | Core staple | 838 |
| 49[126] | TAGAGTGAGAATAGCCAAAAAAAAGGCTGTTTAGTAAGCCCACGCA | Core staple | 839 |
| 48[37] | ATATTAACAACGCCAACATGTATTGATTTGT | Core staple | 840 |
| 48[48] | ATCATCGTAGAAACCCTGTTTATTTGCCAAAATAG | Core staple | 841 |
| 48[58] | GGAAGTTAATTTCATCTCTTTTTCATAAACAACCC | Core staple | 842 |
| 48[69] | CAAAGTACTGTCTTGTTCAGCCAGCCATTTTTGTTTAACGTCGAGG | Core staple | 843 |
| 48[90] | TTGCTTTAGAACGGACCAGTATCTCACAAACAAATCCGTATA | Core staple | 844 |
| 48[100] | GTTCCTTTTTAACCTCCTGCTGATGCGTAACCCTT | Core staple | 845 |
| 50[104] | TGATATAAGTATATTAAACCACCTTAATGCCCCCTGCCTATT | Core staple | 846 |
| 51[46] | CCGGTTGCTATTTTGCAGAGCCTAATCAACAGTAA | Core staple | 847 |
| 51[109] | AACTTGAGTAACAGTGCAAATCCTCACTGAGATAG | Core staple | 848 |
| 51[130] | AAAAGTTTTAACGGGGTTGGAAAGATAGGAAAGTTTTGTAAC | Core staple | 849 |
| 53[134] | AATTTAATGGTTTGAATTTATCAAAA | Core staple | 850 |
| 52[167] | ACGCTGAGAAGAGTCAATAGTGAAATACCGACCGTGTGATAA | Core staple | 851 |
| 52[188] | ATAGCGATAGCTTAGATTAAGATAAGGCGTTAAATAAGAATA | Core staple | 852 |
| 52[209] | TCCCTTAGAATCCTTGAAAACAACACCGGAATCATAATTACT | Core staple | 853 |
| 52[221] | ATTAATTAATTTAGAAAAAGCCTG | Core staple | 854 |
| 0[166] | GTAGCAATACTTCTTTGATTTGAAATGGAT | Core staple | 855 |
| 2[163] | GCAGATTCACCAGTCACTCGCCATTAA | Core staple | 856 |
| 4[163] | GAACCACCAGCAGAAGATAAAACATAAAACAACGACCAAATC | Core staple | 857 |
| 7[137] | CCCGGTTATCTCGACAACTCGTATAAGTTTGTAATCCTACCT | Core staple | 858 |
| 6[163] | CAACAGTTGAAAGGAATTGAGGAATCAATCAACCATATAGTTACATACC | Core staple | 859 |
| 8[166] | TAATGGAAGGGTTAGAACCTATATCTGGTC | Core staple | 860 |
| 10[142] | TGAAAGAGTCTGTCCATCACGCA | Core staple | 861 |
| 10[160] | TTATTCATTTCAATAAATCGC | Core staple | 862 |
| 12[142] | TTTATCAAGAAAACAAAATT | Core staple | 863 |
| 12[163] | ACAATTTCATTTGAATTGATTGTTTGGATT | Core staple | 864 |
| 14[160] | GTTATTAATTTTAATAAATCC | Core staple | 865 |
| 18[166] | ATCAATTCTACTAATAGTAGTATTTCAACG | Core staple | 866 |
| 20[163] | ATTTTTAGAACCCTCATTTTTGAGAGA | Core staple | 867 |
| 22[163] | TATCAGGTCATTGCCTGAGAGTCTTAGCTATATATTTTAAGC | Core staple | 868 |
| 25[137] | AGCTGTTAAATAACAACCCGTCGGTAATGGGAGCCAGCTAGA | Core staple | 869 |
| 24[163] | TTGTTAAAATTCGCATTAAATTTTAAATATTTCGCCATGACGGCCGGAA | Core staple | 870 |
| 26[166] | GAAACCAGGCAAAGCGCCATTAAATTGTAA | Core staple | 871 |
| 28[142] | CGGTTTCATTTGGGGCGCGAGCT | Core staple | 872 |
| 28[160] | GTGGAGCCGCCACGAGTGCCA | Core staple | 873 |
| 30[142] | CTTGAAACGTACAGCGCCAT | Core staple | 874 |
| 30[163] | CCGGAATTTGTGAGAGATTTCCGGCACCGC | Core staple | 875 |
| 32[160] | CGGCGGATTGACCGATTCTCC | Core staple | 876 |
| 36[166] | AAATATTGACGGAAATTATTGTAGCGACAG | Core staple | 877 |
| 38[163] | CCTTTAGCGTCAGACTGTCAGAGCCAC | Core staple | 878 |
| 40[163] | CCGCCACCAGAACCACCACCAGAGGCCACCCTAGCGCGGTAA | Core staple | 879 |
| 42[135] | ATAGTATTAAGAGGCTGGGTTTTGCCCTCAGAAAA | Core staple | 880 |
| 42[163] | CTTTTGATGATACAGGAGTGTACTTTACCGTTTTTCAGGTTAGTAACTT | Core staple | 881 |
| 44[166] | CCTCAGAGCCACCACCCTCATCCAGTAAGC | Core staple | 882 |
| 46[142] | TCAGCGACATTCAACCGATTGAG | Core staple | 883 |
| 46[160] | GATTTTGCTAAACAAATGAAT | Core staple | 884 |
| 48[142] | TCTAAAGGAACAACTAAAGG | Core staple | 885 |
| 48[163] | ATAATTTTTTCACGTTGAACCGCCACCCTC | Core staple | 886 |
| 50[160] | ATTAGGATTAGCGGAGACTCC | Core staple | 887 |
| 13[157] | AATTACATTTA | Connector | 888 |
| | | staple | |
| 31[157] | GTTTACCAGTA | Connector | 889 |
| | | staple | |
| 49[157] | AATTGCGAATC | Connector | 890 |
| | | staple | |
| 9[160] | ATACTTCTGAA | Connector | 891 |
| | | staple | |
| 27[160] | TTCTGGTGCGG | Connector | 892 |
| | | staple | |
| 45[160] | AGAACCGCCAG | Connector | 893 |
| | | staple | |
| 11[154] | GCAGAGGCGAA | Connector | 894 |
| | | staple | |
| 29[154] | AGCTTTCAGAC | Connector | 895 |
| | | staple | |
| 47[154] | TTTCTGTATCG | Connector | 896 |
| | | staple | |
| 7[157] | AGTTGGCAATG | Connector | 897 |
| | | staple | |
| 25[157] | ACGTTAATATG | Connector | 898 |
| | | staple | |
| 43[157] | GTCATACATAA | Connector | 899 |
| | | staple | |
| 5[157] | AAATACCGAAC | Connector | 900 |
| | | staple | |
| 23[157] | TCTACAAAGAG | Connector | 901 |
| | | staple | |
| 41[157] | CACCCTCAGGC | Connector | 902 |
| | | staple | |
| 3[157] | TATTTACATAT | Connector | 903 |
| | | staple | |
| 21[157] | CAAGGATAAGG | Connector | 904 |
| | | staple | |
| 39[157] | AATCAAGTTTT | Connector | 905 |
| | | staple | |
| 15[154] | TTTGCCCGATT | Connector | 906 |
| | | staple | |
| 33[154] | GTGGGAACAGT | Connector | 907 |
| | | staple | |
| 51[154] | TCAAGAGAAGC | Connector | 908 |
| | | staple | |
| 1[160] | AATTAACCGAC | Connector | 909 |
| | | staple | |
| 19[160] | GAAAAGGTGGG | Connector | 910 |
| | | staple | |
| 37[160] | GGAGGGAAGAA | Connector | 911 |
| | | staple | |
| 9[12] | TTTTTCAGAATGCGGCGGGCCTCTGTGGCGC | Vertex staple | 912 |
| 15[16] | TTTTTTCCGCTCACAATCGTGCCAGCTGCATTAATGTTTTT | Vertex staple | 913 |
| 38[30] | AAAACAAAAGATAGATAAATTTACGAATCATTACCGCGCCCAATTTTT | Vertex staple | 914 |
| 36[34] | ACTCCTTCATACATCGAGCCAGCCATATAATTGTGTCGAAATCCGCGACT | Vertex staple | 915 |
| | TTTT | | |
| 49[14] | TTTTTCTTAATTGAGAATCGTAATAAGAGAATTTTT | Vertex staple | 916 |
| 45[12] | TTTTTAATAATATCCCATCCTAGTCCTGCGA | Vertex staple | 917 |
| 51[16] | TTTTTTAGCAAGCAAATACAATTTTATCCTGAATCTTTTTT | Vertex staple | 918 |
| 37[12] | TTTTTGCAAACGTAGAAAATAATTACGCCCCTTTTTAAGAAACAAG | Vertex staple | 919 |
| 39[9] | TTTTTATCTTACCGAAGAGTATGTTATTTTT | Vertex staple | 920 |
| 20[31] | TTTTTGTACAGCGTAACAGACGAGAAGAAAAATCTACGTTAATATTTTT | Vertex staple | 921 |
| 18[34] | TGTAGCTTGTCTGGTGACCAATTAGCCGGCGGTTGCGGTATGAGCCGGG | Vertex staple | 922 |
| | TTTTT | | |
| 31[14] | TTTTTCTGCTCCATGTTACCTTTGAAAGAGGTTTTT | Vertex staple | 923 |
| 27[12] | TTTTTGAATAAGGCTTGCCCTAAGCTGCAAA | Vertex staple | 924 |
| 33[16] | TTTTTAAACGAACTAACATCATAACCCTCGTTTACCTTTTT | Vertex staple | 925 |
| 19[12] | TTTTTTGCAACTAAAGTACGGCAACATGGCAAACTCCAACAGGCG | Vertex staple | 926 |
| 1[12] | TTTTTTATAACGTGCTTTCCTTGCTTTGTCAAGCGAAAGGAGAACG | Vertex staple | 927 |
| 21[9] | TTTTTACCAGACCGGAATTTTAAATATTTTT | Vertex staple | 928 |
| 2[30] | TGGGCATCAGTGTGCACGTTTTCATTCCTGTGTGAAATTGTTATTTTT | Vertex staple | 929 |
| 0[34] | CTATGGTCGTTAGATTACACTCGGCTGGAGCCAACGCTCAACAGTAGGG | Vertex staple | 930 |
| | TTTTT | | |
| 13[14] | TTTTTTCACTGTTGCCCTGGGTGTGTTCAGCTTTTT | Vertex staple | 931 |
| 3[9] | TTTTTAAAAACCGTCTAACGAGCACGTTTTT | Vertex staple | 932 |
| 7[24] | GGGGTGGTTTGCCCCAGCAGGCGTTCACTAATCTGATGGAAGCGCATTAGA | Vertex | 933 |
| | TAGCAATAGCTTTTTT | bundle | |
| | | strand | |
| 25[24] | CCAAAATGCTTTAAACAGTTCAGGCAAAATTCTCATTGAAAATCCTGTTTC | Vertex | 934 |
| | GTCAAAGGGCGTTTTT | bundle | |
| | | strand | |
| 43[24] | GCGTAGAATAACATAAAAACAGGAATGTCGATATCTAGAAAACGAGAATGG | Vertex | 935 |
| | CTTCAAAGCGATTTTT | bundle | |
| | | strand | |
| 7[9] | TTTTTAATCGGCCAACGTGCTGCGGCTTCACTAATCTGATGTATAAAGTAC | Vertex | 936 |
| | CGCAATGAAACGG | bundle | |
| | | strand | |
| 25[9] | TTTTTAGACGACGATAATCATTCAGTGCAAAATTCTCATTGAAATCGTTAA | Vertex | 937 |
| | CGACTCCAAGATG | bundle | |
| | | strand | |
| 43[9] | TTTTTTACCAACGCTAAAACAAGAAAAATGTCGATATCTAGACAGATGAAC | Vertex | 938 |
| | GGAATTCGAACCA | bundle | |
| | | strand | |
| | CATCAGATTAGTGAA | Vertex | 939 |
| | | bundle | |
| | | strand | |
| | | (complementary) | |
| | CAATGAGAATTTTGC | Vertex | 940 |
| | | bundle | |
| | | strand | |
| | | (complementary) | |
| | CTAGATATCGACATT | Vertex | 941 |
| | | bundle | |
| | | strand | |
| | | (complementary) | |

TABLE 8
Sequences of the pentagonal prism.

| | | | SEQ ID |
|---|---|---|---|
| 5′-end | Sequence | Note | NO: |
| 1[53] | CGCCAACCGCAAGAAAAGTTACCTGTCC | Core staple | 942 |
| 1[84] | AGTGAGGAAAACGCTCATGCGCGTACTAGTGTTTTTGGT | Core staple | 943 |
| 0[44] | CGTCCACCACACCCGCCAACAAGAGCAG | Core staple | 944 |
| 3[102] | AATCCATTGCAACAGGACCACCGACGGACTTGCGGTCCCTTAGAA | Core staple | 945 |
| 3[144] | CACTATCGGCCTTGCTGGTAGCAAATTAATTACATTGCATTA | Core staple | 946 |
| 2[44] | ACTAAAATCCCTTATAATGAGAGACGCCAGGCTGC | Core staple | 947 |
| 2[65] | TCCGAATAGCCCGAGATTTGCCCTCACC | Core staple | 948 |
| 2[72] | GTGCCAACGGATTCGCCGTCAGCGTATAATC | Core staple | 949 |
| 2[93] | GAATTTGAATGTACCTTTCTCATCAATATAAATTT | Core staple | 950 |
| 2[107] | CAGAACATCGCCATTAAAAATGAATCTGGTCAATA | Core staple | 951 |
| 2[114] | CGTTCGCGCATCAGATGTGTTTGGATTCCTGATTATCAGTAT | Core staple | 952 |
| 2[135] | TGAATTTCAACGTAGATTAATGGAAAGGAGCGGAATTACGTT | Core staple | 953 |
| 5[25] | GTGGTTCCGATCCACGCAGAG | Core staple | 954 |
| 5[60] | AAAAGTTTGGGCGCTTATTTGACGAGCACGTGGTA | Core staple | 955 |
| 5[123] | ACCGCGTAAGTATTTACCCAGAACAATATTACCATCACCATC | Core staple | 956 |
| 4[41] | CAAGCGGAATCGGCATTAAAGCGCGTAAGCTTTCC | Core staple | 957 |
| 4[97] | ACCTTGCTGAACAACAGCTGAAGTTTAATGCGCGAACTGATA | Core staple | 958 |
| 4[135] | CGCCAGTTGAAGATTAGAATTTTAAAAGTTTCCAC | Core staple | 959 |
| 7[32] | GCGAACCTGTTCCACACAACATACTAGCTGTCGGTCATTGAG | Core staple | 960 |
| 7[60] | TTTACGATCCGCGGTGCTCAG | Core staple | 961 |
| 7[74] | AGTACATTAAGGGTGCCTAATGAGGAGGATCCGCGTCCAAAC | Core staple | 962 |
| 7[109] | ATAAAATCTAAAGCATCGCCCTAAACAATATGCTC | Core staple | 963 |
| 6[51] | CCGAAGCATAAAGTGTATCGAATTCCAG | Core staple | 964 |
| 6[90] | ACTTTAGCTAACTCGAGACGGGGGAGAAACAATCTTGTTCTTCCCGG | Core staple | 965 |
| | GT | | |
| 6[114] | CATATCCTTTGCCCGAATCATCATATTATACGTAA | Core staple | 966 |
| 8[65] | CAGTTCTTTTTCACCGCCTGGCCCATCA | Core staple | 967 |
| 9[60] | CACCGCTCAACACCGTCGGTGATGGGTCTGGCGGTGCCTTGT | Core staple | 968 |
| 9[130] | GAATTTCAGGAAATCAATGAGAGCCAGCAGCAAAT | Core staple | 969 |
| 11[39] | CGGACATCCCTTTTAGACAGGAACATAA | Core staple | 970 |
| 11[53] | CCAAGCGCAGGTTTCTGCGTAATCATGGTCAGAGC | Core staple | 971 |
| 11[88] | TGCTGGCTATTAGTCGGGGGAAATACCTACATTTTGACTTTT | Core staple | 972 |
| 11[130] | TTCCCTGAAAGAACGAACCACCAGGCCA | Core staple | 973 |
| 10[58] | CAGCAGAATCCTGAGAATGGTTGCATGCGCCGCTACAGTTGA | Core staple | 974 |
| 10[72] | GCTCTGATTGCCGTTCCGGCAAACGTAGAACTGAT | Core staple | 975 |
| 10[100] | TGCGTAAAAGAGTCTGTCCGCCAGCGTCTGAAATGGATAATA | Core staple | 976 |
| 10[114] | CTCTCGCTGGGTCGCTATTAATTATCCTGATAATATACATCA | Core staple | 977 |
| 10[121] | GCAGCAAATTAACCGTTGTAATATATTGGCAGATTCACCTTC | Core staple | 978 |
| 12[37] | AATGCTCGTCATTGCCAACGGCAGCAGTAGG | Core staple | 979 |
| 12[48] | GCTTAATACCGGGGTGTCACTTATTGGGGTTGCAG | Core staple | 980 |
| 12[79] | ATAGCGATAGCTTACAAGCGTGCCGCAT | Core staple | 981 |
| 12[90] | TCCTTGAGTGAGCCTTACATCGCCTCAAATATCAAGTATTAG | Core staple | 982 |
| 12[100] | TCCGTTTTTTCGTCTCGATAACGGTACAAAAGGCA | Core staple | 983 |
| 12[121] | ATCCAGCCTCCGTAACAATTTCATATAACCTTGCTTCTTTCT | Core staple | 984 |
| 14[69] | ACCGAGCAAGCCTGTTGCGTTGCGCTCAGTGG | Core staple | 985 |
| 15[46] | CGGCTTTCCAGTCGGGAGTTTGCGGCGCGCCATGC | Core staple | 986 |
| 15[98] | ACAACTCGATGATGGCAATCTCACAGTTTGACAAACAATTCG | Core staple | 987 |
| 15[109] | TAATTGAGGATTTAGAAACCCTCAAGTAACAACCAAGTAACG | Core staple | 988 |
| 15[130] | ATTAGCCGTCAATAGATAGTTGGCTTTAACGGAGGCGACAGA | Core staple | 989 |
| 17[130] | GTGCCATCCCACGCAACAAGGGTAAAGTTAAACG | Core staple | 990 |
| 16[167] | CACAGGCGGCCTTTAGTGATGCAGCTTACGGCTGGAGGTGTC | Core staple | 991 |
| 16[188] | AAAATCCCGTAAAAAAAGCCGCAGCATCAGCGGGGTCATTGC | Core staple | 992 |
| 16[205] | GTGTACATCGACATAAAAGGCGCTTTCGCACTCA | Core staple | 993 |
| 19[53] | GAGCACCAACCTAAAGAAGAGTAATCGA | Core staple | 994 |
| 19[84] | TCGCAAAAAATCGGTTGTATTAATTGCTCCATTAGTACG | Core staple | 995 |
| 18[44] | TTTTTTTGATAAGAGGTTTTTAATTCTT | Core staple | 996 |
| 21[102] | TACCAGAGCATAAAGCTTGGTCAAGTTTCCAACAGCATTCTGCTC | Core staple | 997 |
| 21[144] | ATTACAGGCAAGGCAAAGCTGAAAGAAACGTACAGCTTGCCA | Core staple | 998 |
| 20[44] | GCTAAGCAAAGCGGATTCTCAAATTAGTAAACACT | Core staple | 999 |
| 20[65] | AAAAAAGATTAAGAGGAATAAATATAGC | Core staple | 1000 |
| 20[72] | AGACAAGTTGGGTAACGGGTAAAAATACATT | Core staple | 1001 |
| 20[93] | CCATTTCCCAAAGGGGGAACGGCCTCAGGAATTAA | Core staple | 1002 |
| 20[107] | AGAGCCGGAGAGGGTAGGTCAATCAAGCAAATAAT | Core staple | 1003 |
| 20[114] | AGGAAACGACCGCTATTCTCCAGCCCAGTTTGAGGGGACGAG | Core staple | 1004 |
| 20[135] | AAATTTCAGAGGCGATCCGCTTCTCGCATCGTAACCGTCTCC | Core staple | 1005 |
| 23[25] | CTGACTATTAAGAAAACAAGT | Core staple | 1006 |
| 23[60] | CAATATCGCGCATTTTTATGCTGTAGCTCAAGAAC | Core staple | 1007 |
| 23[123] | TTTAAGGGTGCCTTTATCAAAATTAAGCAATATATTTTTAAA | Core staple | 1008 |
| 22[41] | ACAGTTCTAGTCAGTCAAAGCTTGCTCCTAAATAT | Core staple | 1009 |
| 22[97] | TGATAATCAGAAGGAATCGTCAGTCAACCGTTCTAGCTGATA | Core staple | 1010 |
| 22[135] | AATACGTTAACAATAGGGGAACAAACGGCGGAGAT | Core staple | 1011 |
| 25[32] | TTTCCAGACGAGATTCATCAGTTGTAAAACGGGCTTGAGAGC | Core staple | 1012 |
| 25[60] | TTATCAACGTAAGAACCACGA | Core staple | 1013 |
| 25[74] | GTCTACGAGGGCAGATACATAACGCATTATACCTTATGGCCA | Core staple | 1014 |
| 24[51] | ATCGGAATACCACATTCGGGAAGAAACT | Core staple | 1015 |
| 24[90] | GCTTTAAAAGGAATCAATACTGCAAGGCGATTATTTGAATTACCAGT | Core staple | 1016 |
| | CA | | |
| 24[114] | TCGCAACCCGTCGGATTGCATCTGCAGCTTTCGCA | Core staple | 1017 |
| 26[65] | AAAGACTGGATTCATTGAATCCCCGCAT | Core staple | 1018 |
| 26[107] | CAGATTGTATATATGTACCCCGGTAATTAATCAGTCAAGTAA | Core staple | 1019 |
| 27[60] | TTACGCCGGGAAAGAATACACGATTGCCACTGGATATTCTTC | Core staple | 1020 |
| 27[129] | GCACGGTGCGGATTGTAACGTAAAACTAGCATCTAT | Core staple | 1021 |
| 29[39] | TCAGGACAGAATTCCCAATTCTGCCATG | Core staple | 1022 |
| 29[53] | GACAACAAAGTAATTTCAAAATCTACGTTAAAGAT | Core staple | 1023 |
| 29[88] | GGTTCAATATGATATCCGCCCAAAAACATTATGACCCTATCA | Core staple | 1024 |
| 29[130] | AGCGATTCAATGAGAGATCTACAACGGT | Core staple | 1025 |
| 28[58] | AGGTAGATTTAGTTTGAGAATATAGCGGATGGCTTAGACGAA | Core staple | 1026 |
| 28[72] | TAACGTCACCCTCAGCAGCGAAAGTTAAACGCCAG | Core staple | 1027 |
| 28[100] | GAATAACCTGTTTAGCTAAAGCCTTTTTGCGGGAGAAGAGAA | Core staple | 1028 |
| 28[114] | GACCAACGGCACAGCGGATCAAACGATCGCAACGC | Core staple | 1029 |
| 28[121] | GACCATTTGGGGCGCGAGAATTAGTTCAACGCAAGGATAGGT | Core staple | 1030 |
| 30[37] | CGGACTTTGAAAACGAAAGAGGCACGCGGTT | Core staple | 1031 |
| 30[48] | GCGGTATGATGGTTCTGCTCAGGGGTAAGCTTTAA | Core staple | 1032 |
| 30[79] | GCAGTTGGGCGGTTATCATCATTGACCC | Core staple | 1033 |
| 30[90] | ATTTGCCCGATTTTATGTGCTGCAAGCCCCAAAAAGTAGCCA | Core staple | 1034 |
| 30[100] | ATTCGGAACGAGGGTAGTTTTTCACGTTGTACCGG | Core staple | 1035 |
| 30[121] | GAATACAGAGGCGCCATGTTTACCCACGGAAAAAGAGACCG | Core staple | 1036 |
| 32[69] | GGACGTTAACTAATCATAGTAAGAGCAAATGT | Core staple | 1037 |
| 33[46] | TTAATAACCCTCGTTTAGCCAGAGTTCAGTGTTCA | Core staple | 1038 |
| 33[98] | ATGTGAGCGACGACAGTATGAACTGGCTCCCATCAACATTAA | Core staple | 1039 |
| 33[109] | TAACGTCTGGCCTTCCTCAGGAAGCTGGCGAGTCACGATGAG | Core staple | 1040 |
| 33[130] | GTGAACGCCATCAAAAATATTTAAGCCTCTTGGCCAGTTGAG | Core staple | 1041 |
| 35[132] | TAAAACACTCATCTTAGGCCGCTTTTGCGG | Core staple | 1042 |
| 34[224] | TAGTTGCGCCGACAATAAATTGTGTCGAAA | Core staple | 1043 |
| 37[53] | CACCGACCGTGTGATCAGACGACACAAG | Core staple | 1044 |
| 37[84] | AATAGAAGCACCATTACCAGGAATACCCATTTTGTAAAT | Core staple | 1045 |
| 36[44] | CTTAGTTACCAGAAGGAATAAGAGATAA | Core staple | 1046 |
| 36[65] | GAAGAAACGCAATAATAAGAA | Core staple | 1047 |
| 39[102] | AATCAAAATCACCAGTAAATTCATGTTAATTTGTAAATCGAGGTG | Core staple | 1048 |
| 39[144] | ATCTATCACCGTCACCGTCAACCGGTGAGAATAGAAACGTTA | Core staple | 1049 |
| 38[44] | AAAGAGGGTAATTGAGCCAGCCTTCAGCCATTTTT | Core staple | 1050 |
| 38[65] | AAGTCAGAGAGATAACCTAACGTCTCCA | Core staple | 1051 |
| 38[72] | TTGTGCAGACAGCCCTCCTGACCTCACAATC | Core staple | 1052 |
| 38[93] | AAAGCGTAACCAAACTAACGTATCACCGTACTTGC | Core staple | 1053 |
| 38[107] | TCTAGAGCCGCCACCCTAGACGATCGCAGTCACAG | Core staple | 1054 |
| 38[114] | TTTTCGTCTTCACTGAGGTTTAGTTGATATAAGTATAGTCTG | Core staple | 1055 |
| 38[135] | GTCAATGAATATAGGAAAACCGCCGATAAGTGCCGTCGGAGG | Core staple | 1056 |
| 41[25] | CACCCTGAACCATAAAAATTT | Core staple | 1057 |
| 41[60] | ATACCCAATAAACCGAGCTGGCATGATTAAGAAGA | Core staple | 1058 |
| 41[123] | ACCCCTTATTCAGCACCCCATTTGGGAATTACCAAAGAAACT | Core staple | 1059 |
| 40[41] | AGAATAAAAAGTCACAATGAACGAACAAATTACGC | Core staple | 1060 |
| 40[97] | ACAAACAAATAATTTTTTGTTCAGAGCCACCACCGGAACCGC | Core staple | 1061 |
| 40[135] | GGATCCAGTAACGGGGTAGACTCCTCAAGAGCCAG | Core staple | 1062 |
| 43[32] | GCCTATCCTGTTATCCGGTATTCTTACCGCGCAATCAAAGCC | Core staple | 1063 |
| 43[60] | TTTCCTGTTTACATGTTGAAA | Core staple | 1064 |
| 43[74] | AATTTAAATCCCGACTTGCGGGAGCGAGAACGTATTAATAAA | Core staple | 1065 |
| 42[51] | GCACGAGGCGTTTTAGCTATTTTCTCCT | Core staple | 1066 |
| 42[90] | CCTGCTTTGAAGCCAAGAAACTGTAGCATTCCACAAGAACGGAAGCA | Core staple | 1067 |
| | AG | | |
| 42[114] | TGCCATGAAAGTATTAAAGAGGGTACCGCCATAAT | Core staple | 1068 |
| 44[65] | GCGATCCCAAAAAAATGAAAATAGGCTA | Core staple | 1069 |
| 44[107] | GTCTGGAAAGTGGCCTTGATATTCCTCCCTCTTTCATACACC | Core staple | 1070 |
| 45[60] | TATGCGACCTAAATAAGAATACTTATGGTTTCAGCTAAAGTT | Core staple | 1071 |
| 45[129] | TCAGCCCATGTTTACCGTGGTTGAGGCAGGTCCAGA | Core staple | 1072 |
| 47[39] | GACGTAATAAATAAAAGAAACGCAACTC | Core staple | 1073 |
| 47[53] | ACAATCAACACTGTCTTATCGTAGGAATCATAAGA | Core staple | 1074 |
| 47[88] | TTATCACCGGAACCACAACTTAGCAAGGCCGGAAACGTATCA | Core staple | 1075 |
| 47[130] | GTAATAGCCCGCCACCCTCAGAGCGACA | Core staple | 1076 |
| 46[58] | TACCACGGAATAAGTTTAAAA | Core staple | 1077 |
| 46[72] | TTAAGGTTGGGTTATATAACTATATCATCTTATAG | Core staple | 1078 |
| 46[100] | TTAATGGTTTACCAGCGGAGCCAGGAAACCATCGATAGAGCG | Core staple | 1079 |
| 46[114] | TTTAATCGCAATCGGTTTATCAGCTCAGGAGTTTC | Core staple | 1080 |
| 46[121] | GAACAAAAGGGCGACATACTTGAGGTAATCAGTAGCGATTCG | Core staple | 1081 |
| 48[37] | GGATTTTCGAGCAAATAAGGCGTTGCTCCAT | Core staple | 1082 |
| 48[48] | GTTACTTTAATCGGATAGATAAAATAAATACAGAG | Core staple | 1083 |
| 48[79] | CAGCTTGATACCGATCCCATTCCAGAAC | Core staple | 1084 |
| 48[90] | AATTTCTACCAAGTCAACGCCGAATCCTCATTAAAAATGCCC | Core staple | 1085 |
| 48[100] | TTTGCTGATGCAAATCCTCAAATAAGTTTTGGCCA | Core staple | 1086 |
| 48[121] | TGTAGACAAAGAAGGAACAACTAACCAAAAGGAGCCTTCCC | Core staple | 1087 |
| 50[69] | CCGTTTTGAACCTCAAGATTAGTTGCTAATTA | Core staple | 1088 |
| 51[46] | ACGCCCAGCTACAATTTAGTTACAAGTCCTGTCCA | Core staple | 1089 |
| 51[98] | CTATTATCCCGGAATAGGTCGCACTCATGTCTATTTCGGAAC | Core staple | 1090 |
| 51[109] | AAACCGTATAAACAGTTGCCAGAAACCAGTAGATCTAATATT | Core staple | 1091 |
| 51[130] | CTGCAGTGCCTTGAGTATCTGAATACCGTAATCCAGACGCGA | Core staple | 1092 |
| 53[130] | AACACCGGAATCATAATACCTTTTTAACCTCCGG | Core staple | 1093 |
| 52[167] | AAATCATAGGTCTGAGAGACTTACTAGAAAAAGCCTGTTTAG | Core staple | 1094 |
| 52[188] | GAGTCAATAGTGAATTTATCATATCATATGCGTTATACAAAT | Core staple | 1095 |
| 52[205] | GATTAAGACGCTGAGAATCTTACCAGTATAAAGC | Core staple | 1096 |
| 34[167] | CTGAGGCTTGCAGGGAGTTAATGACCCCCAGCGATTATACCA | Core staple | 1097 |
| 34[188] | CATAACCGATATATTCGGTCGAGCGCGAAACAAAGTACAACG | Core staple | 1098 |
| 34[209] | TGACAACAACCATCGCCCACGGAGATTTGTATCATCGCCTGA | Core staple | 1099 |
| 5[25] | GTGGTTCCGATCCACGCAGAG | Core staple | 1100 |
| 23[25] | CTGACTATTAAGAAAACAAGT | Core staple | 1101 |
| 41[25] | CACCCTGAACCATAAAAATTT | Core staple | 1102 |
| 0[166] | CTGAGTAGAAGAACTCAAACACGACCAGTA | Core staple | 1103 |
| 2[163] | ATTCTGGCCAACAGAGATAAAACAGAG | Core staple | 1104 |
| 4[163] | AGTATTAACACCGCCTGCAACAGTCAGAAGATAGAACCCAGT | Core staple | 1105 |
| 6[163] | TCTTTAGGAGCACTAACAACTAATAAGGAATGAAA | Core staple | 1106 |
| 8[142] | TTGTTACCTGAAACAAATACTTCTTTGATTAGTAATA | Core staple | 1107 |
| 8[166] | GCACGTAAAACAGAAATAAATGAGGAAGGT | Core staple | 1108 |
| 10[160] | AACAAACATCAAGAAGCAAAA | Core staple | 1109 |
| 12[163] | ACATAAATCAATATATGGAACCTACCATAT | Core staple | 1110 |
| 14[142] | CAGAGGGTTATGAGTGATTGAATTACCTTTTTTA | Core staple | 1111 |
| 14[160] | GCGGAACAAAGAAAGAGTAAC | Core staple | 1112 |
| 18[166] | ATTAACATCCAATAAATCATTTTAGAACCC | Core staple | 1113 |
| 20[163] | AAATGCAATGCCTGAGTCAGGTCATTG | Core staple | 1114 |
| 22[163] | GGAGCAAACAAGAGAATCGATGAAAGGCTATAATGTGTAAAA | Core staple | 1115 |
| 24[163] | TGTTAAATCAGCTCATTTTTTAACTATTTTGTGGG | Core staple | 1116 |
| 26[142] | AAGGGTGGAGAATCGGCAGGTGGCATCAATTCTACTA | Core staple | 1117 |
| 26[166] | CATTCAGGCTGCGCAACTGTTTAAAATTCG | Core staple | 1118 |
| 28[160] | ACCTCACCGGAAACCCGCCAC | Core staple | 1119 |
| 30[163] | TCTCCGTGGTGAAGGGAGAAACCAGGCAAA | Core staple | 1120 |
| 32[142] | GGGGGTGCCGTAGCTCTAGTCCCGGAATTTGTGA | Core staple | 1121 |
| 32[160] | GGTCACGTTGGTGTATTGACC | Core staple | 1122 |
| 36[166] | ATTATTCATTAAAGGTGAATAAGTTTGCCT | Core staple | 1123 |
| 38[163] | CTGTAGCGCGTTTTCATCTCAGAGCCG | Core staple | 1124 |
| 40[163] | ACCACCAGAGCCGCCGCCAGCATTCACCACCCGGCATTCAGA | Core staple | 1125 |
| 42[163] | GGAGTGTACTGGTAATAAGTTTTAAGCGTCAAAGC | Core staple | 1126 |
| 44[142] | CCATTTCTGTCAGCGGAATTGAGGGAGGGAAGGTAAA | Core staple | 1127 |
| 44[166] | CCCTCATTTTCAGGGATAGCTACATGGCTT | Core staple | 1128 |
| 46[160] | ACTTTCAACAGTTTATGGGAT | Core staple | 1129 |
| 48[163] | TTGAAAATCTCCAAAAAGAACCGCCACCCT | Core staple | 1130 |
| 50[142] | GCGACCCTCAAAAGGCTAGGAATTGCGAATAATA | Core staple | 1131 |
| 50[160] | GGTTTTGCTCAGTAAAGGATT | Core staple | 1132 |
| 9[160] | CAAAATTATGA | Connector staple | 1133 |
| 27[160] | GCGCCATTCCA | Connector staple | 1134 |
| 45[160] | CAGAGCCACTA | Connector staple | 1135 |
| 11[154] | GAAGATGATTT | Connector staple | 1136 |
| 29[154] | GGGAACGGACA | Connector staple | 1137 |
| 47[154] | TTTGCTAAAGC | Connector staple | 1138 |
| 7[157] | TATCTAAAAAC | Connector staple | 1139 |
| 25[157] | CATTAAATTGA | Connector staple | 1140 |
| 43[157] | TTGATGATATT | Connector staple | 1141 |
| 1[160] | ACATCACTTTT | Connector staple | 1142 |
| 19[160] | ATAGTAGTAGG | Connector staple | 1143 |
| 37[160] | TATTGACGGTA | Connector staple | 1144 |
| 13[157] | ATGGAAACAGT | Connector staple | 1145 |
| 31[157] | GAGATAGACCG | Connector staple | 1146 |
| 49[157] | ATTTTTTCATT | Connector staple | 1147 |
| 3[157] | ATAAAAGGGTA | Connector staple | 1148 |
| 5[157] | GTGAGGCGGTC | Connector staple | 1149 |
| 15[154] | ATTATCATTGC | Connector staple | 1150 |
| 21[157] | TCATATATTCA | Connector staple | 1151 |
| 23[157] | CCTGAGAGTCC | Connector staple | 1152 |
| 33[154] | GTAATGGGAAA | Connector staple | 1153 |
| 39[157] | TTAGCGTCATT | Connector staple | 1154 |
| 41[157] | CCACCAGAACT | Connector staple | 1155 |
| 51[154] | AGGATTAGCGC | Connector staple | 1156 |
| 1[12] | TTTTTAAACAGGAGGCCGATTAATCAGATCACGGTCACGCTGAACG | Vertex staple | 1157 |
| 0[34] | TCGTTAGAAAGGGATTACACTTTTCTTTCGCCATATTTAACAACGCCA | Vertex staple | 1158 |
| | ATTTTT | | |
| 3[9] | TTTTTAAAAACCGTCTAGCGGGAGCTTTTTT | Vertex staple | 1159 |
| 2[30] | TGGGCATCAGTGTGCACGTTTTCATTCCTGTGTGAAATTGTTATTTTT | Vertex staple | 1160 |
| 9[12] | TTTTTCAGAATGCGGCGGGCCTCTGTGGCGC | Vertex staple | 1161 |
| 13[14] | TTTTTGTAATGGGTAAAGGGGTGTGTTCAGCTTTTT | Vertex staple | 1162 |
| 15[16] | TTTTTTCCGCTCACAATCGTGCCAGCTGCATTAATGTTTTT | Vertex staple | 1163 |
| 19[12] | TTTTTAGTTTCATTCCATATAAAGTACGGAGAGTACCTTTAAGAA | Vertex staple | 1164 |
| 18[34] | GCAACTAACAGTTGTGAACGGCTGACCAGTCACTGTTGCCCTGCGGC | Vertex staple | 1165 |
| | TGTTTTT | | |
| 21[9] | TTTTTAGGTCAGGATTAGTGTCTGGATTTTT | Vertex staple | 1166 |
| 20[31] | CCAGGCTGACCAATAAGGTAAATTGAACTAACGGAACAACATTATTT | Vertex staple | 1167 |
| | TT | | |
| 27[12] | TTTTTACACCAGAACGAGTAGCTTGCCCGCA | Vertex staple | 1168 |
| 31[14] | TTTTTATAAGGGAACCGAATGTACAGACCAGTTTTT | Vertex staple | 1169 |
| 33[16] | TTTTTTTACAGGTAGAAACGATAAAAACCAAAATAGTTTTT | Vertex staple | 1170 |
| 37[12] | TTTTTTACATACATAAAGGTGTAGCAAAAGTAAGCAGATAGCATAG | Vertex staple | 1171 |
| 36[34] | AGTATGTGCAACATGAGAATAAGAGGCAACGAGGCGCAGACGGTCA | Vertex staple | 1172 |
| | ATCTTTTT | | |
| 39[9] | TTTTTCTTTTTAAGAAACGTAGAAAATTTTT | Vertex staple | 1173 |
| 38[30] | CAAAATTCTGAACAAGATAGAAACCCCAATAGCAAGCAAATCATTTT | Vertex staple | 1174 |
| | T | | |
| 45[12] | TTTTTCTAATTTACGAGCATGAAAATAAGAG | Vertex staple | 1175 |
| 49[14] | TTTTTCATGTAATTTAGGCTAAAGTACCGACTTTTT | Vertex staple | 1176 |
| 51[16] | TTTTTGATATAGAAGGCAATCTTACCAACGCTAACGTTTTT | Vertex staple | 1177 |
| 5[9] | TTTTTAAAATCCTGTTTCGTCAAAGGGCGTTTTT | Vertex staple | 1178 |
| 7[24] | GGGGTGGTTTGCCCCAGCAGGCGTTTTT | Vertex staple | 1179 |
| 23[9] | TTTTTAAATCAGGTCTTGCAAACTCCAACTTTTT | Vertex staple | 1180 |
| 25[24] | AAAGGAGAATGACCATAAATCAATTTTT | Vertex staple | 1181 |
| 41[9] | TTTTTGGGAGAATTAACCTTACCGAAGCCTTTTT | Vertex staple | 1182 |
| 43[24] | CCTAACAGGGAAGCGCATTAGACTTTTT | Vertex staple | 1183 |
| 7[9] | TTTTTAATCGGCCAACGTGCTGCGGCTTCACTAATCTGATGAAAAGGT | Vertex bundle | 1184 |
| | AAAGTTAGCTATTGAA | strand | |
| 25[9] | TTTTTCGAGAGGCTTTTTGACGAGAAGCAAAATTCTCATTGAAATCGT | Vertex bundle | 1185 |
| | TAACGACTCCAAGATG | strand | |
| 43[9] | TTTTTAGCGTCTTTCCATATCCCATCAGTGGCGATATCGCGCATAGGC | Vertex bundle | 1186 |
| | TGACCGGAATACC | strand | |
| | CATCAGATTAGTGAA | Vertex bundle | 1187 |
| | | strand | |
| | | (complementary) | |
| | CAATGAGAATTTTGC | Vertex bundle | 1188 |
| | | strand | |
| | | (complementary) | |
| | GATATCGCCACT | Vertex bundle | 1189 |
| | | strand | |
| | | (complementary) | |

TABLE 9
Sequences of the hexagonal prism.

| | | | SEQ ID |
|---|---|---|---|
| 5′-end | Sequence | Note | NO: |
| 1[53] | CCGAGCGTGGTGCTGAAGTTACCTGTCC | Core staple | 1190 |
| 1[84] | GTACTATTCCATCACGCAAGACGGGGAACCGCTACGTGC | Core staple | 1191 |
| 0[44] | AGGAATCGGAACCCTAAAACAAGAGCAG | Core staple | 1192 |
| 3[102] | TTTAGTAAAAGAGTCTGGGTTGCTAGCACATGATGCTGAAACATC | Core staple | 1193 |
| 3[144] | AACCCAGAATCCTGAGAATCAGAGCTTTTACATCGGTTAAAT | Core staple | 1194 |
| 2[44] | ACTAAAATCCCTTATAATGAGAGACGCCAGGCTGC | Core staple | 1195 |
| 2[65] | TCCGAATAGCCCGAGATTTGCCCTCACC | Core staple | 1196 |
| 2[72] | GTGCCGAATAATGGAAGACGGAACAGGGCGC | Core staple | 1197 |
| 2[93] | AATACCTACCATCCTGATCGACAACTCGTATATGA | Core staple | 1198 |
| 2[107] | ACATCACACGACCAGTATCTTTAACCAGCAGTTGC | Core staple | 1199 |
| 2[114] | AATTGCACGTTGATGGCTTTGCCCGAAGTATTAGACTTTCAA | Core staple | 1200 |
| 2[135] | AACGAAATTGATCATATTTAAAAGGATAATACATTTGAGGAA | Core staple | 1201 |
| 5[25] | GTGGTTCCGATCCACGCAGAGGCGAACCTGTTCCACACAACATACTAG | Core staple | 1202 |
| 5[39] | GGCATTAAAGAGCACTAGAAGAAAGCGAAAGGTCACGCTTAC | Core staple | 1203 |
| 5[60] | AAAAGTTTGGAGGGAGCGAACGTGGCGAGAAACAC | Core staple | 1204 |
| 5[123] | AAGACGCTCATCACTTGTTATAATCAGTGAGTAACGTGTCGC | Core staple | 1205 |
| 4[97] | GCCCTAAAACATAACAGCTGAAGATTATTTACATTGGCAGAT | Core staple | 1206 |
| 4[135] | TTTGTGAGGCTGAAAAATATCTAAAATATCTGTCA | Core staple | 1207 |
| 7[60] | TTTACGATCCGCGGTGCGAAC | Core staple | 1208 |
| 7[74] | AGTACATTAAGGGTGCCTAATGAGGAGGATCCGCGTCCCAAA | Core staple | 1209 |
| 7[109] | CCATGCGCGAACTGATATCACCAGTTTTGACCTTC | Core staple | 1210 |
| 6[51] | CCGAAGCATAAAGTGTATCGAATTCCAG | Core staple | 1211 |
| 6[90] | ATCAAAGCTAACTCGAGACGGGATTATACTTCTCTTGTTCTTCCCGGGT | Core staple | 1212 |
| 6[114] | TGATTGAAAGGAATTGAGGATTTAGAACGTTTTAC | Core staple | 1213 |
| 8[65] | CAGTTCTTTTTCACCGCCTGGCCCATCA | Core staple | 1214 |
| 9[60] | CACTGATAAAGCAACCGCAAGTAGACTTGTACGGTGCCTTGT | Core staple | 1215 |
| 9[130] | ATTTCCTGATAACAGAGTGAATGGCTATTAGATAA | Core staple | 1216 |
| 11[39] | CGGACATCCCTGCGCGTAACCACCAGGA | Core staple | 1217 |
| 11[53] | CCAAGCGCAGGTTTCTGCGTAATCATGGTCAGAGC | Core staple | 1218 |
| 11[88] | AGACGTCTGAAATGGGGTTATTAACCGTTGTAGCAATAGCTC | Core staple | 1219 |
| 11[130] | AAAAGGAAAAGGACATTCTGGCCAATAT | Core staple | 1220 |
| 10[58] | GTCCCGCGCTTAATGCGAGCCGGCCCCCGATTTAGAGCTTGA | Core staple | 1221 |
| 10[72] | CGGTGATGAAGGGTAAAGTTAAACCCTCATAGGTT | Core staple | 1222 |
| 10[100] | CAGTTGACGAGCACGTAGCCACCGGATTAGTAATAACATGGA | Core staple | 1223 |
| 10[114] | TGGAAACGCGAGCAAAAGAAGATGTAAATCCAATTCATCGAA | Core staple | 1224 |
| 10[121] | TCGCTTTCCTCGTTAGAAGTGTTTCCTGAGTAGAAGAATTGC | Core staple | 1225 |
| 12[48] | TTAAATAACCGGGGTGTCACTTATTGGGGTTGCAGCAAGCGGAATC | Core staple | 1226 |
| 12[79] | ATTAATTACATTTAGTGGCGTGCCGCAT | Core staple | 1227 |
| 12[90] | AAGAAAAGTGAGCCTTGTTTGGCCGCCATTAAAAAACCCTCA | Core staple | 1228 |
| 12[100] | AACATTGCCGTTCCGGCCAGCCTCAATTATTACCT | Core staple | 1229 |
| 12[121] | CTGGTCCGTTTTGAGAAACAATAAATTATTCATTTCAAATTA | Core staple | 1230 |
| 14[38] | CTGTCGGTCATAGAATAAGCTCGTCATGTCTGGTCAGCATAAGGCG | Core staple | 1231 |
| 14[69] | ACCGAGCAAGCCTGTTGCGTTGCGCTCAGTGG | Core staple | 1232 |
| 15[46] | CGGCTTTCCAGTCGGGAGTTTGCGGCGCGCCATGC | Core staple | 1233 |
| 15[98] | TGGCAAATACAAACAATTCCTCACAGTTTGTATCTGGTCAGT | Core staple | 1234 |
| 15[109] | CAGACCTCAAATATCAATACCGAACAATATAATATCAACGGC | Core staple | 1235 |
| 15[130] | GGTTCTAAAGCATCACCAAGATAATATCAGAAAAACAGCGTC | Core staple | 1236 |
| 17[91] | AATGCCAACGGCAGGCACAGGCGGCCTT | Core staple | 1237 |
| 17[105] | CACCGTCGGTGCATCCCAAAAATCCCGTAAAGCC | Core staple | 1238 |
| 17[126] | ACGCAACCAGCTTACGGCTGGCGGTTGTGTACATCGACATAA | Core staple | 1239 |
| 17[147] | AGGTGTCCAGCGCGGGGCATTTGCCGCCGTTGGG | Core staple | 1240 |
| 16[181] | CTTAAATTTCTGCTTCATTGCAGGCGCT | Core staple | 1241 |
| 19[53] | GTTCTTTGAGGACTAACGGTGTACTAAG | Core staple | 1242 |
| 19[84] | TCTGCGAATTAGCAAAATTTCCTTTTGAAGTTGATGGGT | Core staple | 1243 |
| 18[44] | TAGCTCCAACAGGTCAGAAAAGATAGAC | Core staple | 1244 |
| 21[102] | AAGAGGCAAGGCAAAGAACGAGTACGAAAGAATATATTCGGAAAA | Core staple | 1245 |
| 21[144] | CTTATTCTACTAATAGTGTCAATAGCCGCCACGGGACCAGGG | Core staple | 1246 |
| 20[44] | AGGAAATCAAAAATCAGCCAATACCGAGAGGACAT | Core staple | 1247 |
| 20[65] | GATCCCTGACTATTATAAATGTTTGTTT | Core staple | 1248 |
| 20[72] | CAATGACGCCAGCTGGCGGAACGATCCCAAT | Core staple | 1249 |
| 20[93] | AGAGGATGTGCGATCGGATTAACCGTGCATCGCTC | Core staple | 1250 |
| 20[107] | TAACATCAATATGATATAAACAAGGTTGATAAATC | Core staple | 1251 |
| 20[114] | GCCAGTTGGGCTGCGCATTGAGGGTCACGTTGGTGTAGGGCC | Core staple | 1252 |
| 20[135] | CTCTCCCAGTAAGCGCCCGGCCTCGATTGACCGTAATGCATC | Core staple | 1253 |
| 23[25] | AAAACGAGAAAAATATTCGACGATCGAGGCAAATAAAACGAACTATTA | Core staple | 1254 |
| 23[39] | CATAAGCCCGAAGCAAAAGCTTAATTGCTGATGCAACTCATA | Core staple | 1255 |
| 23[60] | TTATGCATCAGATTAGATCATTTTTGCGGATGGAA | Core staple | 1256 |
| 23[123] | CCGTTAAATGCCAAAAATTAACATCCAATAAATTAGATCGGG | Core staple | 1257 |
| 22[97] | GTAATCGTAAAATAATAGTAAGTAGAAAGGCCGGAGACAGTC | Core staple | 1258 |
| 22[135] | GCCAAAAACAATTCGCAATTAAATGTGAGCGAACG | Core staple | 1259 |
| 25[60] | TGCAAGAGTAGCGCATAACAG | Core staple | 1260 |
| 25[74] | TGCCCACATTATTCATCAGTTGAGAATCATTCTTGAGACAGA | Core staple | 1261 |
| 24[51] | AACAACATTATTACAGGGCGATTTCAGA | Core staple | 1262 |
| 24[90] | CGCCATTAGGAATACAGAGGGCTCTTCGCTATTACAATTGGGGTGAATT | Core staple | 1263 |
| 24[114] | AGCCTGTAGCCAGCTTTGGATAGGGACGACGTTTC | Core staple | 1264 |
| 26[65] | ATCAAAAGAAAGACTGGATAGCGTGTCT | Core staple | 1265 |
| 26[107] | TTGTACCCCGAGAATCGATGAACGAAATCACTGTGTAGCATA | Core staple | 1266 |
| 27[60] | ACGGCACTCATGAGGAAGTTTACAAACGGCTGGCTGGCAGCG | Core staple | 1267 |
| 27[129] | GTATATTCGCCAAGCCCCTGAGAGTCTGGAGCTCAA | Core staple | 1268 |
| 29[39] | AACGGTCAATAAAGTACGGTGTCTGGCT | Core staple | 1269 |
| 29[53] | CAGATCTTGAGAAACACTAAGAACTGGCTCAACGG | Core staple | 1270 |
| 29[88] | GGGTTCAAAAGGGTGCAGCAAGCAATAAAGCCTCAGAGGTAA | Core staple | 1271 |
| 29[130] | TTTATATATTTTCTAGCTGATAAACATT | Core staple | 1272 |
| 28[58] | AGGTCATTCCATATAACTAAGAGGGAGTACCTTTAATTGAAG | Core staple | 1273 |
| 28[72] | AGCACCATCGCCCACGCATAACCGCAGCATCGAAA | Core staple | 1274 |
| 28[100] | CAGGATTTAGTTTGACCATCATACCTAAATCGGTTGTACAAT | Core staple | 1275 |
| 28[114] | ATCTGCAGGGGTGGTGAAGGGATATGCCAGTACTG | Core staple | 1276 |
| 28[121] | TTGACATTTCGCAAATGAGTAGCACATTATGACCCTGTAACC | Core staple | 1277 |
| 30[48] | GGGCGCGCTGACGACAAGAACAAAATAGTGCGGAATCGTCATTGAC | Core staple | 1278 |
| 30[79] | AACAGCGGATCAAATTCAGTAGTACTTC | Core staple | 1279 |
| 30[90] | AGAGACGTGGTTTATGCGGGCGGCTAGCATGTCAAATAGGAA | Core staple | 1280 |
| 30[100] | TCACGGTCGCTGAGGCTGTCACCCGCGATTATGAG | Core staple | 1281 |
| 30[121] | TCCAGTTAAAGGACGGATAACCTCTGTGAGAGATAGACACA | Core staple | 1282 |
| 32[38] | TACCGCTTGCCGTTGCGGGAGGCGCAGAAGACTTTTTCAATCCGCC | Core staple | 1283 |
| 32[69] | ACCTTATTAGAAAGCAACTAATGCAGATCTTT | Core staple | 1284 |
| 33[46] | AACGCCAAAAGGAATTAAAAAACCCGGATATGATG | Core staple | 1285 |
| 33[98] | CGCGTCTATGGGCGCATCGTTCAACTTTATTCAAAAATAATT | Core staple | 1286 |
| 33[109] | TTCTCATTTTTTAACCATCATATGGGAAGGGCTGCAAGTCAG | Core staple | 1287 |
| 33[130] | AACTTAAATTTTTGTTAATCAGAAATTCAGGTAACGCCGCTT | Core staple | 1288 |
| 35[131] | CCATTAAACGGGTAAATGCGCCGACAATGACA | Core staple | 1289 |
| 35[147] | ATACGTAATGCCACTACGAAGAAACAGCTTGATACCGATAGT | Core staple | 1290 |
| 35[168] | GCACCAACCTAAAACGAAAAAGAATACACTAAAAC | Core staple | 1291 |
| 34[209] | AATTGTATCGGTTTATCTTTCGAGGTGAATTTCTT | Core staple | 1292 |
| 34[230] | AAGGCTCCAAAAGGAGCCTTTACTCATCTTTGACCCCCAGCG | Core staple | 1293 |
| 34[246] | GAAAATCTCCAAAAAAATTATACCAAGCGCGA | Core staple | 1294 |
| 37[53] | AGATATATAACTATATATAACAACGAAT | Core staple | 1295 |
| 37[84] | CAGTATGGAAGGTAAATATATAGCAATAGACTCCTAACC | Core staple | 1296 |
| 36[44] | GAATGAGTTAAGCCCAAGACGGGAGCCA | Core staple | 1297 |
| 36[65] | TCTAGCAAGAAACAATGTAAA | Core staple | 1298 |
| 39[102] | TGACCGATTGAGGGAGGTTAGCAAGGTCTGATGAAAACAAAGGAA | Core staple | 1299 |
| 39[144] | GCCCATATGGTTTACCAAAAAGAAAGCGTAACGATCAGAGTT | Core staple | 1300 |
| 38[44] | TAATCAAAAATGAAAATAGAGCCTTAGTTGCTAGA | Core staple | 1301 |
| 38[65] | AAGTTTACAGAGAGAATAACGCTACTAC | Core staple | 1302 |
| 38[72] | AACAGACCCTCATTTTCCCTTTTTTATTACG | Core staple | 1303 |
| 38[93] | GAAGCAAGCCTCAGAACAATCCTCAAGAGAAAACA | Core staple | 1304 |
| 38[107] | AATATCGGCATTTTCGGCTCAGAAAGCCGCCTCTC | Core staple | 1305 |
| 38[114] | GCAGTACCGTCCACCCTGATTAGCACATGAAAGTATTAGAGT | Core staple | 1306 |
| 38[135] | CCATCACCAGTACTCAGTACCAGGTTCGGAACCTATTATAAC | Core staple | 1307 |
| 41[25] | CGATTTTTTGAAAATAATTTGAAGTAAGAACCAAGTACCGCACTCGCT | Core staple | 1308 |
| 41[39] | ACGCTGAACACAAGAATAAGTAAGCAGATAGACGCAATAAAG | Core staple | 1309 |
| 41[60] | GCCCGCATTATAATAAGTACCGAAGCCCTTTCAAA | Core staple | 1310 |
| 41[123] | AGCCATCGATCGACTTGAGACAAAAGGGCGATACATAAAGTG | Core staple | 1311 |
| 40[97] | GCCACCACCCTCAATCTTACCAATTAGCGTCAGACTGTAGCG | Core staple | 1312 |
| 40[135] | CCCGAGGTTGAAGCCAGGTCAGTGCCTTGAGTGCC | Core staple | 1313 |
| 43[60] | TTGAGCCAGTTGTAATTGTTG | Core staple | 1314 |
| 43[74] | AATCAATAGCTCATCGTAGGAATCCCCATCCAAGTCCTTAAT | Core staple | 1315 |
| 42[51] | AGGACAAGCAAGCCGTTGTAGAAAGCCT | Core staple | 1316 |
| 42[90] | CATACTACCGCGCCTTTATCCCTCAGAGCCACCGCAATAGATTAATTTA | Core staple | 1317 |
| 42[114] | TGACTGGTAATAAGTTTTTCTGAAGGGGTTTAGCG | Core staple | 1318 |
| 44[65] | TCGCACCCAGACGAGCGTCTTTCCAGCA | Core staple | 1319 |
| 44[107] | ACCCCACCAGCCGCCACCCTCAGACGTTTTCCAGTAGCAAGG | Core staple | 1320 |
| 45[60] | GTTAAAGTACTGCAAATCCAATAAGGCTTAGTAGGCAGAGGG | Core staple | 1321 |
| 45[129] | TCAGGAGGTTTTTGACAGTCAGAGCCGCCACCTCAT | Core staple | 1322 |
| 47[39] | ATTCCAGTATAATAACGGAATACCTTAA | Core staple | 1323 |
| 47[53] | ACAAATAAGAAGAACGCCCAATCAATAATCGATCG | Core staple | 1324 |
| 47[88] | ATATCAAGTTTGCCTCAAATGACGGAAATTATTCATTAGACA | Core staple | 1325 |
| 47[130] | TCGATGAAACCCCCTTATTAGCGTGCCT | Core staple | 1326 |
| 46[58] | GGTACTGGCATGATTAAGCTA | Core staple | 1327 |
| 46[72] | TCCTTAATTTTCCCTTAGAATCCTGAGACTAAGGG | Core staple | 1328 |
| 46[100] | ATAACGTAGAAAATACACATTCAAATTATCACCGTCACAGCA | Core staple | 1329 |
| 46[114] | AATGATTAAGTGAGAATAGAAAGGGGATTAGCAGA | Core staple | 1330 |
| 46[121] | AATAGGTGGCAACATATGCGCCAAAGCCATTTGGGAATGTCA | Core staple | 1331 |
| 48[48] | ATTTGTACTAATGCGAATATATCAAGATAATTTGCCAGTTACTTTA | Core staple | 1332 |
| 48[79] | AATTTTTTCACGTTAACTATCAACATTT | Core staple | 1333 |
| 48[90] | TTGCGAAGAACAAGCGCCACCTGAGAGCCGCCACCTAAGCGT | Core staple | 1334 |
| 48[100] | ACTATAGCGATAGCTTATTATCAAAACCCATCCGT | Core staple | 1335 |
| 48[121] | GAGACGCTGAGATAAAGTTTTGTCCTTTCAACAGTTTCTGC | Core staple | 1336 |
| 50[38] | GTCTTGTTCAGTCATCGCACAAATTCTTGTAAATGCTGAAACGGAG | Core staple | 1337 |
| 50[69] | CGAGCATTTTATTTAAGCAAATCAGATATATT | Core staple | 1338 |
| 51[46] | AGACTTATCCGGTATTCCCTTAAAAAGTACCCCAT | Core staple | 1339 |
| 51[98] | GATACAGAGAGGCTGAGACAAATAATATATATGGCTTTTGAT | Core staple | 1340 |
| 51[109] | GTAATTTACCGTTCCAGAGAACCAGCCACCCCAATAGGAATC | Core staple | 1341 |
| 51[130] | GGGAATGGAAAGCGCAGGCCAGCAAGTACCGAACACTGAGTC | Core staple | 1342 |
| 53[91] | TCGCAAGACAAAGATAAATCGTCGCTAT | Core staple | 1343 |
| 53[105] | ACGCGAGAAAATTCAAAGAGTGAATAACCTTCTG | Core staple | 1344 |
| 53[126] | TATATTTTAGTTAATTTCATCAGTACATAAATCAATATATGT | Core staple | 1345 |
| 53[147] | TTCTGACCTAAAATGGTATTACCTTTTTGGAAAC | Core staple | 1346 |
| 52[181] | ACAATTTCATTTGATTGAAATACCGACC | Core staple | 1347 |
| 0[166] | TTTTAGACAGGAACGGTACGTATCGGCCTT | Core staple | 1348 |
| 2[163] | CCAGAACAATATTACCGTAGAACCCTT | Core staple | 1349 |
| 4[163] | GCGTAAGAATACGTGGCACAGACAACAGAGACCAGCCACTCA | Core staple | 1350 |
| 6[163] | GCCACGCTGAGAGCCAGCAGCAAAGGTCAGTAATT | Core staple | 1351 |
| 8[142] | ATCCGTAGATACAGTACCGGGAGCTAAACAGGAGGCC | Core staple | 1352 |
| 8[166] | GAAACCACCAGAAGGAGCGGATTAACACCG | Core staple | 1353 |
| 10[160] | ATGAATATACAGTATTTCAGG | Core staple | 1354 |
| 12[163] | AGTTACAAAATCGCGCAAACATTATCATTT | Core staple | 1355 |
| 14[142] | ATATTTGAGTGAGGCGACGGATTCGCCTGATTGC | Core staple | 1356 |
| 14[160] | AATAGATTAGAGCCTTAGGAG | Core staple | 1357 |
| 18[166] | GAGCTGAAAAGGTGGCATCATTGCGGGAGA | Core staple | 1358 |
| 20[163] | CAACGCAAGGATAAAAACGGAGAGGGT | Core staple | 1359 |
| 22[163] | AGAGATCTACAAAGGCTATCAGGTTTAATGCTTTTTAGAATA | Core staple | 1360 |
| 24[163] | TGTAAACGTTAATATTTTGTTAAAGGAAGATCCAG | Core staple | 1361 |
| 26[142] | GCACACGACGAGGTGGAACCTGTTTAGCTATATTTTC | Core staple | 1362 |
| 26[166] | ACCGCTTCTGGTGCCGGAAATGTATAAGCA | Core staple | 1363 |
| 28[160] | TGCCAAGCTTTCAGTTGTAAA | Core staple | 1364 |
| 30[163] | GCCATGTTTACCAGTCCTCGCACTCCAGCC | Core staple | 1365 |
| 32[142] | GCGAGGAAGACGGAATTACCGGAAACAATCGGCG | Core staple | 1366 |
| 32[160] | TCTCCGTGGGAACAAGTAACA | Core staple | 1367 |
| 36[166] | GTCACAATCAATAGAAAATTAGCAAAATCA | Core staple | 1368 |
| 38[163] | ATTACCATTAGCAAGGCCTTTTCATAA | Core staple | 1369 |
| 40[163] | GGAACCAGAGCCACCACCGGAACCTTGCCATCGGAAACTAGA | Core staple | 1370 |
| 42[163] | TCACAAACAAATAAATCCTCATTAAGGCAGGATCA | Core staple | 1371 |
| 44[142] | CCGTACAAACCATAGTTACGCAAAGACACCACGGAAT | Core staple | 1372 |
| 44[166] | GTATAGCCCGGAATAGGTGTTCAGACGATT | Core staple | 1373 |
| 46[160] | CCACAGACAGCCCTTACAACG | Core staple | 1374 |
| 48[163] | TCTGTATGGGATTTTGCGTGCCGTCGAGAG | Core staple | 1375 |
| 50[142] | TATCGGATAATAAACAAGTCTTTCCAGACGTTAG | Core staple | 1376 |
| 50[160] | CAGTTAATGCCCCCTAACAGT | Core staple | 1377 |
| 13[157] | TTTGAATACCA | Connector staple | 1378 |
| 31[157] | AAACGTACATT | Connector staple | 1379 |
| 49[157] | TAAATGAATGC | Connector staple | 1380 |
| 9[160] | TGCGGAACAAG | Connector staple | 1381 |
| 27[160] | AGCTTTCCGTT | Connector staple | 1382 |
| 45[160] | GGTTGATATAG | Connector staple | 1383 |
| 11[154] | TTTAACGTCAA | Connector staple | 1384 |
| 29[154] | ACGACGGCCAA | Connector staple | 1385 |
| 47[154] | CCTGTAGCAGC | Connector staple | 1386 |
| 1[160] | GATTAAAGGCT | Connector staple | 1387 |
| 3[157] | GCTGGTAATGT | Connector staple | 1388 |
| 5[157] | CTGACCTGAAA | Connector staple | 1389 |
| 7[157] | CCTGCAACAAT | Connector staple | 1390 |
| 15[154] | CACTAACAAGA | Connector staple | 1391 |
| 19[160] | ATTTGGGGCAA | Connector staple | 1392 |
| 21[157] | AGCCTTTATAT | Connector staple | 1393 |
| 23[157] | AGCTATTTTCC | Connector staple | 1394 |
| 25[157] | AATATTTAACC | Connector staple | 1395 |
| 33[154] | ACCCGTCGGTT | Connector staple | 1396 |
| 37[160] | AAGTTTATTAT | Connector staple | 1397 |
| 39[157] | CCAGTAGCAAT | Connector staple | 1398 |
| 41[157] | TCAAAATCATG | Connector staple | 1399 |
| 43[157] | GGCCTTGATTT | Connector staple | 1400 |
| 51[154] | GCCCGTATAGC | Connector staple | 1401 |
| 1[12] | TTTTTGCTGGCAAGTGTAGCGGAGCGGGTCAAGGTGCCGTAAAACG | Vertex staple | 1402 |
| 3[9] | TTTTTAAAAACCGTCTACGCTAGGGCTTTTT | Vertex staple | 1403 |
| 2[30] | TGGGCATCAGTGTGCACGTTTTCATTCCTGTGTGAAATTGTTATTTTT | Vertex staple | 1404 |
| 9[12] | TTTTTCAGAATGCGGCGGGCCTCTGTGGCGC | Vertex staple | 1405 |
| 10[30] | ACTTTTCTTTACACCGGAATCATAATTACTAGAAAATTTTT | Vertex staple | 1406 |
| 13[9] | TTTTTGGCTGGTAATGGGTAAAGGGGTGTGTTCAGCTTTTT | Vertex staple | 1407 |
| 15[16] | TTTTTTCCGCTCACAATCGTGCCAGCTGCATTAATGTTTTT | Vertex staple | 1408 |
| 19[12] | TTTTTCAACATGTTTTAAATAATATAATGCGAACCAGACCGGAAA | Vertex staple | 1409 |
| 21[9] | TTTTTTCGAGCTTCAAAGCTGTAGCTTTTTT | Vertex staple | 1410 |
| 20[31] | GACTGAGGACATCATTACGAATAAGAGTCAGGACGTTGGGAAGATTTTT | Vertex staple | 1411 |
| 27[12] | TTTTTAAGCTGCTCATTCAGTCCAAATCTAC | Vertex staple | 1412 |
| 28[30] | AGGCCGGAACTATGAGCCGGGTCACTGTTGCCCTGCTTTTT | Vertex staple | 1413 |
| 31[9] | TTTTTCCTGCTCCATGTTACTTAGGAACCGAACTGATTTTT | Vertex staple | 1414 |
| 33[16] | TTTTTAAAATCTACGTTTAGTAAGAGCAACACTATCTTTTT | Vertex staple | 1415 |
| 37[12] | TTTTTGAAGGAAACCGAGGAACCGAACAAGAGAGATAACCCACCCT | Vertex staple | 1416 |
| 39[9] | TTTTTAGCGCTAATATCAAGTTACCATTTTT | Vertex staple | 1417 |
| 38[30] | GAAAGAATCGGACAAAAAACAACATTCCTTATCATTCCAAGAATTTTT | Vertex staple | 1418 |
| 45[12] | TTTTTCCAGACGACGACAATAGGTAAAGGGG | Vertex staple | 1419 |
| 46[30] | CCAGCGTTATCTGATAAATTGTGTCGAAATCCGCGATTTTT | Vertex staple | 1420 |
| 49[9] | TTTTTAGCCTGTTTAGTATCATATACGCTCAACAGTTTTTT | Vertex staple | 1421 |
| 51[16] | TTTTTCGGGTATTAAACGCGAGGCGTTTTAGCGAACTTTTT | Vertex staple | 1422 |
| 7[24] | GGGGTGGTTTGCCCCAGCAGGCGACAGTTAAAATTCTCATTGCAATCCAA | Vertex bundle | 1423 |
| | ATAAAGAGGGTAATTGTTTTT | strand | |
| 25[24] | CAGACATTGAATCCCCCTCAAATAATAGTAGTCTAATCTATGAAAATCCT | Vertex bundle | 1424 |
| | GTTTCGTCAAAGGGCGTTTTT | strand | |
| 43[24] | AGGTACAGCCATATTATTTATCCCACTAATCTTATGTAGCTTTAAACAGT | Vertex bundle | 1425 |
| | TCGCGTTTTAATTTTTT | strand | |
| 7[9] | TTTTTAATCGGCCAACGTGCTGCGGCCACA AGTT AAAGAT TCGTC | Vertex bundle | 1426 |
| | ATTGAAGGGCTTAATTGCAAAGTCGAAA | strand | |
| 25[9] | TTTTTATAACCCTCGTTAACGTAACAGTAA TAGT AGTCTA CATCT | Vertex bundle | 1427 |
| | ATGGCAAATCGTTAACGACTCCAAGATG | strand | |
| 43[9] | TTTTTCTCCCGACTTGCTAATTCTGTTAA TCT TAT | Vertex bundle | 1428 |
| | GTACCAACTTTGAAATCAAATATCAG | strand | |
| | CAATGAGAATTTTAACTGT | Vertex bundle | 1429 |
| | | strand | |
| | | (complementary) | |
| | CATAGATTAGACTACTATT | Vertex bundle | 1430 |
| | | strand | |
| | | (complementary) | |
| | TACATAAGATTAGTG | Vertex bundle | 1431 |
| | | strand | |
| | | (complementary) | |
| | TCAAT GACGA ATCTTT AACT TGTG | Vertex bundle | 1432 |
| | | strand | |
| | | (complementary) | |
| | GCCAT AGATG TAGACT ACTA TTAC | Vertex bundle | 1433 |
| | | strand | |
| | | (complementary) | |
| | TAC ATA AGA TTA | Vertex bundle | 1434 |
| | | strand | |
| | | (complementary) | |

### REFERENCES

1. J. H. Chen, N. C. Seeman, Synthesis from DNA of a molecule with the connectivity of a cube. Nature 350, 631-633 (1991).
2. E. Winfree, F. Liu, L. A. Wenzler, N. C. Seeman, Design and self-assembly of two-dimensional DNA crystals. Nature 394, 539-544 (1998).
3. P. W. Rothemund, N. Papadakis, E. Winfree, Algorithmic self-assembly of DNA Sierpinski triangles. PLoS biology 2, e424 (2004).

4. P. W. Rothemund, Folding DNA to create nanoscale shapes and patterns. Nature 440, 297-302 (2006).

5. S. M. Douglas, H. Dietz, T. Liedl, B. Hogberg, F. Graf, W. M. Shih, Self-assembly of DNA into nanoscale three-dimensional shapes. Nature 459, 414-418 (2009).
6. J. Zheng, J. J. Birktoft, Y. Chen, T. Wang, R. Sha, P. E. Constantinou, S. L. Ginell, C. Mao, N. C. Seeman, From molecular to macroscopic via the rational design of a self-assembled 3D DNA crystal. Nature 461, 74-77 (2009).
7. B. Wei, M. Dai, P. Yin, Complex shapes self-assembled from single-stranded DNA tiles. Nature 485, 623-626 (2012).

8. Y. Ke, L. L. Ong, W. M. Shih, P. Yin, Three-dimensional structures self-assembled from DNA bricks. Science 338, 1177-1183 (2012).
9. D. Han, S. Pal, Y. Yang, S. Jiang, J. Nangreave, Y. Liu, H. Yan, DNA gridiron nanostructures based on four-arm junctions. Science 339, 1412-1415 (2013).
10. V. Linko, H. Dietz, The enabled state of DNA nanotechnology. Current opinion in biotechnology 24, 555-561 (2013).
11. Y. Zhang, N. C. Seeman, Construction of a DNA-Truncated Octahedron. Journal of the American Chemical Society 116, 1661-1669 (1994).
12. W. M. Shih, J. D. Quispe, G. F. Joyce, A 1.7-kilobase single-stranded DNA that folds into a nanoscale octahedron. Nature 427, 618-621 (2004).
13. R. P. Goodman, I. A. Schaap, C. F. Tardin, C. M. Erben, R. M. Berry, C. F. Schmidt, A. J. Turberfield, Rapid chiral assembly of rigid DNA building blocks for molecular nanofabrication. Science 310, 1661-1665 (2005).
14. F. A. Aldaye, H. F. Sleiman, Modular access to structurally switchable 3D discrete DNA assemblies. Journal of the American Chemical Society 129, 13376-13377 (2007).
15. C. M. Erben, R. P. Goodman, A. J. Turberfield, A self-assembled DNA bipyramid. Journal of the American Chemical Society 129, 6992-6993 (2007).
16. Y. He, T. Ye, M. Su, C. Zhang, A. E. Ribbe, W. Jiang, C. Mao, Hierarchical self-assembly of DNA into symmetric supramolecular polyhedra. Nature 452, 198-201 (2008).
17. C. Zhang, M. Su, Y. He, X. Zhao, P. A. Fang, A. E. Ribbe, W. Jiang, C. Mao, Conformational flexibility facilitates self-assembly of complex DNA nanostructures. Proceedings of the National Academy of Sciences of the United States of America 105, 10665-10669 (2008).
18. J. Zimmermann, M. P. Cebulla, S. Monninghoff, G. von Kiedrowski, Self-assembly of a DNA dodecahedron from 20 trisoligonucleotides with C(3h) linkers. Angewandte Chemie 47, 3626-3630 (2008).
19. E. S. Andersen, M. Dong, M. M. Nielsen, K. Jahn, R. Subramani, W. Mamdouh, M. M. Golas, B. Sander, H. Stark, C. L. Oliveira, J. S. Pedersen, V. Birkedal, F. Besenbacher, K. V. Gothelf, J. Kjems, Self-assembly of a nanoscale DNA box with a controllable lid. Nature 459, 73-76 (2009).
20. Y. Ke, J. Sharma, M. Liu, K. Jahn, Y. Liu, H. Yan, Scaffolded DNA origami of a DNA tetrahedron molecular container. Nano letters 9, 2445-2447 (2009).
21. D. Bhatia, S. Mehtab, R. Krishnan, S. S. Indi, A. Basu, Y. Krishnan, Icosahedral DNA nanocapsules by modular assembly. Angewandte Chemie 48, 4134-4137 (2009).
22. H. Yang, C. K. McLaughlin, F. A. Aldaye, G. D. Hamblin, A. Z. Rys, I. Rouiller, H. F. Sleiman, Metal-nucleic acid cages. Nature chemistry 1, 390-396 (2009).
23. C. Zhang, S. H. Ko, M. Su, Y. Leng, A. E. Ribbe, W. Jiang, C. Mao, Symmetry controls the face geometry of DNA polyhedra. Journal of the American Chemical Society 131, 1413-1415 (2009).
24. D. M. Smith, V. Schuller, C. Forthmann, R. Schreiber, P. Tinnefeld, T. Liedl, A structurally variable hinged tetrahedron framework from DNA origami. Journal of nucleic acids 2011, 360954 (2011).
25. S. M. Douglas, I. Bachelet, G. M. Church, A logic-gated nanorobot for targeted transport of molecular payloads. Science 335, 831-834 (2012).
26. Z. Nie, X. Li, Y. Li, C. Tian, P. Wang, C. Mao, Self-assembly of DNA nanoprisms with only two component strands. Chemical communications 49, 2807-2809 (2013).
27. Materials and methods are available as supplementary material on Science Online.
28. R. Jungmann, C. Steinhauer, M. Scheible, A. Kuzyk, P. Tinnefeld, F. C. Simmel, Single-molecule kinetics and super-resolution microscopy by fluorescence imaging of transient binding on DNA origami. Nano letters 10, 4756-4761 (2010).
29. R. Jungmann, M. S. Avendano, J. B. Woehrstein, M. Dai, W. M. Shih, P. Yin, Multiplexed 3D cellular super-resolution imaging with DNA-PAINT and Exchange-PAINT. Nature methods, (2014).
30. A. Sharonov, R. M. Hochstrasser, Wide-field subdiffraction imaging by accumulated binding of diffusing probes. Proceedings of the National Academy of Sciences of the United States of America 103, 18911-18916 (2006).
31. B. Huang, W. Wang, M. Bates, X. Zhuang, Three-dimensional super-resolution imaging by stochastic optical reconstruction microscopy. Science 319, 810-813 (2008).
32. G. Shtengel, J. A. Galbraith, C. G. Galbraith, J. Lippincott-Schwartz, J. M. Gillette, S. Manley, R. Sougrat, C. M. Waterman, P. Kanchanawong, M. W. Davidson, R. D. Fetter, H. F. Hess, Interferometric fluorescent super-resolution microscopy resolves 3D cellular ultrastructure. Proceedings of the National Academy of Sciences of the United States of America 106, 3125-3130 (2009).
33. J. J. Schmied, C. Forthmann, E. Pibiri, B. Lalkens, P. Nickels, T. Liedl, P. Tinnefeld, DNA origami nanopillars as standards for three-dimensional superresolution microscopy. Nano letters 13, 781-785 (2013).
34. S. W. Hell, Far-field optical nanoscopy. Science 316, 1153-1158 (2007).
35. C. Lin, R. Jungmann, A. M. Leifer, C. Li, D. Levner, G. M. Church, W. M. Shih, P. Yin, Submicrometre geometrically encoded fluorescent barcodes self-assembled from DNA. Nature chemistry 4, 832-839 (2012).
36. H. P. Kao, A. S. Verkman, Tracking of single fluorescent particles in three dimensions: use of cylindrical optics to encode particle position. Biophysical journal 67, 1291-1300 (1994).
37. J. Fu, M. Liu, Y. Liu, N. W. Woodbury, H. Yan, Interenzyme substrate diffusion for an enzyme cascade organized on spatially addressable DNA nanostructures. Journal of the American Chemical Society 134, 5516-5519 (2012).
38. G. P. Acuna, F. M. Moller, P. Holzmeister, S. Beater, B. Lalkens, P. Tinnefeld, Fluorescence enhancement at docking sites of DNA-directed self-assembled nanoantennas. Science 338, 506-510 (2012).
39. A. Kuzyk, R. Schreiber, Z. Fan, G. Pardatscher, E. M. Roller, A. Hogele, F. C. Simmel, A. 0. Govorov, T. Liedl, DNA-based self-assembly of chiral plasmonic nanostructures with tailored optical response. Nature 483, 311-314 (2012).
40. M. Langecker, V. Arnaut, T. G. Martin, J. List, S. Renner, M. Mayer, H. Dietz, F. C. Simmel, Synthetic lipid membrane channels formed by designed DNA nanostructures. Science 338, 932-936 (2012).

What is claimed is:

1. A nucleic acid structure comprising
a first (x), a second (y), and a third (z) nucleic acid arm, each connected at one end to the other arms to form a vertex, and
a first, a second, and a third nucleic strut, wherein the first nucleic acid strut connects the first (x) nucleic arm to the second (y) nucleic arm, the second nucleic acid strut connects the second (y) nucleic arm to the third (z) nucleic arm, and the third nucleic acid strut connects the third (z) arm to the first (x) nucleic acid strut.

2. A nucleic acid structure comprising
three nucleic acid arms radiating from a vertex at fixed angles.

3. A nucleic acid structure comprising
N nucleic acid arms radiating from a vertex, wherein N is the number of nucleic acid arms and is 3 or more, and
M nucleic acid struts, each strut connecting two nucleic acid arms to each other, wherein M is the number of nucleic acid struts and is 3 or more.

4. The nucleic acid structure of claim 3, wherein N is equal to M.

5. The nucleic acid structure of claim 3, wherein N is less than M.

6. The nucleic acid structure of claim 1, wherein the nucleic acid structure comprises 4 nucleic acids and at least 4 nucleic acid struts, or 5 nucleic acid arms and at 5 nucleic acid struts.

7. The nucleic acid structure of claim 1, wherein the nucleic acid arms are equally spaced apart from each other (or the arms are separated from each other by the same angle).

8. The nucleic acid structure of claim 1, wherein the nucleic acid arms are not equally separated from each other (or the arms are separated from each other by different angles).

9. The nucleic acid structure of claim 1, further comprising a vertex nucleic acid.

10. The nucleic acid structure of claim 1, further comprising a connector nucleic acid.

11. The nucleic acid structure of claim 1, wherein the nucleic acid arms, nucleic acid struts, and/or vertex nucleic acid are comprised of parallel double helices.

12. The nucleic acid structure of claim 1, wherein nucleic acid arms are of identical length.

13. The nucleic acid structure of claim 1, wherein the nucleic acid struts are of identical length.

14. The nucleic acid structure of claim 1, wherein the nucleic acid struts are of different lengths.

15. The nucleic acid structure of claim 1, wherein at least one nucleic acid arm comprises a blunt end.

16. The nucleic acid structure of claim 1, wherein at least one nucleic acid arm comprises a connector nucleic acid at its free (non-vertex) end that is up to 16 nucleotides in length.

17. The nucleic acid structure of claim 1, wherein at least one nucleic acid arm comprises a connector nucleic acid at its free (non-vertex) end, thereby comprising a 1 or 2 nucleotide overhang.

18. The nucleic acid structure of claim 1, wherein the nucleic acid structure is up to 5 megadaltons (MD) in size.

19. The nucleic acid structure of claim 1, wherein the nucleic acid arms are 50 nm in length.

20. The nucleic acid structure of claim 1, wherein the nucleic acid structure comprises three nucleic acid arms separated from each other by 60°-60°-60° (tetrahedron).

21. The nucleic acid structure of claim 1, wherein the nucleic acid structure comprises three nucleic acid arms separated from each other by 60°-90°-90° (triangular prism).

22. The nucleic acid structure of claim 1, wherein the nucleic acid structure comprises three nucleic acid arms separated from each other by 90°-90°-90° (cube).

23. The nucleic acid structure of claim 1, wherein the nucleic acid structure comprises three nucleic acid arms separated from each other by 108°-90°-90° (pentagonal prism).

24. The nucleic acid structure of claim 1, wherein the nucleic acid structure comprises three nucleic acid arms separated from each other by 120°-90°-90° (hexagonal prism).

25. A composite nucleic acid structure comprising L nucleic acid structures selected from the nucleic acid structures of claim 1, wherein L is an even number of nucleic acid structures, and wherein the L nucleic acid structures are connected to each other at free (non-vertex) ends of the nucleic acid arms.

26. The composite nucleic acid structure of claim 25, wherein the two more nucleic acid structures are two, four, six, eight, ten, twelve or more nucleic acid structures.

27. The composite nucleic acid structure of claim 25, wherein the composite nucleic acid structure is a tetrahedron, a triangular prism, a cube, a pentagonal prism, or a hexagonal prism.

28. The composite nucleic acid structure of claim 25, wherein the composite nucleic acid structure is 20 megadaltons (MD), 30 MD, 40 MD, 50 MD, or 60 MD in size.

29. The composite nucleic acid structure of claim 25, wherein the composite nucleic acid structure has edge widths, comprised of two nucleic acid arms from adjacent nucleic acid structures, of 100 nm.
